(19) United States
(12) Patent Application Publication
(10) Pub. No.: US 2019/0221750 A1
(43) Pub. Date: Jul. 18, 2019

(54) CONDENSED CYCLIC COMPOUND, COMPOSITION INCLUDING THE SAME, AND ORGANIC LIGHT-EMITTING DEVICE INCLUDING THE CONDENSED CYCLIC COMPOUND

(71) Applicant: Samsung Electronics Co., Ltd., Suwon-si (KR)

(72) Inventors: Keisuke KORAI, Kanagawa (JP); Rie SAKURAI, Suwon-si (KR); Mitsunori ITO, Kanagawa (JP); Masaki NUMATA, Hwaseong-si, Gyeonggi-do (JP)

(21) Appl. No.: 16/244,172

(22) Filed: Jan. 10, 2019

(30) Foreign Application Priority Data
Jan. 12, 2018 (JP) 2018-003750
Jul. 9, 2018 (KR) 10-2018-0079580

(51) Int. Cl. H01L 51/00; C07D 209/86

(52) U.S. Cl. CPC H01L 51/0072; H01L 51/5016; C07D 209/86

(57) ABSTRACT

A condensed cyclic compound represented by Formula 1:

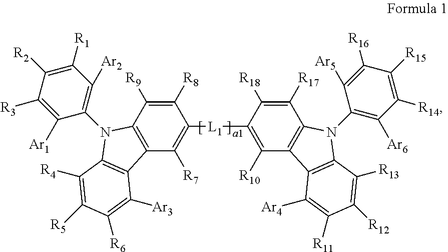

wherein, in Formula 1, groups and variables are the same as described in the specification.

### CROSS-REFERENCE TO RELATED APPLICATIONS

This application claims priority to Japanese Patent Application No. 2018-003750, filed on Jan. 12, 2018, in the Japanese Patent Office and Korean Patent Application No. 10-2018-0079580, filed on Jul. 9, 2018, in the Korean Intellectual Property Office, and all the benefits accruing therefrom under 35 U.S.C. § 119, the contents of which are incorporated herein in their entireties by reference.

### BACKGROUND

### 1. Field

The present disclosure relates to a condensed cyclic compound, a composition including the same for an organic light-emitting device, and an organic light-emitting device including the condensed cyclic compound.

### 2. Description of the Related Art

Organic light-emitting devices (OLEDs) are self-emission devices that produce full-color images, and that also have wide viewing angles, high contrast ratios, short response times, and excellent characteristics in terms of brightness, driving voltage, and response speed, compared to devices in the art.

In an example, an organic light-emitting device includes an anode, a cathode, and an organic layer disposed between the anode and the cathode, wherein the organic layer includes an emission layer. A hole transport region may be disposed between the anode and the emission layer, and an electron transport region may be disposed between the emission layer and the cathode. Holes provided from the anode may move toward the emission layer through the hole transport region, and electrons provided from the cathode may move toward the emission layer through the electron transport region. The holes and the electrons recombine in the emission layer to produce excitons. These excitons transit from an excited state to a ground state, thereby generating light.

Various types of organic light emitting devices are known. However, there still remains a need in OLEDs having low driving voltage, high efficiency, high brightness, and long lifespan.

### SUMMARY

Provided are a condensed cyclic compound, a composition including the condensed cyclic compound, and an organic light-emitting device including the condensed cyclic compound.

The organic light-emitting device including the condensed cyclic compound may provide high current efficiency and a long lifespan. In addition, the condensed cyclic compound may provide characteristics suitable for use in solution coating.

Additional aspects will be set forth in part in the description which follows and, in part, will be apparent from the description, or may be learned by practice of the presented embodiments.

An aspect of an embodiment provides a condensed cyclic compound represented by Formula 1:

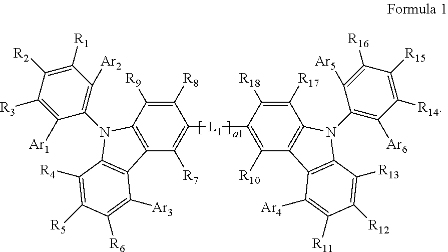

In Formula 1,

Ar₁ to Ar₆ and R₁ to R₁₈ may each independently be selected from a group represented by *-(L₂₁)ₐ₂₁-R₂₁, hydrogen, deuterium, —F, —Cl, —Br, —I, a hydroxyl group, a cyano group, a nitro group, an amidino group, a hydrazine group, a hydrazone group, a carboxylic acid group or a salt thereof, a sulfonic acid group or a salt thereof, a phosphoric acid group or a salt thereof, a substituted or unsubstituted C₁-C₆₀ alkyl group, a substituted or unsubstituted C₂-C₆₀ alkenyl group, a substituted or unsubstituted C₂-C₆₀ alkynyl group, a substituted or unsubstituted alkoxy group, a substituted or unsubstituted C₃-C₁₀ cycloalkyl group, a substituted or unsubstituted heterocycloalkyl group, a substituted or unsubstituted C₃-C₁₀ cycloalkenyl group, a substituted or unsubstituted heterocycloalkenyl group, a substituted or unsubstituted C₆-C₆₀ aryl group, a substituted or unsubstituted C₆-C₆₀ aryloxy group, a substituted or unsubstituted C₆-C₆₀ arylthio group, a substituted or unsubstituted C₁-C₆₀ heteroaryl group, a substituted or unsubstituted monovalent non-aromatic condensed polycyclic group, a substituted or unsubstituted monovalent non-aromatic condensed heteropolycyclic group, —Si(Q₁)(Q₂)(Q₃), —N(Q₄)(Q₅), and —B(Q₆)(Q₇),

wherein i) each of at least one selected from Ar₁, Ar₂, Ar₅, and Ar₆ may independently be a group represented by *-(L₂₁)ₐ₂₁-R₂₁, and one selected from Ar₃ and Ar₄ may be a group represented by *-(L₂₁)ₐ₂₁-R₂₁; or

ii) Ar₃ and Ar₄ may each independently be a group represented by *-(L₂₁)ₐ₂₁-R₂₁,

L₁ and L₂₁ may each independently be selected from a substituted or unsubstituted C₅-C₃₀ carbocyclic group and a substituted or unsubstituted C₁-C₃₀ heterocyclic group,

a1 and a21 may each independently be 0 or 1, wherein, when a1 is 0, *-(L₁)ₐ₁-*′ may be a single bond, and when a21 is 0, *-(L₂₁)ₐ₂₁-*′ may be a single bond,

R₂₁ may be selected from a substituted or unsubstituted C₆-C₆₀ aryl group, a substituted or unsubstituted heteroaryl group, a substituted or unsubstituted monovalent non-aromatic condensed polycyclic group, and a substituted or unsubstituted monovalent non-aromatic condensed heteropolycyclic group,

when a1 is 0, one selected from Ar₃ and Ar₄ may be selected from hydrogen and deuterium,

at least one substituent of the substituted C₅-C₃₀ carbocyclic group, the substituted C₁-C₃₀ heterocyclic group, the substituted C₁-C₆₀ alkyl group, the substituted C₂-C₆₀ alkenyl group, the substituted C₂-C₆₀ alkynyl group, the substituted C₁-C₆₀ alkoxy group, the substituted C₃-C₁₀ cycloalkyl group, the substituted C₁-C₁₀ heterocycloalkyl group, the substituted C₃-C₁₀ cycloalkenyl group, the substituted C₁-C₁₀ heterocycloalkenyl group, the substituted C₆-C₆₀ aryl group, the substituted C₆-C₆₀ aryloxy group, the substituted C₆-C₆₀ arylthio group, the substituted C₁-C₆₀ heteroaryl group, the substituted monovalent non-aromatic condensed polycyclic group, and the substituted monovalent non-aromatic condensed heteropolycyclic group may be selected from:

deuterium, —F, —Cl, —Br, —I, —CD₃, —CD₂H, —CDH₂, —CF₃, —CF₂H, —CFH₂, a hydroxyl group, a cyano group, a nitro group, an amidino group, a hydrazine group, a hydrazone group, a carboxylic acid group or a salt thereof, a sulfonic acid group or a salt thereof, a phosphoric acid group or a salt thereof, a C₁-C₆₀ alkyl group, a C₂-C₆₀ alkenyl group, a C₂-C₆₀ alkynyl group, and a C₁-C₆₀ alkoxy group;

a C₁-C₆₀ alkyl group, a C₂-C₆₀ alkenyl group, a C₂-C₆₀ alkynyl group, and a C₁-C₆₀ alkoxy group, each substituted with at least one selected from deuterium, —F, —Cl, —Br, —I, —CD₃, —CD₂H, —CDH₂, —CF₃, —CF₂H, —CFH₂, a hydroxyl group, a cyano group, a nitro group, an amidino group, a hydrazine group, a hydrazone group, a carboxylic acid group or a salt thereof, a sulfonic acid group or a salt thereof, a phosphoric acid group or a salt thereof, a C₃-C₁₀ cycloalkyl group, a C₁-C₁₀ heterocycloalkyl group, a C₃-C₁₀ cycloalkenyl group, a C₁-C₁₀ heterocycloalkenyl group, a C₆-C₆₀ aryl group, a C₆-C₆₀ aryloxy group, a C₆-C₆₀ arylthio group, a C₁-C₆₀ heteroaryl group, a monovalent non-aromatic condensed polycyclic group, a monovalent non-aromatic condensed heteropolycyclic group, —N(Q₁₁)(Q₁₂), —Si(Q₁₃)(Q₁₄)(Q₁₅), —B(Q₁₆)(Q₁₇), and —P(═O)(Q₁₈)(Q₁₉);

a C₃-C₁₀ cycloalkyl group, a C₁-C₁₀ heterocycloalkyl group, a C₃-C₁₀ cycloalkenyl group, a C₁-C₁₀ heterocycloalkenyl group, a C₆-C₆₀ aryl group, a C₆-C₆₀ aryloxy group, a C₆-C₆₀ arylthio group, a C₁-C₆₀ heteroaryl group, a monovalent non-aromatic condensed polycyclic group, and a monovalent non-aromatic condensed heteropolycyclic group;

a C₃-C₁₀ cycloalkyl group, a C₁-C₁₀ heterocycloalkyl group, a C₃-C₁₀ cycloalkenyl group, a C₁-C₁₀ heterocycloalkenyl group, a C₆-C₆₀ aryl group, a C₆-C₆₀ aryloxy group, a C₆-C₆₀ arylthio group, a C₁-C₆₀ heteroaryl group, a monovalent non-aromatic condensed polycyclic group, and a monovalent non-aromatic condensed heteropolycyclic group, each substituted with at least one selected from deuterium, —F, —Cl, —Br, —I, —CD₃, —CD₂H, —CDH₂, —CF₃, —CF₂H, —CFH₂, a hydroxyl group, a cyano group, a nitro group, an amidino group, a hydrazine group, a hydrazone group, a carboxylic acid group or a salt thereof, a sulfonic acid group or a salt thereof, a phosphoric acid group or a salt thereof, a C₁-C₆₀ alkyl group, a C₂-C₆₀ alkenyl group, a C₂-C₆₀ alkynyl group, a C₁-C₆₀ alkoxy group, a C₃-C₁₀ cycloalkyl group, a C₁-C₁₀ heterocycloalkyl group, a C₃-C₁₀ cycloalkenyl group, a heterocycloalkenyl group, a C₆-C₆₀ aryl group, a C₆-C₆₀ aryloxy group, a C₆-C₆₀ arylthio group, a C₁-C₆₀ heteroaryl group, a monovalent non-aromatic condensed polycyclic group, a monovalent non-aromatic condensed heteropolycyclic group, —N(Q₂₁)(Q₂₂), —Si(Q₂₃)(Q₂₄)(Q₂₅), —B(Q₂₆)(Q₂₇), and —P(═O)(Q₂₈)(Q₂₉); and

—N(Q₃₁)(Q₃₂), —Si(Q₃₃)(Q₃₄)(Q₃₅), —B(Q₃₆)(Q₃₇), and —P(═O)(Q₃₈)(Q₃₉), and

Q₁ to Q₉, Q₁₁ to Q₁₉, Q₂₁ to Q₂₉, and Q₃₁ to Q₃₉ may each independently be selected from hydrogen, deuterium, —F, —Cl, —Br, —I, a hydroxyl group, a cyano group, a nitro group, an amino group, an amidino group, a hydrazine group, a hydrazone group, a carboxylic acid group or a salt thereof, a sulfonic acid group or a salt thereof, a phosphoric acid group or a salt thereof, a C₁-C₆₀ alkyl group, a C₂-C₆₀ alkenyl group, a C₂-C₆₀ alkynyl group, a C₁-C₆₀ alkoxy group, a C₃-C₁₀ cycloalkyl group, a C₁-C₁₀ heterocycloalkyl group, a C₃-C₁₀ cycloalkenyl group, a C₁-C₁₀ heterocycloalkenyl group, a C₆-C₆₀ aryl group, a C₆-C₆₀ aryl group substituted with at least one selected from a C₁-C₆₀ alkyl group and a C₆-C₆₀ aryl group, a C₆-C₆₀ aryloxy group, a C₆-C₆₀ arylthio group, a C₁-C₆₀ heteroaryl group, a monovalent non-aromatic condensed polycyclic group, and a monovalent non-aromatic condensed heteropolycyclic group.

An aspect of another embodiment provides a composition including the condensed cyclic compound.

An aspect of another embodiment provides an organic light-emitting device including:

a first electrode;

a second electrode; and

an organic layer disposed between the first electrode and the second electrode,

wherein the organic layer includes an emission layer and at least one condensed cyclic compound.

### BRIEF DESCRIPTION OF THE DRAWING

These and/or other aspects will become apparent and more readily appreciated from the following description of the embodiments, taken in conjunction with the FIGURE which is a schematic view of an organic light-emitting device according to an embodiment.

### DETAILED DESCRIPTION

Reference will now be made in detail to embodiments, examples of which are illustrated in the accompanying drawings, wherein like reference numerals refer to like elements throughout. In this regard, the present embodiments may have different forms and should not be construed as being limited to the descriptions set forth herein. Accordingly, the embodiments are merely described below, by referring to the FIGURES, to explain aspects of the present description. As used herein, the term “and/or” includes any and all combinations of one or more of the associated listed items. Expressions such as “at least one of,” when preceding a list of elements, modify the entire list of elements and do not modify the individual elements of the list.

Reference will now be made in detail to embodiments, examples of which are illustrated in the accompanying drawings, wherein like reference numerals refer to like elements throughout. In this regard, the present embodiments may have different forms and should not be construed as being limited to the descriptions set forth herein. Accordingly, the embodiments are merely described below, by referring to the FIGURES, to explain aspects of the present description. As used herein, the term “and/or” includes any and all combinations of one or more of the associated listed items. Expressions such as “at least one of,” when preceding a list of elements, modify the entire list of elements and do not modify the individual elements of the list.

It will be understood that when an element is referred to as being “on” another element, it can be directly in contact with the other element or intervening elements may be present therebetween. In contrast, when an element is referred to as being “directly on” another element, there are no intervening elements present.

It will be understood that, although the terms first, second, third etc. may be used herein to describe various elements, components, regions, layers, and/or sections, these elements, components, regions, layers, and/or sections should not be limited by these terms. These terms are only used to distinguish one element, component, region, layer, or section from another element, component, region, layer, or section. Thus, a first element, component, region, layer, or section discussed below could be termed a second element, component, region, layer, or section without departing from the teachings of the present embodiments.

The terminology used herein is for the purpose of describing particular embodiments only and is not intended to be limiting. As used herein, the singular forms “a,” “an,” and “the” are intended to include the plural forms as well, unless the context clearly indicates otherwise.

The term “or” means “and/or.” It will be further understood that the terms “comprises” and/or “comprising,” or “includes” and/or “including” when used in this specification, specify the presence of stated features, regions, integers, steps, operations, elements, and/or components, but do not preclude the presence or addition of one or more other features, regions, integers, steps, operations, elements, components, and/or groups thereof.

Unless otherwise defined, all terms (including technical and scientific terms) used herein have the same meaning as commonly understood by one of ordinary skill in the art to which this general inventive concept belongs. It will be further understood that terms, such as those defined in commonly used dictionaries, should be interpreted as having a meaning that is consistent with their meaning in the context of the relevant art and the present disclosure, and will not be interpreted in an idealized or overly formal sense unless expressly so defined herein.

Exemplary embodiments are described herein with reference to cross section illustrations that are schematic illustrations of idealized embodiments. As such, variations from the shapes of the illustrations as a result, for example, of manufacturing techniques and/or tolerances, are to be expected. Thus, embodiments described herein should not be construed as limited to the particular shapes of regions as illustrated herein but are to include deviations in shapes that result, for example, from manufacturing. For example, a region illustrated or described as flat may, typically, have rough and/or nonlinear features. Moreover, sharp angles that are illustrated may be rounded. Thus, the regions illustrated in the figures are schematic in nature and their shapes are not intended to illustrate the precise shape of a region and are not intended to limit the scope of the present claims.

“About” or “approximately” as used herein is inclusive of the stated value and means within an acceptable range of deviation for the particular value as determined by one of ordinary skill in the art, considering the measurement in question and the error associated with measurement of the particular quantity (i.e., the limitations of the measurement system). For example, “about” can mean within one or more standard deviations, or within ±30%, 20%, 10%, 5% of the stated value.

Condensed Cyclic Compound

According to an aspect of the present disclosure, there is provided a condensed cyclic compound represented by Formula 1:

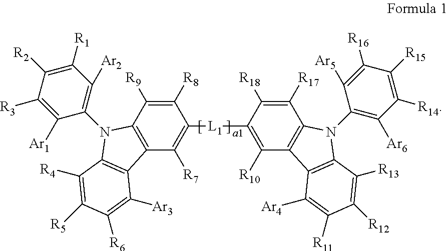

In Formula 1, Ar₁ to Ar₆ and R₁ to R₁₈ may each independently be selected from a group represented by *-(L₂₁)ₐ₂₁-R₂₁, hydrogen, deuterium, —F, —Cl, —Br, —I, a hydroxyl group, a cyano group, a nitro group, an amidino group, a hydrazine group, a hydrazone group, a carboxylic acid group or a salt thereof, a sulfonic acid group or a salt thereof, a phosphoric acid group or a salt thereof, a substituted or unsubstituted C₁-C₆₀ alkyl group, a substituted or unsubstituted C₂-C₆₀ alkenyl group, a substituted or unsubstituted C₂-C₆₀ alkynyl group, a substituted or unsubstituted C₁-C₆₀ alkoxy group, a substituted or unsubstituted C₃-C₁₀ cycloalkyl group, a substituted or unsubstituted C₁-C₁₀ heterocycloalkyl group, a substituted or unsubstituted C₃-C₁₀ cycloalkenyl group, a substituted or unsubstituted C₁-C₁₀ heterocycloalkenyl group, a substituted or unsubstituted C₆-C₆₀ aryl group, a substituted or unsubstituted C₆-C₆₀ aryloxy group, a substituted or unsubstituted C₆-C₆₀ arylthio group, a substituted or unsubstituted C₁-C₆₀ heteroaryl group, a substituted or unsubstituted monovalent non-aromatic condensed polycyclic group, a substituted or unsubstituted monovalent non-aromatic condensed heteropolycyclic group, —Si(Q₁)(Q₂)(Q₃), —N(Q₄)(Q₅), and —B(Q₆)(Q₇),

wherein i) at least one selected from Ar₁, Ar₂, Ar₅, and Ar₆ may each independently be a group represented by *-(L₂₁)ₐ₂₁-R₂₁, and one selected from Ar₃ and Ar₄ may be a group represented by *-(L₂₁)ₐ₂₁-R₂₁; or

ii) Ar₃ and Ar₄ may each independently be a group represented by *-(L₂₁)ₐ₂₁-R₂₁.

When a1 is 0, one selected from Ar₃ and Ar₄ may be selected from hydrogen and deuterium.

In one or more embodiments, in Formula 1,

i) a1 may be 0 or 1, and Ar₁ and Ar₄ may each independently be a group represented by *-(L₂₁)ₐ₂₁-R₂₁;

ii) a1 may be 1, and Ar₃ and Ar₄ may each independently be a group represented by *-(L₂₁)ₐ₂₁-R₂₁; or

iii) a1 may be 1, and Ar₁, Ar₃, and Ar₄ may each independently be a group represented by *-(L₂₁)ₐ₂₁-R₂₁.

In an embodiment, Ar₁ to Ar₆ and R₁ to R₁₈ in Formula 1 may each independently be selected from:

a group represented by *-(L₂₁)ₐ₂₁-R₂₁, hydrogen, deuterium, —F, —Cl, —Br, —I, a cyano group, a methyl group, an ethyl group, an n-propyl group, an iso-propyl group, an n-butyl group, an iso-butyl group, a sec-butyl group, a tert-butyl group, an n-pentyl group, an iso-pentyl group, a tert-pentyl group, a neo-pentyl group, a 1,2-dimethylpropyl group, an n-hexyl group, an iso-hexyl group, a 1,3-dimethylbutyl group, a 1-iso-propylpropyl group, a 1,2-dimethylbutyl group, an n-heptyl group, a 1,4-dimethylpentyl group, a 3-ethylpentyl group, a 2-methyl-1-iso-propylpropyl group, a 1-ethyl-3-methylbutyl group, an n-octyl group, a 2-ethylhexyl group, a 3-methyl-1-iso-propylbutyl group, a 2-methyl-1-iso-propyl group, a 1-tert-butyl-2-methylpropyl group, an n-nonyl group, a 3,5,5-trimethyldecyl group, an n-decyl group, an iso-decyl group, an n-undecyl group, a 1-methyldecyl group, an n-dodecyl group, an n-tridecyl group, an n-tetradecyl group, an n-pentadecyl group, an n-hexadecyl group, an n-heptadecyl group, an n-octadecyl group, an n-nonadecyl group, an n-eicosyl group, an n-heneicosyl group, an n-docosyl group, an n-tricosyl group, an n-tetracosyl group, a methoxy group, an ethoxy group, a propoxy group, an iso-propoxy group, an n-butoxy group, an iso-butoxy group, a sec-butoxy group, tert-butoxy group, an n-pentoxy group, an iso-pentoxy group, a tert-pentoxy group, a neo-pentoxy group, an n-hexyloxy group, an iso-hexyloxy group, a heptyloxy group, an octyloxy group, a nonyloxy group, a decyloxy group, an undecyloxy group, a dodecyloxy group, a tridecyloxy group, a tetradecyloxy group, a pentadecyloxy group, a hexadecyl group, a heptadecyl group, an octadecyl group, a 2-ethylhexyloxy group, a 3-ethylpentyloxy group, a phenoxy group, a 1-naphthyloxy group, a 2-naphthyloxy group, a 2-azulenyloxy group, a 2-furanyloxy group, a 2-thienyloxy group, a 2-indolyloxy group, a 3-indolyloxy group, a 2-benzofuryloxy group, a 2-benzothienyloxy group, an N-methylamino group, an n-ethylamino group, an n-propylamino group, an n-iso-propylamino group, an n-butylamino group, an n-iso-butylamino group, an n-sec-butylamino group, an n-tert-butylamino group, an n-pentylamino group, an n-hexylamino group, an N,N-dimethylamino group, an N,N-diethylamino group, an N,N-dipropylamino group, an N,N-di-iso-propylamino group, an N,N-dibutylamino group, an N,N-di-iso-butylamino group, an N,N-dipentylamino group, and an N,N-dihexylamino group;

a phenyl group, a pentalenyl group, an indenyl group, a naphthyl group, an anthracenyl group, an azulenyl group, a heptalenyl group, an acenaphthyl group, a phenalenyl group, a fluorenyl group, a spiro-bifluorenyl group, a benzofluorenyl group, a dibenzofluorenyl group, a phenanthrenyl group, a biphenyl group, a terphenyl group, a triphenylenyl group, a fluoranthenyl group, a pyrenyl group, a chrysenyl group, a picenyl group, a perylenyl group, a pentaphenyl group, a pentacenyl group, a tetraphenyl group, a hexaphenyl group, a hexacenyl group, a rubicenyl group, a trinaphthyl group, a heptaphenyl group, a pyranthrenyl group, a pyridinyl group, a pyrazinyl group, a pyridazinyl group, a pyrimidinyl group, a triazinyl group, a quinolinyl group, an isoquinolinyl group, a quinoxalinyl group, a quinazolinyl group, a naphthyridinyl group, an acridinyl group, a phenazinyl group, a benzoquinolinyl group, a benzoisoquinolinyl group, a phenanthridinyl group, a phenanthrolinyl group, a benzoquinonyl group, a cumarinyl group, an anthraquinonyl group, a fluorenonyl group, a furanyl group, a thienyl group, a silolyl group, a benzofuranyl group, a benzothienyl group, a benzosilolyl group, a dibenzofuranyl group, a dibenzothiophenyl group, a dibenzosilolyl group, a pyrrolyl group, an indolyl group, an isoindolyl group, a carbazolyl group, a benzocarbazolyl group, a dibenzocarbazolyl group, an imidazolyl group, a benzimidazolyl group, a pyrazolyl group, a triazolyl group, a tetrazolyl group, an indazolyl group, an oxazolyl group, an isoxazolyl group, a benzoxazolyl group, a benzisoxazolyl group, a thiazolyl group, an isothiazolyl group, a benzothiazolyl group, a benzisothiazolyl group, an imidazopyridinyl group, an imidazopyrimidinyl group, an imidazophenanthridinyl group, a benzimidazophenanthridinyl group, an azadibenzofuranyl group, an azacarbazolyl group, an azadibenzothienyl group, a diazadibenzofuranyl group, a diazacarbazolyl group, a diazadibenzothienyl group, a xanthonyl group, and a thioxanthonyl group; and

a phenyl group, a pentalenyl group, an indenyl group, a naphthyl group, an anthracenyl group, an azulenyl group, a heptalenyl group, an acenaphthyl group, a phenalenyl group, a fluorenyl group, a spiro-bifluorenyl group, a benzofluorenyl group, a dibenzofluorenyl group, a phenanthrenyl group, a biphenyl group, a terphenyl group, a triphenylenyl group, a fluoranthenyl group, a pyrenyl group, a chrysenyl group, a picenyl group, a perylenyl group, a pentaphenyl group, a pentacenyl group, a tetraphenyl group, a hexaphenyl group, a hexacenyl group, a rubicenyl group, a trinaphthyl group, a heptaphenyl group, a pyranthrenyl group, a pyridinyl group, a pyrazinyl group, a pyridazinyl group, a pyrimidinyl group, a triazinyl group, a quinolinyl group, an isoquinolinyl group, a quinoxalinyl group, a quinazolinyl group, a naphthyridinyl group, an acridinyl group, a phenazinyl group, a benzoquinolinyl group, a benzoisoquinolinyl group, a phenanthridinyl group, a phenanthrolinyl group, a benzoquinonyl group, a cumarinyl group, an anthraquinonyl group, a fluorenonyl group, a furanyl group, a thienyl group, a silolyl group, a benzofuranyl group, a benzothienyl group, a benzosilolyl group, a dibenzofuranyl group, a dibenzothiophenyl group, a dibenzosilolyl group, a pyrrolyl group, an indolyl group, an isoindolyl group, a carbazolyl group, a benzocarbazolyl group, a dibenzocarbazolyl group, an imidazolyl group, a benzimidazolyl group, a pyrazolyl group, a triazolyl group, a tetrazolyl group, an indazolyl group, an oxazolyl group, an isoxazolyl group, a benzoxazolyl group, a benzisoxazolyl group, a thiazolyl group, an isothiazolyl group, a benzothiazolyl group, a benzisothiazolyl group, an imidazopyridinyl group, an imidazopyrimidinyl group, an imidazophenanthridinyl group, a benzimidazophenanthridinyl group, an azadibenzofuranyl group, an azacarbazolyl group, an azadibenzothienyl group, a diazadibenzofuranyl group, a diazacarbazolyl group, a diazadibenzothienyl group, a xanthonyl group, and a thioxanthonyl group, each substituted with at least one selected from deuterium, —F, —Cl, —Br, —I, a hydroxyl group, a cyano group, a nitro group, an amidino group, a hydrazine group, a hydrazone group, a C₁-C₃₀ alkyl group, a C₁-C₃₀ alkoxy group, a phenyl group, a pentalenyl group, an indenyl group, a naphthyl group, an anthracenyl group, an azulenyl group, a heptalenyl group, an acenaphthyl group, a phenalenyl group, a fluorenyl group, a spiro-bifluorenyl group, a benzofluorenyl group, a dibenzofluorenyl group, a phenanthrenyl group, a biphenyl group, a terphenyl group, a triphenylenyl group, a fluoranthenyl group, a pyrenyl group, a chrysenyl group, a picenyl group, a perylenyl group, a pentaphenyl group, a pentacenyl group, a tetraphenyl group, a hexaphenyl group, a hexacenyl group, a rubicenyl group, a trinaphthyl group, a heptaphenyl group, a pyranthrenyl group, a pyridinyl group, a pyrazinyl group, a pyridazinyl group, a pyrimidinyl group, a triazinyl group, a quinolinyl group, an isoquinolinyl group, a quinoxalinyl group, a quinazolinyl group, a naphthyridinyl group, an acridinyl group, a phenazinyl group, a benzoquinolinyl group, a benzoisoquinolinyl group, a phenanthridinyl group, a phenanthrolinyl group, a benzoquinonyl group, a cumarinyl group, an anthraquinonyl group, a fluorenonyl group, a furanyl group, a thienyl group, a silolyl group, a benzofuranyl group, a benzothienyl group, a benzosilolyl group, a dibenzofuranyl group, a dibenzothiophenyl group, a dibenzosilolyl group, a pyrrolyl group, an indolyl group, an isoindolyl group, a carbazolyl group, a benzocarbazolyl group, a dibenzocarbazolyl group, an imidazolyl group, a benzimidazolyl group, a pyrazolyl group, a triazolyl group, a tetrazolyl group, an indazolyl group, an oxazolyl group, an isoxazolyl group, a benzoxazolyl group, a benzisoxazolyl group, a thiazolyl group, an isothiazolyl group, a benzothiazolyl group, a benzisothiazolyl group, an imidazopyridinyl group, an imidazopyrimidinyl group, an imidazophenanthridinyl group, a benzimidazophenanthridinyl group, an azadibenzofuranyl group, an azacarbazolyl group, an azadibenzothienyl group, a diazadibenzofuranyl group, a diazacarbazolyl group, a diazadibenzothienyl group, a xanthonyl group, and a thioxanthonyl group.

In one or more embodiments, Ar₁ to Ar₆ and R₁ to R₁₈ in Formula 1 may each independently be selected from:

a group represented by *-(L₂₁)ₐ₂₁-R₂₁, hydrogen, deuterium, —F, —Cl, —Br, —I, a cyano group, a methyl group, an ethyl group, an n-propyl group, an iso-propyl group, an n-butyl group, an iso-butyl group, a sec-butyl group, and a tert-butyl group;

a phenyl group, a naphthyl group, a fluorenyl group, a phenanthrenyl group, a biphenyl group, a terphenyl group, a triphenylenyl group, a pyridinyl group, a pyrazinyl group, a pyridazinyl group, a pyrimidinyl group, a triazinyl group, a quinolinyl group, an isoquinolinyl group, a quinoxalinyl group, a quinazolinyl group, a naphthyridinyl group, a furanyl group, a thienyl group, a silolyl group, a benzofuranyl group, a benzothienyl group, a benzosilolyl group, a dibenzofuranyl group, a dibenzothiophenyl group, a dibenzosilolyl group, a pyrrolyl group, an indolyl group, a carbazolyl group, an imidazolyl group, and a benzimidazolyl group; and

a phenyl group, a naphthyl group, a fluorenyl group, a phenanthrenyl group, a biphenyl group, a terphenyl group, a triphenylenyl group, a pyridinyl group, a pyrazinyl group, a pyridazinyl group, a pyrimidinyl group, a triazinyl group, a quinolinyl group, an isoquinolinyl group, a quinoxalinyl group, a quinazolinyl group, a naphthyridinyl group, a furanyl group, a thienyl group, a silolyl group, a benzofuranyl group, a benzothienyl group, a benzosilolyl group, a dibenzofuranyl group, a dibenzothiophenyl group, a dibenzosilolyl group, a pyrrolyl group, an indolyl group, a carbazolyl group, an imidazolyl group, and a benzimidazolyl group, each substituted with at least one selected from deuterium, —F, —Cl, —Br, —I, a hydroxyl group, a cyano group, a nitro group, an amidino group, a hydrazine group, a hydrazone group, a C₁-C₃₀ alkyl group, a C₁-C₃₀ alkoxy group, a phenyl group, a naphthyl group, a biphenyl group, and a terphenyl group.

In one or more embodiments, Ar₁ to Ar₆ and R₁ to R₁₈ in Formula 1 may each independently be selected from:

a group represented by *-(L₂₁)ₐ₂₁-R₂₁, hydrogen, deuterium, —F, —Cl, —Br, —I, a cyano group, a methyl group, an ethyl group, an n-propyl group, an iso-propyl group, an n-butyl group, an iso-butyl group, a sec-butyl group, and a tert-butyl group;

a phenyl group, a biphenyl group, and a terphenyl group; and

a phenyl group, a biphenyl group, and a terphenyl group, each substituted with at least one selected from deuterium, —F, —Cl, —Br, —I, a cyano group, a C₁-C₂₀ alkyl group, and a C₁-C₂₀ alkoxy group.

L₁ and L₂₁ In Formula 1 may each independently be selected from a substituted or unsubstituted C₅-C₃₀ carbocyclic group and a substituted or unsubstituted C₁-C₃₀ heterocyclic group.

In an embodiment, L₁ and L₂₁ in Formula 1 may each independently be a substituted or unsubstituted C₅-C₃₀ carbocyclic group.

In an embodiment, L₁ and L₂₁ in Formula 1 may each independently be selected from:

a benzene group, a pentalene group, an indene group, a naphthalene group, an anthracene group, an azulene group, a heptalene group, an acenaphthylene group, a phenalene group, a fluorene group, a spiro-bifluorene group, a benzofluorene group, a dibenzofluorene group, a phenanthrene group, a biphenyl group, a terphenyl group, a triphenylene group, a fluoranthene group, a pyrene group, a chrysene group, a picene group, a perylene group, a pentaphene group, a pentacene group, a tetraphene group, a hexaphene group, a hexacene group, a rubicene group, a trinaphthalene group, a heptaphene group, a pyranthrene group, a pyridine group, a pyrazine group, a pyridazine group, a pyrimidine group, a triazine group, a quinoline group, an isoquinoline group, a quinoxaline group, a quinazoline group, a naphthyridine group, an acridine group, a phenazine group, a benzoquinoline group, a benzoisoquinoline group, a phenanthridine group, a phenanthroline group, a benzoquinone group, a coumarin group, an anthraquinone group, a fluorenone group, a furan group, a thiophene group, a silole group, a benzofuran group, a benzothiophene group, a benzosilole group, a dibenzofuran group, a dibenzothiophene group, a dibenzosilole group, a pyrrole group, an indole group, an isoindole group, a carbazole group, a benzocarbazole group, a dibenzocarbazole group, an imidazole group, a benzimidazole group, a pyrazole group, a triazole group, a tetrazole group, an indazole group, an oxazole group, an isoxazole group, a benzoxazole group, a benzisoxazole group, a thiazole group, an isothiazole group, a benzothiazole group, a benzisothiazole group, an imidazolinone group, a benzoimidazolinone group, an imidazopyridine group, an imidazopyrimidine group, an imidazophenanthridine group, a benzimidazophenanthridine group, an azadibenzofuran group, an azacarbazole group, an azadibenzothiophene group, a diazadibenzofuran group, a diazacarbazole group, a diazadibenzothiophene group, a xanthone group, and a thioxanthone group; and

a benzene group, a pentalene group, an indene group, a naphthalene group, an anthracene group, an azulene group, a heptalene group, an acenaphthylene group, a phenalene group, a fluorene group, a spiro-bifluorene group, a benzofluorene group, a dibenzofluorene group, a phenanthrene group, a biphenyl group, a terphenyl group, a triphenylene group, a fluoranthene group, a pyrene group, a chrysene group, a picene group, a perylene group, a pentaphene group, a pentacene group, a tetraphene group, a hexaphene group, a hexacene group, a rubicene group, a trinaphthalene group, a heptaphene group, a pyranthrene group, a pyridine group, a pyrazine group, a pyridazine group, a pyrimidine group, a triazine group, a quinoline group, an isoquinoline group, a quinoxaline group, a quinazoline group, a naphthyridine group, an acridine group, a phenazine group, a benzoquinoline group, a benzoisoquinoline group, a phenanthridine group, a phenanthroline group, a benzoquinone group, a coumarin group, an anthraquinone group, a fluorenone group, a furan group, a thiophene group, a silole group, a benzofuran group, a benzothiophene group, a benzosilole group, a dibenzofuran group, a dibenzothiophene group, a dibenzosilole group, a pyrrole group, an indole group, an isoindole group, a carbazole group, a benzocarbazole group, a dibenzocarbazole group, an imidazole group, a benzimidazole group, a pyrazole group, a triazole group, a tetrazole group, an indazole group, an oxazole group, an isoxazole group, a benzoxazole group, a benzisoxazole group, a thiazole group, an isothiazole group, a benzothiazole group, a benzisothiazole group, an imidazolinone group, a benzoimidazolinone group, an imidazopyridine group, an imidazopyrimidine group, an imidazophenanthridine group, a benzimidazophenanthridine group, an azadibenzofuran group, an azacarbazole group, an azadibenzothiophene group, a diazadibenzofuran group, a diazacarbazole group, a diazadibenzothiophene group, a xanthone group, and a thioxanthone group, each substituted with at least one selected from deuterium, —F, —C₁, —Br, —I, a hydroxyl group, a cyano group, a nitro group, an amidino group, a hydrazine group, a hydrazone group, a C₁-C₃₀ alkyl group, a C₁-C₃₀ alkoxy group, a phenyl group, a pentalenyl group, an indenyl group, a naphthyl group, an anthracenyl group, an azulenyl group, a heptalenyl group, an acenaphthyl group, a phenalenyl group, a fluorenyl group, a spiro-bifluorenyl group, a benzofluorenyl group, a dibenzofluorenyl group, a phenanthrenyl group, a biphenyl group, a terphenyl group, a triphenylenyl group, a fluoranthenyl group, a pyrenyl group, a chrysenyl group, a picenyl group, a perylenyl group, a pentaphenyl group, a pentacenyl group, a tetraphenyl group, a hexaphenyl group, a hexacenyl group, a rubicenyl group, a trinaphthyl group, a heptaphenyl group, a pyranthrenyl group, a pyridinyl group, a pyrazinyl group, a pyridazinyl group, a pyrimidinyl group, a triazinyl group, a quinolinyl group, an isoquinolinyl group, a quinoxalinyl group, a quinazolinyl group, a naphthyridinyl group, an acridinyl group, a phenazinyl group, a benzoquinolinyl group, a benzoisoquinolinyl group, a phenanthridinyl group, a phenanthrolinyl group, a benzoquinonyl group, a cumarinyl group, an anthraquinonyl group, a fluorenonyl group, a furanyl group, a thienyl group, a silolyl group, a benzofuranyl group, a benzothienyl group, a benzosilolyl group, a dibenzofuranyl group, a dibenzothiophenyl group, a dibenzosilolyl group, a pyrrolyl group, an indolyl group, an isoindolyl group, a carbazolyl group, a benzocarbazolyl group, a dibenzocarbazolyl group, an imidazolyl group, a benzimidazolyl group, a pyrazolyl group, a triazolyl group, a tetrazolyl group, an indazolyl group, an oxazolyl group, an isoxazolyl group, a benzoxazolyl group, a benzisoxazolyl group, a thiazolyl group, an isothiazolyl group, a benzothiazolyl group, a benzisothiazolyl group, an imidazopyridinyl group, an imidazopyrimidinyl group, an imidazophenanthridinyl group, a benzimidazophenanthridinyl group, an azadibenzofuranyl group, an azacarbazolyl group, an azadibenzothienyl group, a diazadibenzofuranyl group, a diazacarbazolyl group, a diazadibenzothienyl group, a xanthonyl group, and a thioxanthonyl group.

In one or more embodiments, L₁ and L₂₁ in Formula 1 each independently be selected from:

a benzene group, a naphthalene group, a fluorene group, a carbazole group, a pyridine group, a pyrazine group, a pyridazine group, a pyrimidine group, and a triazine group; and

a benzene group, a naphthalene group, a fluorene group, a carbazole group, a pyridine group, a pyrazine group, a pyridazine group, a pyrimidine group, and a triazine group, each substituted with at least one selected from deuterium, —F, —Cl, —Br, —I, a hydroxyl group, a cyano group, a nitro group, an amidino group, a hydrazino group, a hydrazono group, a C₁-C₃₀ alkyl group, a C₁-C₃₀ alkoxy group, a phenyl group, a naphthyl group, a biphenyl group, and a terphenyl group.

In one or more embodiments, L₁ and L₂₁ in Formula 1 may each independently be selected from groups represented by Formulae 3-1 to 3-3:

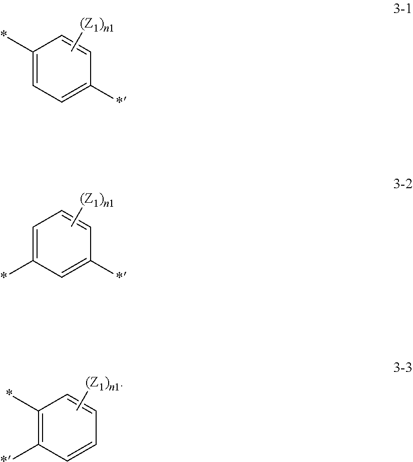

In Formulae 3-1 to 3-3,

Z₁ may be selected from hydrogen, deuterium, —F, —Cl, —Br, —I, a hydroxyl group, a cyano group, a nitro group, an amino group, an amidino group, a hydrazine group, a hydrazone group, a carboxylic acid group or a salt thereof, a sulfonic acid group or a salt thereof, a phosphoric acid group or a salt thereof, a C₁-C₃₀ alkyl group, a C₁-C₃₀ alkoxy group, a phenyl group, a naphthyl group, a biphenyl group, and a terphenyl group,

n1 may be 1, 2, 3, or 4, and

* and *′ each independently indicate a binding site to a neighboring atom.

In an embodiment, in Formula 1, L₁ may be a group represented by Formula 3-1, and L₂₁ may be a group represented by Formula 3-2.

In Formula 1, a1 and a21 each indicate the number of L₁ and the number of L₂₁, respectively, and may each independently be 0 or 1. When a1 is 0, *-(L₁)ₐ₁-*′ may be a single bond, and when a21 is 0, *-(L₂₁)ₐ₂₁-*′ may be a single bond.

In Formula 1, R₂₁ may be selected from a substituted or unsubstituted C₆-C₆₀ aryl group, a substituted or unsubstituted C₁-C₆₀ heteroaryl group, a substituted or unsubstituted monovalent non-aromatic condensed polycyclic group, and a substituted or unsubstituted monovalent non-aromatic condensed heteropolycyclic group.

In an embodiment, R₂₁ may be selected from:

a phenyl group, a pentalenyl group, an indenyl group, a naphthyl group, an anthracenyl group, an azulenyl group, a heptalenyl group, an acenaphthyl group, a phenalenyl group, a fluorenyl group, a spiro-bifluorenyl group, a benzofluorenyl group, a dibenzofluorenyl group, a phenanthrenyl group, a biphenyl group, a terphenyl group, a triphenylenyl group, a fluoranthenyl group, a pyrenyl group, a chrysenyl group, a picenyl group, a perylenyl group, a pentaphenyl group, a pentacenyl group, a tetraphenyl group, a hexaphenyl group, a hexacenyl group, a rubicenyl group, a trinaphthyl group, a heptaphenyl group, a pyranthrenyl group, a pyridinyl group, a pyrazinyl group, a pyridazinyl group, a pyrimidinyl group, a triazinyl group, a quinolinyl group, an isoquinolinyl group, a quinoxalinyl group, a quinazolinyl group, a naphthyridinyl group, an acridinyl group, a phenazinyl group, a benzoquinolinyl group, a benzoisoquinolinyl group, a phenanthridinyl group, a phenanthrolinyl group, a benzoquinonyl group, a cumarinyl group, an anthraquinonyl group, a fluorenonyl group, a furanyl group, a thienyl group, a silolyl group, a benzofuranyl group, a benzothienyl group, a benzosilolyl group, a dibenzofuranyl group, a dibenzothiophenyl group, a dibenzosilolyl group, a pyrrolyl group, an indolyl group, an isoindolyl group, a carbazolyl group, a benzocarbazolyl group, a dibenzocarbazolyl group, an imidazolyl group, a benzimidazolyl group, a pyrazolyl group, a triazolyl group, a tetrazolyl group, an indazolyl group, an oxazolyl group, an isoxazolyl group, a benzoxazolyl group, a benzisoxazolyl group, a thiazolyl group, an isothiazolyl group, a benzothiazolyl group, a benzisothiazolyl group, an imidazopyridinyl group, an imidazopyrimidinyl group, an imidazophenanthridinyl group, a benzimidazophenanthridinyl group, an azadibenzofuranyl group, an azacarbazolyl group, an azadibenzothienyl group, a diazadibenzofuranyl group, a diazacarbazolyl group, a diazadibenzothienyl group, a xanthonyl group, and a thioxanthonyl group; and

a phenyl group, a pentalenyl group, an indenyl group, a naphthyl group, an anthracenyl group, an azulenyl group, a heptalenyl group, an acenaphthyl group, a phenalenyl group, a fluorenyl group, a spiro-bifluorenyl group, a benzofluorenyl group, a dibenzofluorenyl group, a phenanthrenyl group, a biphenyl group, a terphenyl group, a triphenylenyl group, a fluoranthenyl group, a pyrenyl group, a chrysenyl group, a picenyl group, a perylenyl group, a pentaphenyl group, a pentacenyl group, a tetraphenyl group, a hexaphenyl group, a hexacenyl group, a rubicenyl group, a trinaphthyl group, a heptaphenyl group, a pyranthrenyl group, a pyridinyl group, a pyrazinyl group, a pyridazinyl group, a pyrimidinyl group, a triazinyl group, a quinolinyl group, an isoquinolinyl group, a quinoxalinyl group, a quinazolinyl group, a naphthyridinyl group, an acridinyl group, a phenazinyl group, a benzoquinolinyl group, a benzoisoquinolinyl group, a phenanthridinyl group, a phenanthrolinyl group, a benzoquinonyl group, a cumarinyl group, an anthraquinonyl group, a fluorenonyl group, a furanyl group, a thienyl group, a silolyl group, a benzofuranyl group, a benzothienyl group, a benzosilolyl group, a dibenzofuranyl group, a dibenzothiophenyl group, a dibenzosilolyl group, a pyrrolyl group, an indolyl group, an isoindolyl group, a carbazolyl group, a benzocarbazolyl group, a dibenzocarbazolyl group, an imidazolyl group, a benzimidazolyl group, a pyrazolyl group, a triazolyl group, a tetrazolyl group, an indazolyl group, an oxazolyl group, an isoxazolyl group, a benzoxazolyl group, a benzisoxazolyl group, a thiazolyl group, an isothiazolyl group, a benzothiazolyl group, a benzisothiazolyl group, an imidazopyridinyl group, an imidazopyrimidinyl group, an imidazophenanthridinyl group, a benzimidazophenanthridinyl group, an azadibenzofuranyl group, an azacarbazolyl group, an azadibenzothienyl group, a diazadibenzofuranyl group, a diazacarbazolyl group, a diazadibenzothienyl group, a xanthonyl group, and a thioxanthonyl group, each substituted with at least one selected from deuterium, —F, —Cl, —Br, —I, a hydroxyl group, a cyano group, a nitro group, an amidino group, a hydrazine group, a hydrazone group, a C₁-C₃₀ alkyl group, a C₁-C₃₀ alkoxy group, a phenyl group, a pentalenyl group, an indenyl group, a naphthyl group, an anthracenyl group, an azulenyl group, a heptalenyl group, an acenaphthyl group, a phenalenyl group, a fluorenyl group, a spiro-bifluorenyl group, a benzofluorenyl group, a dibenzofluorenyl group, a phenanthrenyl group, a biphenyl group, a terphenyl group, a triphenylenyl group, a fluoranthenyl group, a pyrenyl group, a chrysenyl group, a picenyl group, a perylenyl group, a pentaphenyl group, a pentacenyl group, a tetraphenyl group, a hexaphenyl group, a hexacenyl group, a rubicenyl group, a trinaphthyl group, a heptaphenyl group, a pyranthrenyl group, a pyridinyl group, a pyrazinyl group, a pyridazinyl group, a pyrimidinyl group, a triazinyl group, a quinolinyl group, an isoquinolinyl group, a quinoxalinyl group, a quinazolinyl group, a naphthyridinyl group, an acridinyl group, a phenazinyl group, a benzoquinolinyl group, a benzoisoquinolinyl group, a phenanthridinyl group, a phenanthrolinyl group, a benzoquinonyl group, a cumarinyl group, an anthraquinonyl group, a fluorenonyl group, a furanyl group, a thienyl group, a silolyl group, a benzofuranyl group, a benzothienyl group, a benzosilolyl group, a dibenzofuranyl group, a dibenzothiophenyl group, a dibenzosilolyl group, a pyrrolyl group, an indolyl group, an isoindolyl group, a carbazolyl group, a benzocarbazolyl group, a dibenzocarbazolyl group, an imidazolyl group, a benzimidazolyl group, a pyrazolyl group, a triazolyl group, a tetrazolyl group, an indazolyl group, an oxazolyl group, an isoxazolyl group, a benzoxazolyl group, a benzisoxazolyl group, a thiazolyl group, an isothiazolyl group, a benzothiazolyl group, a benzisothiazolyl group, an imidazopyridinyl group, an imidazopyrimidinyl group, an imidazophenanthridinyl group, a benzimidazophenanthridinyl group, an azadibenzofuranyl group, an azacarbazolyl group, an azadibenzothienyl group, a diazadibenzofuranyl group, a diazacarbazolyl group, a diazadibenzothienyl group, a xanthonyl group, and a thioxanthonyl group.

In one or more embodiments, R₂₁ may be selected from:

a phenyl group, a naphthyl group, a fluorenyl group, a phenanthrenyl group, a biphenyl group, a terphenyl group, a triphenylenyl group, a pyridinyl group, a pyrazinyl group, a pyridazinyl group, a pyrimidinyl group, a triazinyl group, a quinolinyl group, an isoquinolinyl group, a quinoxalinyl group, a quinazolinyl group, a naphthyridinyl group, a furanyl group, a thienyl group, a silolyl group, a benzofuranyl group, a benzothienyl group, a benzosilolyl group, a dibenzofuranyl group, a dibenzothiophenyl group, a dibenzosilolyl group, a pyrrolyl group, an indolyl group, a carbazolyl group, an imidazolyl group, and a benzimidazolyl group; and

a phenyl group, a naphthyl group, a fluorenyl group, a phenanthrenyl group, a biphenyl group, a terphenyl group, a triphenylenyl group, a pyridinyl group, a pyrazinyl group, a pyridazinyl group, a pyrimidinyl group, a triazinyl group, a quinolinyl group, an isoquinolinyl group, a quinoxalinyl group, a quinazolinyl group, a naphthyridinyl group, a furanyl group, a thienyl group, a silolyl group, a benzofuranyl group, a benzothienyl group, a benzosilolyl group, a dibenzofuranyl group, a dibenzothiophenyl group, a dibenzosilolyl group, a pyrrolyl group, an indolyl group, a carbazolyl group, an imidazolyl group, and a benzimidazolyl group, each substituted with at least one selected from deuterium, —F, —Cl, —Br, —I, a hydroxyl group, a cyano group, a nitro group, an amidino group, a hydrazine group, a hydrazone group, a C₁-C₃₀ alkyl group, a C₁-C₃₀ alkoxy group, a phenyl group, a naphthyl group, a biphenyl group, and a terphenyl group.

In one or more embodiments, R₂₁ may be selected from:

a phenyl group, a biphenyl group, and a terphenyl group; and

a phenyl group, a biphenyl group, and a terphenyl group, each substituted with at least one selected from deuterium, —F, —Cl, —Br, —I, a cyano group, a C₁-C₂₀ alkyl group, and a C₁-C₂₀ alkoxy group.

In an embodiment, in Formula 1, at least one selected from R₁ to R₃ and R₁₄ to R₁₆ may each independently be selected from a substituted or unsubstituted C₆-C₆₀ aryl group, a substituted or unsubstituted C₁-C₆₀ heteroaryl group, a substituted or unsubstituted monovalent non-aromatic condensed polycyclic group, and a substituted or unsubstituted monovalent non-aromatic condensed heteropolycyclic group.

In one or more embodiments, in Formula 1, at least one selected from R₁ to R₃ and/or at least one selected from R₁₄ to R₁₆ may each independently be selected from a substituted or unsubstituted C₆-C₆₀ aryl group, a substituted or unsubstituted C₁-C₆₀ heteroaryl group, a substituted or unsubstituted monovalent non-aromatic condensed polycyclic group, and a substituted or unsubstituted monovalent non-aromatic condensed heteropolycyclic group.

In one or more embodiments,

i) at least one selected from R₁ and R₂ may each independently be selected from a substituted or unsubstituted C₆-C₆₀ aryl group, a substituted or unsubstituted C₁-C₆₀ heteroaryl group, a substituted or unsubstituted monovalent non-aromatic condensed polycyclic group, and a substituted or unsubstituted monovalent non-aromatic condensed heteropolycyclic group;

ii) at least one selected from R₁₄ and R₁₆ may each independently be selected from a substituted or unsubstituted C₆-C₆₀ aryl group, a substituted or unsubstituted C₁-C₆₀ heteroaryl group, a substituted or unsubstituted monovalent non-aromatic condensed polycyclic group, and a substituted or unsubstituted monovalent non-aromatic condensed heteropolycyclic group; or

iii) at least one selected from R₁ and R₂ and at least one selected from R₁₄ and R₁₅ may each independently be selected from a substituted or unsubstituted C₆-C₆₀ aryl group, a substituted or unsubstituted C₁-C₆₀ heteroaryl group, a substituted or unsubstituted monovalent non-aromatic condensed polycyclic group, and a substituted or unsubstituted monovalent non-aromatic condensed heteropolycyclic group.

In one or more embodiments,

i) one selected from R₁ and R₂ may be selected from a substituted or unsubstituted C₆-C₆₀ aryl group, a substituted or unsubstituted C₁-C₆₀ heteroaryl group, a substituted or unsubstituted monovalent non-aromatic condensed polycyclic group, and a substituted or unsubstituted monovalent non-aromatic condensed heteropolycyclic group;

ii) one selected from R₁₄ and R₁₅ may be selected from a substituted or unsubstituted C₆-C₆₀ aryl group, a substituted or unsubstituted C₁-C₆₀ heteroaryl group, a substituted or unsubstituted monovalent non-aromatic condensed polycyclic group, and a substituted or unsubstituted monovalent non-aromatic condensed heteropolycyclic group; or

ii) one selected from R₁ and R₂ and one selected from R₁₄ and R₁₅ may each independently be selected from a substituted or unsubstituted C₆-C₆₀ aryl group, a substituted or unsubstituted C₁-C₆₀ heteroaryl group, a substituted or unsubstituted monovalent non-aromatic condensed polycyclic group, and a substituted or unsubstituted monovalent non-aromatic condensed heteropolycyclic group.

In one or more embodiments, in Formula 1,

i) a1 may be 0 or 1, Ar₁ and Ar₄ may each independently be a group represented by *-(L₂₁)ₐ₂₁-R₂₁, and one selected from R₁ and R₂ and/or one selected from R₁₄ and R₁₅ may each independently be selected from a substituted or unsubstituted C₆-C₆₀ aryl group, a substituted or unsubstituted C₁-C₆₀ heteroaryl group, a substituted or unsubstituted monovalent non-aromatic condensed polycyclic group, and a substituted or unsubstituted monovalent non-aromatic condensed heteropolycyclic group;

ii) a1 may be 1, Ar₃ and Ar₄ may each independently be a group represented by *-(L₂₁)ₐ₂₁-R₂₁, and one selected from R₁ and R₂ and/or one selected from R₁₄ and R₁₅ may each independently be selected from a substituted or unsubstituted C₆-C₆₀ aryl group, a substituted or unsubstituted C₁-C₆₀ heteroaryl group, a substituted or unsubstituted monovalent non-aromatic condensed polycyclic group, and a substituted or unsubstituted monovalent non-aromatic condensed heteropolycyclic group; or

iii) a1 may be 1, Ar₁, Ar₃, and Ar₄ may each independently be a group represented by *-(L₂₁)ₐ₂₁-R₂₁, and one selected from R₁ and R₂ and/or one selected from R₁₄ and R₁₅ may each independently be selected from a substituted or unsubstituted C₆-C₆₀ aryl group, a substituted or unsubstituted C₁-C₆₀ heteroaryl group, a substituted or unsubstituted monovalent non-aromatic condensed polycyclic group, and a substituted or unsubstituted monovalent non-aromatic condensed heteropolycyclic group.

In one or more embodiments, in Formula 1,

i) a1 may be 0 or 1, Ar₁ and Ar₄ may each independently be selected from a group represented by *-(L₂₁)ₐ₂₁-R₂₁, and one selected from R₁ and R₂ may be selected from a substituted or unsubstituted C₆-C₆₀ aryl group, a substituted or unsubstituted C₁-C₆₀ heteroaryl group, a substituted or unsubstituted monovalent non-aromatic condensed polycyclic group, and a substituted or unsubstituted monovalent non-aromatic condensed heteropolycyclic group;

ii) a1 may be 0 or 1, Ar₁ and Ar₄ may each independently be a group represented by *-(L₂₁)ₐ₂₁-R₂₁, and one selected from R₁₄ and R₁₅ may be selected from a substituted or unsubstituted C₆-C₆₀ aryl group, a substituted or unsubstituted C₁-C₆₀ heteroaryl group, a substituted or unsubstituted monovalent non-aromatic condensed polycyclic group, and a substituted or unsubstituted monovalent non-aromatic condensed heteropolycyclic group;

iii) a1 may be 0 or 1, Ar₁ and Ar₄ may each independently be a group represented by *-(L₂₁)ₐ₂₁-R₂₁, and one selected from R₁ and R₂ and one selected from R₁₄ and R₁₅ may each independently be selected from a substituted or unsubstituted C₆-C₆₀ aryl group, a substituted or unsubstituted C₁-C₆₀ heteroaryl group, a substituted or unsubstituted monovalent non-aromatic condensed polycyclic group, and a substituted or unsubstituted monovalent non-aromatic condensed heteropolycyclic group;

iv) a1 may be 1, Ar₃ and Ar₄ may each independently be a group represented by *-(L₂₁)ₐ₂₁-R₂₁, and one selected from R₁ and R₂ may be selected from a substituted or unsubstituted C₆-C₆₀ aryl group, a substituted or unsubstituted C₁-C₆₀ heteroaryl group, a substituted or unsubstituted monovalent non-aromatic condensed polycyclic group, and a substituted or unsubstituted monovalent non-aromatic condensed heteropolycyclic group;

v) a1 may be 1, Ar₃ and Ar₄ may each independently be selected from a group represented by *-(L₂₁)ₐ₂₁-R₂₁, and one selected from R₁₄ and R₁₅ may be selected from a substituted or unsubstituted C₆-C₆₀ aryl group, a substituted or unsubstituted C₁-C₆₀ heteroaryl group, a substituted or unsubstituted monovalent non-aromatic condensed polycyclic group, and a substituted or unsubstituted monovalent non-aromatic condensed heteropolycyclic group;

vi) a1 may be 1, Ar₃ and Ar₄ may each independently be selected from a group represented by *-(L₂₁)ₐ₂₁-R₂₁, and one selected from R₁ and R₂ and one selected from R₁₄ and R₁₅ may each independently be selected from a substituted or unsubstituted C₆-C₆₀ aryl group, a substituted or unsubstituted C₁-C₆₀ heteroaryl group, a substituted or unsubstituted monovalent non-aromatic condensed polycyclic group, and a substituted or unsubstituted monovalent non-aromatic condensed heteropolycyclic group;

vii) a1 may be 1, Ar₁, Ar₃, and Ar₄ may each independently be selected from a group represented by *-(L₂₁)ₐ₂₁-R₂₁, and one selected from R₁ and R₂ may be selected from a substituted or unsubstituted C₆-C₆₀ aryl group, a substituted or unsubstituted C₁-C₆₀ heteroaryl group, a substituted or unsubstituted monovalent non-aromatic condensed polycyclic group, and a substituted or unsubstituted monovalent non-aromatic condensed heteropolycyclic group; or

viii) a1 may be 1, Ar₁, Ar₃, and Ar₄ may each independently be selected from a group represented by *-(L₂₁)ₐ₂₁-R₂₁, and one selected from R₁₄ and R₁₅ may be selected from a substituted or unsubstituted C₆-C₆₀ aryl group, a substituted or unsubstituted C₁-C₆₀ heteroaryl group, a substituted or unsubstituted monovalent non-aromatic condensed polycyclic group, and a substituted or unsubstituted monovalent non-aromatic condensed heteropolycyclic group.

In an embodiment, in Formula 1, at least one selected from R₁ to R₃ and R₁₄ to R₁₆ may each independently be selected from:

a phenyl group, a pentalenyl group, an indenyl group, a naphthyl group, an anthracenyl group, an azulenyl group, a heptalenyl group, an acenaphthyl group, a phenalenyl group, a fluorenyl group, a spiro-bifluorenyl group, a benzofluorenyl group, a dibenzofluorenyl group, a phenanthrenyl group, a biphenyl group, a terphenyl group, a triphenylenyl group, a fluoranthenyl group, a pyrenyl group, a chrysenyl group, a picenyl group, a perylenyl group, a pentaphenyl group, a pentacenyl group, a tetraphenyl group, a hexaphenyl group, a hexacenyl group, a rubicenyl group, a trinaphthyl group, a heptaphenyl group, a pyranthrenyl group, a pyridinyl group, a pyrazinyl group, a pyridazinyl group, a pyrimidinyl group, a triazinyl group, a quinolinyl group, an isoquinolinyl group, a quinoxalinyl group, a quinazolinyl group, a naphthyridinyl group, an acridinyl group, a phenazinyl group, a benzoquinolinyl group, a benzoisoquinolinyl group, a phenanthridinyl group, a phenanthrolinyl group, a benzoquinonyl group, a cumarinyl group, an anthraquinonyl group, a fluorenonyl group, a furanyl group, a thienyl group, a silolyl group, a benzofuranyl group, a benzothienyl group, a benzosilolyl group, a dibenzofuranyl group, a dibenzothiophenyl group, a dibenzosilolyl group, a pyrrolyl group, an indolyl group, an isoindolyl group, a carbazolyl group, a benzocarbazolyl group, a dibenzocarbazolyl group, an imidazolyl group, a benzimidazolyl group, a pyrazolyl group, a triazolyl group, a tetrazolyl group, an indazolyl group, an oxazolyl group, an isoxazolyl group, a benzoxazolyl group, a benzisoxazolyl group, a thiazolyl group, an isothiazolyl group, a benzothiazolyl group, a benzisothiazolyl group, an imidazopyridinyl group, an imidazopyrimidinyl group, an imidazophenanthridinyl group, a benzimidazophenanthridinyl group, an azadibenzofuranyl group, an azacarbazolyl group, an azadibenzothienyl group, a diazadibenzofuranyl group, a diazacarbazolyl group, a diazadibenzothienyl group, a xanthonyl group, and a thioxanthonyl group; and

a phenyl group, a pentalenyl group, an indenyl group, a naphthyl group, an anthracenyl group, an azulenyl group, a heptalenyl group, an acenaphthyl group, a phenalenyl group, a fluorenyl group, a spiro-bifluorenyl group, a benzofluorenyl group, a dibenzofluorenyl group, a phenanthrenyl group, a biphenyl group, a terphenyl group, a triphenylenyl group, a fluoranthenyl group, a pyrenyl group, a chrysenyl group, a picenyl group, a perylenyl group, a pentaphenyl group, a pentacenyl group, a tetraphenyl group, a hexaphenyl group, a hexacenyl group, a rubicenyl group, a trinaphthyl group, a heptaphenyl group, a pyranthrenyl group, a pyridinyl group, a pyrazinyl group, a pyridazinyl group, a pyrimidinyl group, a triazinyl group, a quinolinyl group, an isoquinolinyl group, a quinoxalinyl group, a quinazolinyl group, a naphthyridinyl group, an acridinyl group, a phenazinyl group, a benzoquinolinyl group, a benzoisoquinolinyl group, a phenanthridinyl group, a phenanthrolinyl group, a benzoquinonyl group, a cumarinyl group, an anthraquinonyl group, a fluorenonyl group, a furanyl group, a thienyl group, a silolyl group, a benzofuranyl group, a benzothienyl group, a benzosilolyl group, a dibenzofuranyl group, a dibenzothiophenyl group, a dibenzosilolyl group, a pyrrolyl group, an indolyl group, an isoindolyl group, a carbazolyl group, a benzocarbazolyl group, a dibenzocarbazolyl group, an imidazolyl group, a benzimidazolyl group, a pyrazolyl group, a triazolyl group, a tetrazolyl group, an indazolyl group, an oxazolyl group, an isoxazolyl group, a benzoxazolyl group, a benzisoxazolyl group, a thiazolyl group, an isothiazolyl group, a benzothiazolyl group, a benzisothiazolyl group, an imidazopyridinyl group, an imidazopyrimidinyl group, an imidazophenanthridinyl group, a benzimidazophenanthridinyl group, an azadibenzofuranyl group, an azacarbazolyl group, an azadibenzothienyl group, a diazadibenzofuranyl group, a diazacarbazolyl group, a diazadibenzothienyl group, a xanthonyl group, and a thioxanthonyl group, each substituted with at least one selected from deuterium, —F, —Cl, —Br, —I, a hydroxyl group, a cyano group, a nitro group, an amidino group, a hydrazine group, a hydrazone group, a C₁-C₃₀ alkyl group, a C₁-C₃₀ alkoxy group, a phenyl group, a pentalenyl group, an indenyl group, a naphthyl group, an anthracenyl group, an azulenyl group, a heptalenyl group, an acenaphthyl group, a phenalenyl group, a fluorenyl group, a spiro-bifluorenyl group, a benzofluorenyl group, a dibenzofluorenyl group, a phenanthrenyl group, a biphenyl group, a terphenyl group, a triphenylenyl group, a fluoranthenyl group, a pyrenyl group, a chrysenyl group, a picenyl group, a perylenyl group, a pentaphenyl group, a pentacenyl group, a tetraphenyl group, a hexaphenyl group, a hexacenyl group, a rubicenyl group, a trinaphthyl group, a heptaphenyl group, a pyranthrenyl group, a pyridinyl group, a pyrazinyl group, a pyridazinyl group, a pyrimidinyl group, a triazinyl group, a quinolinyl group, an isoquinolinyl group, a quinoxalinyl group, a quinazolinyl group, a naphthyridinyl group, an acridinyl group, a phenazinyl group, a benzoquinolinyl group, a benzoisoquinolinyl group, a phenanthridinyl group, a phenanthrolinyl group, a benzoquinonyl group, a cumarinyl group, an anthraquinonyl group, a fluorenonyl group, a furanyl group, a thienyl group, a silolyl group, a benzofuranyl group, a benzothienyl group, a benzosilolyl group, a dibenzofuranyl group, a dibenzothiophenyl group, a dibenzosilolyl group, a pyrrolyl group, an indolyl group, an isoindolyl group, a carbazolyl group, a benzocarbazolyl group, a dibenzocarbazolyl group, an imidazolyl group, a benzimidazolyl group, a pyrazolyl group, a triazolyl group, a tetrazolyl group, an indazolyl group, an oxazolyl group, an isoxazolyl group, a benzoxazolyl group, a benzisoxazolyl group, a thiazolyl group, an isothiazolyl group, a benzothiazolyl group, a benzisothiazolyl group, an imidazopyridinyl group, an imidazopyrimidinyl group, an imidazophenanthridinyl group, a benzimidazophenanthridinyl group, an azadibenzofuranyl group, an azacarbazolyl group, an azadibenzothienyl group, a diazadibenzofuranyl group, a diazacarbazolyl group, a diazadibenzothienyl group, a xanthonyl group, and a thioxanthonyl group.

In an embodiment, at least one selected from R₆ and R₁₁ in Formula 1 may each independently be selected from a substituted or unsubstituted C₆-C₆₀ aryl group, a substituted or unsubstituted C₁-C₆₀ heteroaryl group, a substituted or unsubstituted monovalent non-aromatic condensed polycyclic group, and a substituted or unsubstituted monovalent non-aromatic condensed heteropolycyclic group.

In one or more embodiments, at least one selected from R₆ and R₁₁ in Formula 1 may each independently be selected from a substituted or unsubstituted C₆-C₆₀ aryl group, a substituted or unsubstituted heteroaryl group, a substituted or unsubstituted monovalent non-aromatic condensed polycyclic group, and a substituted or unsubstituted monovalent non-aromatic condensed heteropolycyclic group, wherein R₄, R₅, R₇ to R₉, R₁₀, R₁₂, R₁₃, R₁₇, and R₁₈ may each independently be hydrogen.

In one or more embodiments, the condensed cyclic compound may be represented by Formula 1A:

Formula 1A

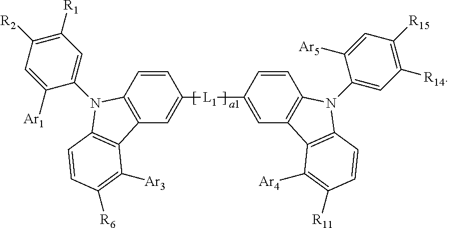

In Formula 1A,

i) at least one selected from Ar₁ and Ar₅ may each independently be a group represented by *-(L₂₁)ₐ₂₁-R₂₁, and one selected from Ar₃ and Ar₄ may be a group represented by *-(L₂₁)ₐ₂₁-R₂₁, or

ii) Ar₃ and Ar₄ may each independently be a group represented by *-(L₂₁)ₐ₂₁-R₂₁,

when a1 is 0, one selected from Ar₃ and Ar₄ may be selected from hydrogen and deuterium, and

L₁, a1, Ar₁, Ar₃ to Ar₅, R₁, R₂, R₆, R₁₁, R₁₄, and R₁₅ may each independently be the same as defined in Formula 1.

In an embodiment, the condensed cyclic compound may be represented by Formula 1-1 or 1-2:

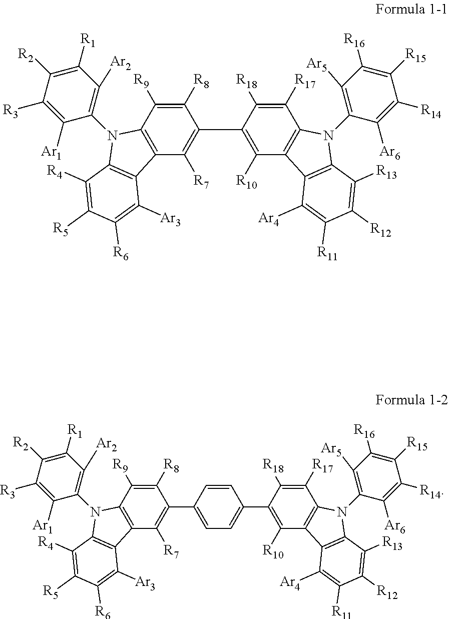

In Formula 1-1, one selected from Ar₃ and Ar₄ may be selected from hydrogen and deuterium, and

Ar₁ to Ar₆ and R₁ to R₁₈ in Formulae 1-1 and 1-2 may each independently be the same as defined in Formula 1.

In an embodiment, in Formulae 1-1 and 1-2, at least one selected from R₁ to R₃ and R₁₄ to R₁₆ may each independently be selected from a substituted or unsubstituted C₆-C₆₀ aryl group, a substituted or unsubstituted C₁-C₆₀ heteroaryl group, a substituted or unsubstituted monovalent non-aromatic condensed polycyclic group, and a substituted or unsubstituted monovalent non-aromatic condensed heteropolycyclic group.

In one or more embodiments, at least one selected from R₆ and R₁₁ in Formulae 1-1 and 1-2 may each independently be selected from a substituted or unsubstituted C₆-C₆₀ aryl group, a substituted or unsubstituted C₁-C₆₀ heteroaryl group, a substituted or unsubstituted monovalent non-aromatic condensed polycyclic group, and a substituted or unsubstituted monovalent non-aromatic condensed heteropolycyclic group.

In one or more embodiments, at least one selected from R₆ and R₁₁ in Formulae 1-1 and 1-2 may each independently be selected from a substituted or unsubstituted C₆-C₆₀ aryl group, a substituted or unsubstituted C₁-C₆₀ heteroaryl group, a substituted or unsubstituted monovalent non-aromatic condensed polycyclic group, and a substituted or unsubstituted monovalent non-aromatic condensed heteropolycyclic group, wherein R₄, R₅, R₇ to R₉, R₁₀, R₁₂, R₁₃, R₁₇, and R₁₈ may each independently be hydrogen.

In an embodiment, the condensed cyclic compound may be selected from Compounds 1 to 154, but embodiments of the present disclosure are not limited thereto:

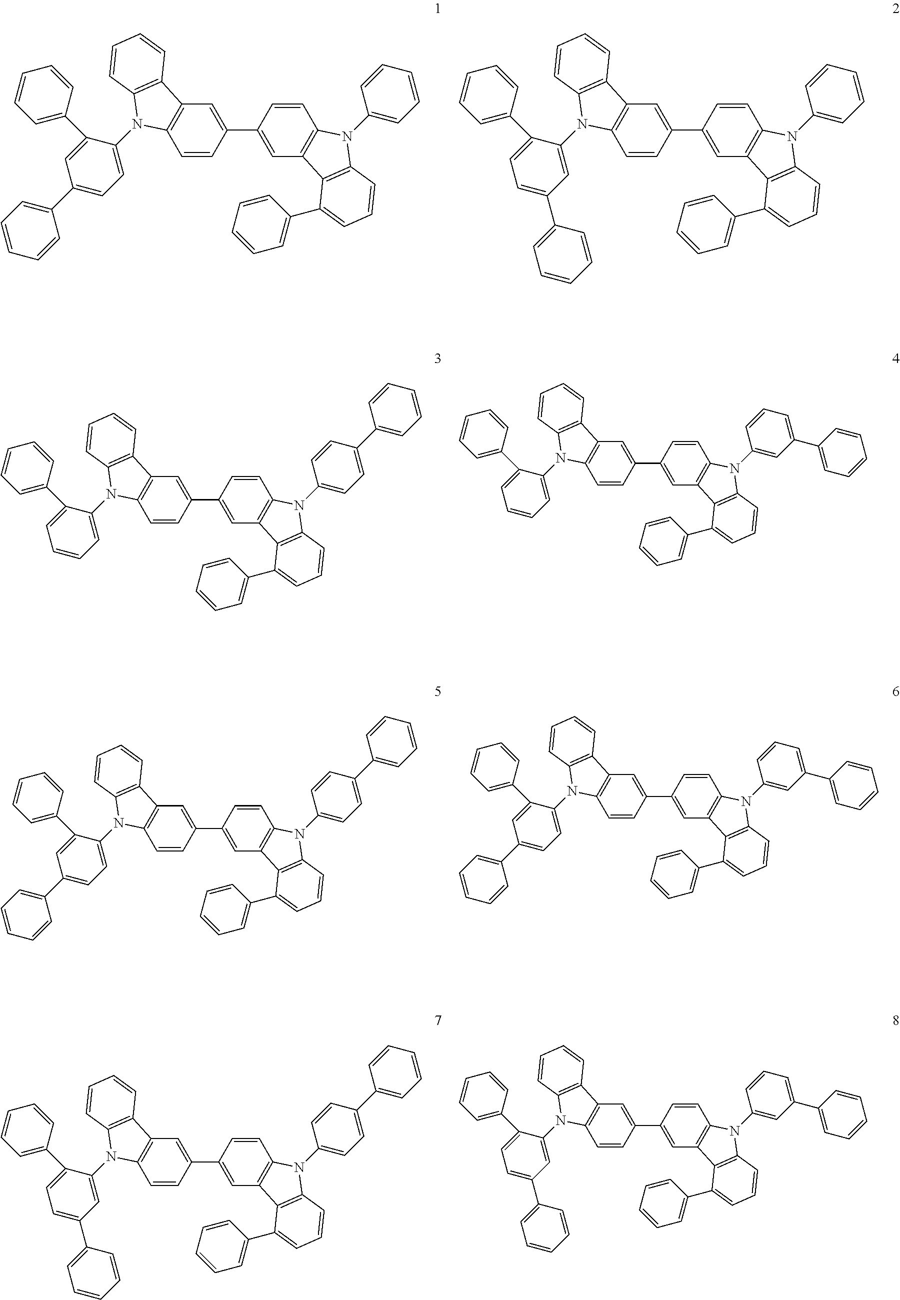

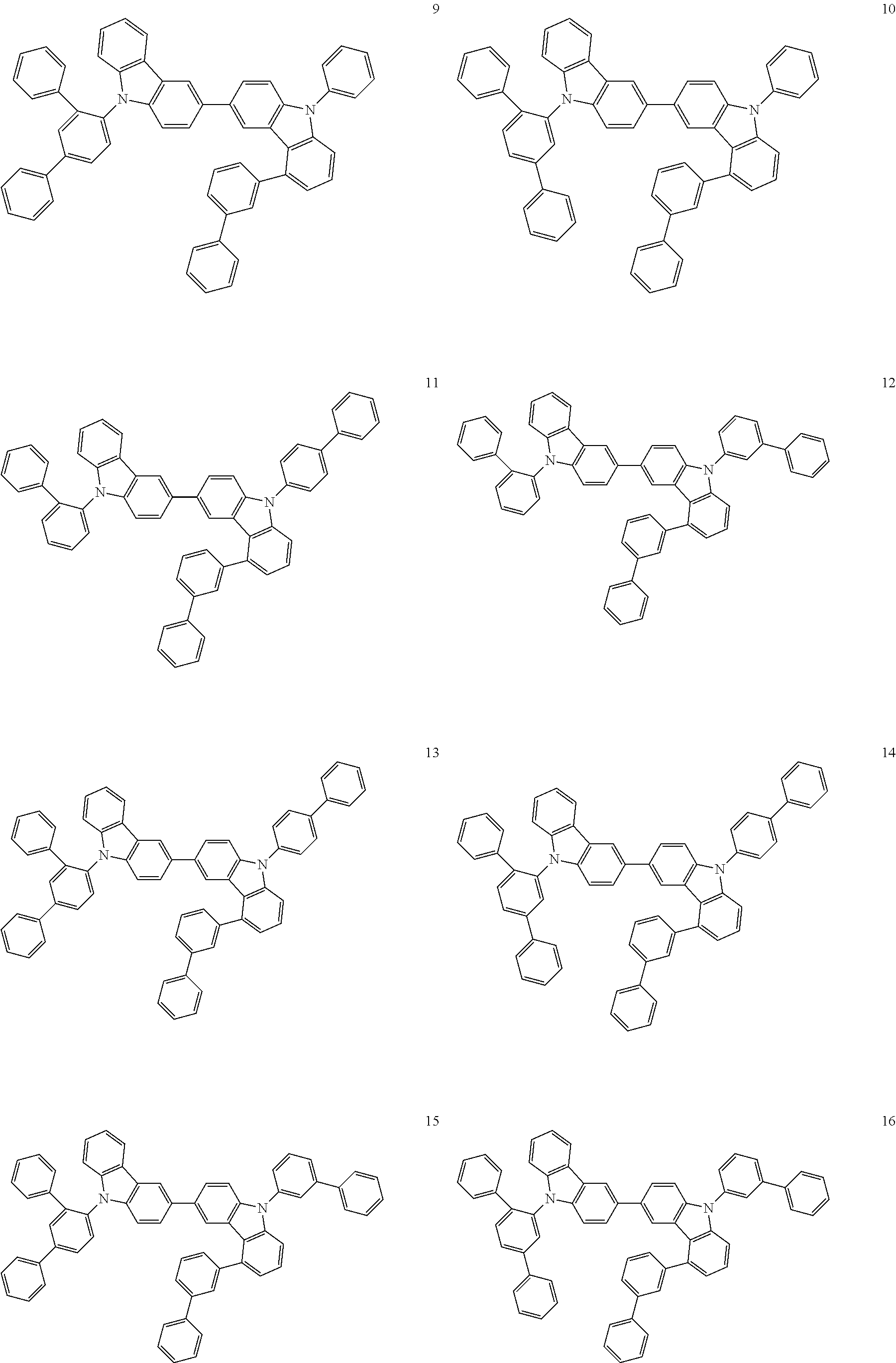

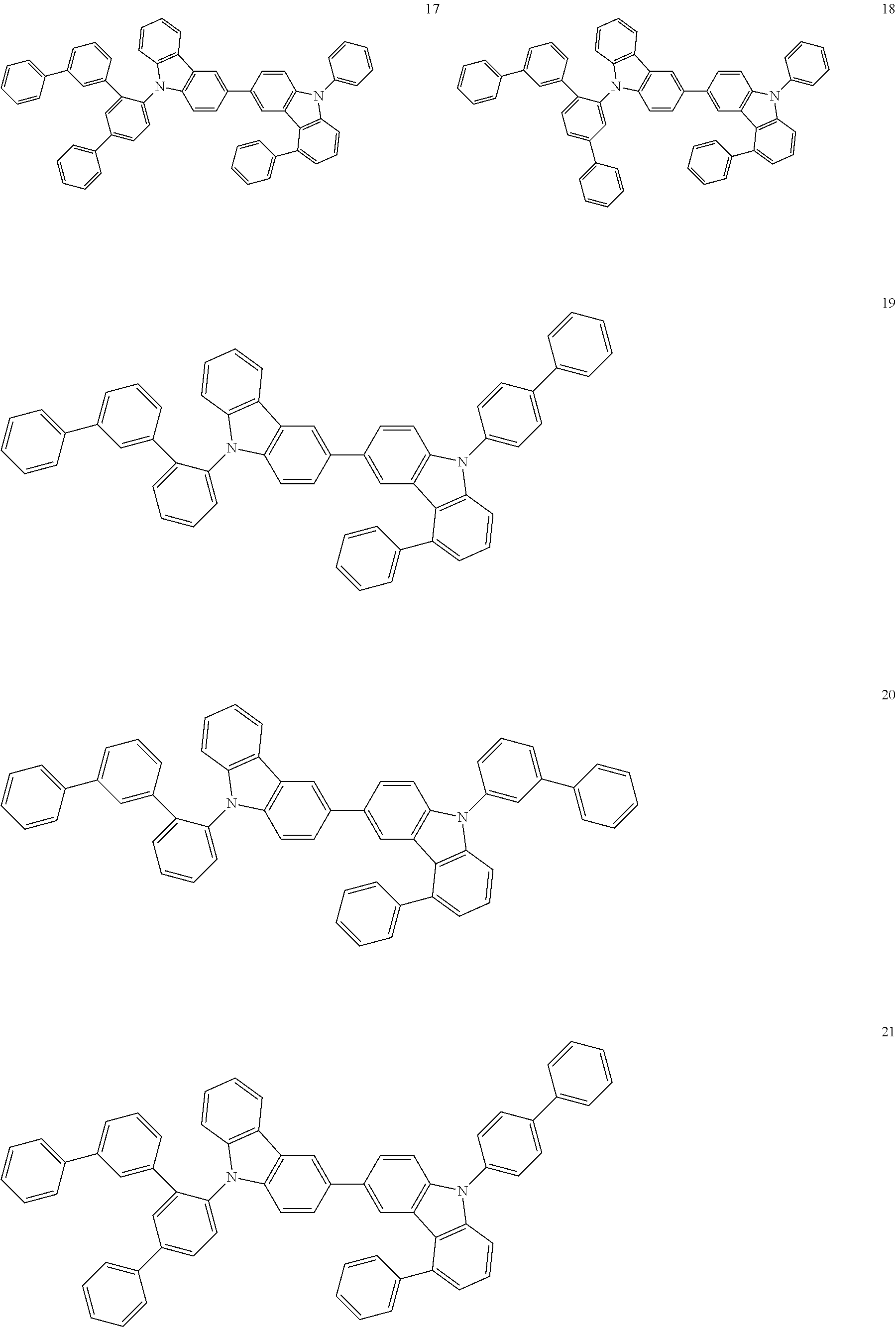

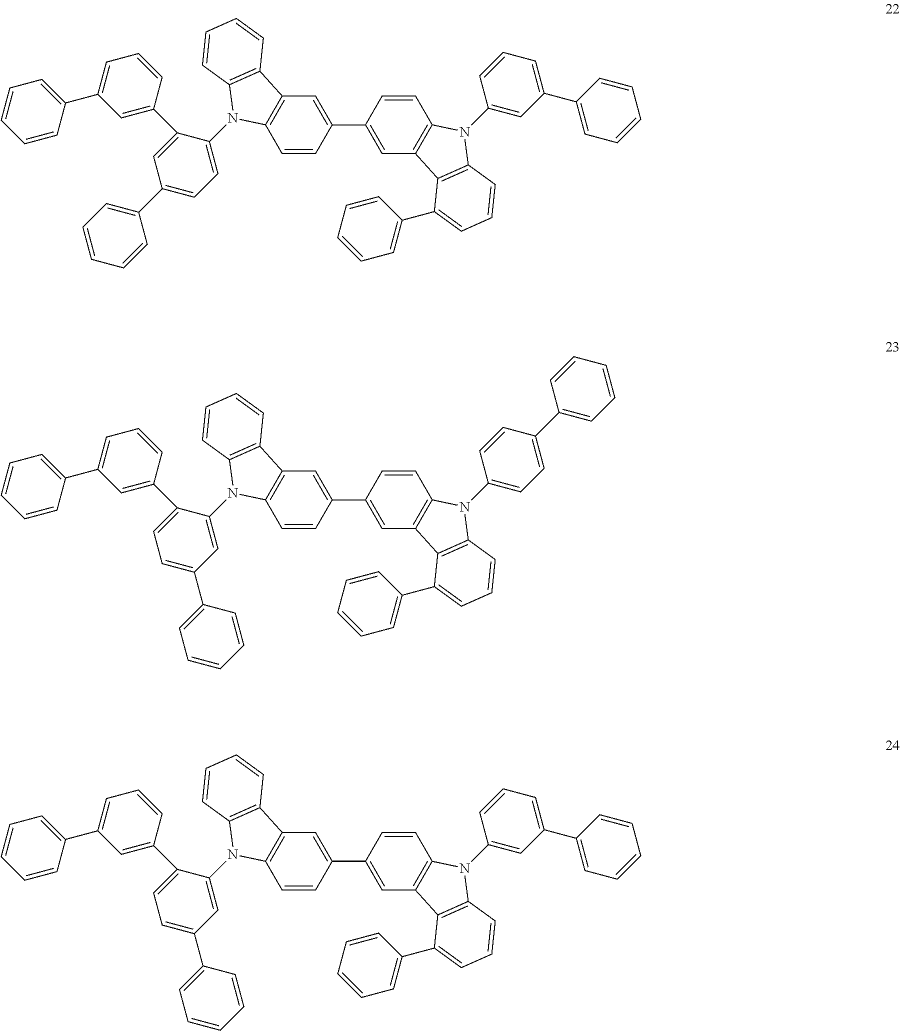 [SMILES: c1ccc(-c2cccc(-c3cc(-c4ccccc4)ccc3-n3c4ccccc4c4cc(-c5ccc6c(c5)c5c(-c7ccccc7)cccc5n6-c5cccc(-c6ccccc6)c5)ccc43)c2)cc1 ; c1ccc(-c2ccc(-n3c4ccc(-c5ccc6c(c5)c5ccccc5n6-c5cc(-c6ccccc6)ccc5-c5cccc(-c6ccccc6)c5)cc4c4c(-c5ccccc5)cccc43)cc2)cc1 ; c1ccc(-c2cccc(-c3ccc(-c4ccccc4)cc3-n3c4ccccc4c4cc(-c5ccc6c(c5)c5c(-c7ccccc7)cccc5n6-c5cccc(-c6ccccc6)c5)ccc43)c2)cc1]

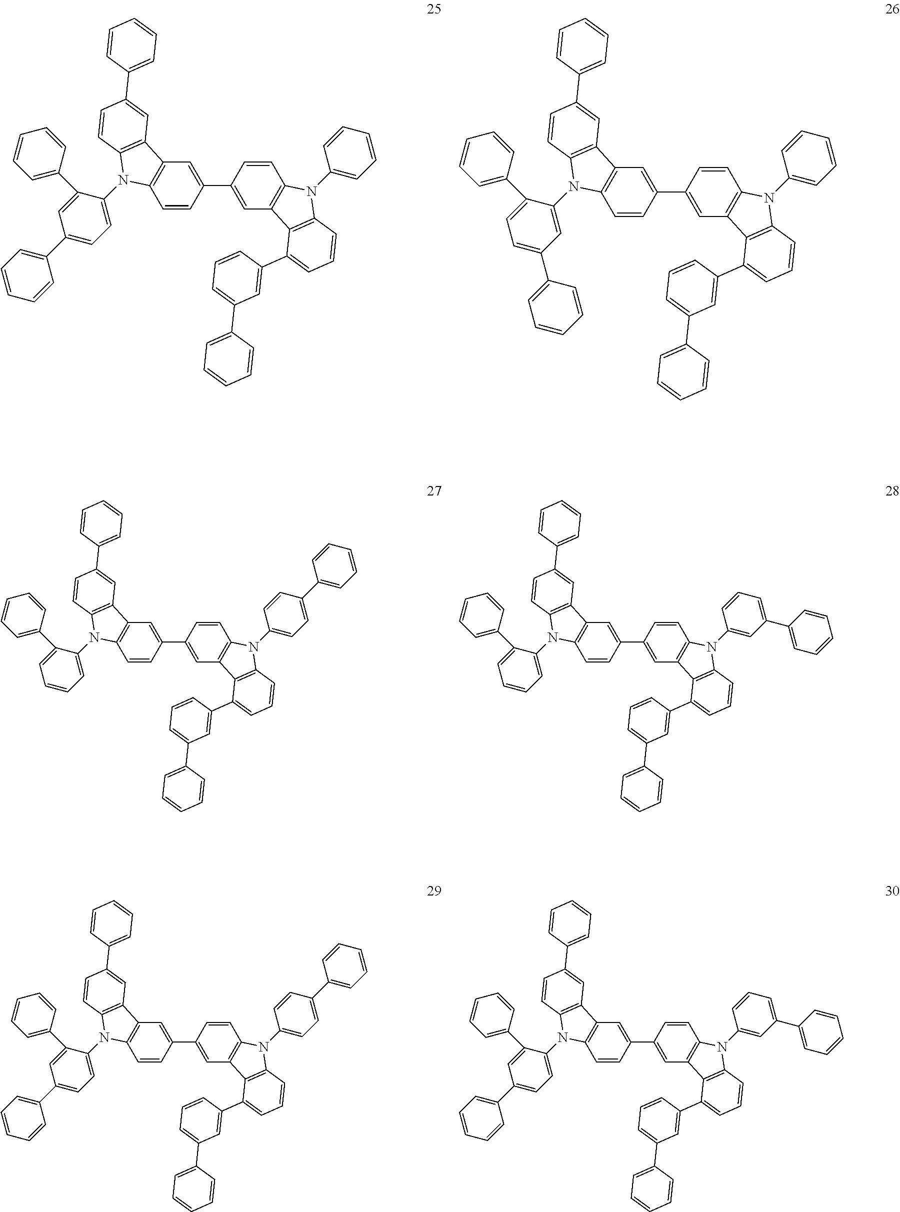

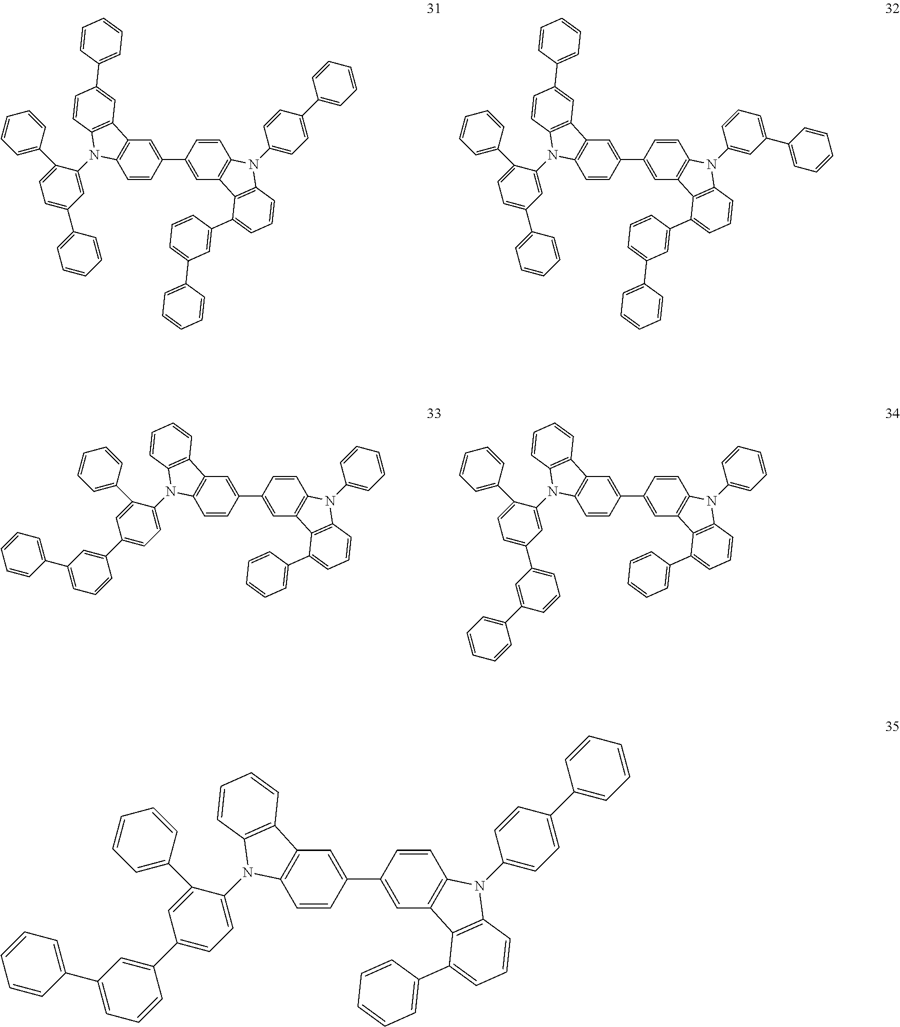

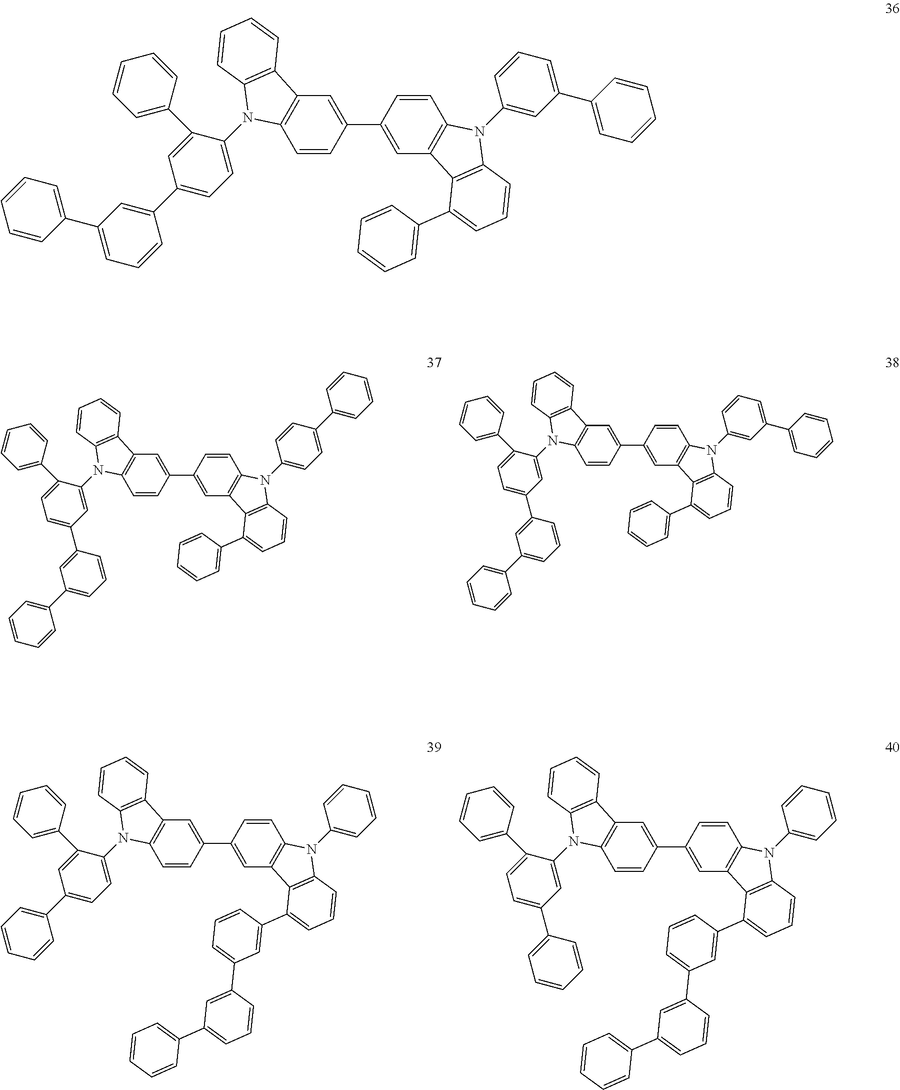

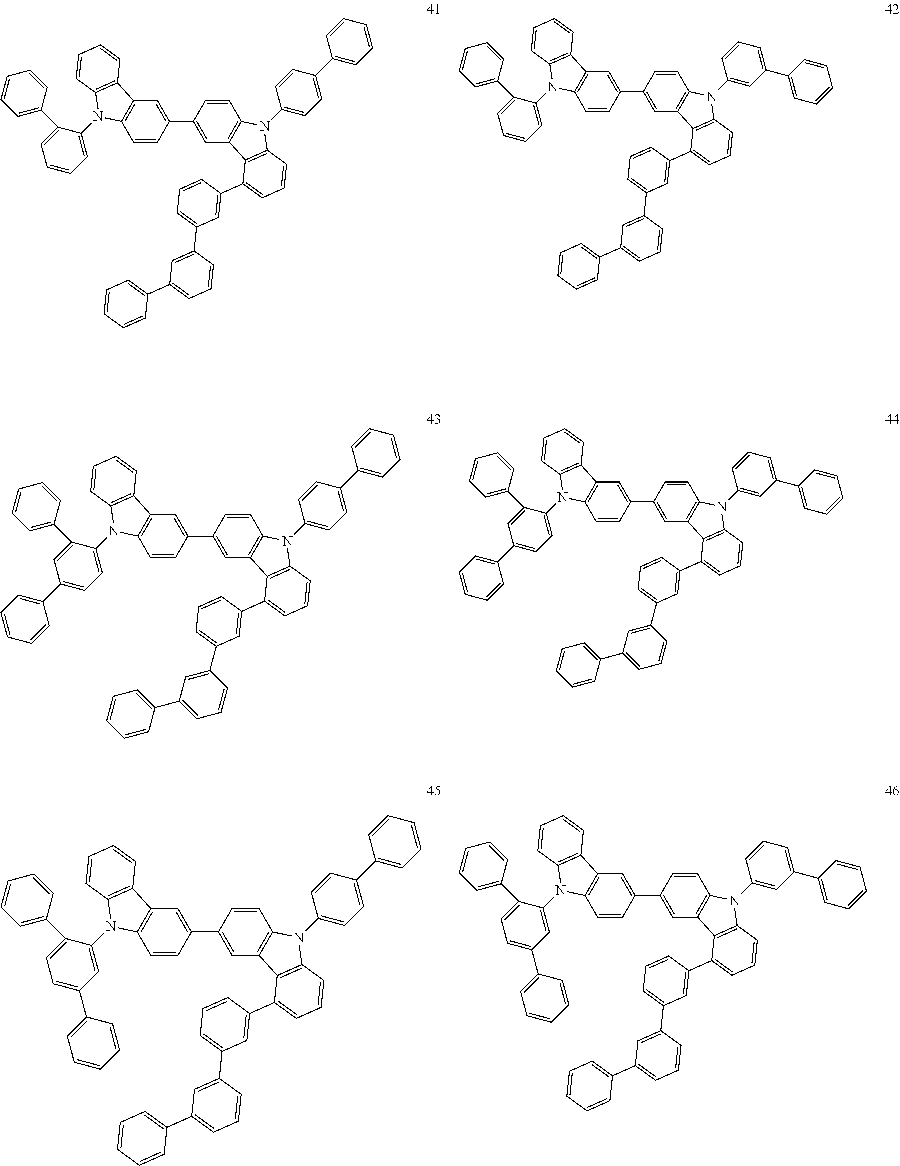

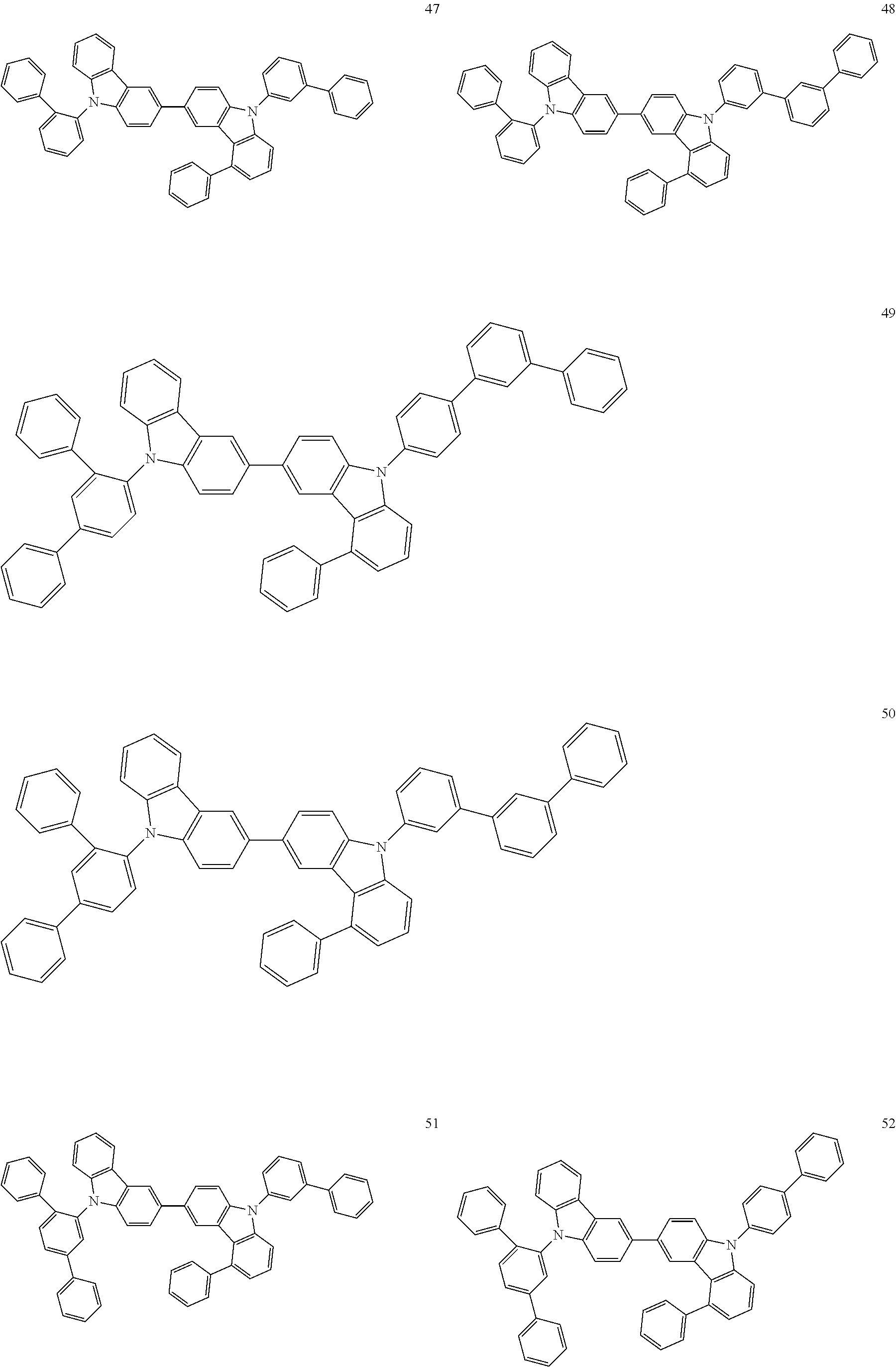

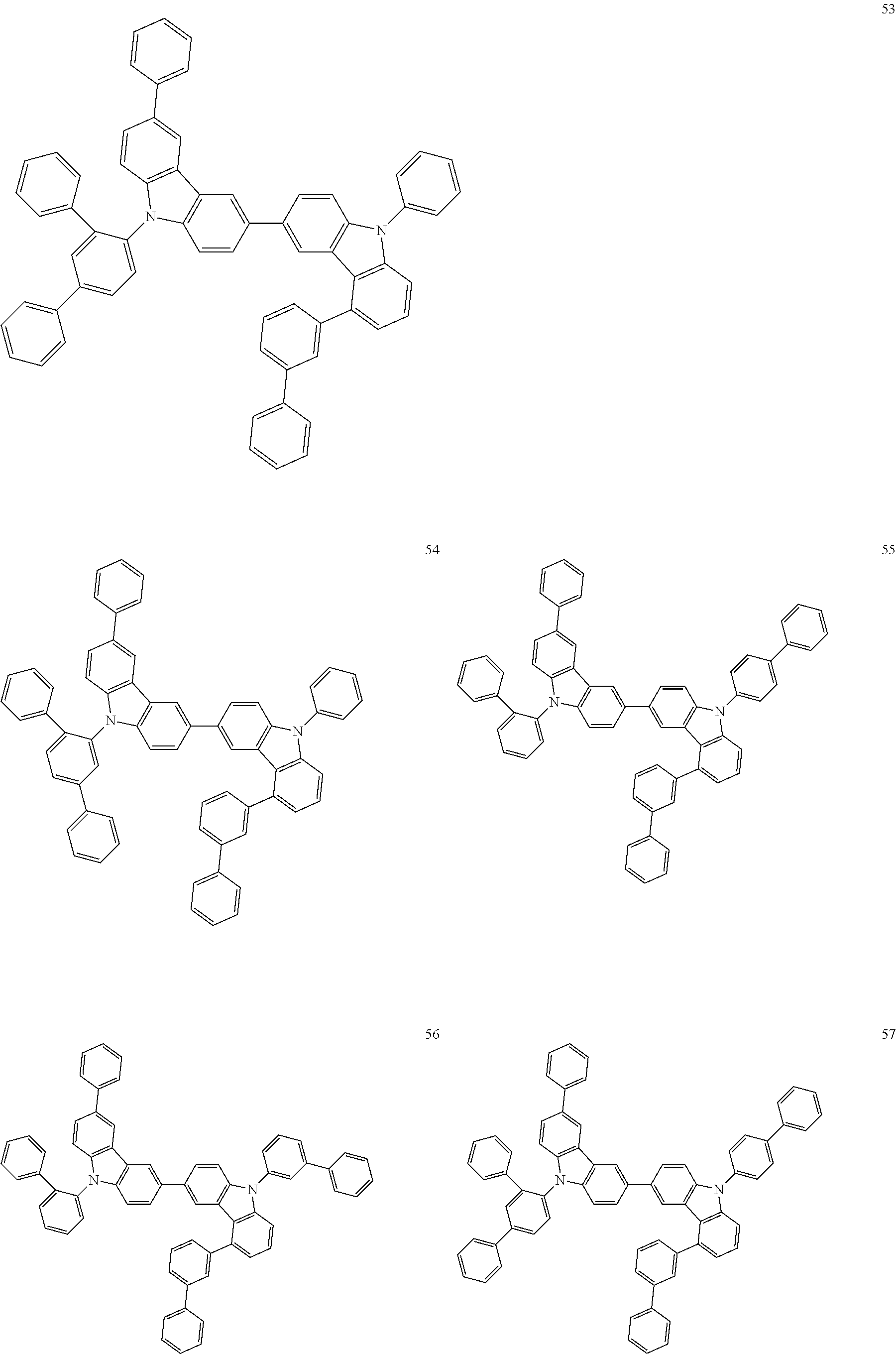

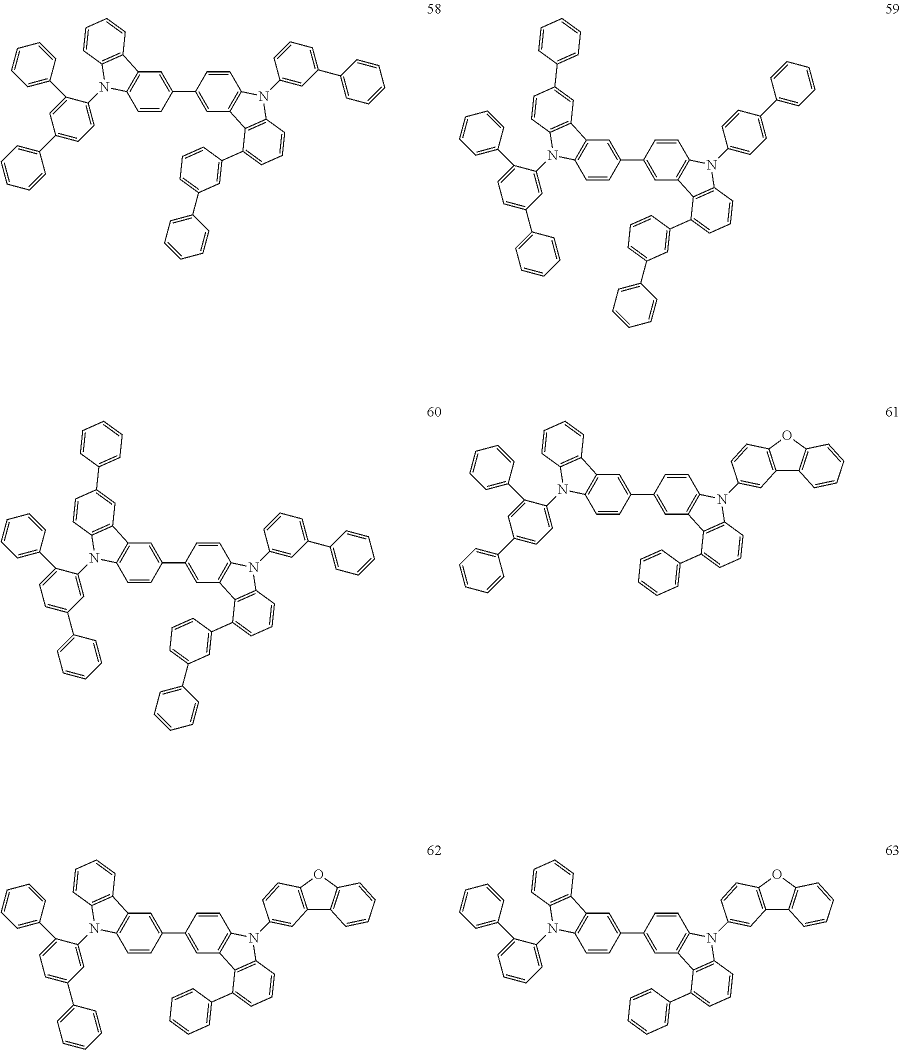

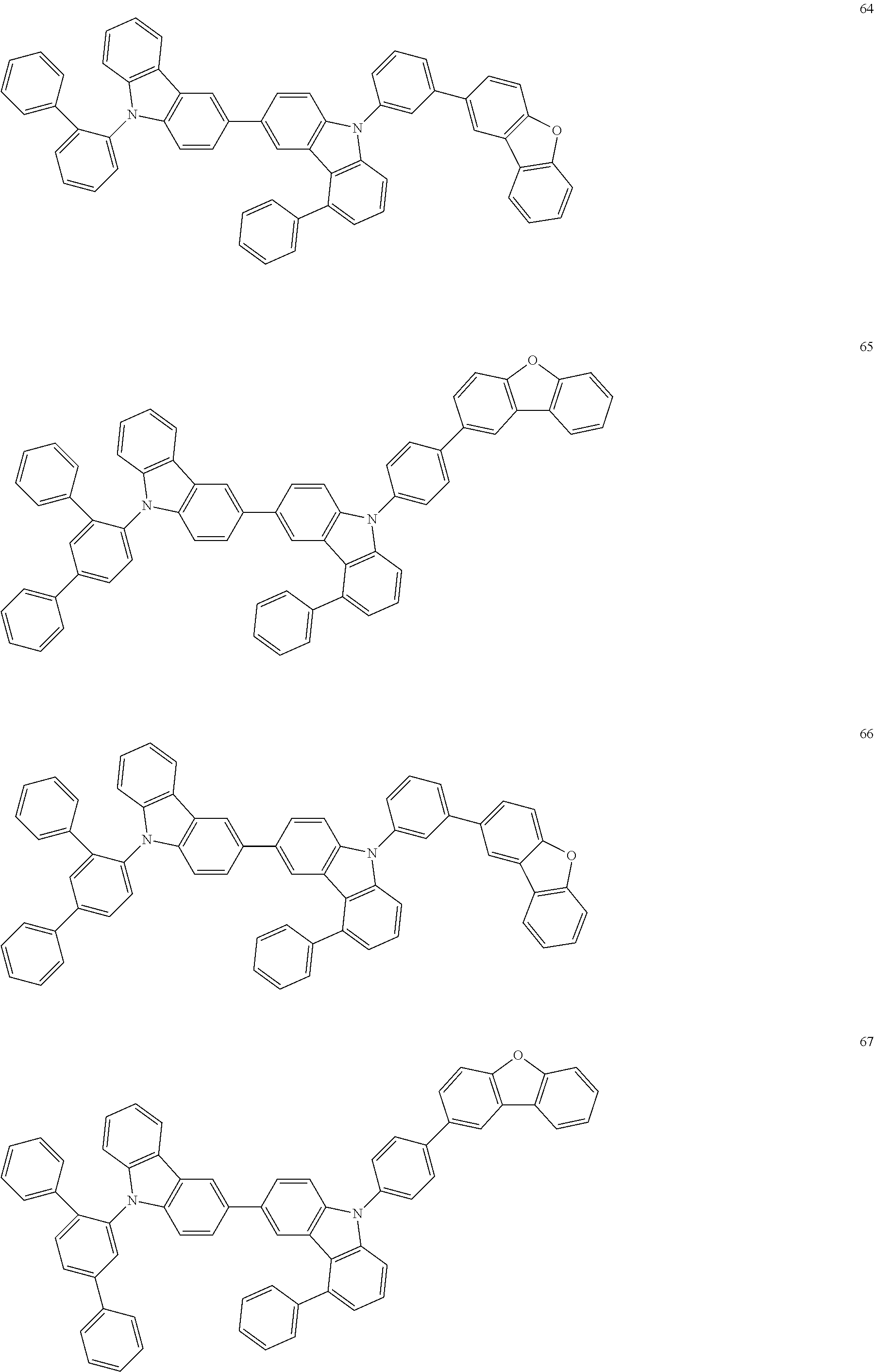 [SMILES: c1ccc(-c2ccccc2-n2c3ccccc3c3cc(-c4ccc5c(c4)c4c(-c6ccccc6)cccc4n5-c4cccc(-c5ccc6oc7ccccc7c6c5)c4)ccc32)cc1 ; c1ccc(-c2ccc(-n3c4ccccc4c4cc(-c5ccc6c(c5)c5c(-c7ccccc7)cccc5n6-c5ccc(-c6ccc7oc8ccccc8c7c6)cc5)ccc43)c(-c3ccccc3)c2)cc1 ; c1ccc(-c2ccc(-n3c4ccccc4c4cc(-c5ccc6c(c5)c5c(-c7ccccc7)cccc5n6-c5cccc(-c6ccc7oc8ccccc8c7c6)c5)ccc43)c(-c3ccccc3)c2)cc1 ; c1ccc(-c2ccc(-c3ccccc3)c(-n3c4ccccc4c4cc(-c5ccc6c(c5)c5c(-c7ccccc7)cccc5n6-c5ccc(-c6ccc7oc8ccccc8c7c6)cc5)ccc43)c2)cc1]

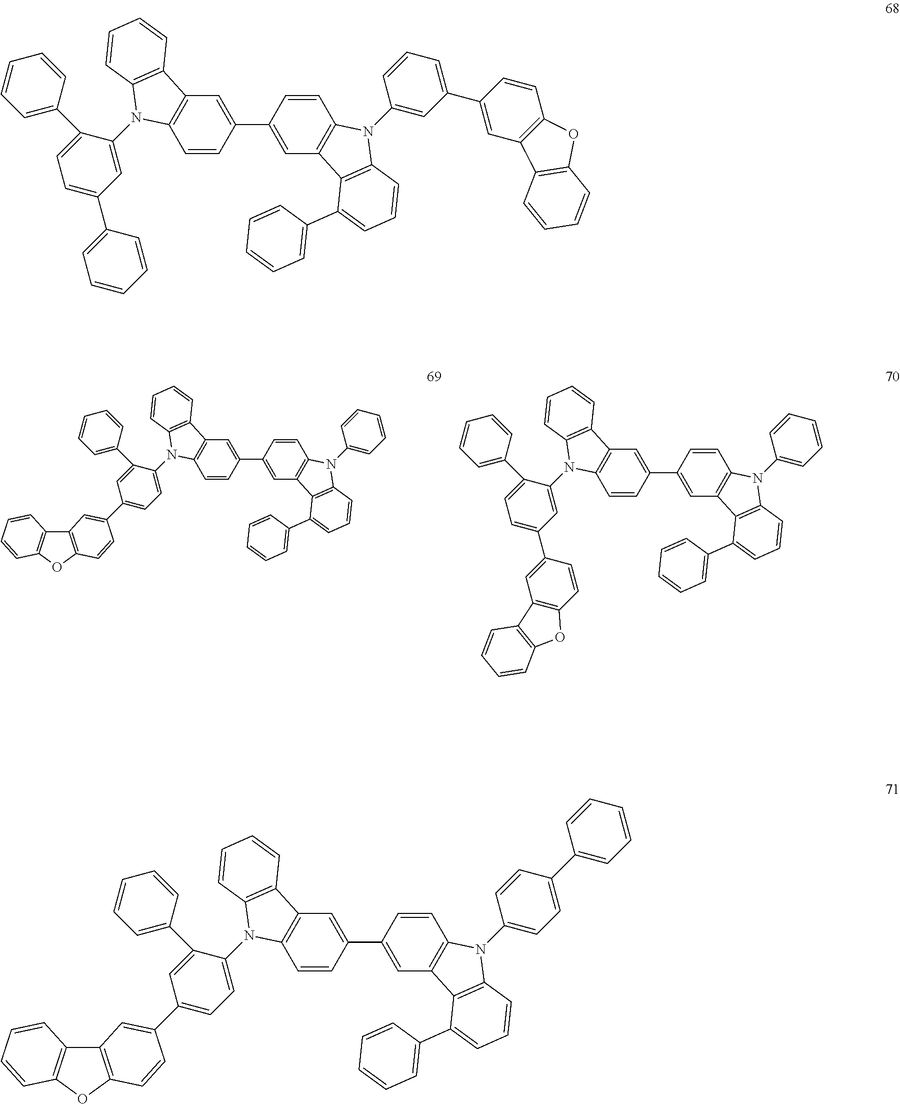 [SMILES: c1ccc(-c2ccc(-c3ccccc3)c(-n3c4ccccc4c4cc(-c5ccc6c(c5)c5c(-c7ccccc7)cccc5n6-c5cccc(-c6ccc7oc8ccccc8c7c6)c5)ccc43)c2)cc1 ; c1ccc(-c2cc(-c3ccc4oc5ccccc5c4c3)ccc2-n2c3ccccc3c3cc(-c4ccc5c(c4)c4c(-c6ccccc6)cccc4n5-c4ccccc4)ccc32)cc1 ; c1ccc(-c2ccc(-c3ccc4oc5ccccc5c4c3)cc2-n2c3ccccc3c3cc(-c4ccc5c(c4)c4c(-c6ccccc6)cccc4n5-c4ccccc4)ccc32)cc1 ; c1ccc(-c2ccc(-n3c4ccc(-c5ccc6c(c5)c5ccccc5n6-c5ccc(-c6ccc7oc8ccccc8c7c6)cc5-c5ccccc5)cc4c4c(-c5ccccc5)cccc43)cc2)cc1]

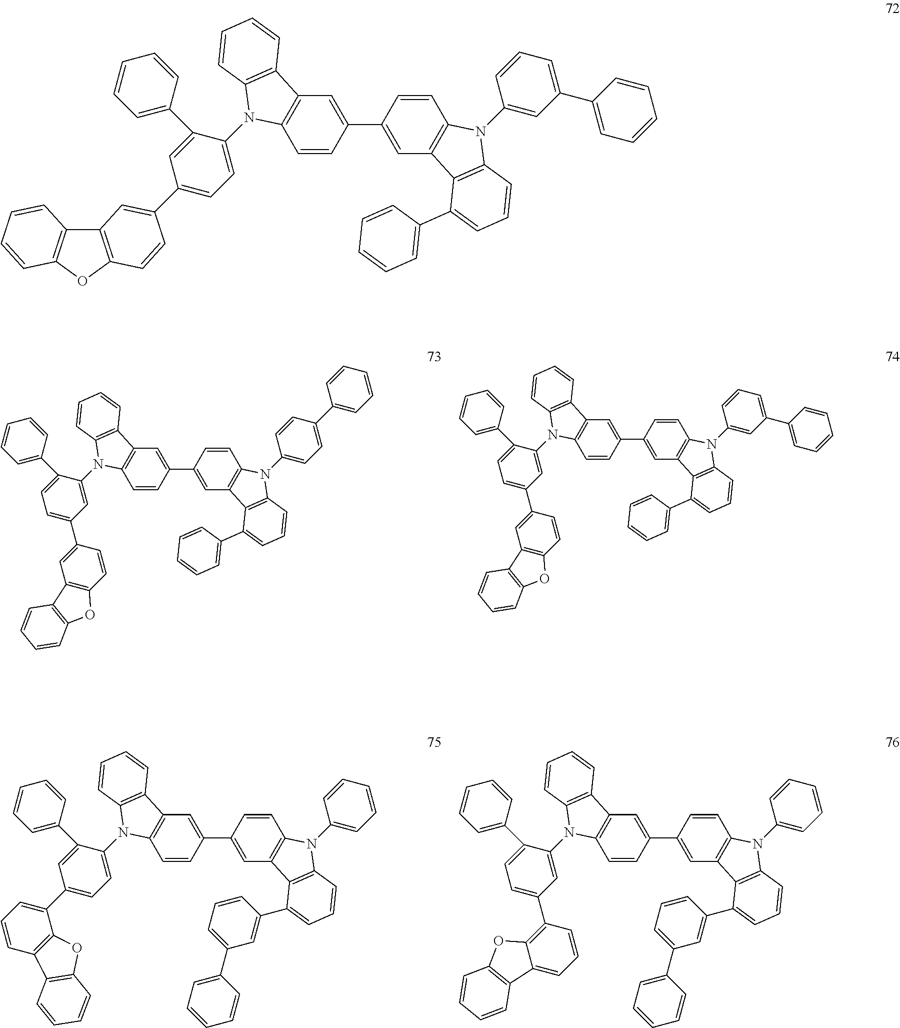

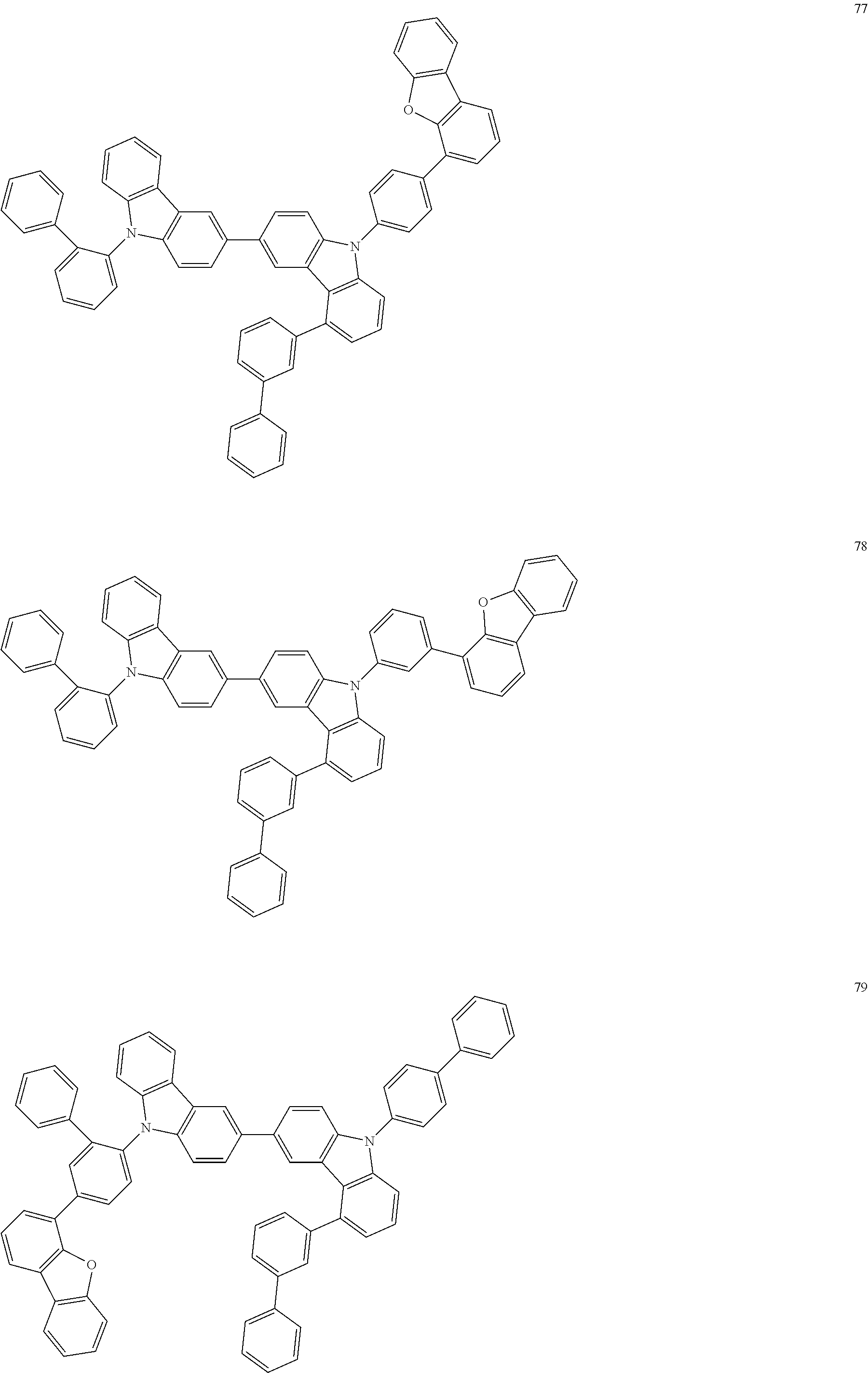 [SMILES: c1ccc(-c2cccc(-c3cccc4c3c3cc(-c5ccc6c(c5)c5ccccc5n6-c5ccccc5-c5ccccc5)ccc3n4-c3ccc(-c4cccc5c4oc4ccccc45)cc3)c2)cc1 ; c1ccc(-c2cccc(-c3cccc4c3c3cc(-c5ccc6c(c5)c5ccccc5n6-c5ccccc5-c5ccccc5)ccc3n4-c3cccc(-c4cccc5c4oc4ccccc45)c3)c2)cc1 ; c1ccc(-c2ccc(-n3c4ccc(-c5ccc6c(c5)c5ccccc5n6-c5ccc(-c6cccc7c6oc6ccccc67)cc5-c5ccccc5)cc4c4c(-c5cccc(-c6ccccc6)c5)cccc43)cc2)cc1]

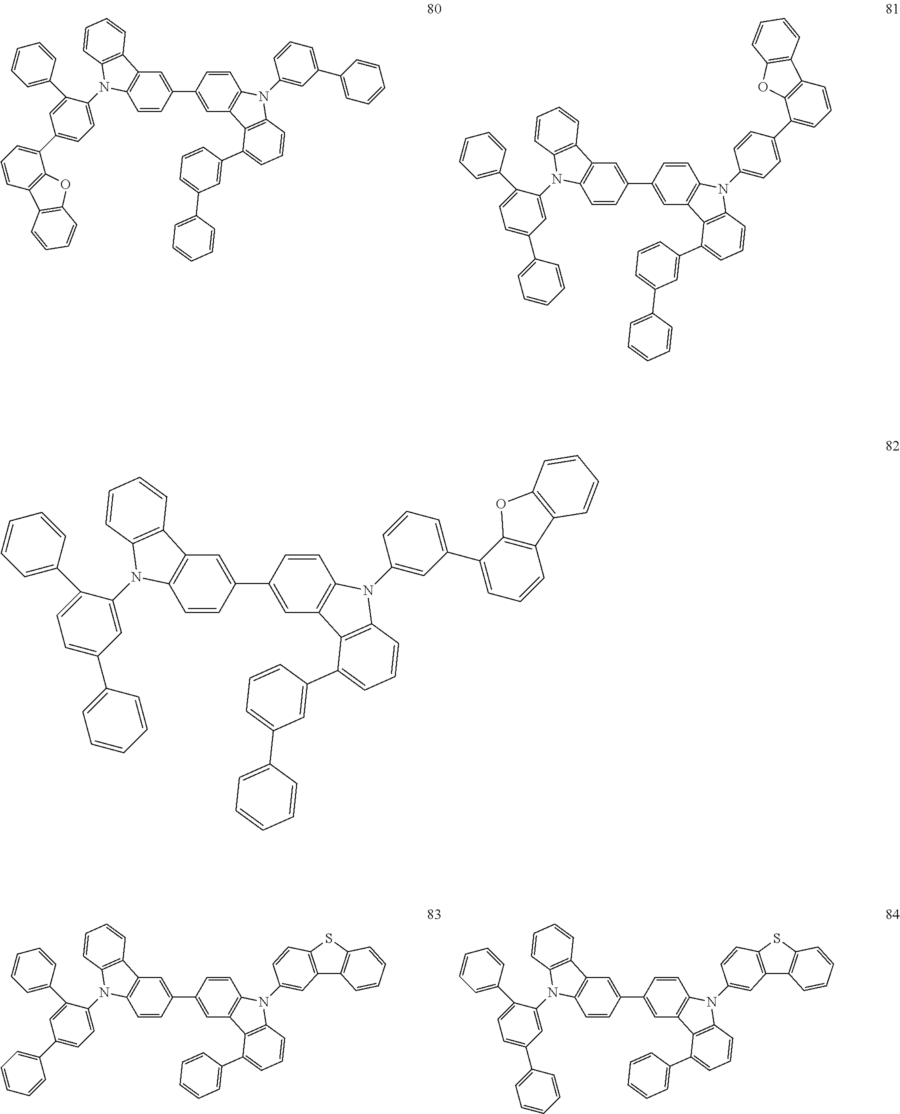

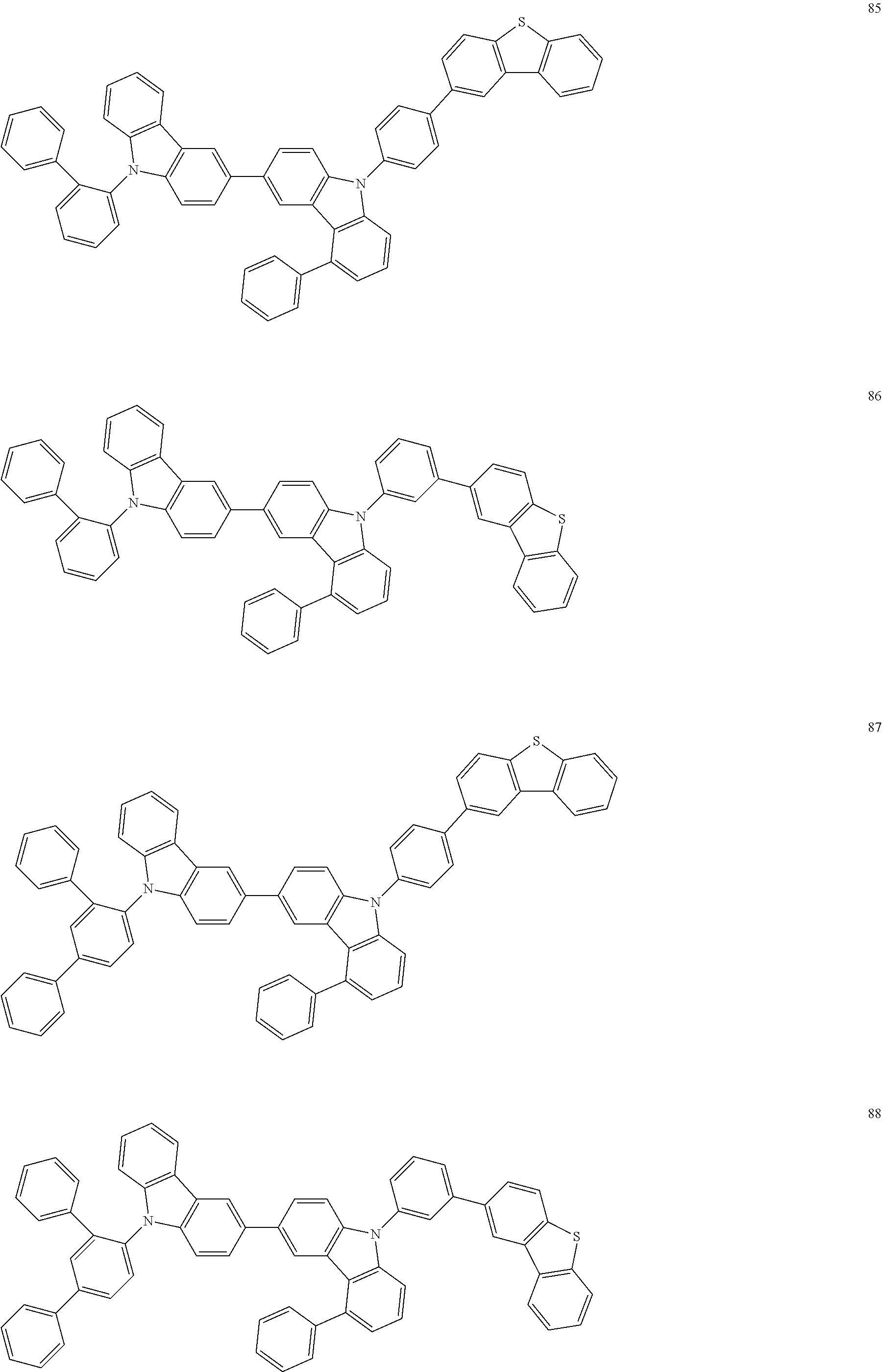 [SMILES: c1ccc(-c2ccccc2-n2c3ccccc3c3cc(-c4ccc5c(c4)c4c(-c6ccccc6)cccc4n5-c4ccc(-c5ccc6sc7ccccc7c6c5)cc4)ccc32)cc1 ; c1ccc(-c2ccccc2-n2c3ccccc3c3cc(-c4ccc5c(c4)c4c(-c6ccccc6)cccc4n5-c4cccc(-c5ccc6sc7ccccc7c6c5)c4)ccc32)cc1 ; c1ccc(-c2ccc(-n3c4ccccc4c4cc(-c5ccc6c(c5)c5c(-c7ccccc7)cccc5n6-c5ccc(-c6ccc7sc8ccccc8c7c6)cc5)ccc43)c(-c3ccccc3)c2)cc1 ; c1ccc(-c2ccc(-n3c4ccccc4c4cc(-c5ccc6c(c5)c5c(-c7ccccc7)cccc5n6-c5cccc(-c6ccc7sc8ccccc8c7c6)c5)ccc43)c(-c3ccccc3)c2)cc1]

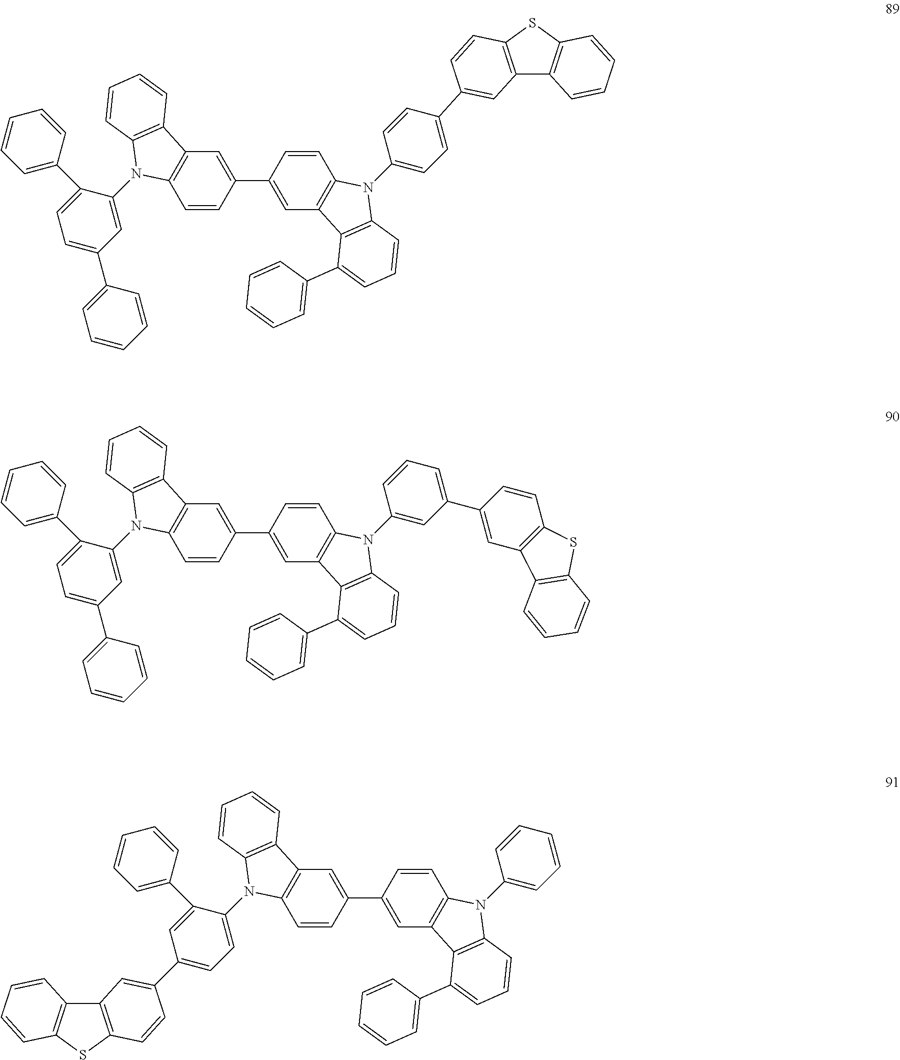 [SMILES: c1ccc(-c2ccc(-c3ccccc3)c(-n3c4ccccc4c4cc(-c5ccc6c(c5)c5c(-c7ccccc7)cccc5n6-c5ccc(-c6ccc7sc8ccccc8c7c6)cc5)ccc43)c2)cc1 ; c1ccc(-c2ccc(-c3ccccc3)c(-n3c4ccccc4c4cc(-c5ccc6c(c5)c5c(-c7ccccc7)cccc5n6-c5cccc(-c6ccc7sc8ccccc8c7c6)c5)ccc43)c2)cc1 ; c1ccc(-c2cc(-c3ccc4sc5ccccc5c4c3)ccc2-n2c3ccccc3c3cc(-c4ccc5c(c4)c4c(-c6ccccc6)cccc4n5-c4ccccc4)ccc32)cc1]

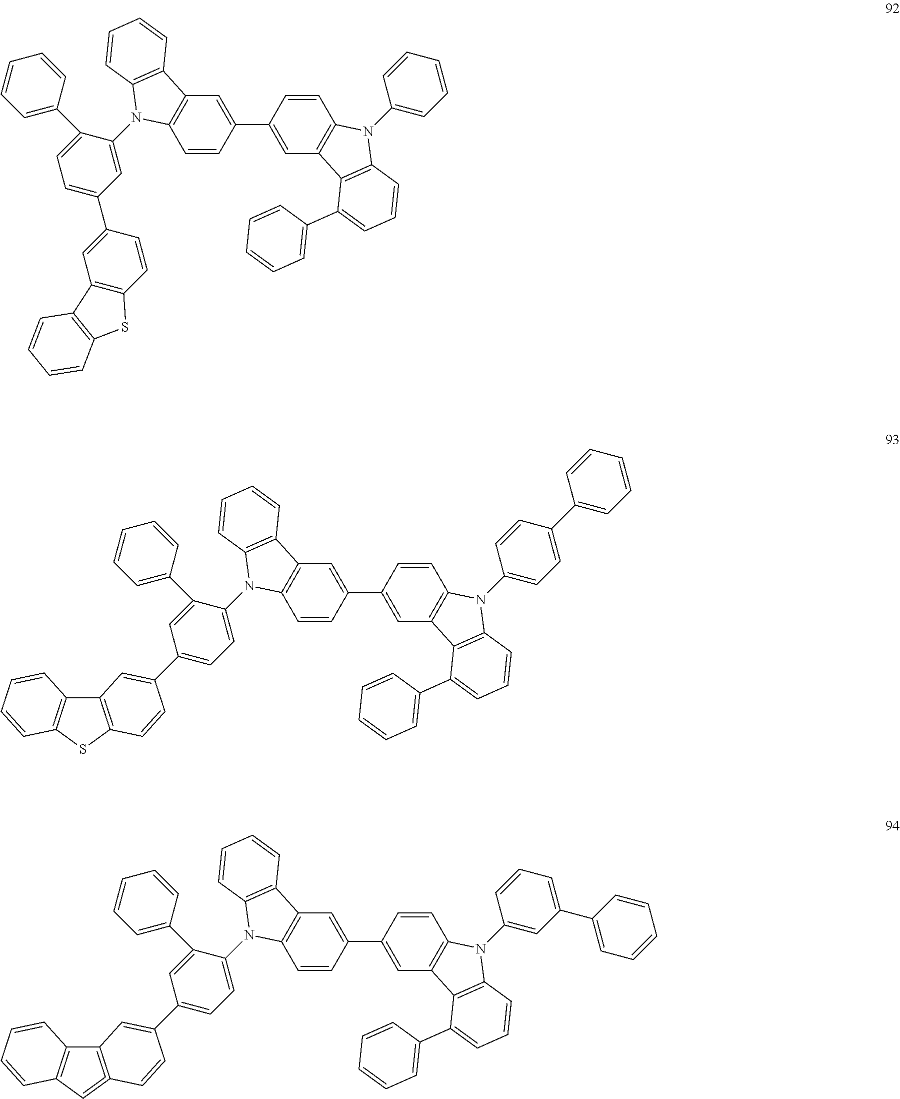

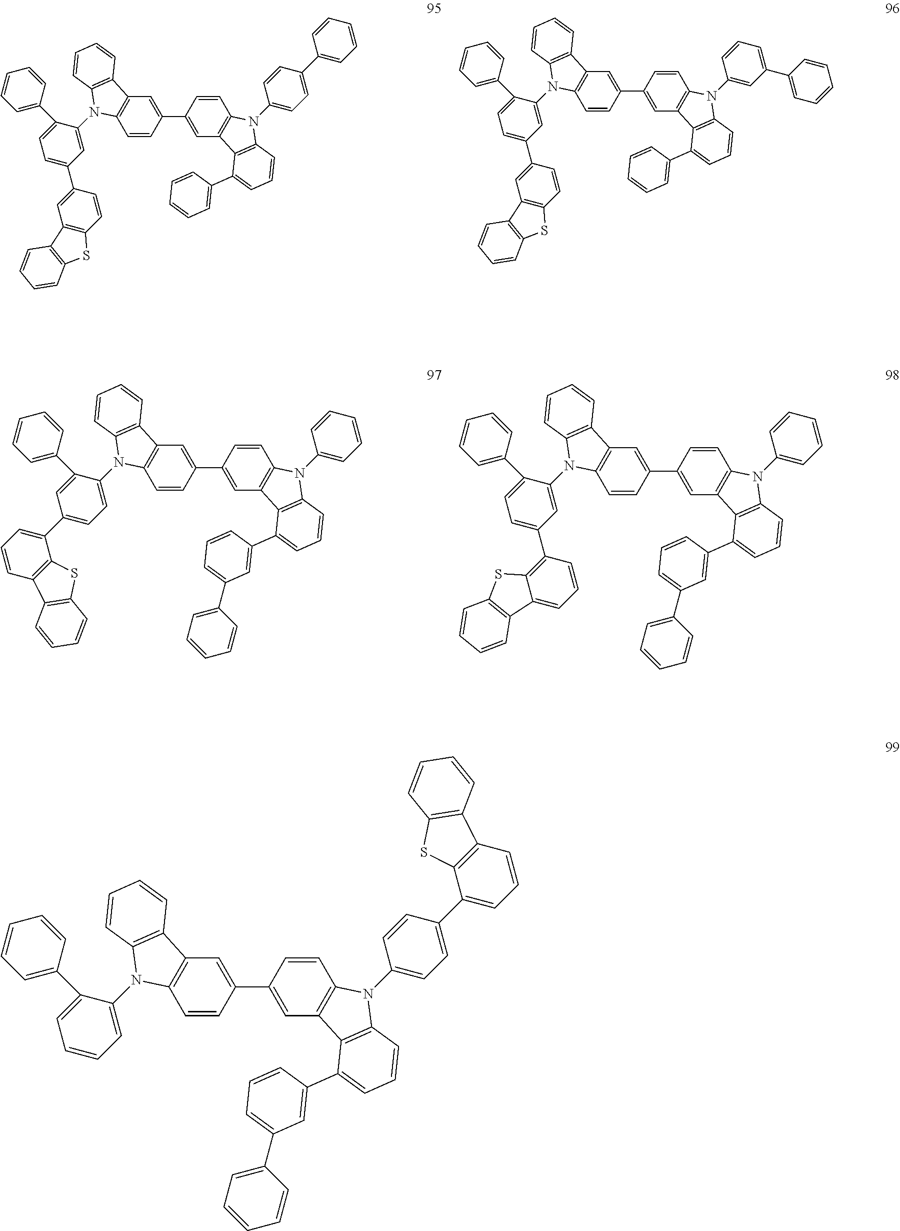

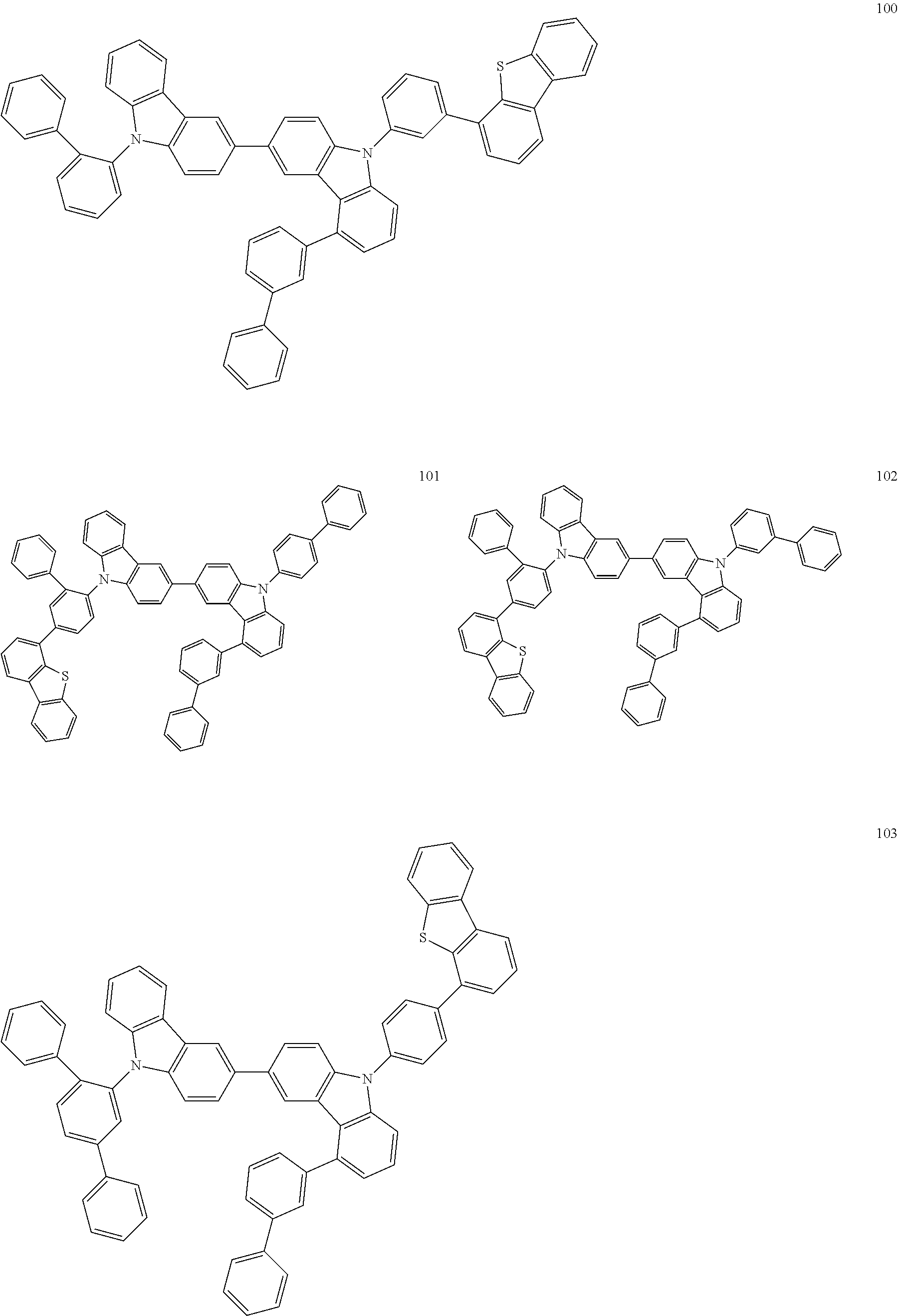 [SMILES: c1ccc(-c2cccc(-c3cccc4c3c3cc(-c5ccc6c(c5)c5ccccc5n6-c5ccccc5-c5ccccc5)ccc3n4-c3cccc(-c4cccc5c4sc4ccccc45)c3)c2)cc1 ; c1ccc(-c2ccc(-n3c4ccc(-c5ccc6c(c5)c5ccccc5n6-c5ccc(-c6cccc7c6sc6ccccc67)cc5-c5ccccc5)cc4c4c(-c5cccc(-c6ccccc6)c5)cccc43)cc2)cc1 ; c1ccc(-c2cccc(-c3cccc4c3c3cc(-c5ccc6c(c5)c5ccccc5n6-c5ccc(-c6cccc7c6sc6ccccc67)cc5-c5ccccc5)ccc3n4-c3cccc(-c4ccccc4)c3)c2)cc1 ; c1ccc(-c2cccc(-c3cccc4c3c3cc(-c5ccc6c(c5)c5ccccc5n6-c5cc(-c6ccccc6)ccc5-c5ccccc5)ccc3n4-c3ccc(-c4cccc5c4sc4ccccc45)cc3)c2)cc1]

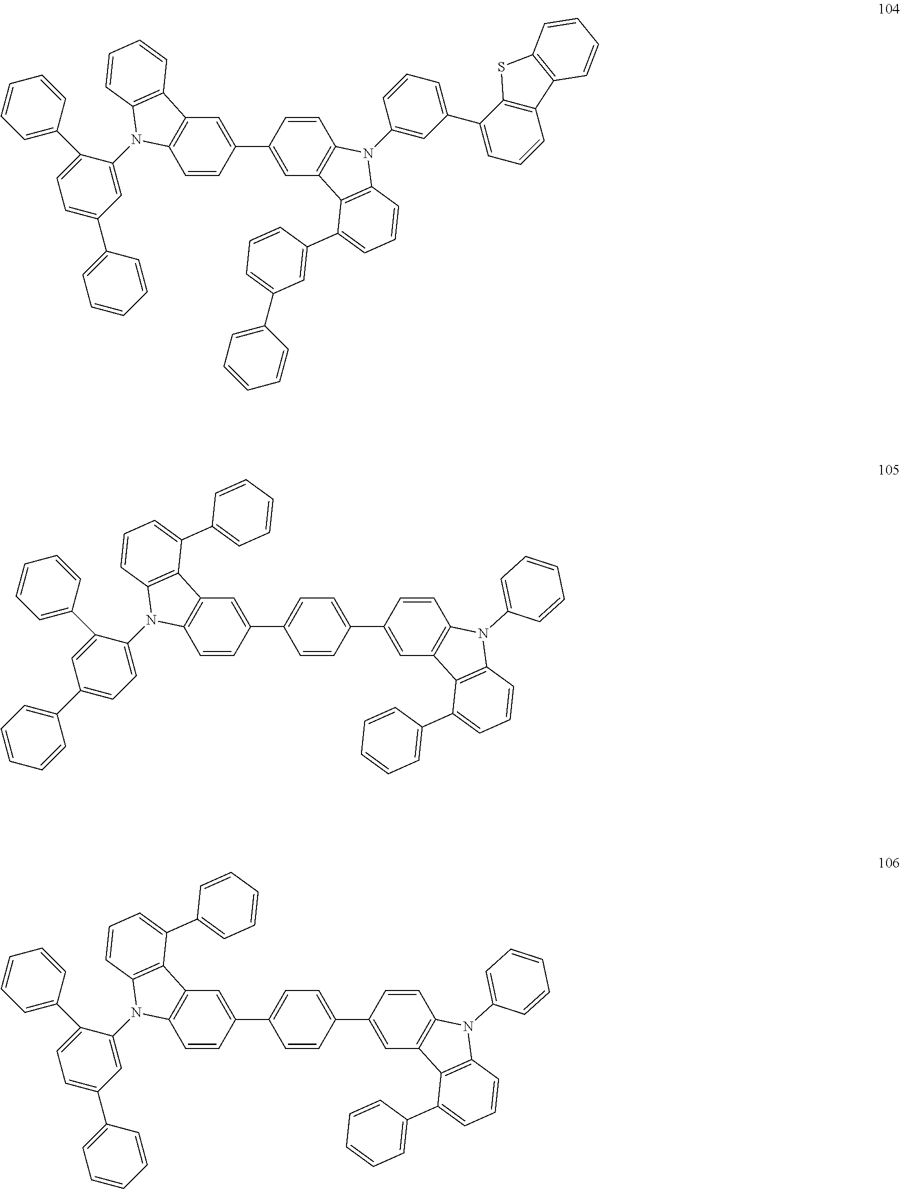 [SMILES: c1ccc(-c2cccc(-c3cccc4c3c3cc(-c5ccc6c(c5)c5ccccc5n6-c5cc(-c6ccccc6)ccc5-c5ccccc5)ccc3n4-c3cccc(-c4cccc5c4sc4ccccc45)c3)c2)cc1 ; c1ccc(-c2ccc(-n3c4ccc(-c5ccc(-c6ccc7c(c6)c6c(-c8ccccc8)cccc6n7-c6ccccc6)cc5)cc4c4c(-c5ccccc5)cccc43)c(-c3ccccc3)c2)cc1 ; c1ccc(-c2ccc(-c3ccccc3)c(-n3c4ccc(-c5ccc(-c6ccc7c(c6)c6c(-c8ccccc8)cccc6n7-c6ccccc6)cc5)cc4c4c(-c5ccccc5)cccc43)c2)cc1]

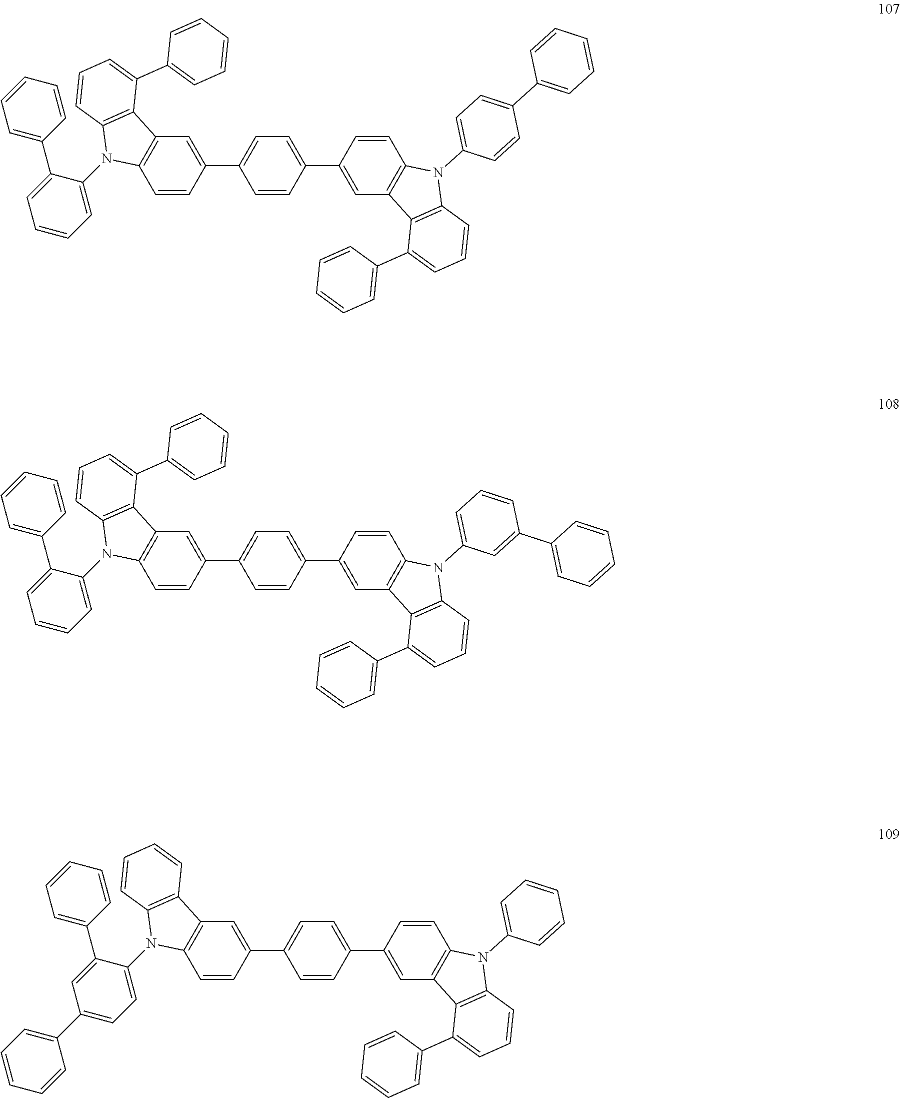 [SMILES: c1ccc(-c2ccc(-n3c4ccc(-c5ccc(-c6ccc7c(c6)c6c(-c8ccccc8)cccc6n7-c6ccccc6-c6ccccc6)cc5)cc4c4c(-c5ccccc5)cccc43)cc2)cc1 ; c1ccc(-c2cccc(-n3c4ccc(-c5ccc(-c6ccc7c(c6)c6c(-c8ccccc8)cccc6n7-c6ccccc6-c6ccccc6)cc5)cc4c4c(-c5ccccc5)cccc43)c2)cc1 ; c1ccc(-c2ccc(-n3c4ccccc4c4cc(-c5ccc(-c6ccc7c(c6)c6c(-c8ccccc8)cccc6n7-c6ccccc6)cc5)ccc43)c(-c3ccccc3)c2)cc1]

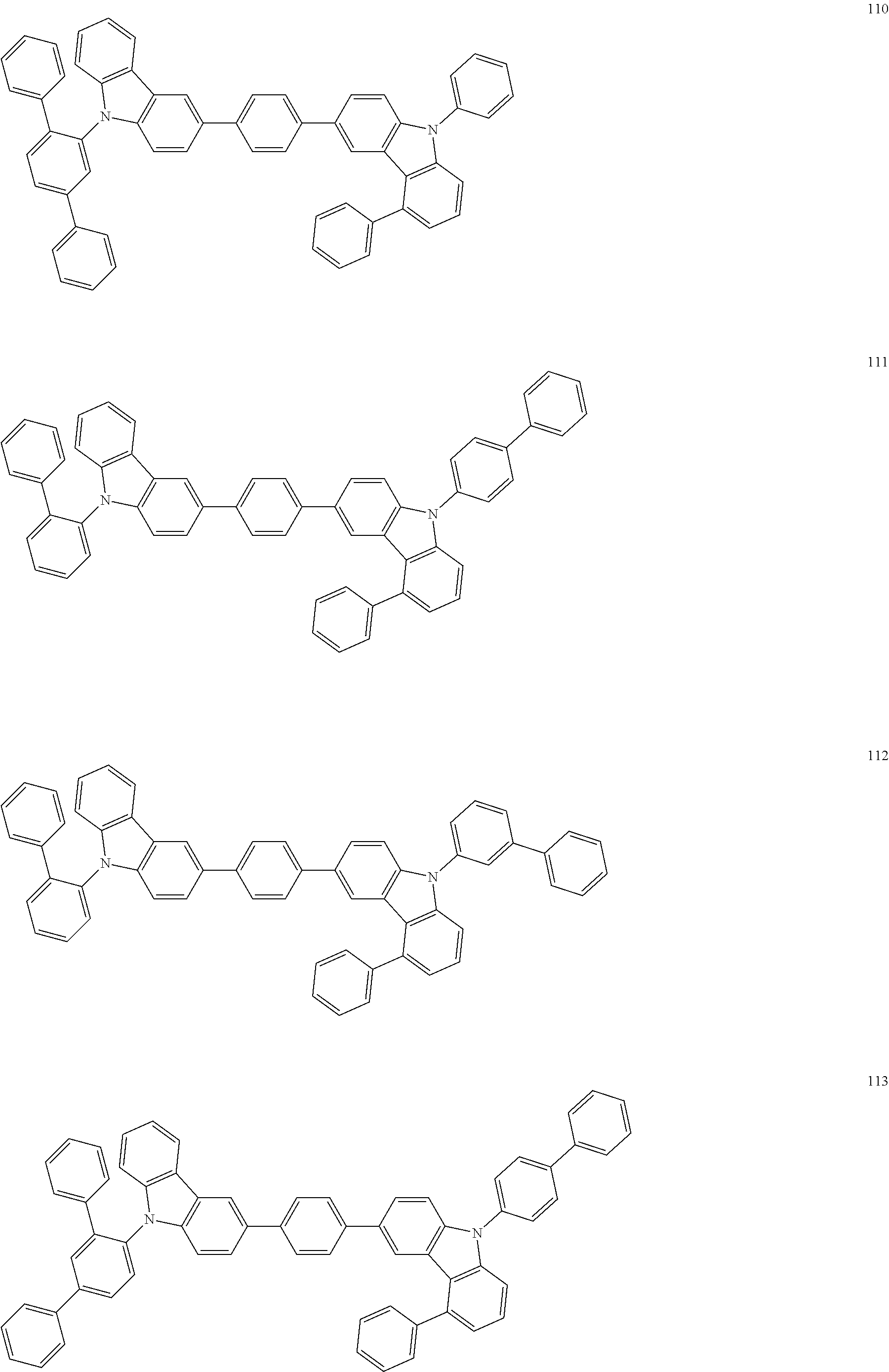 [SMILES: c1ccc(-c2ccc(-c3ccccc3)c(-n3c4ccccc4c4cc(-c5ccc(-c6ccc7c(c6)c6c(-c8ccccc8)cccc6n7-c6ccccc6)cc5)ccc43)c2)cc1 ; c1ccc(-c2ccc(-n3c4ccc(-c5ccc(-c6ccc7c(c6)c6ccccc6n7-c6ccccc6-c6ccccc6)cc5)cc4c4c(-c5ccccc5)cccc43)cc2)cc1 ; c1ccc(-c2cccc(-n3c4ccc(-c5ccc(-c6ccc7c(c6)c6ccccc6n7-c6ccccc6-c6ccccc6)cc5)cc4c4c(-c5ccccc5)cccc43)c2)cc1 ; c1ccc(-c2ccc(-n3c4ccc(-c5ccc(-c6ccc7c(c6)c6ccccc6n7-c6ccc(-c7ccccc7)cc6-c6ccccc6)cc5)cc4c4c(-c5ccccc5)cccc43)cc2)cc1]

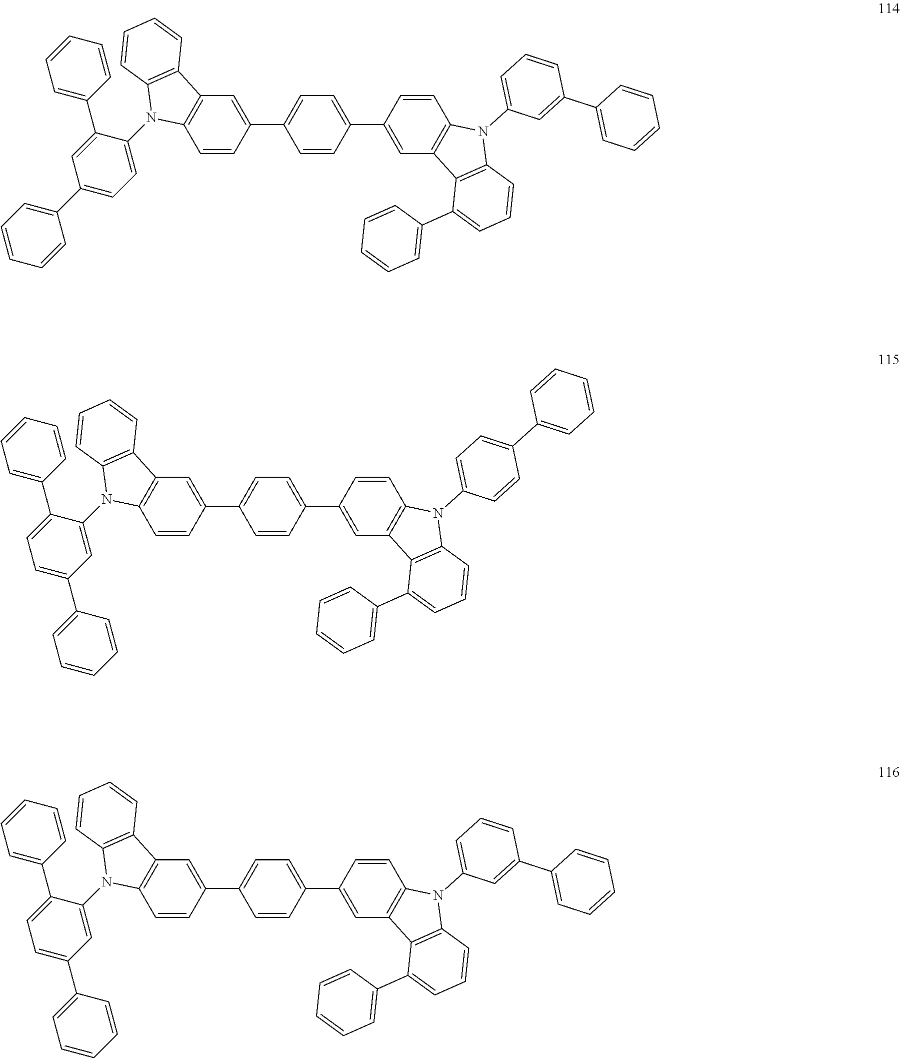 [SMILES: c1ccc(-c2cccc(-n3c4ccc(-c5ccc(-c6ccc7c(c6)c6ccccc6n7-c6ccc(-c7ccccc7)cc6-c6ccccc6)cc5)cc4c4c(-c5ccccc5)cccc43)c2)cc1 ; c1ccc(-c2ccc(-n3c4ccc(-c5ccc(-c6ccc7c(c6)c6ccccc6n7-c6cc(-c7ccccc7)ccc6-c6ccccc6)cc5)cc4c4c(-c5ccccc5)cccc43)cc2)cc1 ; c1ccc(-c2cccc(-n3c4ccc(-c5ccc(-c6ccc7c(c6)c6ccccc6n7-c6cc(-c7ccccc7)ccc6-c6ccccc6)cc5)cc4c4c(-c5ccccc5)cccc43)c2)cc1]

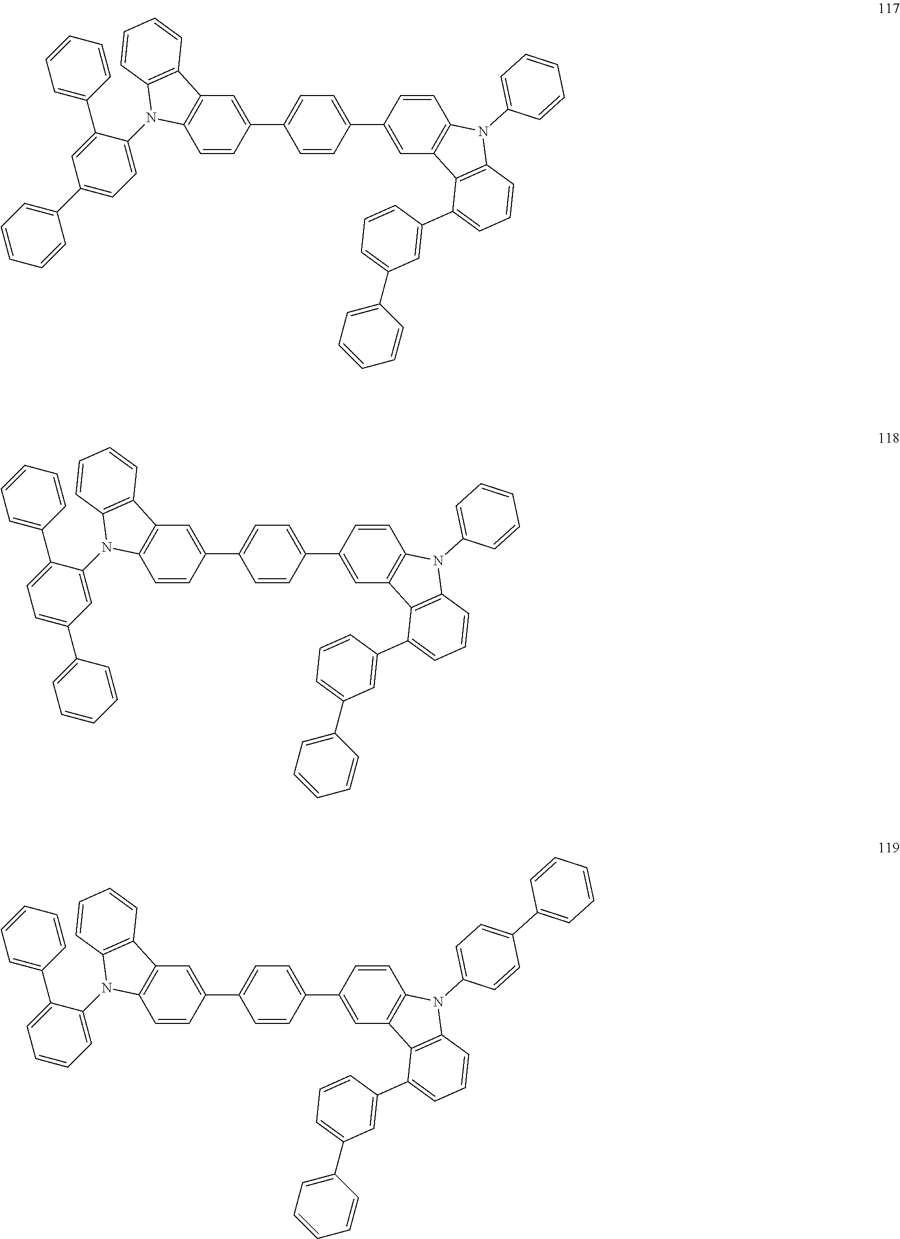 [SMILES: c1ccc(-c2cccc(-c3cccc4c3c3cc(-c5ccc(-c6ccc7c(c6)c6ccccc6n7-c6ccc(-c7ccccc7)cc6-c6ccccc6)cc5)ccc3n4-c3ccccc3)c2)cc1 ; c1ccc(-c2cccc(-c3cccc4c3c3cc(-c5ccc(-c6ccc7c(c6)c6ccccc6n7-c6cc(-c7ccccc7)ccc6-c6ccccc6)cc5)ccc3n4-c3ccccc3)c2)cc1 ; c1ccc(-c2ccc(-n3c4ccc(-c5ccc(-c6ccc7c(c6)c6ccccc6n7-c6ccccc6-c6ccccc6)cc5)cc4c4c(-c5cccc(-c6ccccc6)c5)cccc43)cc2)cc1]

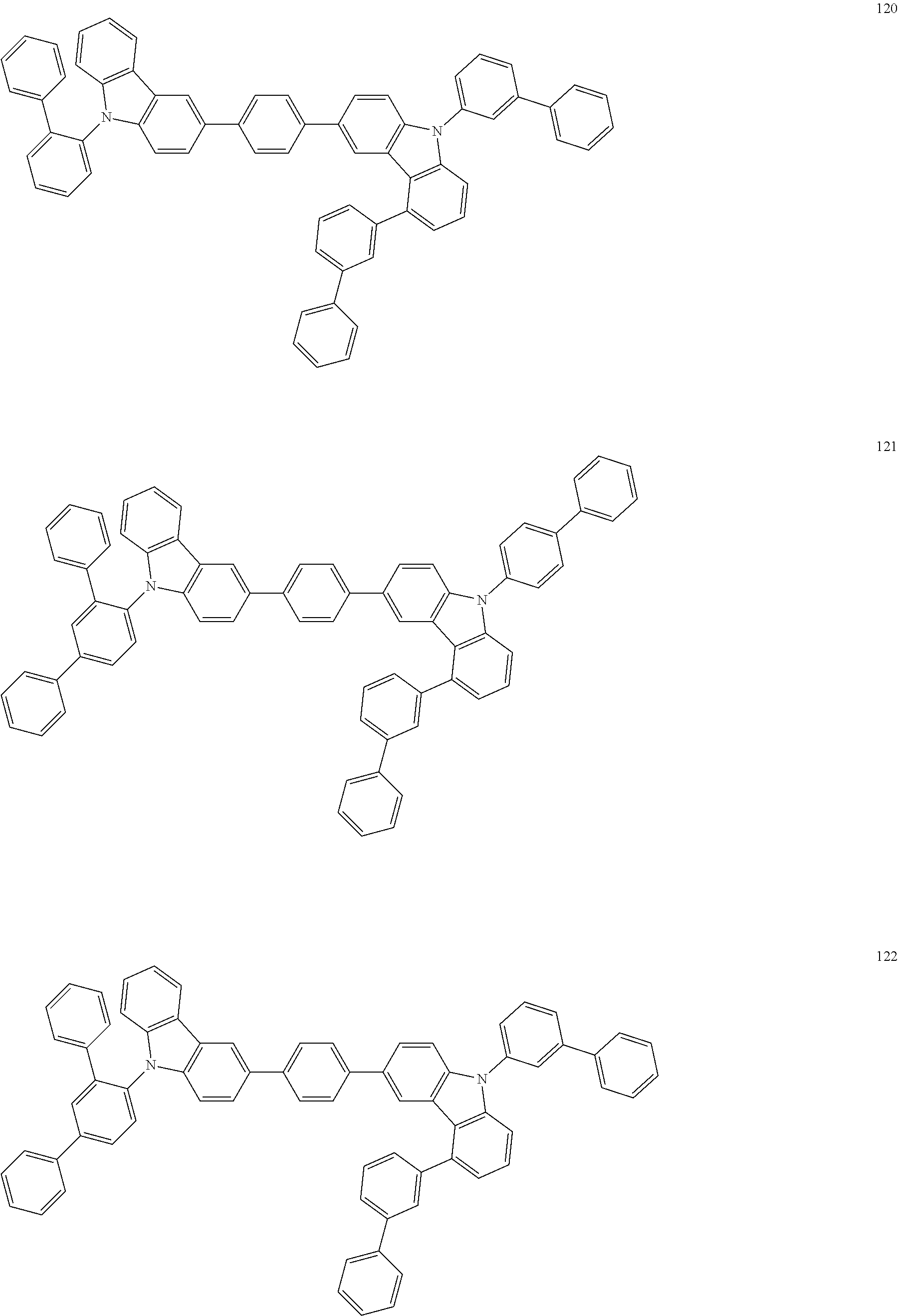 [SMILES: c1ccc(-c2cccc(-c3cccc4c3c3cc(-c5ccc(-c6ccc7c(c6)c6ccccc6n7-c6ccccc6-c6ccccc6)cc5)ccc3n4-c3cccc(-c4ccccc4)c3)c2)cc1 ; c1ccc(-c2ccc(-n3c4ccc(-c5ccc(-c6ccc7c(c6)c6ccccc6n7-c6ccc(-c7ccccc7)cc6-c6ccccc6)cc5)cc4c4c(-c5cccc(-c6ccccc6)c5)cccc43)cc2)cc1 ; c1ccc(-c2cccc(-c3cccc4c3c3cc(-c5ccc(-c6ccc7c(c6)c6ccccc6n7-c6ccc(-c7ccccc7)cc6-c6ccccc6)cc5)ccc3n4-c3cccc(-c4ccccc4)c3)c2)cc1]

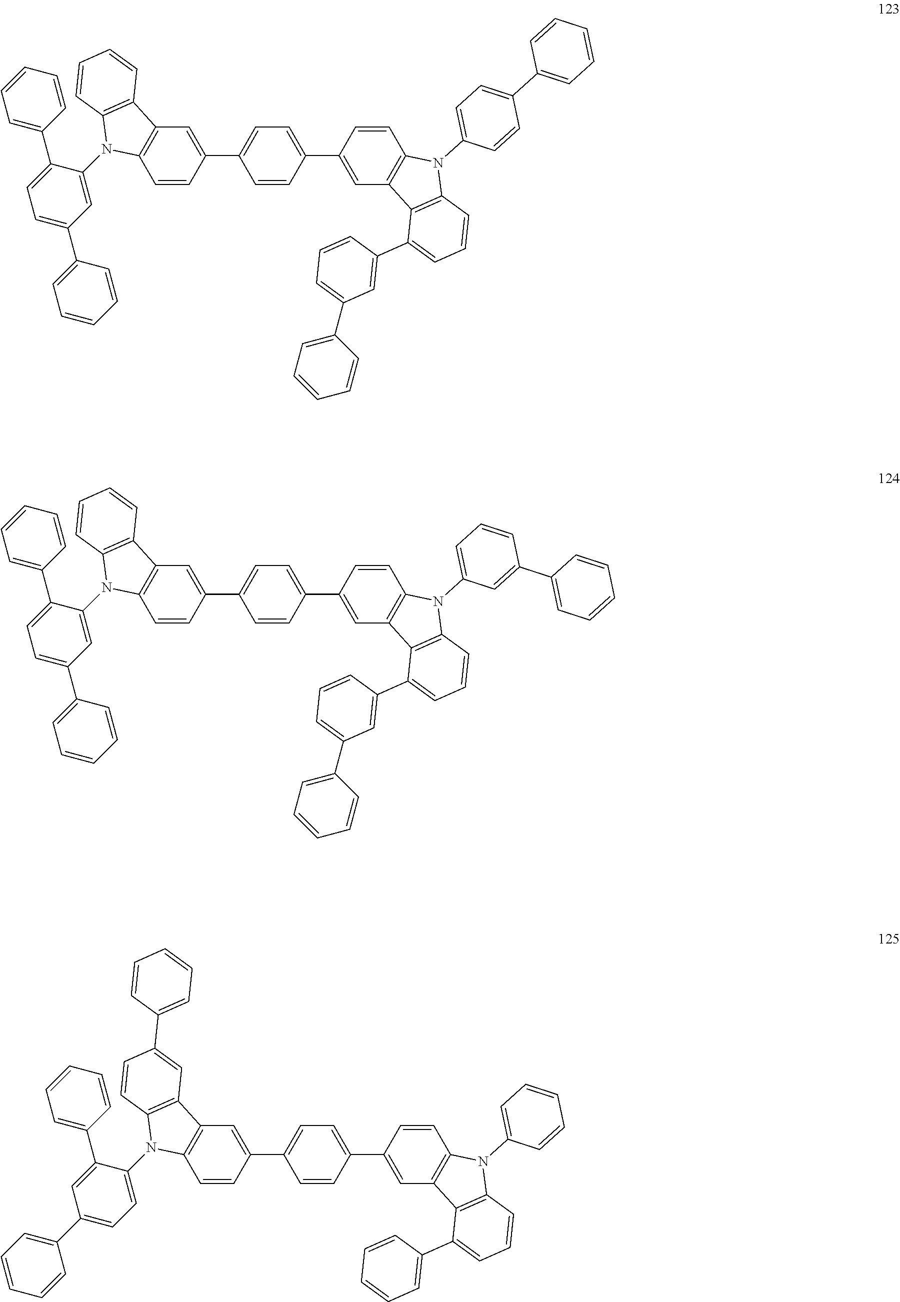 [SMILES: c1ccc(-c2ccc(-n3c4ccc(-c5ccc(-c6ccc7c(c6)c6ccccc6n7-c6cc(-c7ccccc7)ccc6-c6ccccc6)cc5)cc4c4c(-c5cccc(-c6ccccc6)c5)cccc43)cc2)cc1 ; c1ccc(-c2cccc(-c3cccc4c3c3cc(-c5ccc(-c6ccc7c(c6)c6ccccc6n7-c6cc(-c7ccccc7)ccc6-c6ccccc6)cc5)ccc3n4-c3cccc(-c4ccccc4)c3)c2)cc1 ; c1ccc(-c2ccc(-n3c4ccc(-c5ccccc5)cc4c4cc(-c5ccc(-c6ccc7c(c6)c6c(-c8ccccc8)cccc6n7-c6ccccc6)cc5)ccc43)c(-c3ccccc3)c2)cc1]

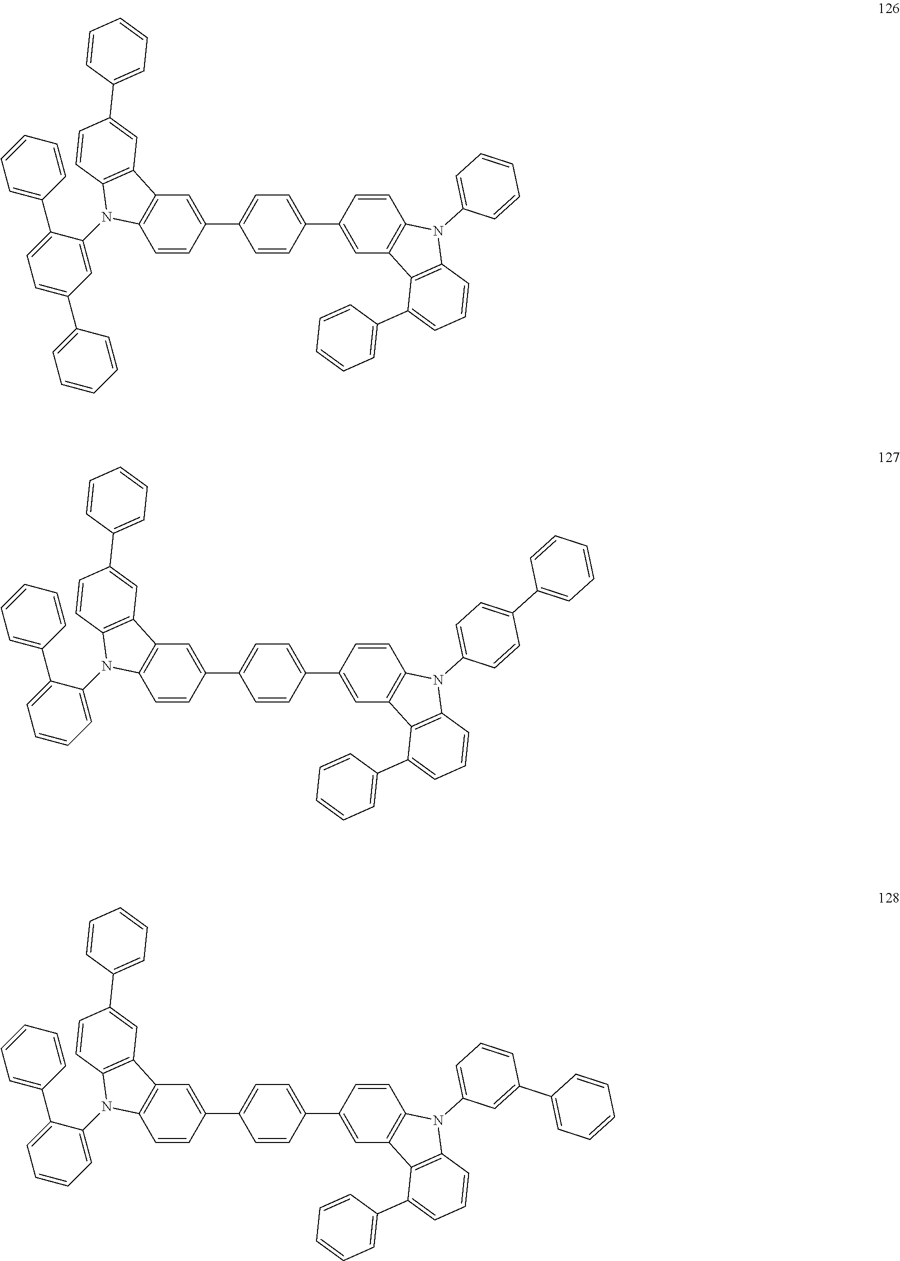 [SMILES: c1ccc(-c2ccc(-c3ccccc3)c(-n3c4ccc(-c5ccccc5)cc4c4cc(-c5ccc(-c6ccc7c(c6)c6c(-c8ccccc8)cccc6n7-c6ccccc6)cc5)ccc43)c2)cc1 ; c1ccc(-c2ccc(-n3c4ccc(-c5ccc(-c6ccc7c(c6)c6cc(-c8ccccc8)ccc6n7-c6ccccc6-c6ccccc6)cc5)cc4c4c(-c5ccccc5)cccc43)cc2)cc1 ; c1ccc(-c2cccc(-n3c4ccc(-c5ccc(-c6ccc7c(c6)c6cc(-c8ccccc8)ccc6n7-c6ccccc6-c6ccccc6)cc5)cc4c4c(-c5ccccc5)cccc43)c2)cc1]

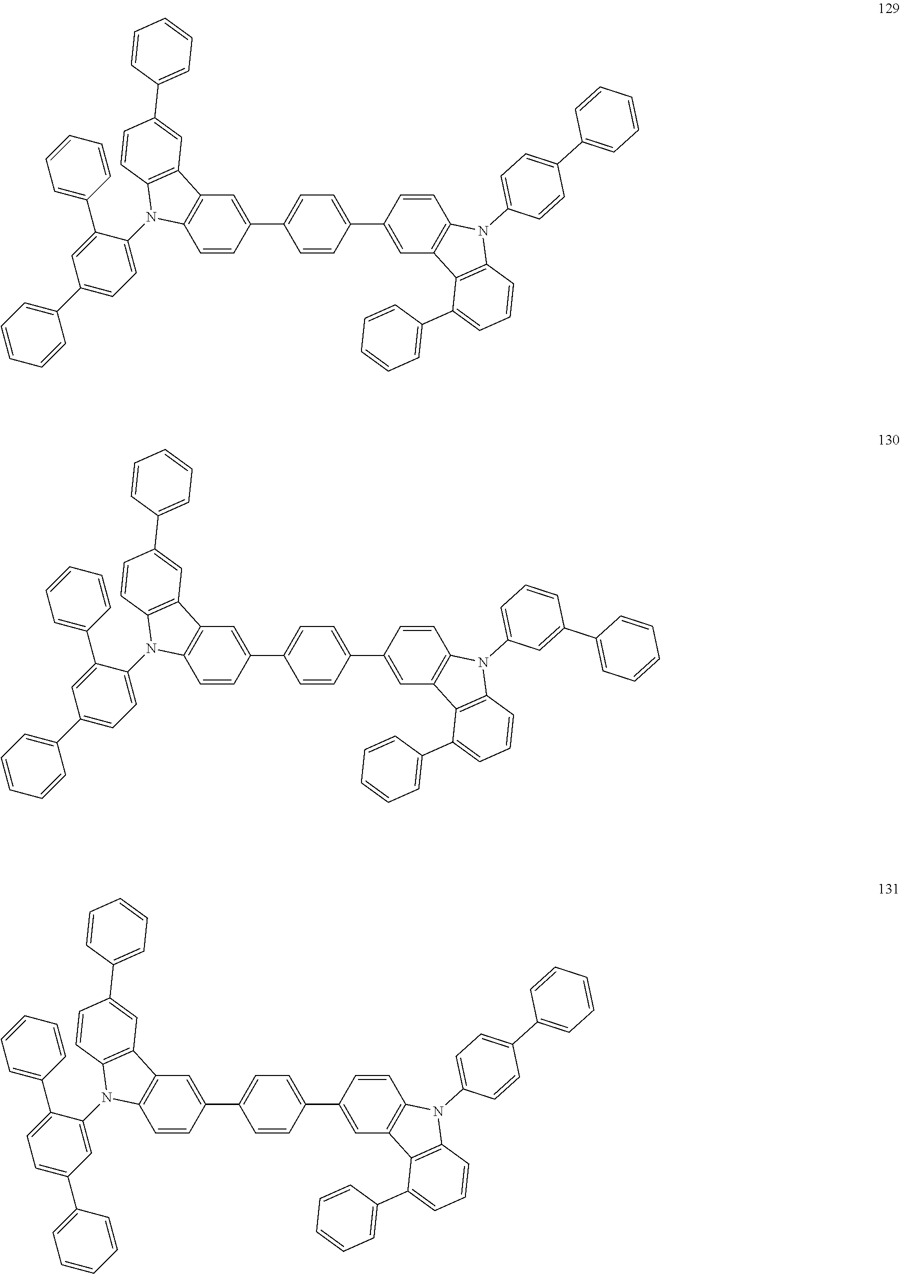 [SMILES: c1ccc(-c2ccc(-n3c4ccc(-c5ccc(-c6ccc7c(c6)c6cc(-c8ccccc8)ccc6n7-c6ccc(-c7ccccc7)cc6-c6ccccc6)cc5)cc4c4c(-c5ccccc5)cccc43)cc2)cc1 ; c1ccc(-c2cccc(-n3c4ccc(-c5ccc(-c6ccc7c(c6)c6cc(-c8ccccc8)ccc6n7-c6ccc(-c7ccccc7)cc6-c6ccccc6)cc5)cc4c4c(-c5ccccc5)cccc43)c2)cc1 ; c1ccc(-c2ccc(-n3c4ccc(-c5ccc(-c6ccc7c(c6)c6cc(-c8ccccc8)ccc6n7-c6cc(-c7ccccc7)ccc6-c6ccccc6)cc5)cc4c4c(-c5ccccc5)cccc43)cc2)cc1]

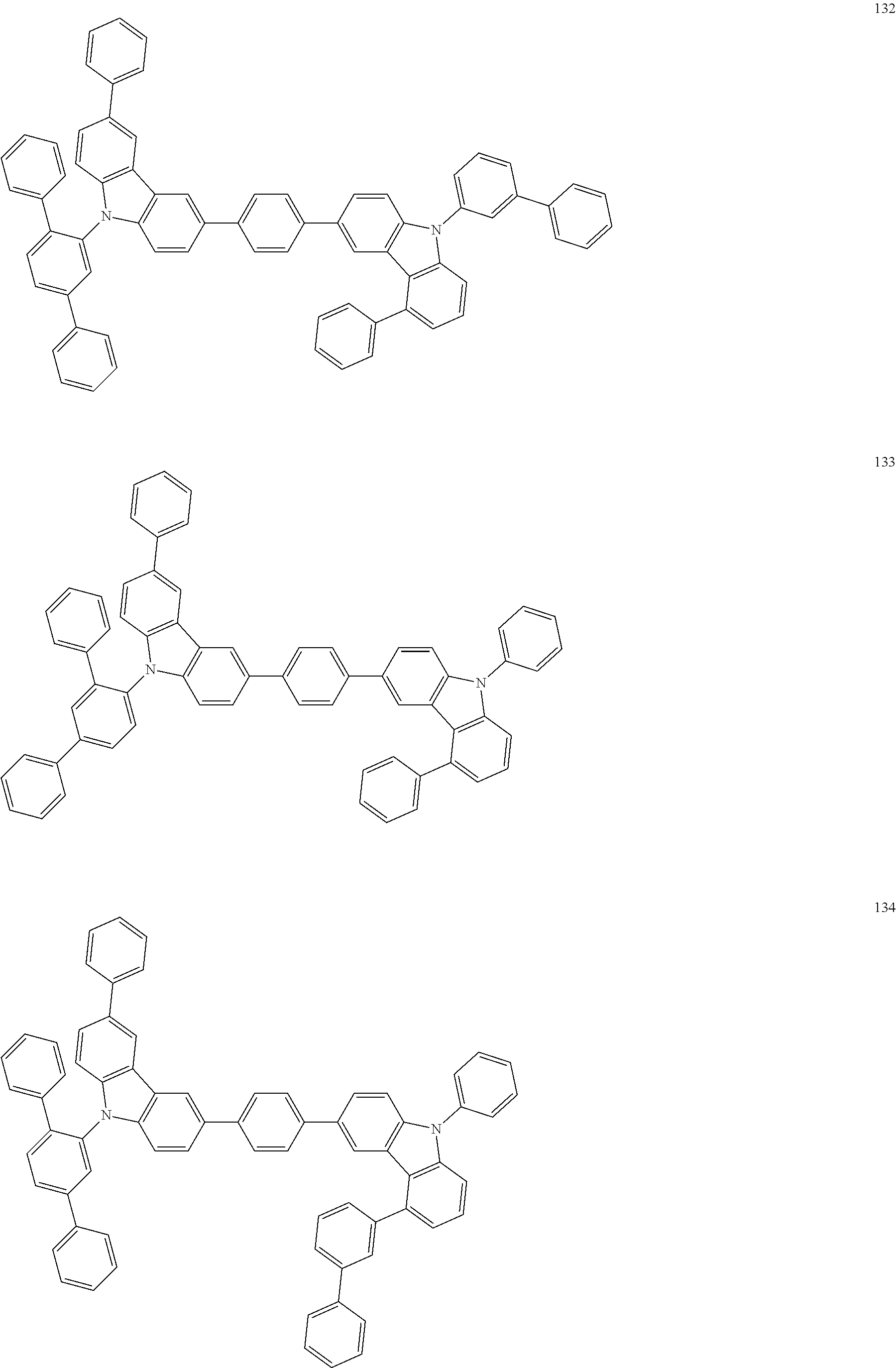 [SMILES: c1ccc(-c2cccc(-n3c4ccc(-c5ccc(-c6ccc7c(c6)c6cc(-c8ccccc8)ccc6n7-c6cc(-c7ccccc7)ccc6-c6ccccc6)cc5)cc4c4c(-c5ccccc5)cccc43)c2)cc1 ; c1ccc(-c2ccc(-n3c4ccc(-c5ccccc5)cc4c4cc(-c5ccc(-c6ccc7c(c6)c6c(-c8ccccc8)cccc6n7-c6ccccc6)cc5)ccc43)c(-c3ccccc3)c2)cc1 ; c1ccc(-c2cccc(-c3cccc4c3c3cc(-c5ccc(-c6ccc7c(c6)c6cc(-c8ccccc8)ccc6n7-c6cc(-c7ccccc7)ccc6-c6ccccc6)cc5)ccc3n4-c3ccccc3)c2)cc1]

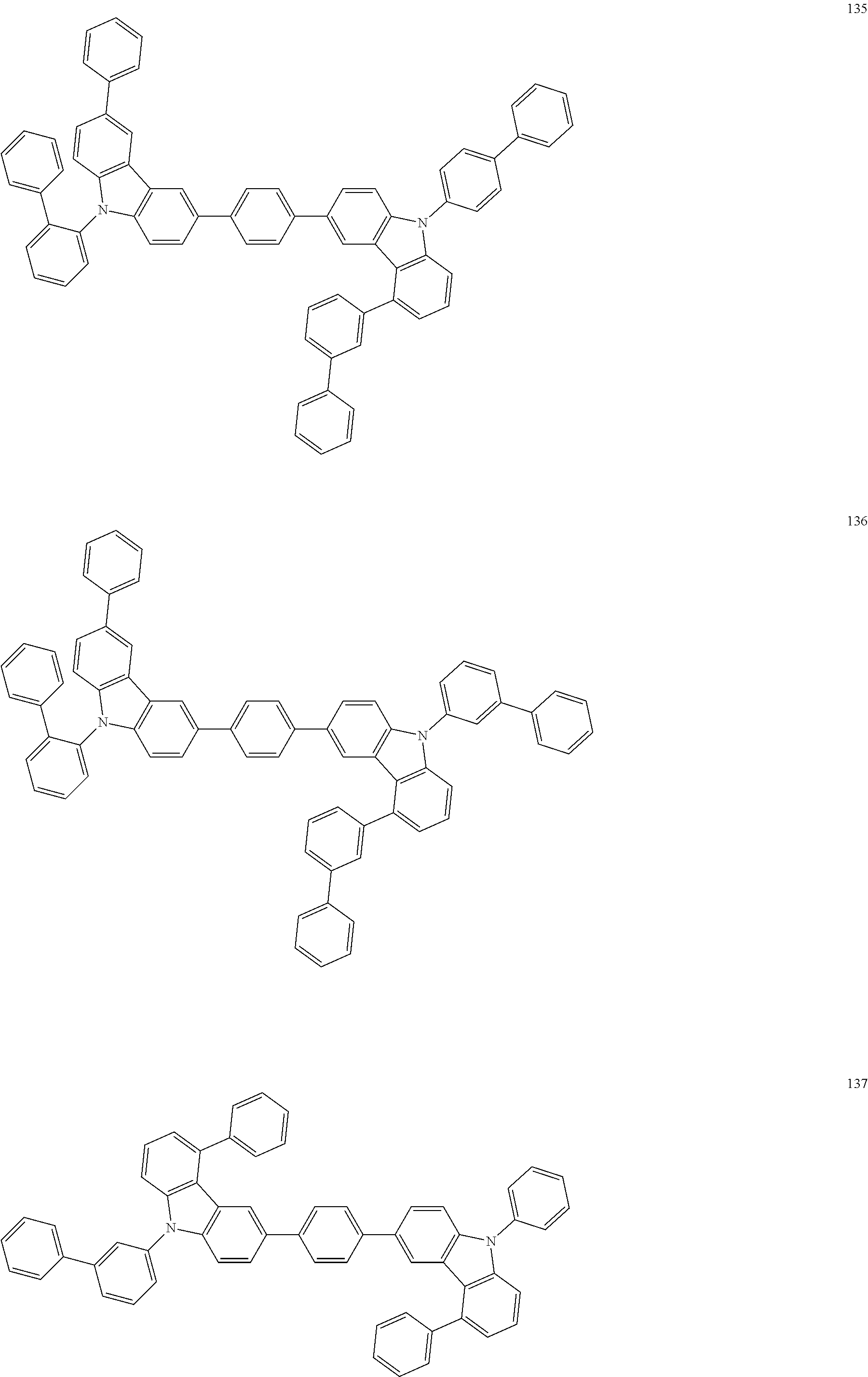 [SMILES: c1ccc(-c2ccc(-n3c4ccc(-c5ccc(-c6ccc7c(c6)c6cc(-c8ccccc8)ccc6n7-c6ccccc6-c6ccccc6)cc5)cc4c4c(-c5cccc(-c6ccccc6)c5)cccc43)cc2)cc1 ; c1ccc(-c2cccc(-c3cccc4c3c3cc(-c5ccc(-c6ccc7c(c6)c6cc(-c8ccccc8)ccc6n7-c6ccccc6-c6ccccc6)cc5)ccc3n4-c3cccc(-c4ccccc4)c3)c2)cc1 ; c1ccc(-c2cccc(-n3c4ccc(-c5ccc(-c6ccc7c(c6)c6c(-c8ccccc8)cccc6n7-c6ccccc6)cc5)cc4c4c(-c5ccccc5)cccc43)c2)cc1]

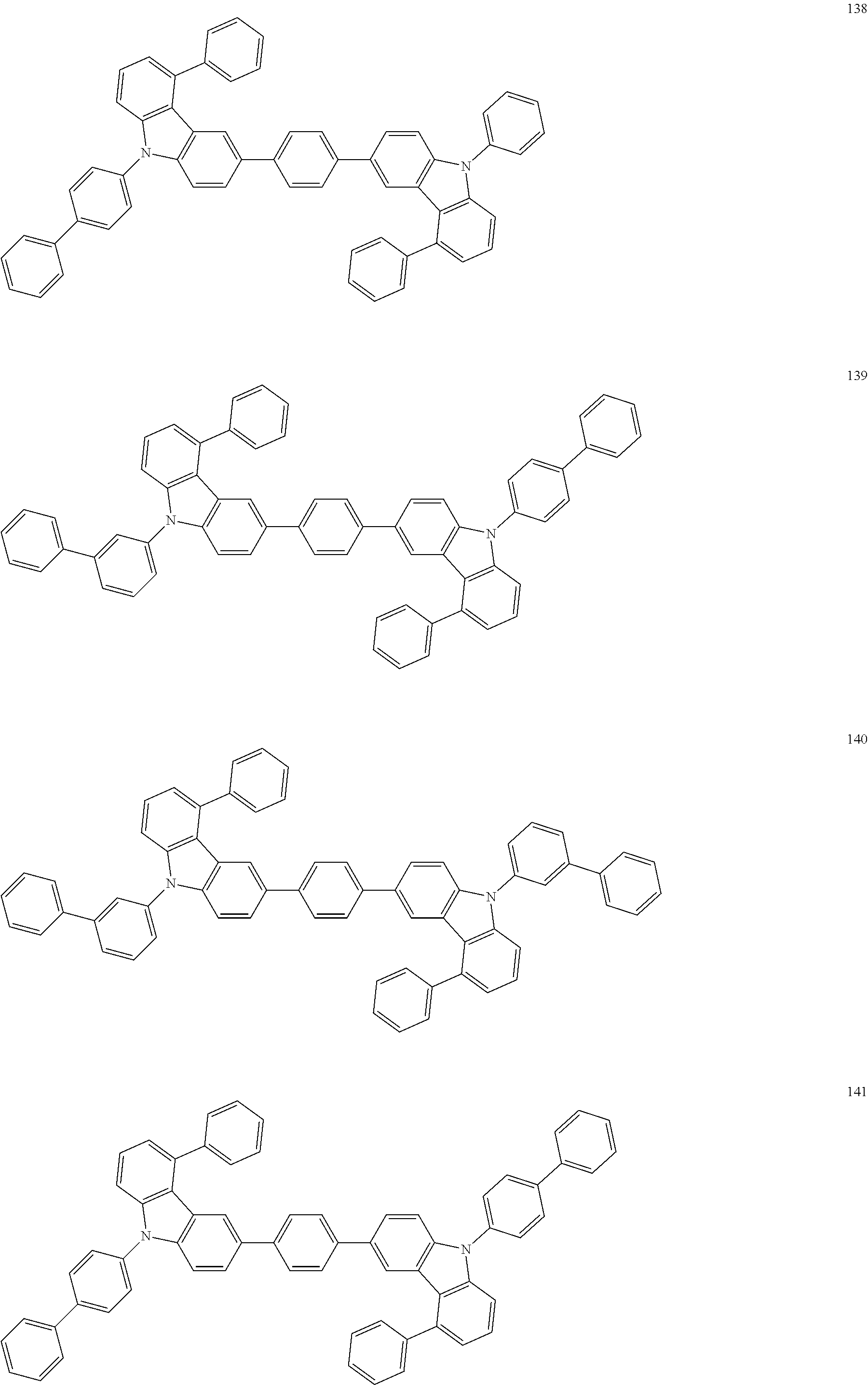 [SMILES: c1ccc(-c2ccc(-n3c4ccc(-c5ccc(-c6ccc7c(c6)c6c(-c8ccccc8)cccc6n7-c6ccccc6)cc5)cc4c4c(-c5ccccc5)cccc43)cc2)cc1 ; c1ccc(-c2ccc(-n3c4ccc(-c5ccc(-c6ccc7c(c6)c6c(-c8ccccc8)cccc6n7-c6cccc(-c7ccccc7)c6)cc5)cc4c4c(-c5ccccc5)cccc43)cc2)cc1 ; c1ccc(-c2cccc(-n3c4ccc(-c5ccc(-c6ccc7c(c6)c6c(-c8ccccc8)cccc6n7-c6cccc(-c7ccccc7)c6)cc5)cc4c4c(-c5ccccc5)cccc43)c2)cc1 ; c1ccc(-c2ccc(-n3c4ccc(-c5ccc(-c6ccc7c(c6)c6c(-c8ccccc8)cccc6n7-c6ccc(-c7ccccc7)cc6)cc5)cc4c4c(-c5ccccc5)cccc43)cc2)cc1]

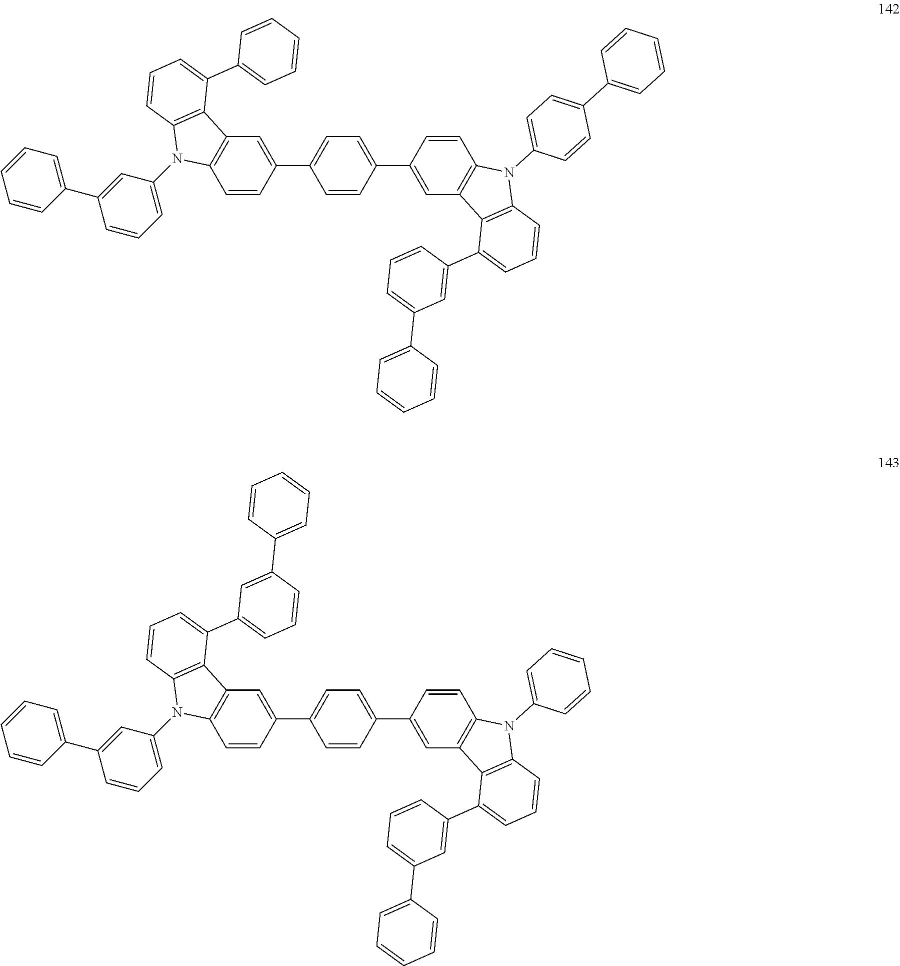 [SMILES: c1ccc(-c2ccc(-n3c4ccc(-c5ccc(-c6ccc7c(c6)c6c(-c8ccccc8)cccc6n7-c6cccc(-c7ccccc7)c6)cc5)cc4c4c(-c5cccc(-c6ccccc6)c5)cccc43)cc2)cc1 ; c1ccc(-c2cccc(-c3cccc4c3c3cc(-c5ccc(-c6ccc7c(c6)c6c(-c8cccc(-c9ccccc9)c8)cccc6n7-c6cccc(-c7ccccc7)c6)cc5)ccc3n4-c3ccccc3)c2)cc1]

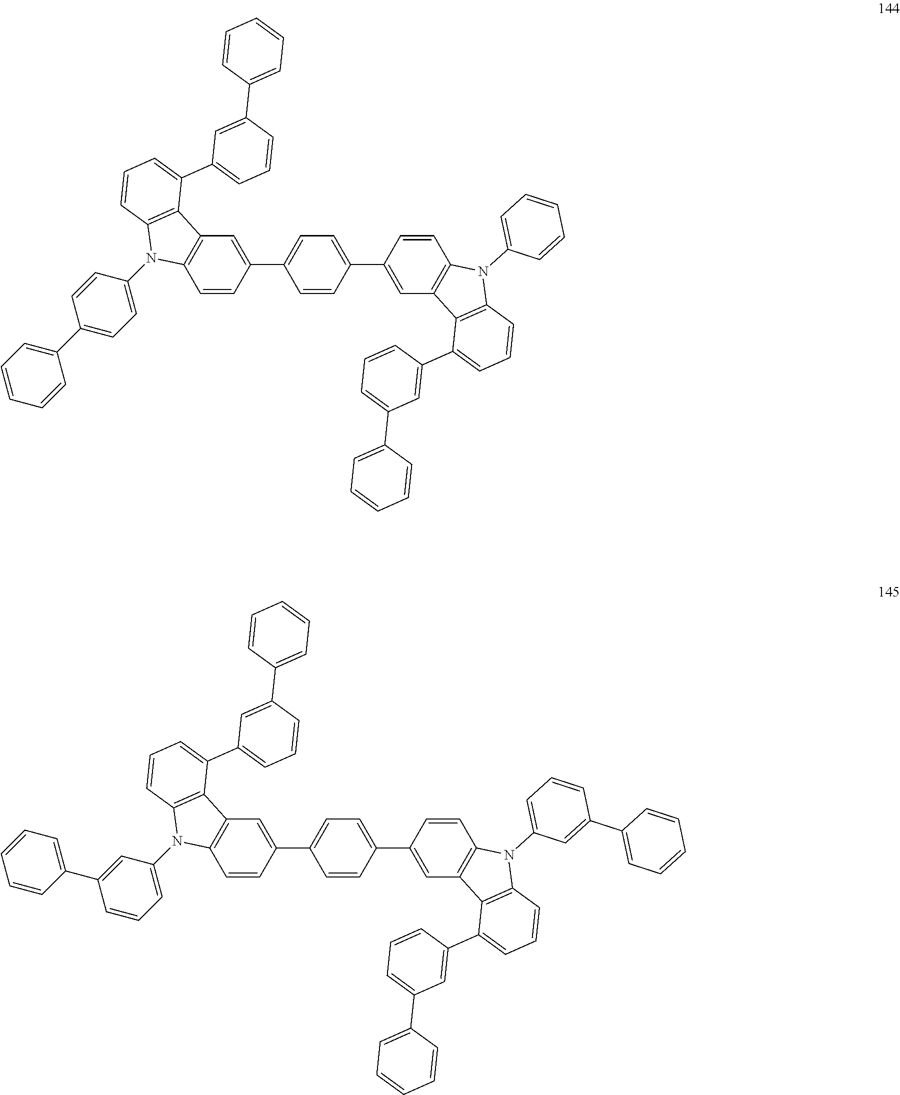 [SMILES: c1ccc(-c2ccc(-n3c4ccc(-c5ccc(-c6ccc7c(c6)c6c(-c8cccc(-c9ccccc9)c8)cccc6n7-c6ccccc6)cc5)cc4c4c(-c5cccc(-c6ccccc6)c5)cccc43)cc2)cc1 ; c1ccc(-c2cccc(-c3cccc4c3c3cc(-c5ccc(-c6ccc7c(c6)c6c(-c8cccc(-c9ccccc9)c8)cccc6n7-c6cccc(-c7ccccc7)c6)cc5)ccc3n4-c3cccc(-c4ccccc4)c3)c2)cc1]

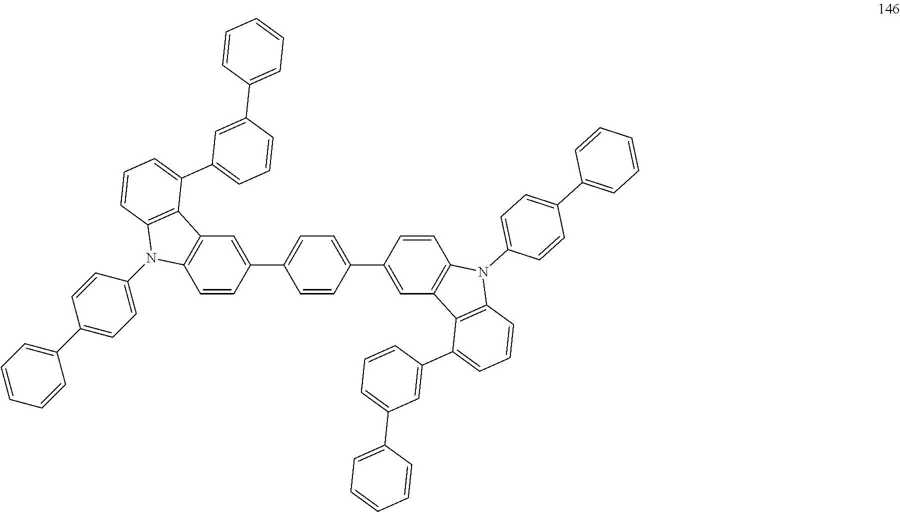 [SMILES: c1ccc(-c2ccc(-n3c4ccc(-c5ccc(-c6ccc7c(c6)c6c(-c8cccc(-c9ccccc9)c8)cccc6n7-c6ccc(-c7ccccc7)cc6)cc5)cc4c4c(-c5cccc(-c6ccccc6)c5)cccc43)cc2)cc1]

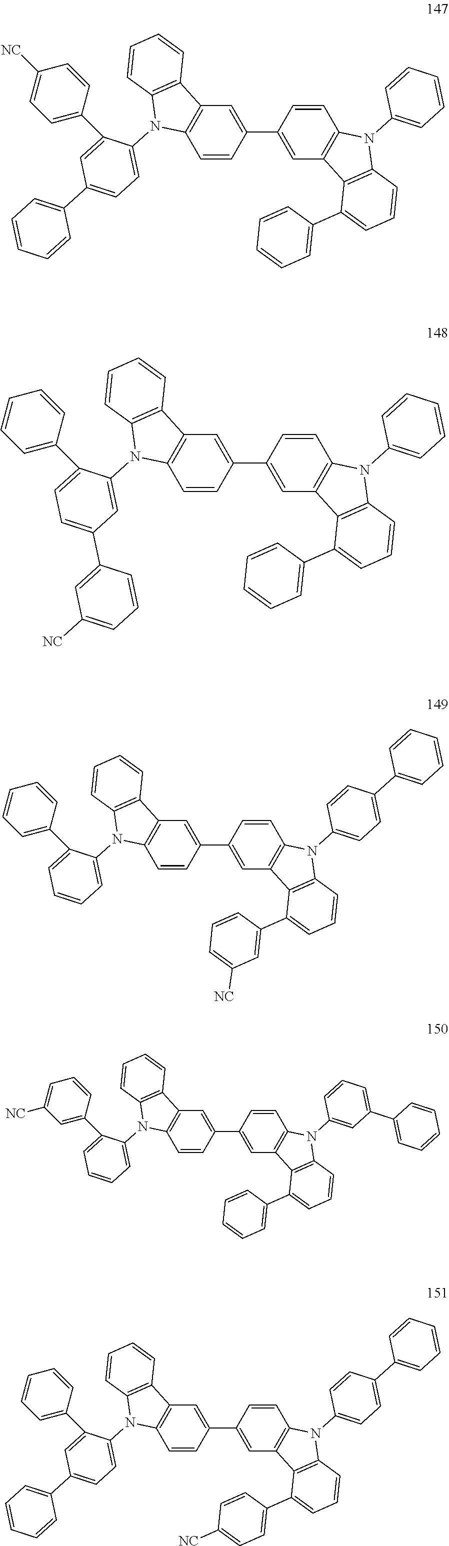

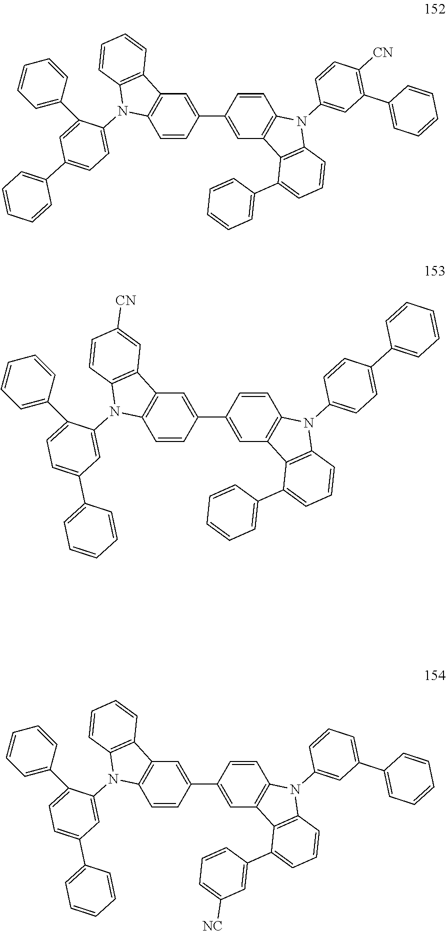 [SMILES: N#Cc1ccc(-n2c3ccc(-c4ccc5c(c4)c4ccccc4n5-c4ccc(-c5ccccc5)cc4-c4ccccc4)cc3c3c(-c4ccccc4)cccc32)cc1-c1ccccc1 ; N#Cc1ccc2c(c1)c1cc(-c3ccc4c(c3)c3c(-c5ccccc5)cccc3n4-c3ccc(-c4ccccc4)cc3)ccc1n2-c1cc(-c2ccccc2)ccc1-c1ccccc1 ; N#Cc1cccc(-c2cccc3c2c2cc(-c4ccc5c(c4)c4ccccc4n5-c4cc(-c5ccccc5)ccc4-c4ccccc4)ccc2n3-c2cccc(-c3ccccc3)c2)c1]

In the condensed cyclic compound represented by Formula 1, most of highest occupied molecular orbital (HOMO) is distributed in a biscarbazole backbone (portion indicated by a dashed line in Formula 1) (hereinafter, referred to as a biscarbazole plane). In the condensed cyclic compound represented by Formula 1, Ar₁ to Ar₆ are arranged around the biscarbazole plane. Therefore, the condensed cyclic compound represented by Formula 1 may suppress interaction with homogeneous molecules or heterogeneous molecules existing around the biscarbazole plane in which a HOMO is distributed:

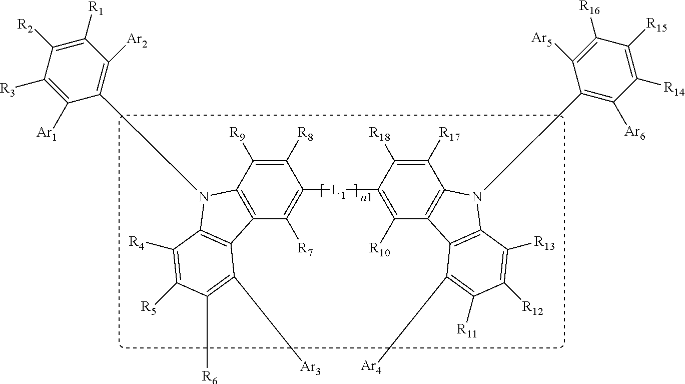

Therefore, the condensed cyclic compound represented by Formula 1 may have excellent solubility in a solvent. In addition, when a layer is formed by using a wet film-forming method, the condensed cyclic compound represented by Formula 1 may suppress the film quality from being deteriorated by aggregation of organic molecules. When a plurality of organic molecules are aggregated, a molecular orbital may expand as compared with that of a monomolecular state. In this manner, energy levels such as HOMO, LUMO, S1, or T1 may be different from the monomolecular state. In addition, such a different energy level may cause adverse effects such as loss of excitons. Therefore, since the compound according to the embodiment is suppressed from aggregating organic molecules, the compound according to the embodiment may maintain or improve the performance of the organic light-emitting device (for example, hole transport capability) even when the organic light-emitting device is manufactured by solution coating. Therefore, the organic light-emitting device may be manufactured without using expensive vacuum deposition. The compound according to the embodiment may be advantageous to manufacturing a large-size organic light-emitting device.

In addition, when n in Formula 1 is 0, one selected from Ar₃ and Ar₄ may be selected from hydrogen and deuterium. In this embodiment, the condensed cyclic compound represented by Formula 1 may have high solubility. When n is 0 and Ar₃ and Ar₄ are substituents other than hydrogen and deuterium, the solubility may be lowered. This may be caused by interaction between Ar₃ and Ar₄. However, when n is 1, the distance between Ar₃ and Ar₄ increases and the interaction between Ar₃ and Ar₄ decreases. Thus, a reduction in solubility may not appear. That is, it is desired to restrict the selection of Ar₃ and Ar₄ only when n is 0.

In addition, in an embodiment, in Formula 1, at least one selected from R₁ to R₃ and R₁₄ to R₁₆ may be selected from substituents other than hydrogen (for example, a C₆-C₆₀ aryl group, a C₁-C₆₀ heteroaryl group, or the like). In this embodiment, since the condensed cyclic compound represented by Formula 1 may improve exciton stability (for example, photo chemical stability), an light-emitting device including the condensed cyclic compound represented by Formula 1 may have excellent emission lifespan characteristics.

In addition, in an embodiment, at least one selected from R₆ and R₁₁ may be selected from substituents other than hydrogen (for example, a C₆-C₆₀ aryl group, a C₁-C₆₀ heteroaryl group, or the like). In this embodiment, a reduction in lowest excitation triplet energy (referred as T1 energy) is suppressed, and HOMO is expanded due to π conjugate effect. Therefore, hole stability or photo chemical stability of the condensed cyclic compound represented by Formula 1 may be improved, and an light-emitting device including the condensed cyclic compound represented by Formula 1 may have improved luminescent efficiency and emission lifespan characteristics.

In addition, in an embodiment, when L₁ in Formula 1 is a 1,4-phenylene group (a group represented by Formula 3-1), a structure in which two carbazole rings are linked to a para position through the aromatic hydrocarbon group may increase a π conjugate length and widen a biscarbazole plane in which HOMO is distributed. Therefore, the HOMO distribution in the condensed cyclic compound is widened. Hence, the charge transport capability (for example, the hole transport capability) of the condensed cyclic compound represented by Formula 1 may increase, thereby improving the luminescent efficiency and emission lifespan of the light-emitting device including the condensed cyclic compound.

The condensed cyclic compound represented by Formula 1 may be included in an organic layer between a pair of electrodes of an organic light-emitting device. For example, the condensed cyclic compound represented by Formula 1 may be included in an emission layer and may be a host.

The condensed cyclic compound represented by Formula 1 may be synthesized by known organic synthesis methods. A specific method of synthesizing the condensed cyclic compound represented by Formula 1 may be understood by those of ordinary skill in the art by referring to Examples provided below.

Method of Preparing Compound

A method of preparing the condensed cyclic compound represented by Formula 1 is not particularly limited, and various preparation methods including known synthesis methods may be used. However, in terms of reliably obtaining the condensed cyclic compound represented by Formula 1, a method of preparing the condensed cyclic compound according to an embodiment includes a reaction between a compound represented by Formula 2 and a compound represented by Formula 3.

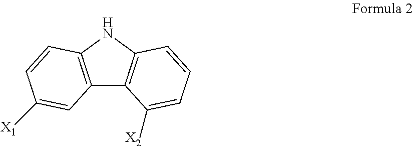

In Formula 2, X₁ and X₂ may each independently be a halogen atom.

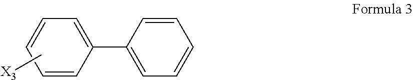

In Formula 3, X₃ may be a halogen atom.

In Formula 2, X₁ and X₂ may each independently indicate a halogen atom, but different halogen atoms are preferable. X₁ and X₂ may each independently be selected from a chlorine atom, a bromine atom, and an iodine atom, and preferably, a chlorine atom and a bromine atom.

In addition, X₃ in Formula 3 may be a halogen atom. The halogen atom indicates a fluorine atom, a chlorine atom, a bromine atom, an iodine atom, or an astatine atom.

A preferable reaction in which the compound represented by Formula 2 is used as an intermediate may be a reaction of substituting a hydrogen atom of a carbazole nitrogen of Formula 2 with an aromatic ring or an aromatic hetero ring. For example, an aromatic nucleophilic substitution reaction using fluorinated biphenyl (an S_{N}Ar reaction or ipso substitution reaction) or the Ullmann reaction using phenyl iodide may be used.

In the case of introducing different substituents to the 3rd, 5th, and 9th positions of the carbazole, it is desired to establish the carbazole ring part and the synthesis step is four steps or more. Therefore, when synthesizing a compound having different substituents at the 3rd, 5th, and 9th positions of the carbazole, the number of steps from the introduction of the first substituent to the introduction of the third substituent is four or more. This is disadvantageous in terms of synthesis in the case of synthesizing several kinds of compounds each having three substituents changed for the purpose of, for example, research and the like.

In the case of using Intermediate having X₁═Br, X₂═Cl in Formula 2, when different substituents are optionally introduced stepwise in the 3rd, 5th, and 9th positions of the carbazole, the side reactions may be suppressed and the synthesis may be performed at high purity or yield. In addition, the synthesis steps may be reduced. Therefore, the carbazole compound having different substituents at the 3rd, 5th, and 9th positions may be synthesized at high yield, high purity, and low cost.

The compound represented by Formula 2 has one carbazole ring, X₁ is linked to the 3rd carbon atom of the carbazole ring, and X₂ is linked to the 5th carbon atom of the carbazole ring. In addition, the hydrogen atom linked to the carbon atom of the carbazole ring to which X₁ and X₂ are not linked in Formula 2 may be substituted with other substituents.

Other substituents of the compound represented by Formula 2 are not particularly limited, and may be, for example, deuterium, —F, —Cl, —Br, —I, a hydroxyl group, a cyano group, a nitro group, an amino group, an amidino group, a hydrazine group, a hydrazone group, a carboxylic acid group or a salt thereof, a sulfonic acid group or a salt thereof, a phosphoric acid group or a salt thereof, a substituted or unsubstituted C₁-C₆₀ alkyl group, a substituted or unsubstituted C₂-C₆₀ alkenyl group, a substituted or unsubstituted C₂-C₆₀ alkynyl group, a substituted or unsubstituted C₁-C₆₀ alkoxy group, a substituted or unsubstituted C₃-C₁₀ cycloalkyl group, a substituted or unsubstituted C₁-C₁₀ heterocycloalkyl group, a substituted or unsubstituted C₃-C₁₀ cycloalkenyl group, a substituted or unsubstituted C₁-C₁₀ heterocycloalkenyl group, a substituted or unsubstituted C₆-C₆₀ aryl group, a substituted or unsubstituted C₆-C₆₀ aryloxy group, a substituted or unsubstituted C₆-C₆₀ arylthio group, a substituted or unsubstituted C₁-C₆₀ heteroaryl group, a substituted or unsubstituted monovalent non-aromatic condensed polycyclic group, a substituted or unsubstituted monovalent non-aromatic condensed heteropolycyclic group, —Si(Q₁)(Q₂)(Q₃), —N(Q₄)(Q₅), and —B(Q₆)(Q₇).

In addition, the compound represented by Formula 3 is phenyl halide. A binding site of X₃ in the benzene ring to which X₃ in the compound represented by Formula 3 is linked is not particularly limited. However, in terms of reliably obtaining the condensed cyclic compound represented by Formula 1, the binding site of X₃ is preferably a meta position or a para position of the phenyl group to which X₃ is not linked.

In addition, in Formula 3, a hydrogen atom linked to a carbon atom of biphenyl halide to which X₃ is not linked may be substituted with other substituents. Since other substituents of the compound represented by Formula 3 are similar to other substituents of the compound represented by Formula 2, a description thereof will be omitted.

A method of preparing the condensed cyclic compound according to the embodiment includes a reaction between the compound represented by Formula 2 and the compound represented by Formula 3, and the condensed cyclic compound represented by Formula 1 may be obtained.

In addition, for example, in terms of reliably obtaining the condensed cyclic compound represented by Formula 1, the compound represented by Formula 2 may be a compound represented by Formula 2-1 or 2-2.

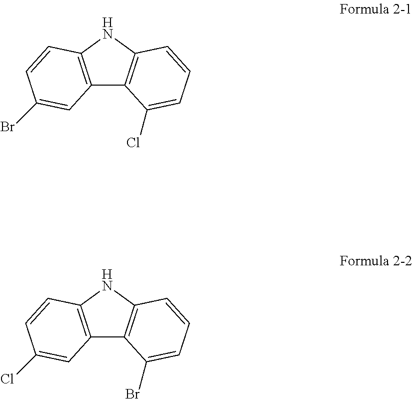 [SMILES: Clc1cccc2[nH]c3ccc(Br)cc3c12 ; Clc1ccc2[nH]c3cccc(Br)c3c2c1]

In the compound represented by Formula 2-1 or 2-2, X₁ and X₂ may each independently be a chlorine atom or a bromine atom. In the compound represented by Formula 2-1, X₁ linked to the 3rd carbon atom of the carbazole ring is a bromine atom, and X₂ linked to the 5th carbon atom of the carbazole ring is a chlorine atom. In addition, in the compound represented by Formula 2-2, X₁ linked to the 3rd carbon atom of the carbazole ring is a chlorine atom, and X₂ linked to the 5th carbon atom of the carbazole ring is a bromine atom.

In addition, the compound represented by Formula 2 may be an intermediate (or precursor) for obtaining the condensed cyclic compound represented by Formula 1. Therefore, even when an organic light-emitting device is manufactured by wet coating, the compound represented by Formula 2 is suitable in terms of capable of obtaining a compound that may maintain or improve current efficiency and emission lifespan.

Material for Light-Emitting Device

A material for a light-emitting device according to an embodiment may include the condensed cyclic compound represented by Formula 1. The condensed cyclic compound represented by Formula 1 has high solubility to a solvent and suppresses aggregation of molecules. Therefore, a thin film having excellent quality may be manufactured even by wet coating using the condensed cyclic compound represented by Formula 1. In addition, the condensed cyclic compound represented by Formula 1 may have high hole transport capability. Therefore, a material for a light-emitting device including the condensed cyclic compound represented by Formula 1 may be used in, for example, a hole injection layer, a hole transport layer, and an emission layer of an organic light-emitting device.

Therefore, even when the organic light-emitting device including the condensed cyclic compound represented by Formula 1 is manufactured by wet coating, the current efficiency and emission lifespan may be improved.

Therefore, the material for the light-emitting device including the condensed cyclic compound represented by Formula 1 may be used for manufacturing the light-emitting device. In this case, the light-emitting device having excellent current efficiency and emission lifespan may be manufactured by a coating method.

Composition

Hereinafter, a composition according to an embodiment will be described in detail.

The composition is used for forming each layer of the light-emitting device by using a solution coating method.

The composition may include the condensed cyclic compound represented by Formula 1.

In an embodiment, the composition may further include a light-emitting material.

The light-emitting material is not particularly limited as long as the light-emitting material has a light-emitting function, and may be a fluorescent dopant, a phosphorescent dopant, a quantum dot, or the like.

The fluorescent dopant is a compound that may emit light from singlet exciton. For example, the fluorescent dopant may be a perylene and a derivative thereof, a rubrene and a derivative thereof, a coumarin and a derivative thereof, and 4-dicyanomethylene-2-(p-dimethylaminostyryl)-6-methyl-4H-pyran (DCM) and a derivative thereof, but embodiments of the present disclosure are not limited thereto.

The phosphorescent dopant is a compound that may emit light from triplet exciton. The phosphorescent dopant may be an organometallic compound. For example, the phosphorescent dopant may be an iridium complex such as bis[2-(4,6-difluorophenyl)pyridinate] picolinate iridium(III) (FIrpic), bis(1-phenylisoquinoline)(acetylacetonate) iridium(III) (Ir(piq)₂(acac)), tris(2-phenylpyridine) iridium(III) (Ir(ppy)₃), and tris(2-(3-p-xylyl)phenyl)pyridine iridium(III)), an osmium complex, a platinum complex, or the like, but embodiments of the present disclosure are not limited thereto.

The quantum dot may be a nanoparticle including group II-VI semiconductor, group III-V semiconductor, or group IV-IV semiconductor. For example, the quantum dot may be CdO, CdS, CdSe, CdTe, ZnO, ZnS, ZnSe, ZnTe, HgS, HgSe, HgTe, MgSe, MgS CdSeS, CdSeTe, CdSTe, ZnSeS, ZnSeTe, ZnSTe, HgSeS, HgSeTe, HgSTe, CdZnS, CdZnSe, CdZnTe, CdHgS, CdHgSe, CdHgTe, HgZnS, HgZnSe, HgZnTe, MgZnSe, MgZnS, GaN, GaP, GaAs, AlN, AlP, AlAs, InN, InP, InAs, InSb, GaNP, GaNAs, GaNSb, GaPAs, GaPSb, AlNP, AlNAs, AlNSb, AlPAs, AlPSb, InNP, InNAs, InPAs, InPSb, GaAlNP, SnS, SnSe, SnTe, PbS, PbSe, PbTe, SnSeS, SnSeTe, SnSTe, PbSeS, PbSeTe, PbSTe, SnPbS, SnPbSe, SnPbTe, or the like, but embodiments of the present disclosure are not limited thereto. In addition, the diameter of the quantum dot is not particularly limited, but may be in a range of about 1 nanometer (nm) to about 20 nm. The quantum dot may be a single core structure, or may be a core-shell structure.

In an embodiment, the composition may further include an electron transport host and a light-emitting material.

The composition may further include a solvent.

The solvent included in the composition is not particularly limited as long as the condensed cyclic compound represented by Formula 1 is dissolved therein. For example, the solvent may be toluene, xylene, ethylbenzene, diethylbenzene, mesitylene, propylbenzene, cyclohexylbenzene, dimethoxybenzene, anisole, ethoxytoluene, phenoxytoluene, iso-propylbiphenyl, dimethylanisole, phenyl acetate, phenyl propionic acid, methyl benzoate, ethyl benzoate, or the like, but embodiments of the present disclosure are not limited thereto.

The concentration of the condensed cyclic compound represented by Formula 1 in the composition may be in a range of about 0.1 percent by weight (weight %) to about 10 weight %, for example, about 0.5 weight % to about 5 weight %, but embodiments of the present disclosure are not limited thereto. When the concentration of the condensed cyclic compound represented by Formula 1 in the composition is within this range, coatability may be improved.

Therefore, the composition may be used as the material for the light-emitting device (for example, an organic light-emitting device, a quantum dot light-emitting device, or the like). The composition may be used for the emission layer, the charge injection layer, and/or the charge transport layer of the light-emitting device. The composition may be used for the emission layer of the light-emitting device. The composition may be used to manufacture the light-emitting device by using a solution coating method. At this time, the current efficiency and emission lifespan of the light-emitting device may be maintained or improved.

Organic Light-Emitting Device

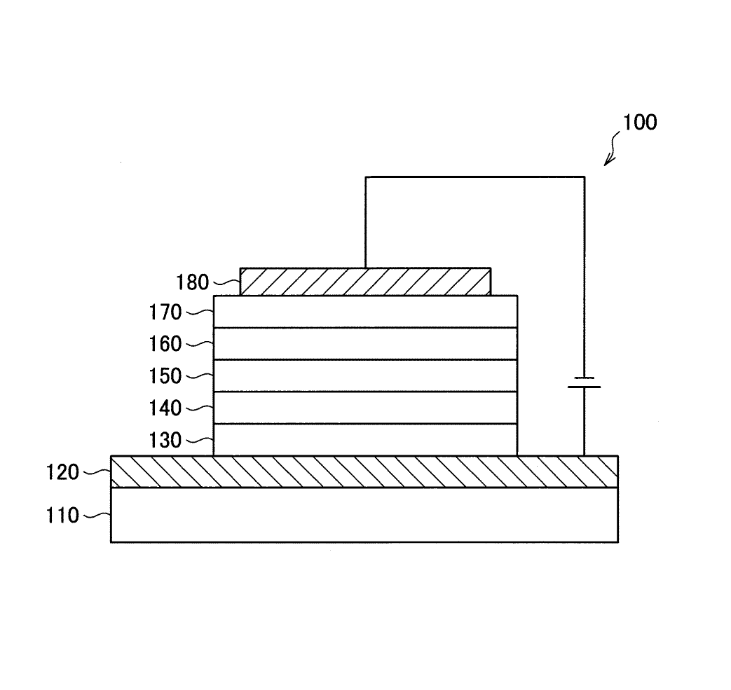

Hereinafter, an organic light-emitting device according to an embodiment will be described in connection with the FIGURE. The FIG. 1s a schematic cross-sectional view of an organic light-emitting device according to an embodiment.

An organic light-emitting device 100 according to an embodiment may include a substrate 110, a first electrode 120 disposed on the substrate 110, a hole injection layer 130 disposed on the first electrode 120, a hole transport layer 140 disposed on the hole injection layer 130, an emission layer 150 disposed on the hole transport layer 140, an electron transport layer 160 disposed on the emission layer 150, an electron injection layer 170 disposed on the electron transport layer 160, and a second electrode 180 disposed on the electron injection layer 170.

The condensed cyclic compound represented by Formula 1 may be, for example, included in any one of the organic layer (for example, the hole injection layer 130, the hole transport layer 140, the emission layer 150, the electron transport layer 160, or the electron injection layer 170) disposed between the first electrode 120 and the second electrode 180. For example, the condensed cyclic compound represented by Formula 1 may be included as a host in the emission layer 150. In an embodiment, the condensed cyclic compound represented by Formula 1 may be included in the organic layer other than the emission layer 150. For example, the condensed cyclic compound represented by Formula 1 may be included as an electron transport material in the hole injection layer 130 and/or the hole transport layer 140.

The term “organic layer” as used herein refers to a single layer and/or a plurality of layers disposed between the first electrode and the second electrode of the organic light-emitting device. The “organic layer” may include, in addition to an organic compound, an organometallic compound including metal.

The expression “(an organic layer) includes at least one organometallic compound” as used herein may include an embodiment in which “(an organic layer) includes identical compounds represented by Formula 1” and an embodiment in which “(an organic layer) includes two or more different organometallic compounds represented by Formula 1.”

For example, the organic layer may include, as the condensed cyclic compound, only Compound 1. In this embodiment, Compound 1 may be included in an emission layer of the organic light-emitting device. In one or more embodiments, the organic layer may include, as the condensed cyclic compound, Compound 1 and Compound 2. In this embodiment, Compound 1 and Compound 2 may be included in the same layer (for example, Compound 1 and Compound 2 may both be included in an emission layer).

The substrate 110 may be any substrate that is used in an organic light-emitting device according to the related art. For example, the substrate 110 may be a glass substrate, a silicon substrate, or a transparent plastic substrate, each having excellent mechanical strength, thermal stability, transparency, surface smoothness, ease of handling, and water resistance, but embodiments of the present disclosure are not limited thereto.

The first electrode 120 may be formed on the substrate 110. The first electrode 120 may be, for example, an anode, and may be formed of a material selected from metals, alloys, or conductive compounds, each having a high work function to facilitate hole injection. The first electrode 120 may be a reflective electrode, a semi-transmissive electrode, or a transmissive electrode. The first electrode 120 may have a single-layered structure or a multi-layered structure including two or more layers. For example, the first electrode 120 may be a transparent electrode formed of indium tin oxide (ITO), indium zinc oxide (IZO), tin oxide (SnO₂), or zinc oxide (ZnO) that has excellent transparency and conductivity. The first electrode 120 may be prepared as a reflective electrode by depositing, on the transparent electrode, magnesium (Mg), aluminum (Al), aluminum-lithium (Al—Li), calcium (Ca), magnesium-indium (Mg—In), or magnesium-silver (Mg—Ag). In an embodiment, the first electrode 120 may have a three-layered structure of ITO/Ag/ITO, but embodiments of the present disclosure are not limited thereto.

The hole transport region may be formed on the first electrode 120.

The hole transport region may include at least one selected from a hole injection layer 130, a hole transport layer 140, an electron blocking layer (not shown), and a buffer layer (not shown).

The hole transport region may include only the hole injection layer 130, or only the hole transport layer 140. In one or more embodiments, the hole transport region may have a hole injection layer/hole transport layer structure or a hole injection layer/hole transport layer/electron blocking layer structure, wherein for each structure, constituting layers are sequentially stacked in this stated order from the first electrode 120.

The hole injection layer 130 may include at least one selected from, for example, poly(ether ketone)-containing triphenylamine (TPAPEK), a 4-iso-propyl-4′-methyldiphenyliodonium tetrakis(pentafluorophenyl) borate (PPBI), N,N′-diphenyl-N,N′-bis-[4-(phenyl-m-tolyl-amino)-phenyl]-biphenyl-4,4′-diamine (DNTPD), copper phthalocyanine, 4,4′,4″-tris(3-methylphenylphenylamino) triphenylamine (m-MTDATA), N, N′-di(1-naphthyl)-N, N′-diphenylbenzidine (NPB), 4,4′,4″-tris(diphenylamino) triphenylamine (TDATA), 4,4′,4″-tris(N,N-2-naphthylphenylamino) triphenylamine (2-TNATA), polyaniline/dodecylbenzene sulphonic acid (PANI/DBSA), poly(3,4-ethylenedioxythiophene)/poly(4-styrene sulfonate) (PEDOT/PSS), polyaniline/10-camphor sulfonic acid (PANI/CSA), and polyaniline/poly(4-styrene sulfonate) (PANI/PSS).

The hole injection layer 130 may be formed to a thickness in a range of about 10 nm to about 1,000 nm, for example, a range of about 10 nm to about 100 nm.

The hole transport layer 140 may include at least one selected from, for example, a carbazole derivative, such as 1,1-bis[(di-4-tolylamino)phenyl] cyclohexane (TAPC), N-phenylcarbazole, and polyvinylcarbazole, N, N′-bis(3-methylphenyl)-N, N′-diphenyl-[1,1-biphenyl]-4,4′-diamine (TPD), 4,4′,4″-tris(N-carbazolyl) triphenylamine (TCTA), N,N′-di(1-naphthyl)-N,N′-diphenylbenzidine (NPB), and poly(9,9-dioctyl-fluorene-co-N-(4-butylphenyl)-diphenylamine (TFB).

The hole transport layer 140 may be formed to a thickness in a range of about 10 nm to about 1,000 nm, for example, a range of about 10 nm to about 150 nm.

The hole transport region may further include, in addition to these materials, a charge-generation material for the improvement of conductive properties. The charge-generation material may be homogeneously or non-homogeneously dispersed in the hole transport region.

The charge-generation material may be, for example, a p-dopant. The p-dopant may be one selected from a quinone derivative, a metal oxide, and a cyano group-containing compound, but embodiments of the present disclosure are not limited thereto. Non-limiting examples of the p-dopant are a quinone derivative, such as tetracyanoquinonedimethane (TCNQ) or 2,3,5,6-tetrafluoro-tetracyano-1,4-benzoquinonedimethane (F4-TCNQ); a metal oxide, such as a tungsten oxide or a molybdenium oxide; and a cyano group-containing compound, such as Compound HT-D1 or Compound HT-D2 below, but are not limited thereto.

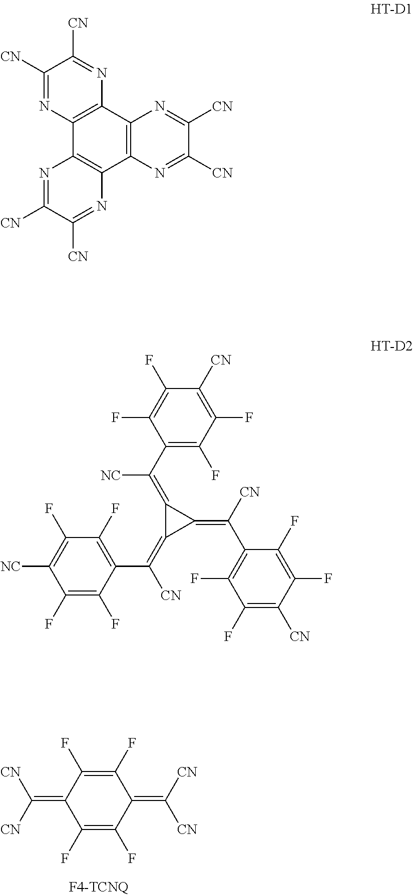

Meanwhile, when the hole transport region includes a buffer layer, a material for the buffer layer may be selected from materials for the hole transport region described above and materials for a host to be explained later. However, the material for the buffer layer is not limited thereto.

Meanwhile, when the hole transport region includes an electron blocking layer, a material for the electron blocking layer may be selected from materials for the hole transport region described above and materials for a host to be explained later. However, the material for the electron blocking layer is not limited thereto. For example, when the hole transport region includes an electron blocking layer, a material for the electron blocking layer may be mCP.

The emission layer 150 may be formed on the hole transport region. The emission layer 150 is a layer emitting light by fluorescence or phosphorescence. The emission layer 150 may include a host and/or a dopant, and such a host may include the condensed cyclic compound represented by Formula 1. In addition, the host and the dopant included in the emission layer 150 may be any material known in the art.

For example, the host may include tris(8-quinolinato)aluminum (Alq₃), 4,4′-bis(carbazol-9-yl)biphenyl (CBP), poly(n-vinylcarbazole) (PVK), 9,10-di(naphthalene)anthracene (ADN), 4,4′,4″-tris(N-carbazolyl)triphenylamine (TCTA), 1,3,5-tris(N-phenyl-benzimidazol-2-yl)benzene (TPBi) 3-tert-butyl-9,10-di(naphth-2-yl)anthracene (TBADN), distyrylarylene (DSA), or 4,4′-bis(9-carbazole)-2,2′-dimethyl-bipheny (dmCBP), but embodiments of the present disclosure are not limited thereto.

In an embodiment, the condensed cyclic compound included in the emission layer 150 may be a hole transport host, and the emission layer 150 may further include an electron transport host.

In detail, the electron transport host may be Compound ETH-1, but embodiments of the present disclosure are not limited thereto:

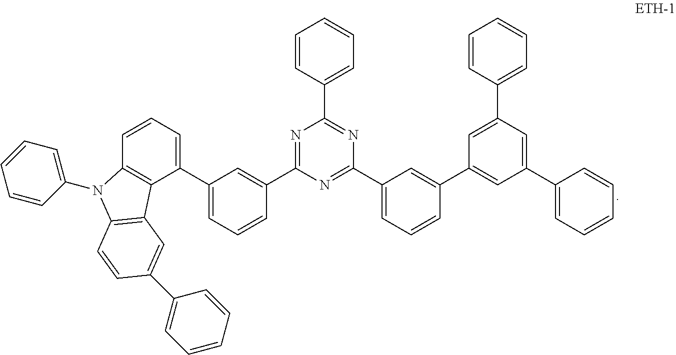 [SMILES: c1ccc(-c2cc(-c3ccccc3)cc(-c3cccc(-c4nc(-c5ccccc5)nc(-c5cccc(-c6cccc7c6c6cc(-c8ccccc8)ccc6n7-c6ccccc6)c5)n4)c3)c2)cc1]

For example, the dopant may include a perylene and a derivative thereof, a rubrene and a derivative thereof, a coumarin and a derivative thereof, 4-dicyanomethylene-2-(p-dimethylaminostyryl)-6-methyl-4H-pyran (DCM) and a derivative thereof, an iridium complex, such as bis[2-(4,6-difluorophenyl)pyridinate] picolinate iridium (III) (FIrpic), bis(1-phenylisoquinoline)(acetylacetonate) iridium (III) (Ir(piq)₂(acac)), tris(2-phenylpyridine) iridium (III) (Ir(ppy)₃), or tris(2-(3-p-xylyl)phenyl)pyridine iridium (III) (dopant), an osmium complex, or a platinum complex, but embodiments of the present disclosure are not limited thereto.

When the emission layer 150 includes the host and the dopant, an amount of the dopant may be in a range of about 0.01 parts by weight to about 15 parts by weight based on 100 parts by weight of the host, but embodiments of the present disclosure are not limited thereto.

The emission layer 150 may be formed to a thickness in a range of about 10 nm to about 60 nm.

When the organic light-emitting device is a full-color organic light-emitting device, the emission layer may be patterned into a red emission layer, a green emission layer, and a blue emission layer. In one or more embodiments, due to a stacked structure including a red emission layer, a green emission layer, and/or a blue emission layer, the emission layer may emit white light.

The electron transport region may be formed on the emission layer 150.

The electron transport region may include at least one selected from a hole blocking layer (not shown), an electron transport layer 160, and an electron injection layer 170.

For example, the electron transport region may have a hole blocking layer/electron transport layer/electron injection layer structure or an electron transport layer/electron injection layer structure, but the structure of the electron transport region is not limited thereto. The electron transport layer may have a single-layered structure or a multi-layered structure including two or more different materials.

For example, organic light-emitting device 100 may include a hole blocking layer between the electron transport layer 160 and the emission layer 150, so as to prevent diffusion of excitons or holes into the electron transport layer 160. The hole blocking layer may include, for example, at least one selected from an oxadiazole derivative, a triazole derivative, BCP, Bphen, BAlq, and Compound HB-1, but embodiments of the present disclosure are not limited thereto:

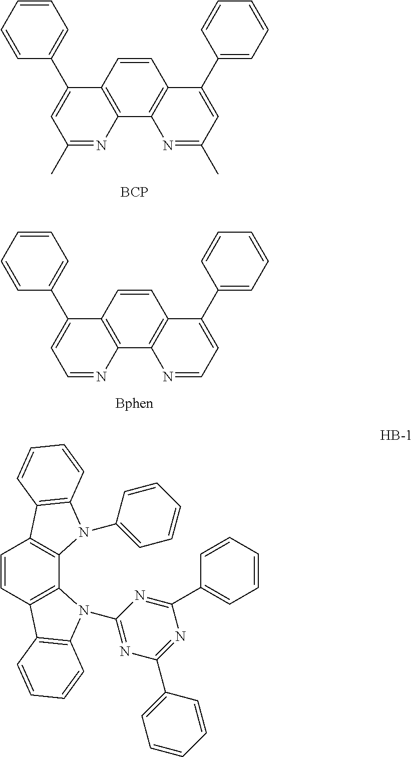

A thickness of the hole blocking layer may be in a range of about 20 Angstroms (Å) to about 1,000 Å, for example, about 30 Å to about 300 Å. While not wishing to be bound by theory, it is understood that when the thickness of the hole blocking layer is within these ranges, the hole blocking layer may have excellent hole blocking characteristics without a substantial increase in driving voltage.

In an embodiment, the electron transport layer 160 may include tris(8-quinolinato) aluminum (Alq₃), BAlq, a compound including a pyridine ring, such as 1,3,5-tri[(3-pyridyl)-phen-3-yl]benzene, a compound including a triazine ring, such as 2,4,6-tris(3′-(pyridin-3-yl)biphenyl-3-yl)-1,3,5-triazine, a compound including an imidazole ring, such as 2-(4-(N-phenylbenzimidazolyl-1-yl-phenyl)-9,10-dinaphthylanthracene, a compound including a triazole ring, such as TAZ and NTAZ, 1,3,5-tris(N-phenyl-benzimidazol-2-yl)benzene (TPBi), BCP, or Bphen:

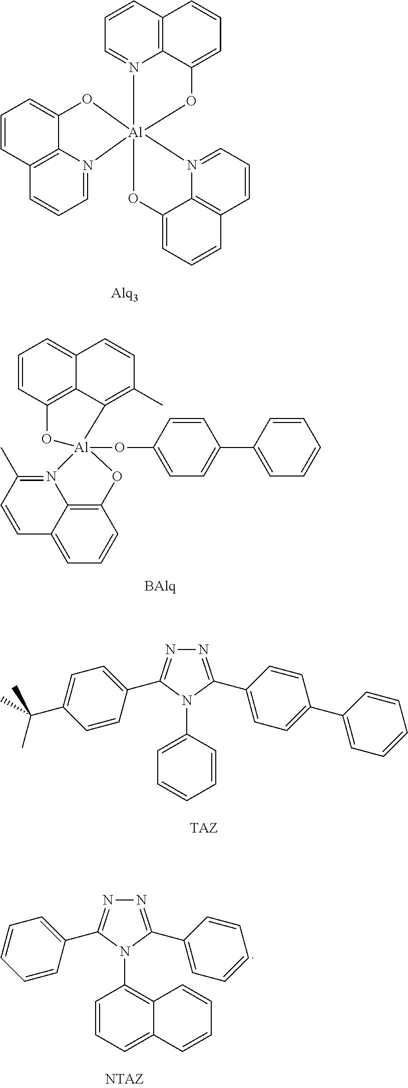

In one or more embodiments, the electron transport layer 160 may include a commercial product, such as KLET-01, KLET-02, KLET-03, KLET-10, or KLET-M1 (these products are available from Chemipro Kasei).

Also, the electron transport layer 160 may further include, in addition to the materials described above, a metal-containing material.

The metal-containing material may include a Li complex. The Li complex may include, for example, Compound ET-D1 (lithium 8-hydroxyquinolate, LiQ) or ET-D2.

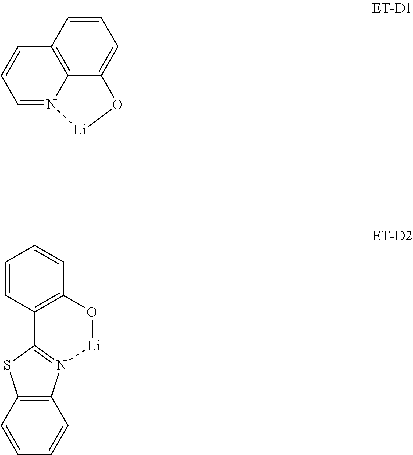

The electron transport layer 160 may be formed to a thickness, for example, in a range of about 15 nm to about 50 nm.

The electron injection layer 170 may be formed on the electron transport layer 160.

The electron injection layer 170 may include, for example, an lithium compound, such as (8-hydroxyquinolinato)lithium (LiQ) and lithium fluoride (LiF), sodium chloride (NaCl), cesium fluoride (CsF), lithium oxide (Li₂O), or barium oxide (BaO).

The electron injection layer 170 may be formed to a thickness in a range of about 0.3 nm to about 9 nm.

The second electrode 180 may be formed on the electron injection layer 170. The second electrode 180 may be a cathode and may be formed by using a material having a low work function among a metal, an alloy, an electrically conductive compound, and any combination thereof. For example, the second electrode 180 may be formed as a reflective electrode by using a metal such as lithium (Li), magnesium (Mg), aluminum (Al), and calcium (Ca), or an alloy such as aluminum-lithium (Al—Li), magnesium-indium (Mg—In), and magnesium-silver (Mg—Ag). Alternatively, the second electrode 180 may be formed as a transparent electrode by using the metal or the alloy thin film having a thickness of 20 nm or less, or a transparent conductive film such as indium tin oxide (In₂O₃—SnO₂) and indium zinc oxide (In₂O₃—ZnO).

In addition, the stacked structure of the organic light-emitting device 100 according to the embodiment is not limited to the above-described stacked structure. The organic light-emitting device 100 according to the embodiment may be formed in other known stacked structures. For example, in the organic light-emitting device 100, at least one selected from the hole injection layer 130, the hole transport layer 140, the electron transport layer 160, and the electron injection layer 170 may be omitted. The organic light-emitting device 100 may further include another layer. In addition, each layer of the organic light-emitting device 100 may be a single layer or a multi-layer.

A method of manufacturing each layer of the organic light-emitting device 100 according to the embodiment is not particularly limited. For example, each layer of the organic light-emitting device 100 according to the embodiment may be manufactured by using various methods, such as vacuum deposition, solution coating, and Langmuir-Blodgett (LB) deposition.

The solution coating may include spin coating, casting, micro gravure coating, gravure coating, bar coating, roll coating, wire bar coating, dip coating, spry coating, screen printing, flexographic printing, offset printing, and ink-jet printing.

Examples of the solvent used in the solution coating may include toluene, xylene, diethyl ether, chloroform, ethyl acetate, dichloromethane, tetrahydrofuran, acetone, acetonitrile, N,N-dimethylformamide, dimethylsulfoxide, anisole, hexamethylphosphoric acid triamide, 1,2-dichloroethane, 1,1,2-trichloroethane, chlorobenzene, o-dichlorobenzene, dioxane, cyclohexane, n-pentane, n-hexane, n-heptane, n-octane, n-nonane, n-decane, methyl ethyl ketone, cyclohexanone, butyl acetate, ethyl cellosolve acetate, ethylene glycol, ethylene glycol monobutyl ether, ethylene glycol monoethyl ether, ethylene glycol monomethyl ether, dimethoxy ethane, propylene glycol, diethoxymethane, triethylene glycol monoethyl ether, glycerin, 1,2-hexanediol, methanol, ethanol, propanol, iso-propanol, cyclohexanol, and N-methyl-2-pyrrolidone, but the solvent is not limited as long as the solvent can dissolve the material used to form each layer.

Considering the coatability, the concentration of the composition used in the solution coating may be in a range from about 0.1 weight % to about 10 weight %, for example, in a range from about 0.5 weight % to about 5 weight %, but embodiments of the present disclosure are not limited thereto.

The compound used in the vacuum deposition may be different according to the structure and thermal characteristics of the target layer, but may be selected from, for example, a deposition temperature of about 100° C. to about 500° C., a vacuum degree of about 10⁻⁸ torr to about 10⁻³ torr, a deposition rate of about 0.01 Angstroms per second (A/sec) to about 100 Å/sec.

In an embodiment, the first electrode 120 may be an anode, and the second electrode 180 may be a cathode.

For example, the first electrode 120 may be an anode; the second electrode 180 may be a cathode; the organic layer may include the emission layer 150 between the first electrode 120 and the second electrode 180; the organic layer may further include a hole transport region disposed between the first electrode 120 and the emission layer 150 and an electron transport region disposed between the emission layer 150 and the second electrode 180; wherein the hole transport region may include at least one selected from a hole injection layer 130, a hole transport layer 140, a buffer layer, and an electron blocking layer; and wherein the electron transport region may include at least one selected from a hole blocking layer, an electron transport layer 160, and an electron injection layer 170.

In one or more embodiments, the first electrode 120 may be a cathode, and the second electrode 180 may be an anode.

Hereinbefore, the organic light-emitting device has been described with reference to the FIGURE, but embodiments of the present disclosure are not limited thereto.

Description of Substituents

The expression “X and Y may each independently be” as used herein refers to a case where X and Y may be identical to each other, or a case where X and Y may be different from each other.

The term “substituted” as used herein refers to a case where hydrogen of a substituent such as R₁ may be further substituted with other substituents.

The term “C₁-C₆₀ alkyl group” as used herein refers to a linear or branched aliphatic hydrocarbon monovalent group having 1 to 60 carbon atoms, and non-limiting examples thereof include a methyl group, an ethyl group, an n-propyl group, an iso-propyl group, an n-butyl group, an iso-butyl group, a sec-butyl group, a tert-butyl group, an n-pentyl group, an iso-pentyl group, a tert-pentyl group, a neo-pentyl group, a 1,2-dimethylpropyl group, an n-hexyl group, an iso-hexyl group, a 1,3-dimethylbutyl group, a 1-iso-propylpropyl group, a 1,2-dimethylbutyl group, an n-heptyl group, a 1,4-dimethylpentyl group, a 3-ethylpentyl group, a 2-methyl-1-iso-propylpropyl group, a 1-ethyl-3-methylbutyl group, an n-octyl group, a 2-ethylhexyl group, a 3-methyl-1-iso-propylbutyl group, a 2-methyl-1-iso-propyl group, a 1-tert-butyl-2-methylpropyl group, an n-nonyl group, a 3,5,5-trimethyldecyl group, an n-decyl group, an iso-decyl group, an n-undecyl group, a 1-methyldecyl group, an n-dodecyl group, an n-tridecyl group, an n-tetradecyl group, an n-pentadecyl group, an n-hexadecyl group, an n-heptadecyl group, n-octadecyl group, an n-nonadecyl group, an n-eicosyl group, an n-heneicosyl group, an n-docosyl group, an n-tricosyl group, and an n-tetracosyl group.

The term “C₁-C₆₀ alkylene group” as used herein refers to a divalent group having the same structure as the C₁-C₆₀ alkyl group.

The term “C₁-C₆₀ alkoxy group” as used herein refers to a monovalent group represented by-OA₁₀₁ (wherein A₁₀₁ is the C₁-C₂₄ alkyl group), and non-limiting examples thereof include a methoxy group, an ethoxy group, a propoxy group, an iso-propoxy group, an n-butoxy group, an iso-butoxy group, a sec-butoxy group, tert-butoxy group, an n-pentoxy group, an iso-pentoxy group, a tert-pentoxy group, a neo-pentoxy group, an n-hexyloxy group, an iso-hexyloxy group, heptyloxy group, an octyloxy group, a nonyloxy group, a decyloxy group, an undecyloxy group, a dodecyloxy group, a tridecyloxy group, a tetradecyloxy group, a pentadecyloxy group, a hexadecyl group, a heptadecyl group, an octadecyl group, a 2-ethylhexyloxy group, and a 3-ethylpentyloxy group.

The term “C₁-C₆₀ alkylthio group” as used herein refers to a monovalent group represented by —SA₁₀₂ (wherein A₁₀₂ is the C₁-C₆₀ alkyl group).

The term “C₃-C₁₀ cycloalkyl group” as used herein refers to a monovalent saturated hydrocarbon monocyclic group having 3 to 10 carbon atoms involved in the ring formation, and examples thereof include a cyclopropyl group, a cyclobutyl group, a cyclopentyl group, a cyclohexyl group, and a cycloheptyl group. The term “C₃-C₁₀ cycloalkylene group” as used herein refers to a divalent group having the same structure as the C₃-C₁ cycloalkyl group.

The term “C₆-C₆₀ aryl group” as used herein refers to a monovalent group having a carbocyclic aromatic system having 6 to 60 carbon atoms involved in the ring formation (that is, when substituted with a substituent, the atom not included in the substituent is not counted as the carbon involved in the ring formation), and the term “C₆-C₆₀ arylene group” as used herein refers to a divalent group having a carbocyclic aromatic system having 6 to 60 carbon atoms. Non-limiting examples of the C₆-C₆₀ aryl group include a phenyl group, a naphthyl group, an anthracenyl group, a phenanthrenyl group, a pyrenyl group, and a chrysenyl group. When the C₆-C₆₀ aryl group and the C₆-C₆₀ arylene group each include two or more rings, the rings may be fused to each other.

The term “C₆-C₆₀ aryloxy group” as used herein refers to a group represented by —OA₁₀₃ (wherein A₁₀₃ is the C₆-C₆₀ aryl group), and non-limiting examples thereof include a 1-naphthyloxy group, a 2-naphthyloxy group, and a 2-azulenyloxy group.

The term “C₆-C₆₀ arylthio group” as used herein refers to a group represented by —SA₁₀₄ (wherein A₁₀₄ is the C₆-C₆₀ aryl group).

The term “C₁-C₆₀ heteroaryl group” as used herein refers to a monovalent group having a heterocyclic aromatic system that has at least one heteroatom selected from N, O, Si, P, and S as a ring-forming atom, and 1 to 60 carbon atoms. The term “C₁-C₆₀ heteroarylene group” as used herein refers to a divalent group having a heterocyclic aromatic system that has at least one heteroatom selected from N, O, Si, P, and S as a ring-forming atom, and 1 to 60 carbon atoms. Non-limiting examples of the C₁-C₆₀ heteroaryl group include a pyridinyl group, a pyrimidinyl group, a pyrazinyl group, a pyridazinyl group, a triazinyl group, a quinolinyl group, and an isoquinolinyl group. When the C₁-C₆₀ heteroaryl group and the C₁-C₆₀ heteroarylene group each include two or more rings, the rings may be fused to each other.

The term “C₁-C₆₀ hetero aryloxy group” as used herein refers to a group represented by-OA₁₀₅ (wherein A₁₀₅ is the C₁-C₆₀ heteroaryl group), and non-limiting examples thereof include a 2-furanyloxy group, a 2-thienyloxy group, a 2-indolyloxy group, a 3-indolyloxy group, a 2-benzofuryloxy group, and a 2-benzothienyloxy group.

The term “C₁-C₆₀ heteroarylthio group” as used herein refers to a group represented by —SA₁₀₆ (wherein A₁₀₆ is the C₁-C₆₀ heteroaryl group).

The term “C₇-C₃₀ arylalkyl group” as used herein refers to an alkyl group substituted with an aryl group, and is a monovalent group in which the sum of carbon atoms constituting the alkyl group and the aryl group is in a range of 7 to 30. Non-limiting examples of the C₇-C₃₀ arylalkyl group include a benzyl group, a phenylethyl group, a phenylpropyl group, and a naphthylmethyl group.

The term “C₆-C₃₀ arylalkyloxy group” as used herein refers to a monovalent group represented by —OA₁₀₅ (wherein A₁₀₅ is the C₇-C₃₀ arylalkyl group).

The term “C₆-C₃₀ arylalkylthio group” as used herein refers to a monovalent group represented by —SA₁₀₆ (wherein A₁₀₆ is the C₇-C₃₀ arylalkyl group).

The term “C₈-C₃₀ arylalkenyl group” as used herein refers to an alkenyl group substituted with an aryl group, and is a monovalent group in which the sum of carbon atoms constituting the alkenyl group and the aryl group is in a range of 8 to 30.

The term “monovalent non-aromatic condensed polycyclic group” as used herein refers to a monovalent group (for example, having 80 to 60 carbon atoms) having two or more rings condensed to each other, only carbon atoms as ring-forming atoms, and no aromaticity in its entire molecular structure. Examples of the monovalent non-aromatic condensed polycyclic group include a fluorenyl group. The term “divalent non-aromatic condensed polycyclic group” as used herein refers to a divalent group having the same structure as the monovalent non-aromatic condensed polycyclic group.

The term “monovalent non-aromatic condensed heteropolycyclic group” as used herein refers to a monovalent group (for example, having 1 to 60 carbon atoms) having two or more rings condensed to each other, has a heteroatom selected from N, O, P, Si, and S, other than carbon atoms, as a ring-forming atom, and no aromaticity in its entire molecular structure. Examples of the monovalent non-aromatic condensed heteropolycyclic group include a carbazolyl group. The term “divalent non-aromatic condensed heteropolycyclic group” as used herein refers to a divalent group having the same structure as the monovalent non-aromatic condensed heteropolycyclic group.

The term “C₅-C₃₀ carbocyclic group” as used herein refers to a saturated or unsaturated cyclic group having, as a ring-forming atom, 5 to 30 carbon atoms only.

The “C₅-C₃₀ carbocyclic group” may be a monocyclic group or a polycyclic group, and depending on its chemical structure, may be a monovalent, divalent, trivalent, tetravalent, pentavalent, or hexavalent group.

The term “C₁-C₃₀ heterocyclic group” as used herein refers to a saturated or unsaturated cyclic group having at least one heteroatom selected from N, O, P, Si, and S as a ring-forming atom, in addition to 1 to 30 carbon atoms. The “C₁-C₃₀ heterocyclic group” as used herein may be a monocyclic group or a polycyclic group, and depending on its chemical structure, may be a monovalent, divalent, trivalent, tetravalent, pentavalent, or hexavalent group.

At least one substituent of the substituted C₅-C₃₀ carbocyclic group, the substituted C₁-C₃₀ heterocyclic group, the substituted C₁-C₆₀ alkyl group, the substituted C₂-C₆₀ alkenyl group, the substituted C₂-C₆₀ alkynyl group, the substituted C₁-C₆₀ alkoxy group, the substituted C₃-C₁₀ cycloalkyl group, the substituted C₁-C₁₀ heterocycloalkyl group, the substituted C₃-C₁₀ cycloalkenyl group, the substituted C₁-C₁₀ heterocycloalkenyl group, the substituted C₆-C₆₀ aryl group, the substituted C₆-C₆₀ aryloxy group, the substituted C₆-C₆₀ arylthio group, the substituted C₁-C₆₀ heteroaryl group, the substituted monovalent non-aromatic condensed polycyclic group, and the substituted monovalent non-aromatic condensed heteropolycyclic group may be selected from:

deuterium, —F, —Cl, —Br, —I, —CD₃, —CD₂H, —CDH₂, —CF₃, —CF₂H, —CFH₂, a hydroxyl group, a cyano group, a nitro group, an amino group, an amidino group, a hydrazine group, a hydrazone group, a carboxylic acid group or a salt thereof, a sulfonic acid group or a salt thereof, a phosphoric acid group or a salt thereof, a C₁-C₆₀ alkyl group, a C₂-C₆₀ alkenyl group, a C₂-C₆₀ alkynyl group, and a C₁-C₆₀ alkoxy group;

a C₁-C₆₀ alkyl group, a C₂-C₆₀ alkenyl group, a C₂-C₆₀ alkynyl group, and a C₁-C₆₀ alkoxy group, each substituted with at least one selected from deuterium, —F, —Cl, —Br, —I, —CD₃, —CD₂H, —CDH₂, —CF₃, —CF₂H, —CFH₂, a hydroxyl group, a cyano group, a nitro group, an amino group, an amidino group, a hydrazine group, a hydrazone group, a carboxylic acid group or a salt thereof, a sulfonic acid group or a salt thereof, a phosphoric acid group or a salt thereof, a C₃-C₁₀ cycloalkyl group, a C₁-C₁₀ heterocycloalkyl group, a C₃-C₁₀ cycloalkenyl group, a C₁-C₁₀ heterocycloalkenyl group, a C₆-C₆₀ aryl group, a C₆-C₆₀ aryloxy group, a C₆-C₆₀ arylthio group, a C₁-C₆₀ heteroaryl group, a monovalent non-aromatic condensed polycyclic group, a monovalent non-aromatic condensed heteropolycyclic group, —N(Q₁₁)(Q₁₂), —Si(Q₁₃)(Q₁₄)(Q₁₅), —B(Q₁₆)(Q₁₇), and —P(═O)(Q₁₈)(Q₁₉);

a C₃-C₁₀ cycloalkyl group, a C₁-C₁₀ heterocycloalkyl group, a C₃-C₁₀ cycloalkenyl group, a C₁-C₁₀ heterocycloalkenyl group, a C₆-C₆₀ aryl group, a C₆-C₆₀ aryloxy group, a C₆-C₆₀ arylthio group, a C₁-C₆₀ heteroaryl group, a monovalent non-aromatic condensed polycyclic group, and a monovalent non-aromatic condensed heteropolycyclic group;

a C₃-C₁₀ cycloalkyl group, a C₁-C₁₀ heterocycloalkyl group, a C₃-C₁₀ cycloalkenyl group, a C₁-C₁₀ heterocycloalkenyl group, a C₆-C₆₀ aryl group, a C₆-C₆₀ aryloxy group, a C₆-C₆₀ arylthio group, a C₁-C₆₀ heteroaryl group, a monovalent non-aromatic condensed polycyclic group, and a monovalent non-aromatic condensed heteropolycyclic group, each substituted with at least one selected from deuterium, —F, —Cl, —Br, —I, —CD₃, —CD₂H, —CDH₂, —CF₃, —CF₂H, —CFH₂, a hydroxyl group, a cyano group, a nitro group, an amino group, an amidino group, a hydrazine group, a hydrazone group, a carboxylic acid group or a salt thereof, a sulfonic acid group or a salt thereof, a phosphoric acid group or a salt thereof, a C₁-C₆₀ alkyl group, a C₂-C₆₀ alkenyl group, a C₂-C₆₀ alkynyl group, a alkoxy group, a C₃-C₁₀ cycloalkyl group, a C₁-C₁₀ heterocycloalkyl group, a C₃-C₁₀ cycloalkenyl group, a C₁-C₁₀ heterocycloalkenyl group, a C₆-C₆₀ aryl group, a C₆-C₆₀ aryloxy group, a C₆-C₆₀ arylthio group, a C₁-C₆₀ heteroaryl group, a monovalent non-aromatic condensed polycyclic group, a monovalent non-aromatic condensed heteropolycyclic group, —N(Q₂₁)(Q₂₂), —Si(Q₂₃)(Q₂₄)(Q₂₅), —B(Q₂₆)(Q₂₇), and —P(═O)(Q₂₈)(Q₂₉); and

—N(Q₃₁)(Q₃₂), —Si(Q₃₃)(Q₃₄)(Q₃₅), —B(Q₃₆)(Q₃₇), and —P(═O)(Q₃₈)(Q₃₉), and

Q₁ to Q₉, Q₁₁ to Q₁₉, Q₂₁ to Q₂₉, and Q₃₁ to Q₃₉ may each independently be selected from hydrogen, deuterium, —F, —Cl, —Br, —I, a hydroxyl group, a cyano group, a nitro group, an amino group, an amidino group, a hydrazine group, a hydrazone group, a carboxylic acid group or a salt thereof, a sulfonic acid group or a salt thereof, a phosphoric acid group or a salt thereof, a C₁-C₆₀ alkyl group, a C₂-C₆₀ alkenyl group, a C₂-C₆₀ alkynyl group, a C₁-C₆₀ alkoxy group, a C₃-C₁₀ cycloalkyl group, a C₁-C₁₀ heterocycloalkyl group, a C₃-C₁₀ cycloalkenyl group, a C₁-C₁₀ heterocycloalkenyl group, a C₆-C₆₀ aryl group, a C₆-C₆₀ aryl group substituted with at least one selected from a C₁-C₆₀ alkyl group, and a C₆-C₆₀ aryl group, a C₆-C₆₀ aryloxy group, a C₆-C₆₀ arylthio group, a C₁-C₆₀ heteroaryl group, a monovalent non-aromatic condensed polycyclic group, and a monovalent non-aromatic condensed heteropolycyclic group.

Others

The expression “A to B” as used herein refers to a range from A to B, including A and B.

While the embodiments of the present disclosure have been described with reference to the accompanying drawings, it is understood that the present disclosure is not limited to these embodiments. It is apparent to those of ordinary skill in the art that various modifications or changes may be made thereto without departing from the spirit and scope of the appended claims. It is understood that various modifications or changes fall within the technical scope of the present disclosure.

Hereinafter, a condensed cyclic compound represented by Formula 1 and an organic light-emitting device including the same will be described in detail with reference to Examples and Comparative Examples. Examples provided below are merely an example, and the condensed cyclic compound and the organic light-emitting device, according to embodiments, are not limited to Examples provided below.

The wording “B was used instead of A” used in describing Synthesis Examples means that a molar equivalent of “A” was identical to a molar equivalent of “B”.

In addition, “%” is percent by weight (weight %) unless specified otherwise.

### EXAMPLES

### Synthesis Example 1: Synthesis of Compound 14

(1) Synthesis of Compound 14-1

Compound 14-1 was synthesized according to the Reaction Scheme (4) below:

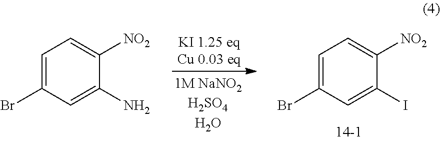 [SMILES: Nc1cc(Br)ccc1[N+](=O)[O-] ; O=[N+]([O-])c1ccc(Br)cc1I]

In a nitrogen atmosphere, 5-bromo-2-nitroaniline (122 grams (g), 562 millimoles, mmol), H₂SO₄ (122 milliliters, ml), and pure water (3.1 liters, L) were mixed and cooled to a temperature of 0° C., and an aqueous solution (620 ml) of NaNO₂ (42.7 g, 619 mmol, 1.1 eq) was added dropwise thereto for 1 hour and stirred at room temperature for 3 hours. A reaction solution was cooled to a temperature of 0° C., an aqueous solution (620 ml) of KI (117 g, 705 mmol, 1.25 equivalents, eq) was added dropwise thereto for 1 hour, and Cu powder (1.07 g, 16.8 mmol, 0.03 eq) was added. The reaction solution was stirred at a temperature of 70° C. for 3 hours. The reaction solution was cooled to room temperature, and an organic layer was extracted therefrom by using CH₂Cl₂. The organic layer was washed with a 10% Na₂S₂O₃ aqueous solution, dried by using anhydrous magnesium sulfate, filtered, and then concentrated. The resultant obtained therefrom was purified by silica gel chromatography (hexane:CH₂Cl₂=5:1 as developing solvent) to provide Compound 14-1 that was a yellow solid. 116.1 g of Compound 14-1 was obtained, and the yield of Compound 14-1 was 63%.

(2) Synthesis of Compound 14-2

Compound 14-2 was synthesized according to the Reaction Scheme (5) below:

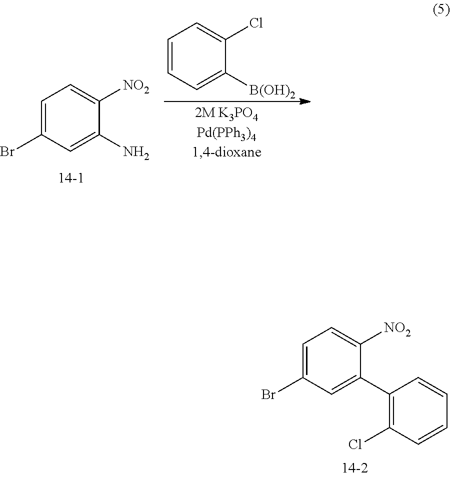

In a nitrogen atmosphere, Compound 14-1 (4-bromo-2-iodo-1-nitrobenzene) (231 g, 704 mmol), 1,4-dioxane (2.3 L), 2-chlorophenylboronic acid (132 g, 844 mmol), a 2 M K₃PO₄ aqueous solution (704 ml), and Pd(PPh₃)₄ (24.4 g, 21.1 mmol) were mixed and stirred at a temperature of 80° C. for 8 hours. The reaction solution was cooled to room temperature, and H₂O was added thereto. The organic layer was extracted therefrom by using ethyl acetate, dried by using anhydrous magnesium sulfate, filtered, and then concentrated. The resultant obtained therefrom was purified by silica gel chromatography (hexane:CH₂Cl₂=10:1 as developing solvent) to provide Compound 14-2 that was a yellow solid. 210 g of Compound 14-2 was obtained, and the yield of Compound 14-2 was 95%.

(3) Synthesis of Compound 14-3

Compound 14-3 was synthesized according to the Reaction Scheme (6) below:

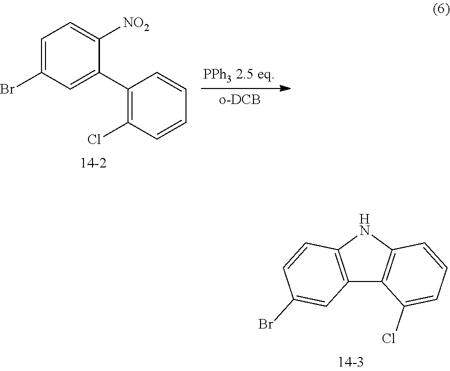 [SMILES: O=[N+]([O-])c1ccc(Br)cc1-c1ccccc1Cl ; Clc1cccc2[nH]c3ccc(Br)cc3c12]

In a nitrogen atmosphere, Compound 14-2 (5-bromo-2′-chloro-2-nitro-1,1′-biphenyl) (225 g, 720 mmol), o-dichlorobenzene (2.3 L), and PPh₃ (472 g, 1.8 mol) were mixed and stirred at a temperature of 180° C. for 6 hours. After the reaction solution was cooled to room temperature, the reaction solution was concentrated. The reaction solution was dissolved in a mixed solvent (hexane:CH₂Cl₂=2:1), and impurities were filtered three times by using a silica gel pad having a thickness of about 15 centimeters (cm), and the filtrate was concentrated. The resultant obtained therefrom was dispersed in 500 ml of a mixed solvent (hexane:CH₂Cl₂=2:1) and treated with ultrasound for 30 minutes. Then, the resultant was filtered to provide Compound 14-3. 67.1 g of Compound 14-3 was obtained, and the yield of Compound 14-3 was 33%.

(4) Synthesis of Compound 14-4

Compound 14-4 was synthesized according to the Reaction Scheme (7) below:

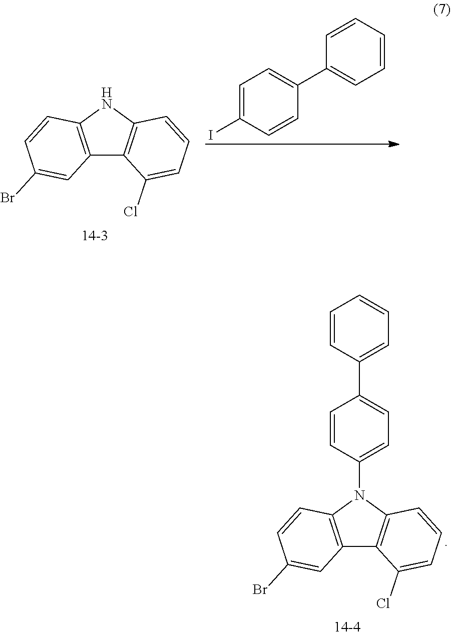

In a nitrogen atmosphere, Compound 14-3 (3-bromo-5-chloro-9H-carbazole) (28.06 g, 100 mmol), 1,4-dioxane (100 ml), 4-iodobiphenyl (30.81 g, 110 mmol), K₃PO₄ (31.84 g, 150 mmol), CuI (0.95 g, 5 mmol), and trans-1,2-diaminocyclohexane (1.71 g, 15 mmol) were mixed and stirred at a temperature of 100° C. for 24 hours. The reaction solution was cooled to room temperature, toluene (300 ml) was added thereto, and the resulting solution was filtered by using Celite. Additional filtration by using a silica gel pad and concentration was performed. The resultant obtained therefrom was purified by silica gel chromatography (hexane:CH₂Cl₂=9:1 as developing solvent), and recrystallized in hexane to provide Compound 14-4. 39.8 g of Compound 14-4 was obtained, and the yield of Compound 14-4 was 92%.

(5) Synthesis of Compound 14-5

Compound 14-5 was synthesized according to the Reaction Scheme (8) below:

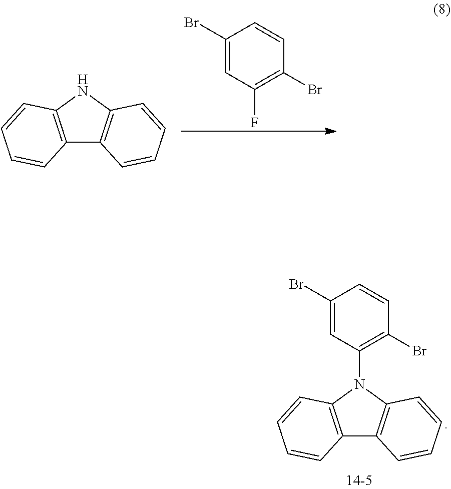 [SMILES: c1ccc2c(c1)[nH]c1ccccc12 ; Fc1cc(Br)ccc1Br ; Brc1ccc(Br)c(-n2c3ccccc3c3ccccc32)c1]

In a nitrogen atmosphere, carbazole (200.7 g, 1,200 mmol), 1,4-dibromo-2-fluorobenzene (335.2 g, 1,320 mmol), and 480 ml of N, N-dimethylformamide were added to a three-neck flask and mixed, and the reaction mixture was cooled in an ice bath. NaH (62 weight % dispersion in paraffin) (NaH content: 28.8 g, 1,200 mmol) was added stepwise while observing the amount of hydrogen gas generated, and the reaction mixture was stirred at a temperature of 150° C. for 12 hours. The reaction product was diluted in toluene (1 L), inactivated with a small amount of methanol, and filtered by using celite. The resultant obtained therefrom was washed with pure water three times in a separatory funnel, dried by using anhydrous magnesium sulfate, filtered by using a silica gel pad, and then concentrated. The resultant was purified by silica gel chromatography (hexane:CHCl₃=8:2 as developing solvent) and recrystallized in hexane to provide Compound 14-5. 341.74 g of Compound 14-5 was obtained, and the yield of Compound 14-5 was 71%.

(6) Synthesis of Compound 14-6

Compound 14-6 was synthesized according to the Reaction Scheme (9) below:

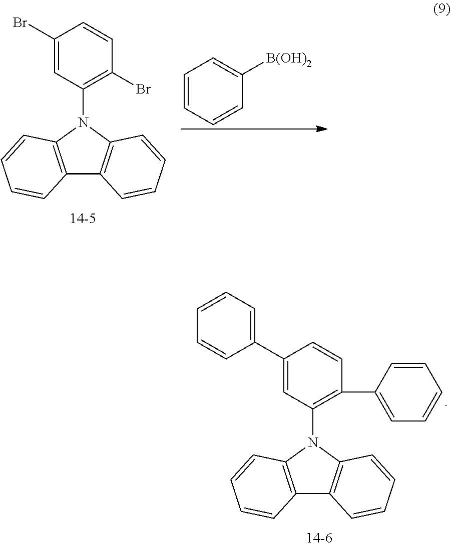

In a nitrogen atmosphere, Compound 14-5 (9-(2,5-dibromophenyl)-9H-carbazole) (20.05 g, 50 mmol), phenyl boronic acid (13.41 g, 110 mmol), toluene (200 ml), ethanol (50 ml), and a 2 M aqueous solution (100 ml) of potassium carbonate were added to a three-neck flask and mixed, and Pd(PPh₃)₄ (2.31 g, 2 mmol) was added thereto. The reaction solution was stirred at a temperature of 80° C. for 12 hours. The reaction solution was diluted by adding toluene (500 ml) thereto. The resultant obtained therefrom was cooled to room temperature, washed with pure water three times in a separatory funnel, dried by using anhydrous magnesium sulfate, filtered by using a silica gel pad, and then concentrated. The resultant was separated by silica gel chromatography (hexane:CHCl₃=8:2 as developing solvent) and recrystallized in hexane to provide Compound 14-6. 12.7 g of Compound 14-6 was obtained, and the yield of Compound 14-6 was 64%.

(7) Synthesis of Compound 14-7

Compound 14-7 was synthesized according to the Reaction Scheme (10) below:

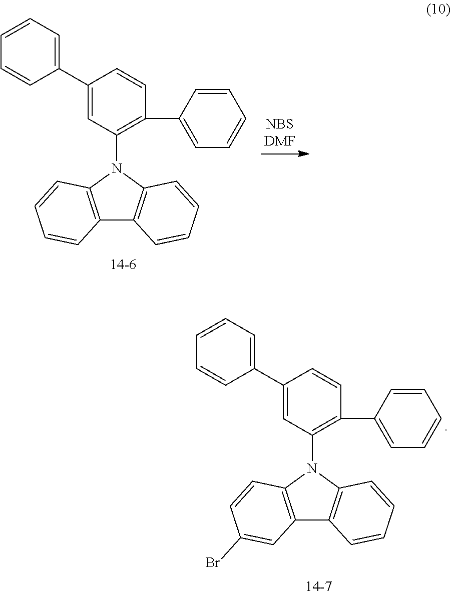 [SMILES: c1ccc(-c2ccc(-c3ccccc3)c(-n3c4ccccc4c4ccccc43)c2)cc1 ; Brc1ccc2c(c1)c1ccccc1n2-c1cc(-c2ccccc2)ccc1-c1ccccc1]

In a nitrogen atmosphere, Compound 14-6 (9-([1,1′,4′,1″-terphenyl]-2′-yl)-9H-carbazole) (12.6 g, 32 mmol), N,N-dimethylformamide (160 ml), and CHCl₃ (160 ml) were added to a three-neck flask and dissolved. While the reaction solution was cooled to a temperature of 0° C. in an ice bath, N-bromosuccinimide (5.70 g, 32 mmol) dissolved in N,N-dimethylformamide (32 ml) was added dropwise thereto for 10 minutes and stirred at room temperature for 12 hours. The reaction solution was diluted by adding toluene (500 ml) thereto. The resultant obtained therefrom was washed with pure water three times in a separatory funnel, dried by using anhydrous magnesium sulfate, filtered by using a silica gel pad, and then concentrated. The resultant was separated by silica gel chromatography (hexane:CHCl₃=8:2 as developing solvent) and recrystallized in hexane to provide Compound 14-7. 13.4 g of Compound 14-7 was obtained, and the yield of Compound 14-7 was 88%.

(8) Synthesis of Compound 14-8

Compound 14-8 was synthesized according to the Reaction Scheme (11) below:

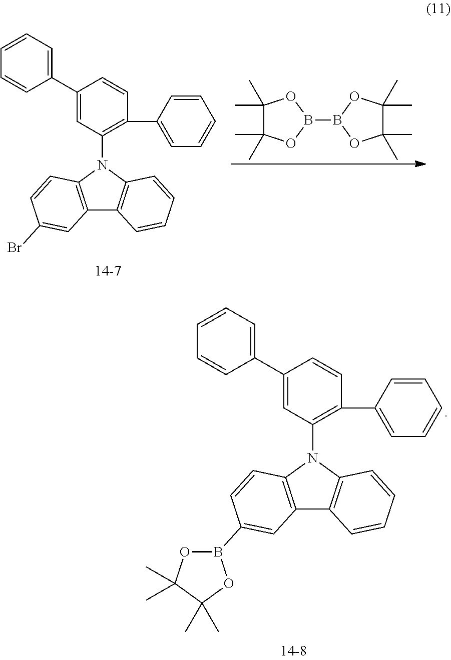

In a nitrogen atmosphere, Compound 14-7 (9-([1,1′,4′,1″-terphenyl]-2′-yl)-3-bromo-9H-carbazole) (13.3 g, 28 mmol), bis(pinacolato)diboron (7.82 g, 30.8 mmol), potassium acetate (5.50 g, 56 mmol), and N,N-dimethylformamide (140 ml) was added to a three-neck flask and mixed, and Pd(dppf)C₁₂ (2.05 g, 2.8 mmol) was added thereto and stirred at a temperature of 80° C. for 12 hours. The reaction product was diluted in toluene (300 ml), cooled to room temperature, filtered by using celite, washed with pure water three times in a separatory funnel, dried by using anhydrous magnesium sulfate, filtered by using a silica gel pad, and then concentrated. The resultant was purified by silica gel chromatography (hexane: CH₂Cl₂=6:4 as developing solvent) and recrystallized in hexane to provide Compound 14-8. 11.5 g of Compound 14-8 was obtained, and the yield of Compound 14-8 was 79%.

(9) Synthesis of Compound 14-9

Compound 14-9 was synthesized according to the Reaction Scheme (12) below:

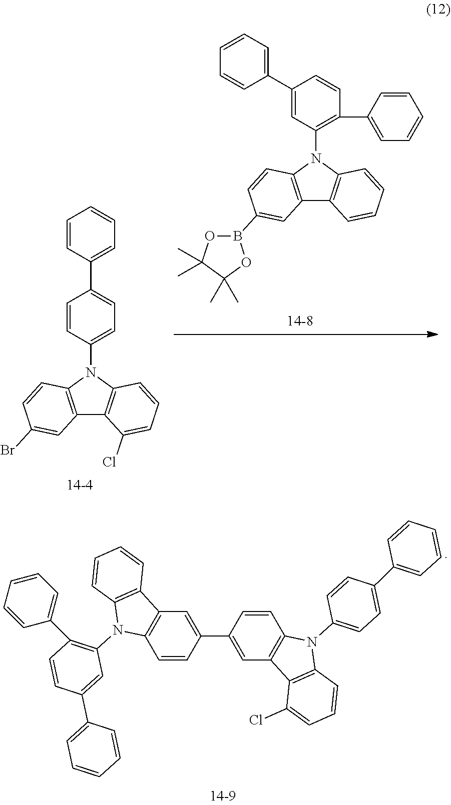 [SMILES: Clc1cccc2c1c1cc(Br)ccc1n2-c1ccc(-c2ccccc2)cc1 ; CC1(C)OB(c2ccc3c(c2)c2ccccc2n3-c2cc(-c3ccccc3)ccc2-c2ccccc2)OC1(C)C ; Clc1cccc2c1c1cc(-c3ccc4c(c3)c3ccccc3n4-c3cc(-c4ccccc4)ccc3-c3ccccc3)ccc1n2-c1ccc(-c2ccccc2)cc1]

In a nitrogen atmosphere, Compound 14-4 (9-([1,1′-biphenyl]-4-yl)-3-bromo-5-chloro-9H-carbazole) (4.33 g, 10 mmol), Compound 14-8 (9-([1,1′:4′,1″-terphenyl]-2′-yl)-3-(4,4,5,5-tetramethyl-1,3,2-dioxaborolan-2-yl)-9H-carbazole) (5.74 g, 11 mmol), toluene (100 ml), ethanol (20 ml), and a 2 M aqueous solution (10 ml) of potassium carbonate were added to a three-neck flask and mixed, and Pd(PPh₃)₄ (0.58 g, 0.5 mmol) was added thereto and stirred at a temperature of 80° C. for 8 hours. Pure water (100 ml) and methanol (200 ml) were added thereto and the reaction solution was cooled to room temperature. Then, a precipitated solid was collected by filtration. The solid was vacuum-dried (50° C., 6 hours), heated and dissolved in toluene (500 ml), filtered by a silica gel pad, and then concentrated. The resultant was recrystallized in a mixed solvent (hexane:ethyl acetate=1:1) twice to provide Compound 14-9. 4.9 g of Compound 14-9 was obtained, and the yield of Compound 14-9 was 65%.

(10) Synthesis of Compound 14

Compound 14 was synthesized according to the Reaction Scheme (13) below:

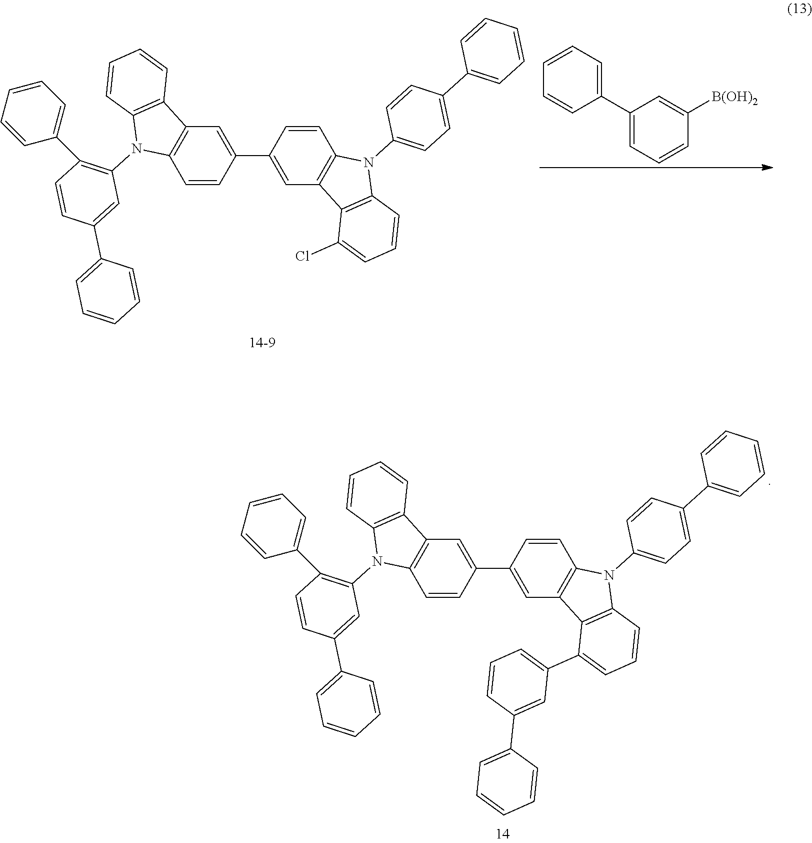

In a nitrogen atmosphere, Compound 14-9 (9-([1,1′-biphenyl]-4-yl)-9′-([1,1′,4′,1″-terphenyl]-2′-yl)-5-chloro-9H,9′H-3,3′-bicarbazole) (4.9 g, 6.5 mmol), 3-biphenylboronic acid (1.54 g, 7.8 mmol), toluene (65 ml), ethanol (13 ml), and a 2 M aqueous solution (6.5 ml) of potassium carbonate were added to a three-neck flask and mixed, and Pd(OAc)₂ (73 mg, 0.325 mmol) and S-Phos (213 mg, 0.52 mmol) were added thereto and stirred at a temperature of 80° C. for 12 hours. The reaction solution was diluted by adding toluene (300 ml) thereto. The resultant obtained therefrom was cooled to room temperature, washed with pure water three times in a separatory funnel, dried by using anhydrous magnesium sulfate, filtered by using a silica gel pad, and then concentrated. The resultant was separated by silica gel chromatography (hexane:toluene=7:3 as developing solvent) and recrystallized in a mixed solvent (hexane:ethyl acetate=1:1) three times to provide Compound 14. 3.71 g of Compound 14 was obtained, and the yield of Compound 14 was 66%.

### Synthesis Example 2: Synthesis of Compound 15

Compound 15-4 was obtained in the same manner as in Synthesis Example 1, except that the Reaction Scheme was changed to Reaction Scheme (14) and the corresponding reagent was changed.

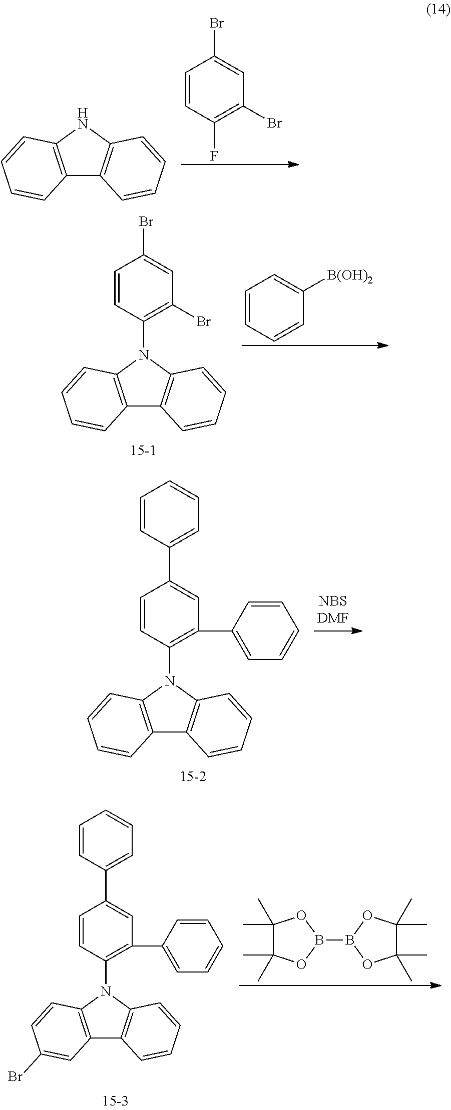

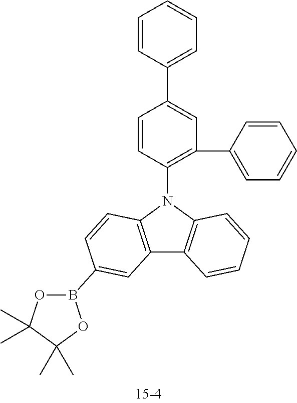 [SMILES: CC1(C)OB(c2ccc3c(c2)c2ccccc2n3-c2ccc(-c3ccccc3)cc2-c2ccccc2)OC1(C)C]

Compound 15 was obtained in the same manner as in Synthesis Example 1, except that the Reaction Scheme was changed to the Reaction Scheme (15) by using Compound 15-4 obtained in the Reaction Scheme (14) and the corresponding reagent was changed.

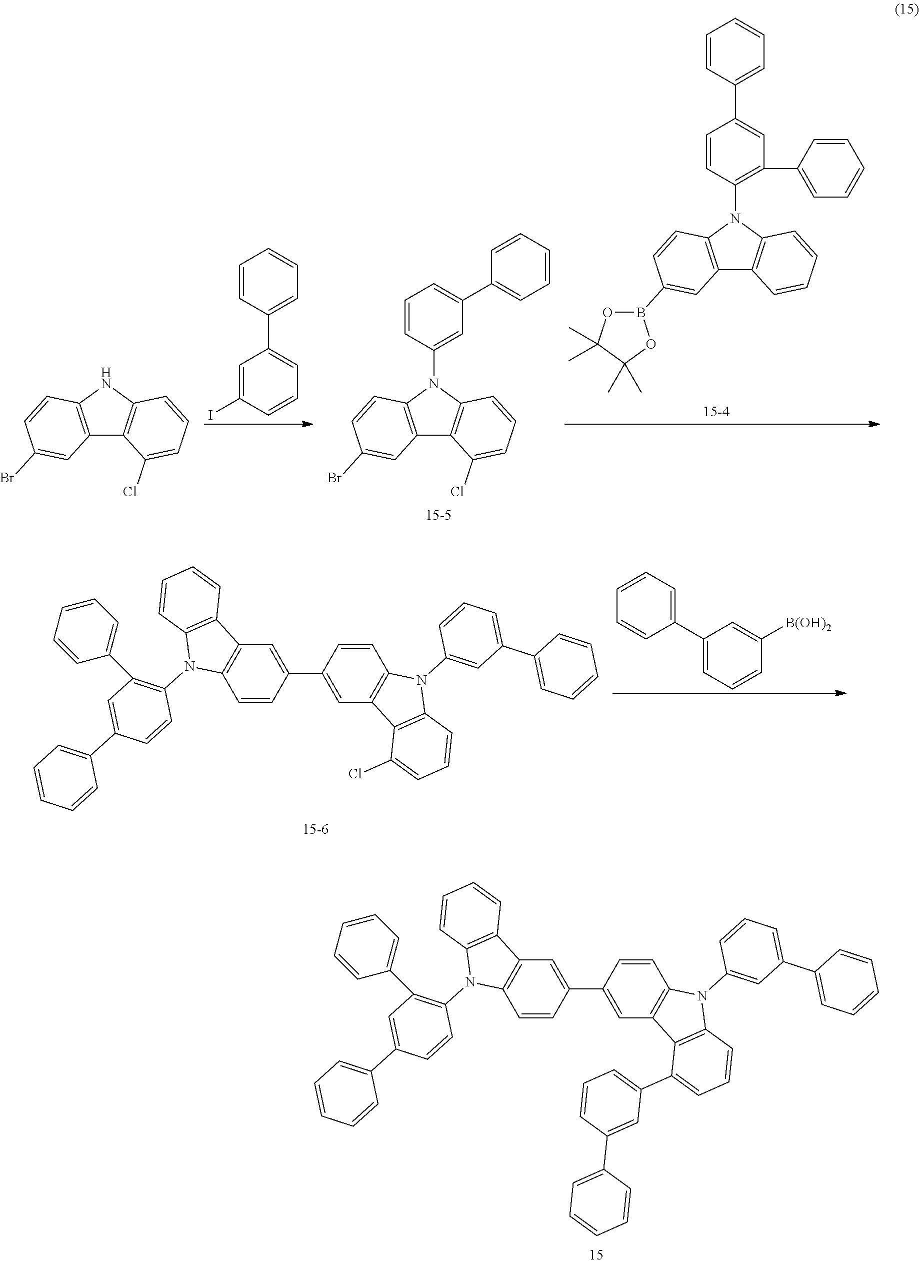

### Synthesis Example 3: Synthesis of Compound 145

(1) Synthesis of Compound 145-1

Compound 145-1 was synthesized according to the Reaction Scheme (16) below:

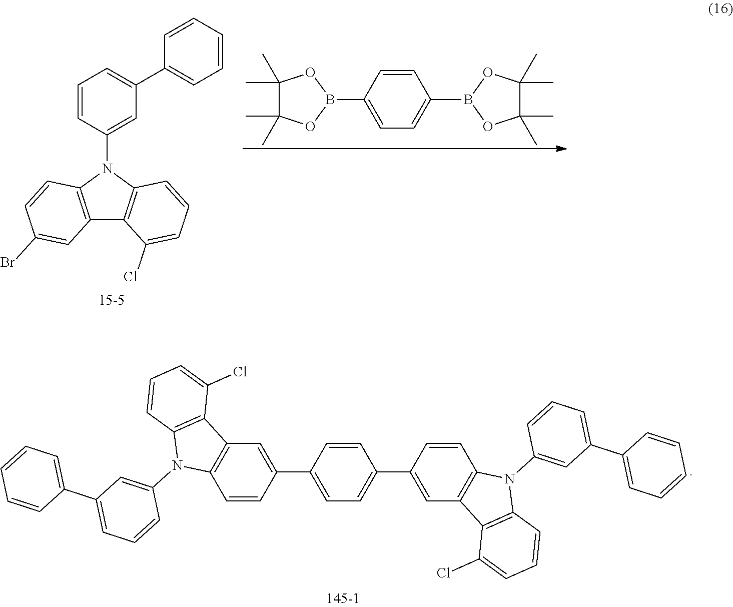

In a nitrogen atmosphere, Compound 15-5 (9-([1,1′-biphenyl]-3-yl)-3-bromo-5-chloro-9H-carbazole) (9.52 g, 22 mmol) obtained in the Reaction Scheme (15), 1,4-bis(4,4,5,5-tetramethyl-1,3,2-dioxaborolan-2-yl)benzene (3.30 g, 10 mmol), toluene (200 ml), ethanol (20 ml), and a 2 M aqueous solution (20 ml) of potassium carbonate were added to a three-neck flask and mixed, and Pd(PPh₃)₄ (0.58 g, 0.5 mmol) was added thereto and stirred at a temperature of 80° C. for 8 hours. Pure water (100 ml) and methanol (200 ml) were added thereto, and the reaction solution was cooled to room temperature. A precipitated solid was filtered and collected. The solid was vacuum-dried (50 ct, 6 hours) and heated in toluene (1 L), and the sample was dissolved therein. The sample was filtered by using a silica gel pad and then concentrated. The resultant was dispersed and washed with ethyl acetate twice to provide Compound 145-1. 4.53 g of Compound 145-1 was obtained, and the yield of Compound 145-1 was 58%.

(2) Synthesis of Compound 145

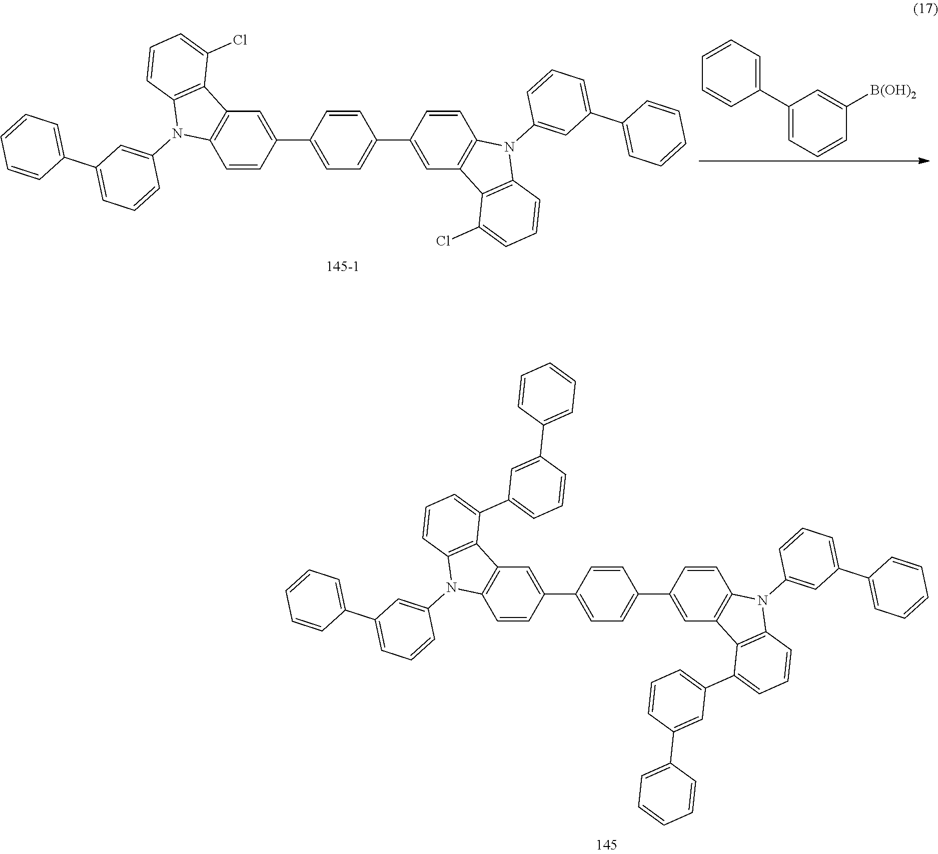

In a nitrogen atmosphere, Compound 145-1 (1,4-bis(9-([1,1′-biphenyl]-3-yl)-5-chloro-9H-carbazol-3-yl)benzene) (4.47 g, 5.72 mmol) obtained in the Reaction Scheme (16), 3-biphenylboronic acid (4.53 g, 22.88 mmol), toluene (229 ml), ethanol (29 ml), and a 2 M aqueous solution (23 ml) of potassium carbonate were added to a three-neck flask and mixed, and Pd(OAc)₂ (64 mg, 0.29 mmol) and S-Phos (188 mg, 0.46 mmol) were added thereto and stirred at a temperature of 80° C. for 12 hours. The reaction solution was diluted by adding toluene (300 ml) thereto, cooled to room temperature, washed with pure water twice in a separatory funnel, dried by using anhydrous magnesium sulfate, filtered by using a silica gel pad, and then concentrated. The resultant obtained therefrom was purified by silica gel chromatography (hexane:toluene=6:4 as developing solvent), and dispersed and washed with ethyl acetate to provide Compound 145. 4.25 g of Compound 145 was obtained, and the yield of Compound 145 was 73%.

### Synthesis Example 4: Synthesis of Comparative Example Compound 1

(1) Synthesis of Comparative Example Compound 1-1

Comparative Example Compound 1-1 was synthesized in the same manner as in Synthesis of Compound 14-8, except that Compound 14-7 (9-([1,1′,4′,1″-terphenyl]-2′-yl)-3-bromo-9H-carbazole) was changed to Compound 15-5 (9-([1,1′-biphenyl]-3-yl)-3-bromo-5-chloro-9H-carbazole).

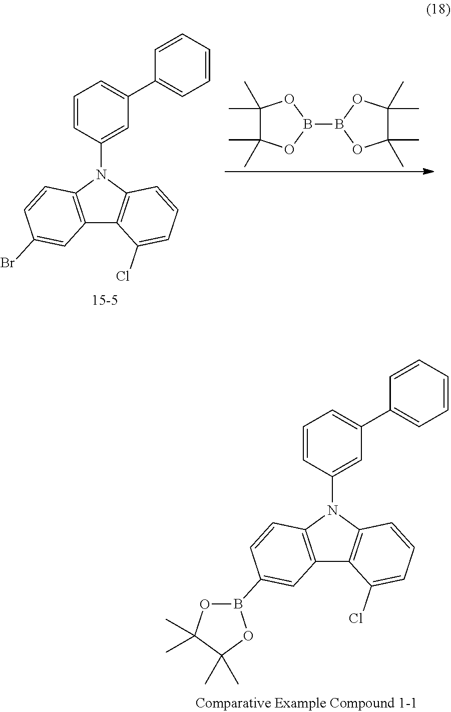

Comparative Example Compound 1-2 was obtained in the same manner as in Synthesis of Compound 14-9, except that the Reaction Scheme was changed to the Reaction Scheme (19) by using Comparative Example Compound 1-1 obtained in the Reaction Scheme (18), and the corresponding reagent was changed.

### Comparative Example

### Compound 1-1

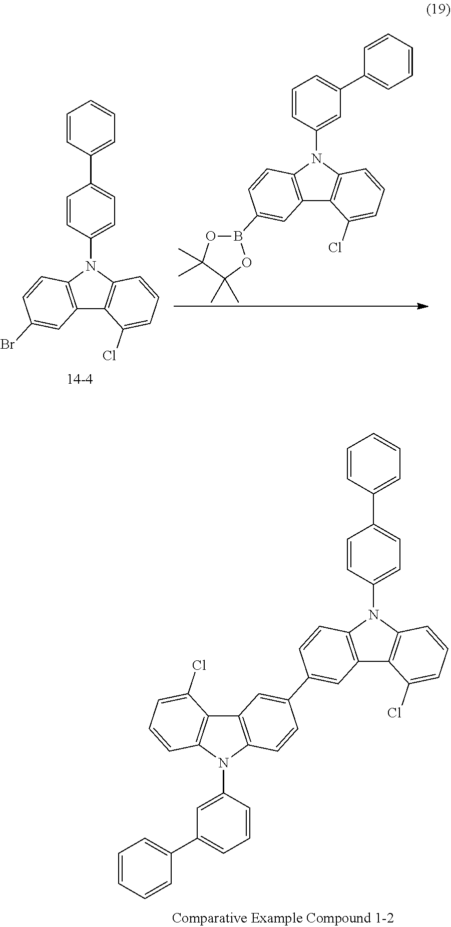

Comparative Example Compound 1 was obtained in the same manner as in Synthesis of Compound 14, except that the Reaction Scheme was changed to the Reaction Scheme (20) by using Comparative Example Compound 1-2 obtained in the Reaction Scheme (19), and the corresponding reagent was changed.

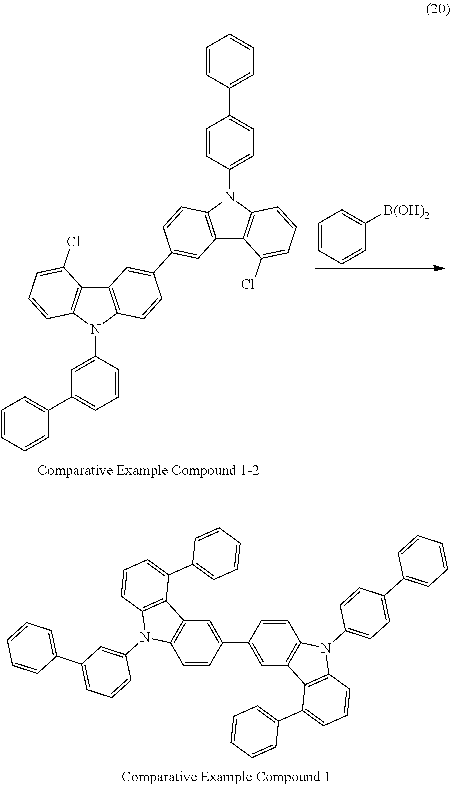

### Synthesis Example 5: Synthesis of Comparative Example Compound 2

Comparative Example Compound 2 was obtained in the same manner as in Synthesis of Compound 14, except that the Reaction Scheme was changed to the Reaction Scheme (21) and the corresponding reagent was changed.

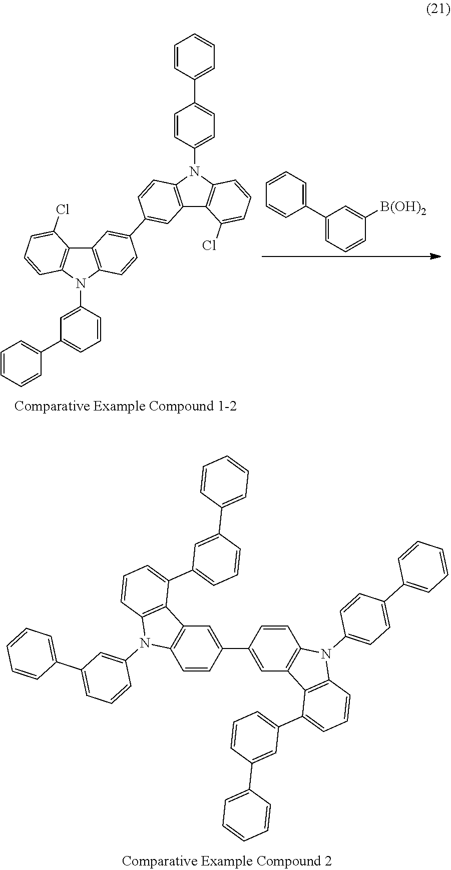

### Evaluation Example 1: Measurement of Solubility

50 milligrams (mg) of a sample solid was added to a colorless sample bottle, and 500 mg of a solvent was added thereto. Ultrasonic waves were irradiated thereon at room temperature for 5 minutes, and the presence or absence of the sample solid was visually confirmed. When the sample solid and is dissolved and can no longer be seen, the solubility is 10 weight % or more. When the sample solid remained, a small amount of a solvent was added thereto, the irradiation with ultrasound was repeated, and the solubility in an amount of the solvent was measured until the sample solid was completely dissolved. Results thereof are shown in Table 1.

TABLE 1

| | Solubility to |
|---|---|
| | methyl benzoate (mass %) |
| Compound 14 | 6 |
| Compound 15 | 6 |
| Compound 145 | 4 |
| Comparative Example Compound 1 | 0.5 |
| Comparative Example Compound 2 | 1.0 |
| Comparative Example Compound 3 | 8 |
| Comparative Example Compound 4 | 0.8 |
| Comparative Example Compound 5 | >10 |
| Comparative Example Compound 6 | >10 |

Comparative Example Compound 3 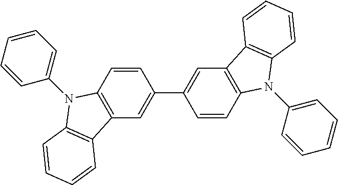 [SMILES: c1ccc(-n2c3ccccc3c3cc(-c4ccc5c(c4)c4ccccc4n5-c4ccccc4)ccc32)cc1]
Comparative Example Compound 4 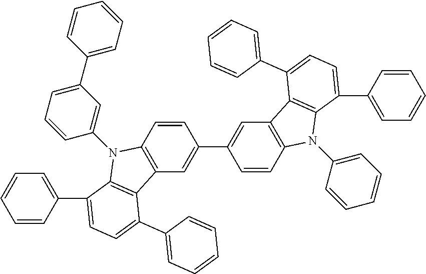 [SMILES: c1ccc(-c2cccc(-n3c4ccc(-c5ccc6c(c5)c5c(-c7ccccc7)ccc(-c7ccccc7)c5n6-c5ccccc5)cc4c4c(-c5ccccc5)ccc(-c5ccccc5)c43)c2)cc1]
Comparative Example Compound 5 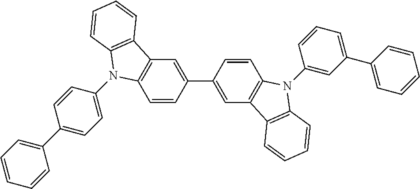 [SMILES: c1ccc(-c2ccc(-n3c4ccccc4c4cc(-c5ccc6c(c5)c5ccccc5n6-c5cccc(-c6ccccc6)c5)ccc43)cc2)cc1]
Comparative Example Compound 6

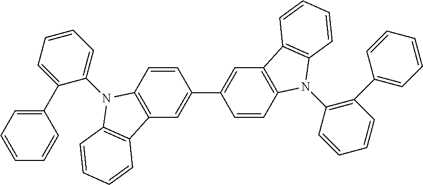 [SMILES: c1ccc(-c2ccccc2-n2c3ccccc3c3cc(-c4ccc5c(c4)c4ccccc4n5-c4ccccc4-c4ccccc4)ccc32)cc1]

Referring to Table 1, it is confirmed that Compounds 14, 15, and 145 have high solubility (mass %) to an organic solvent (methyl benzoate), as compared with Comparative Example Compounds 1, 2, and 4 in which n in Formula 1 is n=0, and Ara and Ar₄ are a phenyl group or an m-phenyl group. Therefore, it is confirmed that the compound according to the embodiment is suitable for manufacturing an organic light-emitting device by using coating.

### Evaluation Example 2: Evaluation of Photo Chemical Stability

Photo chemical stability was evaluated in the following conditions.

Preparation of Measurement Sample

Within a glove box in a nitrogen atmosphere in which a moisture concentration was 1 part per million (ppm) or less and an oxygen concentration was 1 ppm or less, a thin film having a thickness of 50 nanometers (nm) was formed on a quartz substrate by coating at an evaluation host material:green phosphorescent material TEG=100 weight %:5 weight % as a solid fraction in a methyl benzoate having a solid ink concentration of 4 weight %, and processed at a vacuum degree of 1×10⁻³ pascals (Pa) at a temperature of 120° C. for 15 minutes. The thin film was transferred to a vacuum deposition apparatus. An aluminum thin film having a diameter of 2 millimeters (mm) and a round film thickness of 100 nm was vacuum-deposited on the thin film by using a metal mask. This was sealed with a dried glass sealing pipe and an ultraviolet curing resin to prepare a sample for measurement.

Measurement System

A measurement system is mainly configured with the following two systems.

1. PL intensity measurement system

2. UV irradiation deterioration system

1. PL Intensity Measurement System

Light, from which light having a wavelength of 250 nm or less and light having a wavelength of 400 nm or more were removed, was used as excitation light source by using high power UV-Vis optical fiber light source unit (L10290 manufactured by Hamamatsu Photonics) and UV transmission visible absorption filter (S76-U340 manufactured by Suruga Seiki). A small fiber optical spectroscope (USB2000+UV-VIS manufactured by Ocean Photonics) was used as a receiver. Upon measurement, a sample substrate was provided such that excitation light was accurately incident on a sample film measurement portion coated with aluminum at an angle inclined by 20° or more from the front side of the quartz substrate side. The sample film measurement portion was provided at a position at which light was irradiated in the front hemispherical direction of the quartz substrate by excitation light and at a distance at which the receiver was able to capture this light emission and also to avoid the specular reflection of the excitation light by the quartz substrate. The geometric arrangement of the excitation light intensity and the optical system was kept constant such that the intensity of excitation light irradiated on the sample whenever measured was kept constant.

2. UV Irradiation Deterioration System

UV-LED (manufactured by CCS) having maximum emission wavelength of 365 nm was used as a light source, and light with uniform intensity in the irradiated spherical surface was used as excitation light for deterioration through a synthetic quartz light pipe (#65-829 manufactured by Edmund Optics) having a diameter of 2 mm and a length of 75 mm. The excitation light flux intensity was controlled by using a digital power source (PD3-10024-8-PI manufactured by CCS) and an optical power meter (8230E manufactured by ADCMT). The excitation light irradiation sphere and the quartz substrate side of the sample film were aligned and then closely contacted. By irradiating the sample film with the excitation light for a predetermined time on the quartz substrate side, the sample film was optically loaded and deteriorated.

Photo chemical deterioration test of excitation light flux intensity of 10 mW

1. Step 1

The PL intensity of the sample film was measured before deterioration by using the above-described PL intensity measurement system.

2. Step 2

The UV irradiation deterioration system described above was used to perform the excitation light irradiation for the deterioration of the excitation light flux intensity at 10 milliwatts (mW) for 5 minutes, and then the PL intensity of the sample thin film loaded for 5 minutes was measured by using the PL intensity measurement system. Then, the same operation as in step 2 was repeated to measure the PL intensity of the sample thin film with respect to the time during which the excitation light was applied.

Photo Chemical Deterioration Test for Excitation Light Flux Intensity of 20 mW

The PL intensity was measured in the same manner as in the photo chemical deterioration test for the excitation light flux intensity of 10 mW, except that the excitation light flux intensity was changed to 20 mW.

Photo Chemical Deterioration Test for Excitation Light Flux Intensity of 50 mW

The PL intensity was measured in the same manner as in the photo chemical deterioration test for the excitation light flux intensity of 10 mW, except that the excitation light flux intensity was changed to 50 mW.

Photo Chemical Deterioration Test Analysis

First, based on the attenuation data of the PL intensity obtained in the photo chemical deterioration test in which the excitation light flux intensity was changed, the following correspondence relationship was applied to adjust an acceleration coefficient “a” to obtain a decay curve independent of the excitation light flux intensity.

Vertical axis:

R_{I(t)}=I₍ₜ₎/I₀

Horizontal axis:

X_{c}=E^{a}×t

R_{I(t)}: PL intensity ratio of sample film to initial luminance at excitation light irradiation time t

t: Excitation light irradiation time

I₍ₜ₎: PL intensity of sample film at excitation light irradiation time t

I₀: PL intensity of sample film before excitation light irradiation

X_{c}: Corrected excitation light integration intensity

E: Excitation light flux intensity

a: Acceleration coefficient

The corrected excitation light integration intensity Xc90 required for reaching R_{I(t)} of 0.9 (light emission intensity was lowered by 10%) in the “attenuation curve independent of the excitation light flux intensity” obtained by this analysis was taken as an index of the photo chemical stability.

In addition, as the value of Xc90 is higher, larger energy is required to lower the emission intensity, and it is difficult to deteriorate. Table 2 shows the Xc90 results as relative values.

TABLE 2

| | X_{c}90 |
|---|---|
| Compound 14 | 312 |
| Compound 15 | 271 |
| Compound 145 | 240 |
| Comparative Example Compound 1 | 186 |
| Comparative Example Compound 2 | 131 |
| Comparative Example Compound 3 | 100 |
| Comparative Example Compound 4 | 79 |
| Comparative Example Compound 5 | 290 |
| Comparative Example Compound 6 | 80 |

Referring to Table 2, it is confirmed that Compound 14 has larger MO than Comparative Example Compounds 1 to 6, and Compounds 15 and 145 have larger MO than Comparative Example Compounds 1 to 4 and 6 and have high photo chemical stability. Therefore, when the compound according to the embodiment is applied to the organic light-emitting device (for example, the emission layer), lifespan improvement may be expected.

### Evaluation Example 3: Evaluation of Film-Forming Properties

The film-forming properties of the organic light-emitting devices according to Example and Comparative Examples were evaluated by the following methods. A commercially available TBF layer was formed on an ITO glass substrate having ITO deposited to a thickness of 150 nm, and a stacked film was formed by coating a 2 weight % methyl benzoate solution of the obtained compound to have a thickness of 30 nm by spin coating. The obtained stacked film was heated to a temperature of 125° C.

The surface of the stacked film was observed with an optical microscope, and the state of the film was evaluated. In addition, the state of the film was evaluated according to the following criteria.

◯: uniform and defect-free state

Δ: Uneven points or areas are visible, but the device is ready for production

X: State in which the film continuity cannot be obtained and the device cannot be manufactured

TABLE 3

| | State of film |
|---|---|
| Compound 14 | ◯ |
| Compound 15 | ◯ |
| Compound 145 | ◯ |
| Comparative Example Compound 1 | Δ |
| Comparative Example Compound 2 | Δ |
| Comparative Example Compound 3 | Δ |
| Comparative Example Compound 4 | X |
| Comparative Example Compound 5 | ◯ |
| Comparative Example Compound 6 | ◯ |

Referring to Table 3, it is confirmed that Compounds 14, 15, and 145 are in a state in which a film after heated to a temperature of 125° C. is uniform and has no defect, and shows excellent film formation, as compared with Comparative Example Compounds 1 to 4. Therefore, it is confirmed that the compound according to the embodiment is suitable for use as a material for an organic light-emitting device for coating.

Method of Evaluating Organic Light-Emitting Device

Evaluation of Current Efficiency and Durability (Emission Lifespan)

The current efficiency and durability (emission lifespan) of Examples and Comparative Examples were evaluated in the following conditions. First, the voltage and current applied to the organic light-emitting device were measured by using a DC constant voltage power source (Source Meter manufactured by KEYENCE), and the emission luminance of the organic light-emitting device was measured by using a luminance measurement device (SR-3 manufactured by Topcon).

The current density per unit area was calculated from the size and current value of the organic light-emitting device, and the current efficiency (candelas per ampere, cd/A) was calculated by dividing luminance (candelas per square meter, cd/m²) by the current density (amperes per square meter, A/m²). In addition, the current efficiency indicates the efficiency (conversion efficiency) of converting the current into the light emission energy. As the current efficiency is higher, the performance of the organic light-emitting device is higher.

In the durability (emission lifespan), LT₈₀ (hours, h) indicates an amount of time that lapsed when emission luminance decreasing together with the elapse of the continuous operation time was 80% of initial luminance at a current value of 6,000 cd/m² in each organic light-emitting device.

### Example 1

First, an ITO glass substrate was prepared. In the ITO glass substrate, ITO was deposited to a thickness of 150 nm in a stripe form as a first electrode (anode). poly(3,4-ethylene dioxythiophene)/poly(4-styrene sulfonate):PEDOT/PSS) (manufactured by Sigma-Aldrich) was spin-coated on the ITO glass substrate to form a hole injection layer having a dry film thickness of 30 nm.

Then, a hole transport layer coating liquid was prepared by dissolving poly(9,9-dioctyl-fluorene-co-N-(4-butylphenyl)-diphenylamine) (TFB) in 1 weight % of xylene. The hole transport layer coating liquid was spin-coated on the hole injection layer to a dry film thickness of 30 nm and heated to a temperature of 230° C. for 1 hour, thereby forming a hole transport layer.

Then, a methyl benzoate solution including Compound 14 synthesized in Synthesis Example 1 as a hole transport host material, Compound ETH-1 as an electron transport host material, and tris(2-(3-p-xylyl)phenyl)pyridine iridium (TEG) as an emission dopant material was prepared. The solution was spin-coated on the hole transport layer to form an emission layer having a film thickness of 30 nm. The weight ratios of the hole transport host material, the electron transport host material, and the emission dopant material in the emission layer were respectively 85.7 weight %, 9.5 weight %, and 4.8 weight % based on the total weight of the emission layer.

11-(4,6-diphenyl-1,3,5-triazine-2-yl)-12-phenyl-11,12-dihydroindolo[2, 3-a]carbazole (HB-1) was deposited on the emission layer by using a vacuum deposition apparatus to form a hole blocking layer having a thickness of 10 nm. (8-quinolinato)lithium (LiQ) and KLET-03 were co-deposited on the hole blocking layer by using a vacuum deposition apparatus to form an electron transport layer having a thickness of 30 nm. Aluminum was deposited on the electron transport layer by using a vacuum deposition apparatus to form a cathode having a thickness of 100 nm, thereby completing the manufacture of an organic light-emitting device.

The organic light-emitting device was sealed by using a glass sealing tube containing a desiccant and an ultraviolet curable resin in a glove box having a moisture and oxygen concentration of 1 ppm or less, and then used for evaluation.

TFB is a polymer compound having the following structural formula.

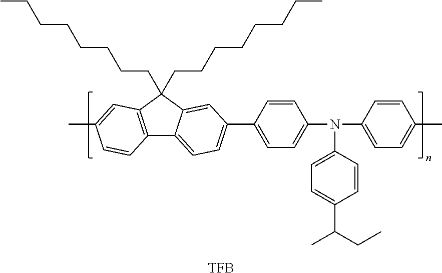

TFB has a weight average molecular weight (Mw) of 320,000 Daltons, a number average molecular weight (Mₙ) of 98,000 Daltons, and PDI of 3.3.

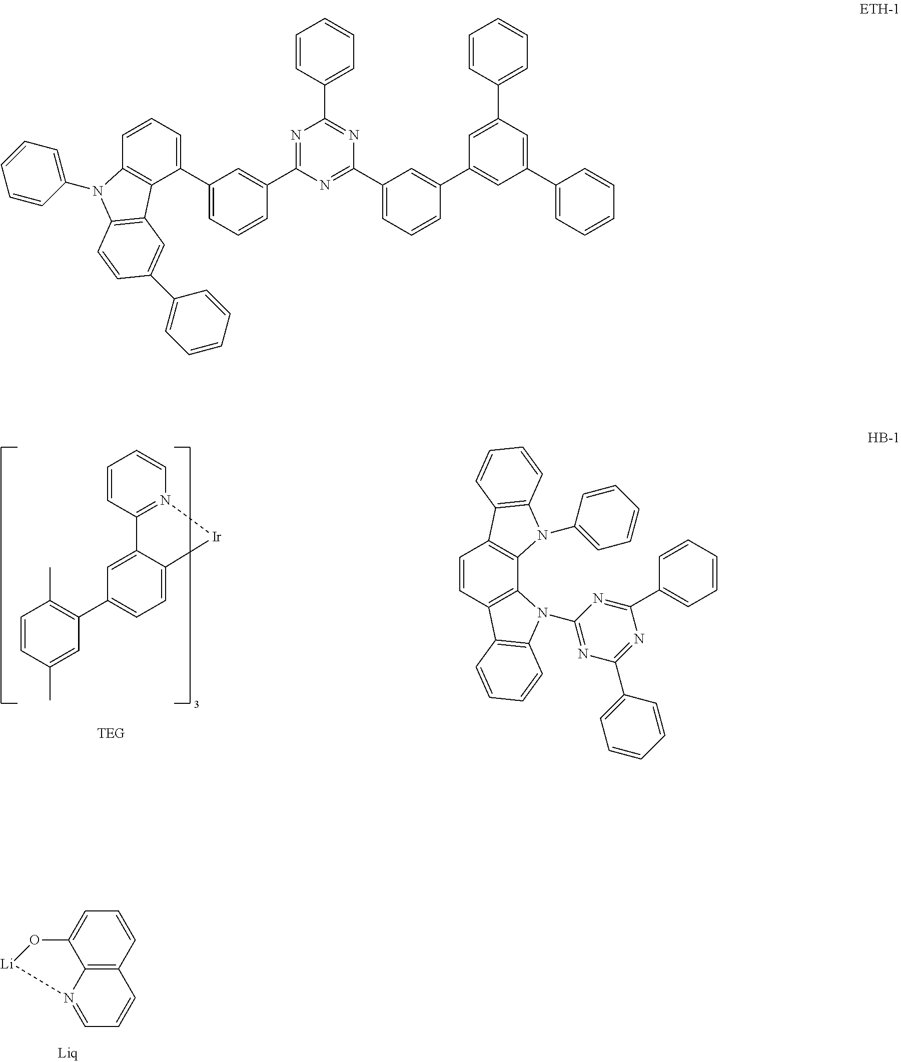

Results thereof are shown in Table 4. In addition, Table 4 shows the relative values of the current efficiency and LT₈₀ (h) of each compound when the Comparative Example Compound 3 is taken as 100.

### Example 2

An organic light-emitting device was manufactured in the same manner as in Example 1, except that Compound 15 synthesized in Synthesis Example 2 was used instead of Compound 14 as a hole transport host material. Results thereof are shown in Table 4.

### Example 3

An organic light-emitting device was manufactured in the same manner as in Example 1, except that Compound 145 synthesized in Synthesis Example 3 was used instead of Compound 14 as a hole transport host material. Results thereof are shown in Table 4.

### Comparative Example 1

An organic light-emitting device was manufactured in the same manner as in Example 1, except that Comparative Example Compound 1 synthesized in Synthesis Example 4 was used instead of Compound 14 as a hole transport host material. Results thereof are shown in Table 4.

### Comparative Example 2

An organic light-emitting device was manufactured in the same manner as in Example 1, except that Comparative Example Compound 2 synthesized in Synthesis Example 5 was used instead of Compound 14 as a hole transport host material. Results thereof are shown in Table 4.

### Comparative Example 3

An organic light-emitting device was manufactured in the same manner as in Example 1, except that Comparative Example Compound 3 represented by the following formula was used instead of Compound 14 as a hole transport host material. Results thereof are shown in Table 4.

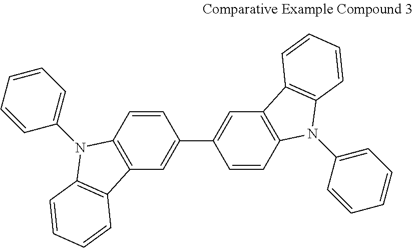 [SMILES: c1ccc(-n2c3ccccc3c3cc(-c4ccc5c(c4)c4ccccc4n5-c4ccccc4)ccc32)cc1]

### Comparative Example 4

An organic light-emitting device was manufactured in the same manner as in Example 1, except that Comparative Example Compound 4 represented by the following formula was used instead of Compound 14 as a hole transport host material. Results thereof are shown in Table 4.

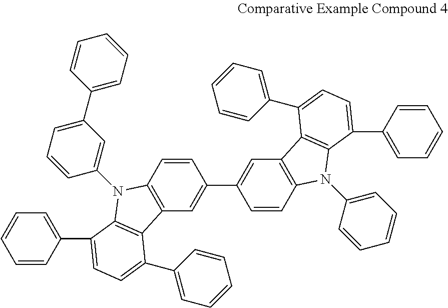 [SMILES: c1ccc(-c2cccc(-n3c4ccc(-c5ccc6c(c5)c5c(-c7ccccc7)ccc(-c7ccccc7)c5n6-c5ccccc5)cc4c4c(-c5ccccc5)ccc(-c5ccccc5)c43)c2)cc1]

### Comparative Example 5

An organic light-emitting device was manufactured in the same manner as in Example 1, except that Comparative Example Compound 5 represented by the following formula was used instead of Compound 14 as a hole transport host material. Results thereof are shown in Table 4.

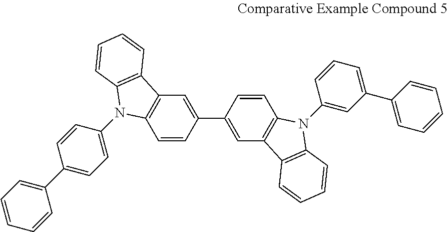 [SMILES: c1ccc(-c2ccc(-n3c4ccccc4c4cc(-c5ccc6c(c5)c5ccccc5n6-c5cccc(-c6ccccc6)c5)ccc43)cc2)cc1]

### Comparative Example 6

An organic light-emitting device was manufactured in the same manner as in Example 1, except that Comparative Example Compound 6 represented by the following formula was used instead of Compound 14 as a hole transport host material. Results thereof are shown in Table 4.

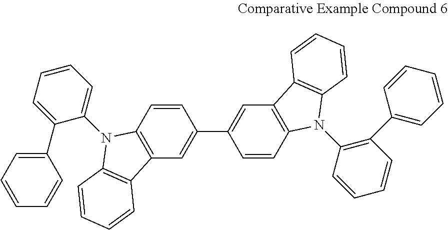 [SMILES: c1ccc(-c2ccccc2-n2c3ccccc3c3cc(-c4ccc5c(c4)c4ccccc4n5-c4ccccc4-c4ccccc4)ccc32)cc1]

TABLE 4

| | | Current efficiency | |
|---|---|---|---|
| | | (cd/A) | LT₈₀ (relative value) |
| Example 1 | Compound 14 | 126 | 275 |
| Example 2 | Compound 15 | 139 | 290 |
| Example 3 | Compound 145 | 119 | 335 |
| Comparative | Comparative | 95 | 65 |
| Example 1 | Example | | |
| | Compound 1 | | |
| Comparative | Comparative | 110 | 105 |
| Example 2 | Example | | |
| | Compound 2 | | |
| Comparative | Comparative | 100 | 100 |
| Example 3 | Example | | |
| | Compound 3 | | |
| Comparative | Comparative | 104 | 15 |
| Example 4 | Example | | |
| | Compound 4 | | |
| Comparative | Comparative | 116 | 210 |
| Example 5 | Example | | |
| | Compound 5 | | |
| Comparative | Comparative | 97 | 35 |
| Example 6 | Example | | |
| | Compound 6 | | |

Referring to Table 4, it is confirmed that the organic light-emitting devices of Examples 1 to 3 in which Compounds 14, 15, and 145 are used as the hole transport host material have current efficiency equal to or higher than the organic light-emitting devices of Comparative Examples 1 to 6, and also have greatly improved emission lifespan.

In addition, since Compounds 14, 15, and 145 used in Examples 1 to 3 may form the organic light-emitting device by using coating, Compounds 14, 15, and 145 are preferable in terms of mass production.

Since the condensed cyclic compound has improved electrical characteristics and/or thermal stability, the organic light-emitting device including the condensed cyclic compound may have improved current efficiency and lifespan characteristics.

It should be understood that embodiments described herein should be considered in a descriptive sense only and not for purposes of limitation. Descriptions of features or aspects within each embodiment should typically be considered as available for other similar features or aspects in other embodiments.

While one or more embodiments have been described with reference to the FIGURES, it will be understood by those of ordinary skill in the art that various changes in form and details may be made therein without departing from the spirit and scope of the present description as defined by the following claims.

What is claimed is:

1. A condensed cyclic compound represented by Formula 1: 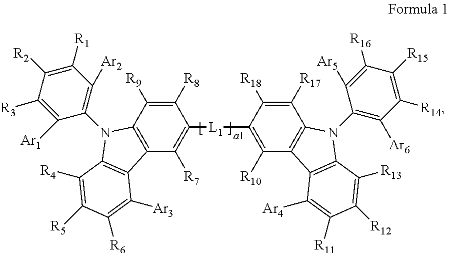
wherein, in Formula 1,
Ar₁ to Ar₆ and R₁ to R₁₈ are each independently selected from a group represented by *-(L₂₁)ₐ₂₁-R₂₁, hydrogen, deuterium, —F, —Cl, —Br, —I, a hydroxyl group, a cyano group, a nitro group, an amidino group, a hydrazine group, a hydrazone group, a carboxylic acid group or a salt thereof, a sulfonic acid group or a salt thereof, a phosphoric acid group or a salt thereof, a substituted or unsubstituted C₁-C₆₀ alkyl group, a substituted or unsubstituted C₂-C₆₀ alkenyl group, a substituted or unsubstituted C₂-C₆₀ alkynyl group, a substituted or unsubstituted C₁-C₆₀ alkoxy group, a substituted or unsubstituted C₃-C₁₀ cycloalkyl group, a substituted or unsubstituted C₁-C₁₀ heterocycloalkyl group, a substituted or unsubstituted C₃-C₁₀ cycloalkenyl group, a substituted or unsubstituted C₁-C₁₀ heterocycloalkenyl group, a substituted or unsubstituted C₆-C₆₀ aryl group, a substituted or unsubstituted C₆-C₆₀ aryloxy group, a substituted or unsubstituted C₆-C₆₀ arylthio group, a substituted or unsubstituted C₁-C₆₀ heteroaryl group, a substituted or unsubstituted monovalent non-aromatic condensed polycyclic group, a substituted or unsubstituted monovalent non-aromatic condensed heteropolycyclic group, —Si(Q₁)(Q₂)(Q₃), —N(Q₄)(Q₅), and —B(Q₆)(Q₇),
wherein i) each of at least one selected from Ar₁, Ar₂, Ar₅, and Ar₆ is independently a group represented by *-(L₂₁)ₐ₂₁-R₂₁, and one selected from Ar₃ and Ar₄ is a group represented by *-(L₂₁)ₐ₂₁-R₂₁; or
ii) Ar₃ and Ar₄ are each independently a group represented by *-(L₂₁)ₐ₂₁-R₂₁,
L₁ and L₂₁ are each independently selected from a substituted or unsubstituted C₅-C₃₀ carbocyclic group and a substituted or unsubstituted C₁-C₃₀ heterocyclic group,
a1 and a21 are each independently 0 or 1, wherein, when a1 is 0, *-(L₁)ₐ₁-′ is a single bond, and when a21 is 0, *-(L₂₁)ₐ₂₁-*′ is a single bond,
R₂₁ is selected from a substituted or unsubstituted C₆-C₆₀ aryl group, a substituted or unsubstituted heteroaryl group, a substituted or unsubstituted monovalent non-aromatic condensed polycyclic group, and a substituted or unsubstituted monovalent non-aromatic condensed heteropolycyclic group,
when a1 is 0, one selected from Ar₃ and Ar₄ is selected from hydrogen and deuterium,
at least one substituent of the substituted C₅-C₃₀ carbocyclic group, the substituted C₁-C₃₀ heterocyclic group, the substituted C₁-C₆₀ alkyl group, the substituted C₂-C₆₀ alkenyl group, the substituted C₂-C₆₀ alkynyl group, the substituted C₁-C₆₀ alkoxy group, the substituted C₃-C₁₀ cycloalkyl group, the substituted C₁-C₁₀ heterocycloalkyl group, the substituted C₃-C₁₀ cycloalkenyl group, the substituted C₁-C₁₀ heterocycloalkenyl group, the substituted C₆-C₆₀ aryl group, the substituted C₆-C₆₀ aryloxy group, the substituted C₆-C₆₀ arylthio group, the substituted C₁-C₆₀ heteroaryl group, the substituted monovalent non-aromatic condensed polycyclic group, and the substituted monovalent non-aromatic condensed heteropolycyclic group is selected from:
deuterium, —F, —Br, —CD₃, —CD₂H, —CDH₂, —CF₃, —CF₂H, —CFH₂, a hydroxyl group, a cyano group, a nitro group, an amidino group, a hydrazine group, a hydrazone group, a carboxylic acid group or a salt thereof, a sulfonic acid group or a salt thereof, a phosphoric acid group or a salt thereof, a C₁-C₆₀ alkyl group, a C₂-C₆₀ alkenyl group, a C₂-C₆₀ alkynyl group, and a C₁-C₆₀ alkoxy group;
a C₁-C₆₀ alkyl group, a C₂-C₆₀ alkenyl group, a C₂-C₆₀ alkynyl group, and a C₁-C₆₀ alkoxy group, each substituted with at least one selected from deuterium, —F, —Cl, —Br, —CD₃, —CD₂H, —CDH₂, —CF₃, —CF₂H, —CFH₂, a hydroxyl group, a cyano group, a nitro group, an amidino group, a hydrazine group, a hydrazone group, a carboxylic acid group or a salt thereof, a sulfonic acid group or a salt thereof, a phosphoric acid group or a salt thereof, a C₃-C₁₀ cycloalkyl group, a C₁-C₁₀ heterocycloalkyl group, a C₃-C₁₀ cycloalkenyl group, a C₁-C₁₀ heterocycloalkenyl group, a C₆-C₆₀ aryl group, a C₆-C₆₀ aryloxy group, a C₆-C₆₀ arylthio group, a C₁-C₆₀ heteroaryl group, a monovalent non-aromatic condensed polycyclic group, a monovalent non-aromatic condensed heteropolycyclic group, —N(Q₁₁)(Q₁₂), —Si(Q₁₃)(Q₁₄)(Q₁₅), —B(Q₁₆)(Q₁₇), and —P(═O)(Q₁₈)(Q₁₉);
a C₃-C₁₀ cycloalkyl group, a C₁-C₁₀ heterocycloalkyl group, a C₃-C₁₀ cycloalkenyl group, a C₁-C₁₀ heterocycloalkenyl group, a C₆-C₆₀ aryl group, a C₆-C₆₀ aryloxy group, a C₆-C₆₀ arylthio group, a C₁-C₆₀ heteroaryl group, a monovalent non-aromatic condensed polycyclic group, and a monovalent non-aromatic condensed heteropolycyclic group;
a C₃-C₁₀ cycloalkyl group, a C₁-C₁₀ heterocycloalkyl group, a C₃-C₁₀ cycloalkenyl group, a C₁-C₁₀ heterocycloalkenyl group, a C₆-C₆₀ aryl group, a C₆-C₆₀ aryloxy group, a C₆-C₆₀ arylthio group, a C₁-C₆₀ heteroaryl group, a monovalent non-aromatic condensed polycyclic group, and a monovalent non-aromatic condensed heteropolycyclic group, each substituted with at least one selected from deuterium, —F, —Cl, —Br, —I, —CD₃, —CD₂H, —CDH₂, —CF₃, —CF₂H, —CFH₂, a hydroxyl group, a cyano group, a nitro group, an amidino group, a hydrazine group, a hydrazone group, a carboxylic acid group or a salt thereof, a sulfonic acid group or a salt thereof, a phosphoric acid group or a salt thereof, a C₁-C₆₀ alkyl group, a C₂-C₆₀ alkenyl group, a C₂-C₆₀ alkynyl group, a C₁-C₆₀ alkoxy group, a C₃-C₁₀ cycloalkyl group, a C₁-C₁₀ heterocycloalkyl group, a C₃-C₁₀ cycloalkenyl group, a heterocycloalkenyl group, a C₆-C₆₀ aryl group, a C₆-C₆₀ aryloxy group, a C₆-C₆₀ arylthio group, a C₁-C₆₀ heteroaryl group, a monovalent non-aromatic condensed polycyclic group, a monovalent non-aromatic condensed heteropolycyclic group, —N(Q₂₁)(Q₂₂), —Si(Q₂₃)(Q₂₄)(Q₂₅), —B(Q₂₆)(Q₂₇), and —P(═O)(Q₂₈)(Q₂₉); and
—N(Q₃₁)(Q₃₂), —Si(Q₃₃)(Q₃₄)(Q₃₅), —B(Q₃₆)(Q₃₇), and —P(═O)(Q₃₈)(Q₃₉), and
Q₁ to Q₉, Q₁₁ to Q₁₉, Q₂₁ to Q₂₉ and Q₃₁ to Q₃₉ are each independently selected from hydrogen, deuterium, —F, —Cl, —Br, —I, a hydroxyl group, a cyano group, a nitro group, an amino group, an amidino group, a hydrazine group, a hydrazone group, a carboxylic acid group or a salt thereof, a sulfonic acid group or a salt thereof, a phosphoric acid group or a salt thereof, a C₁-C₆₀ alkyl group, a C₂-C₆₀ alkenyl group, a C₂-C₆₀ alkynyl group, a C₁-C₆₀ alkoxy group, a C₃-C₁₀ cycloalkyl group, a C₁-C₁₀ heterocycloalkyl group, a C₃-C₁₀ cycloalkenyl group, a C₁-C₁₀ heterocycloalkenyl group, a C₆-C₆₀ aryl group, a C₆-C₆₀ aryl group substituted with at least one selected from a C₁-C₆₀ alkyl group and a C₆-C₆₀ aryl group, a C₆-C₆₀ aryloxy group, a C₆-C₆₀ arylthio group, a C₁-C₆₀ heteroaryl group, a monovalent non-aromatic condensed polycyclic group, and a monovalent non-aromatic condensed heteropolycyclic group.

2. The condensed cyclic compound of claim 1, wherein
L₁ and L₂₁ are each independently selected from:
a benzene group, a pentalene group, an indene group, a naphthalene group, an anthracene group, an azulene group, a heptalene group, an acenaphthylene group, a phenalene group, a fluorene group, a spiro-bifluorene group, a benzofluorene group, a dibenzofluorene group, a phenanthrene group, a biphenyl group, a terphenyl group, a triphenylene group, a fluoranthene group, a pyrene group, a chrysene group, a picene group, a perylene group, a pentaphene group, a pentacene group, a tetraphene group, a hexaphene group, a hexacene group, a rubicene group, a trinaphthalene group, a heptaphene group, a pyranthrene group, a pyridine group, a pyrazine group, a pyridazine group, a pyrimidine group, a triazine group, a quinoline group, an isoquinoline group, a quinoxaline group, a quinazoline group, a naphthyridine group, an acridine group, a phenazine group, a benzoquinoline group, a benzoisoquinoline group, a phenanthridine group, a phenanthroline group, a benzoquinone group, a coumarin group, an anthraquinone group, a fluorenone group, a furan group, a thiophene group, a silole group, a benzofuran group, a benzothiophene group, a benzosilole group, a dibenzofuran group, a dibenzothiophene group, a dibenzosilole group, a pyrrole group, an indole group, an isoindole group, a carbazole group, a benzocarbazole group, a dibenzocarbazole group, an imidazole group, a benzimidazole group, a pyrazole group, a triazole group, a tetrazole group, an indazole group, an oxazole group, an isoxazole group, a benzoxazole group, a benzisoxazole group, a thiazole group, an isothiazole group, a benzothiazole group, a benzisothiazole group, an imidazolinone group, a benzoimidazolinone group, an imidazopyridine group, an imidazopyrimidine group, an imidazophenanthridine group, a benzimidazophenanthridine group, an azadibenzofuran group, an azacarbazole group, an azadibenzothiophene group, a diazadibenzofuran group, a diazacarbazole group, a diazadibenzothiophene group, a xanthone group, and a thioxanthone group; and
a benzene group, a pentalene group, an indene group, a naphthalene group, an anthracene group, an azulene group, a heptalene group, an acenaphthylene group, a phenalene group, a fluorene group, a spiro-bifluorene group, a benzofluorene group, a dibenzofluorene group, a phenanthrene group, a biphenyl group, a terphenyl group, a triphenylene group, a fluoranthene group, a pyrene group, a chrysene group, a picene group, a perylene group, a pentaphene group, a pentacene group, a tetraphene group, a hexaphene group, a hexacene group, a rubicene group, a trinaphthalene group, a heptaphene group, a pyranthrene group, a pyridine group, a pyrazine group, a pyridazine group, a pyrimidine group, a triazine group, a quinoline group, an isoquinoline group, a quinoxaline group, a quinazoline group, a naphthyridine group, an acridine group, a phenazine group, a benzoquinoline group, a benzoisoquinoline group, a phenanthridine group, a phenanthroline group, a benzoquinone group, a coumarin group, an anthraquinone group, a fluorenone group, a furan group, a thiophene group, a silole group, a benzofuran group, a benzothiophene group, a benzosilole group, a dibenzofuran group, a dibenzothiophene group, a dibenzosilole group, a pyrrole group, an indole group, an isoindole group, a carbazole group, a benzocarbazole group, a dibenzocarbazole group, an imidazole group, a benzimidazole group, a pyrazole group, a triazole group, a tetrazole group, an indazole group, an oxazole group, an isoxazole group, a benzoxazole group, a benzisoxazole group, a thiazole group, an isothiazole group, a benzothiazole group, a benzisothiazole group, an imidazolinone group, a benzoimidazolinone group, an imidazopyridine group, an imidazopyrimidine group, an imidazophenanthridine group, a benzimidazophenanthridine group, an azadibenzofuran group, an azacarbazole group, an azadibenzothiophene group, a diazadibenzofuran group, a diazacarbazole group, a diazadibenzothiophene group, a xanthone group, and a thioxanthone group, each substituted with at least one selected from deuterium, —F, —Cl, —Br, —I, a hydroxyl group, a cyano group, a nitro group, an amidino group, a hydrazine group, a hydrazone group, a C₁-C₃₀ alkyl group, a C₁-C₃₀ alkoxy group, a phenyl group, a pentalenyl group, an indenyl group, a naphthyl group, an anthracenyl group, an azulenyl group, a heptalenyl group, an acenaphthyl group, a phenalenyl group, a fluorenyl group, a spiro-bifluorenyl group, a benzofluorenyl group, a dibenzofluorenyl group, a phenanthrenyl group, a biphenyl group, a terphenyl group, a triphenylenyl group, a fluoranthenyl group, a pyrenyl group, a chrysenyl group, a picenyl group, a perylenyl group, a pentaphenyl group, a pentacenyl group, a tetraphenyl group, a hexaphenyl group, a hexacenyl group, a rubicenyl group, a trinaphthyl group, a heptaphenyl group, a pyranthrenyl group, a pyridinyl group, a pyrazinyl group, a pyridazinyl group, a pyrimidinyl group, a triazinyl group, a quinolinyl group, an isoquinolinyl group, a quinoxalinyl group, a quinazolinyl group, a naphthyridinyl group, an acridinyl group, a phenazinyl group, a benzoquinolinyl group, a benzoisoquinolinyl group, a phenanthridinyl group, a phenanthrolinyl group, a benzoquinonyl group, a cumarinyl group, an anthraquinonyl group, a fluorenonyl group, a furanyl group, a thienyl group, a silolyl group, a benzofuranyl group, a benzothienyl group, a benzosilolyl group, a dibenzofuranyl group, a dibenzothiophenyl group, a dibenzosilolyl group, a pyrrolyl group, an indolyl group, an isoindolyl group, a carbazolyl group, a benzocarbazolyl group, a dibenzocarbazolyl group, an imidazolyl group, a benzimidazolyl group, a pyrazolyl group, a triazolyl group, a tetrazolyl group, an indazolyl group, an oxazolyl group, an isoxazolyl group, a benzoxazolyl group, a benzisoxazolyl group, a thiazolyl group, an isothiazolyl group, a benzothiazolyl group, a benzisothiazolyl group, an imidazopyridinyl group, an imidazopyrimidinyl group, an imidazophenanthridinyl group, a benzimidazophenanthridinyl group, an azadibenzofuranyl group, an azacarbazolyl group, an azadibenzothienyl group, a diazadibenzofuranyl group, a diazacarbazolyl group, a diazadibenzothienyl group, a xanthonyl group, and a thioxanthonyl group.

3. The condensed cyclic compound of claim 1, wherein
L₁ and L₂₁ are each independently selected from groups represented by Formulae 3-1 to 3-3: 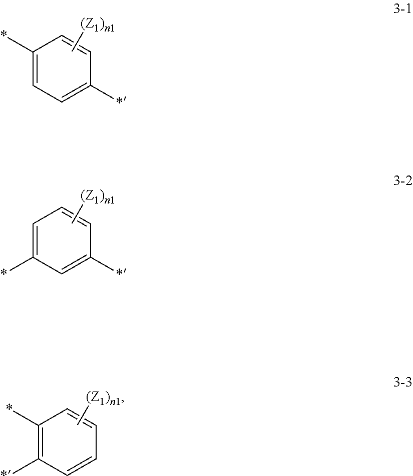
wherein, in Formulae 3-1 to 3-3,
Z₁ is selected from hydrogen, deuterium, —F, —Cl, —Br, —I, a hydroxyl group, a cyano group, a nitro group, an amino group, an amidino group, a hydrazine group, a hydrazone group, a carboxylic acid group or a salt thereof, a sulfonic acid group or a salt thereof, a phosphoric acid group or a salt thereof, a C₁-C₃₀ alkyl group, a C₁-C₃₀ alkoxy group, a phenyl group, a naphthyl group, a biphenyl group, and a terphenyl group,
n1 is 1, 2, 3, or 4, and
* and *′ each indicate a binding site to a neighboring atom.

4. The condensed cyclic compound of claim 1, wherein
Ar₁ to Ar₆ and R₁ to R₁₈ are each independently selected from:
a group represented by *-(L₂₁)ₐ₂₁-R₂₁, hydrogen, deuterium, —F, —Cl, —Br, —I, a cyano group, a methyl group, an ethyl group, an n-propyl group, an iso-propyl group, an n-butyl group, an iso-butyl group, a sec-butyl group, a tert-butyl group, an n-pentyl group, an iso-pentyl group, a tert-pentyl group, a neo-pentyl group, a 1,2-dimethylpropyl group, an n-hexyl group, an iso-hexyl group, a 1,3-dimethylbutyl group, a 1-iso-propylpropyl group, a 1,2-dimethylbutyl group, an n-heptyl group, a 1,4-dimethylpentyl group, a 3-ethylpentyl group, a 2-methyl-1-iso-propylpropyl group, a 1-ethyl-3-methylbutyl group, an n-octyl group, a 2-ethylhexyl group, a 3-methyl-1-iso-propylbutyl group, a 2-methyl-1-iso-propyl group, a 1-tert-butyl-2-methylpropyl group, an n-nonyl group, a 3,5,5-trimethyldecyl group, an n-decyl group, an iso-decyl group, an n-undecyl group, a 1-methyldecyl group, an n-dodecyl group, an n-tridecyl group, an n-tetradecyl group, an n-pentadecyl group, an n-hexadecyl group, an n-heptadecyl group, an n-octadecyl group, an n-nonadecyl group, an n-eicosyl group, an n-heneicosyl group, an n-docosyl group, an n-tricosyl group, an n-tetracosyl group, a methoxy group, an ethoxy group, a propoxy group, an iso-propoxy group, an n-butoxy group, an iso-butoxy group, a sec-butoxy group, tert-butoxy group, an n-pentoxy group, an iso-pentoxy group, a tert-pentoxy group, a neo-pentoxy group, an n-hexyloxy group, an iso-hexyloxy group, heptyloxy group, an octyloxy group, a nonyloxy group, a decyloxy group, an undecyloxy group, a dodecyloxy group, a tridecyloxy group, a tetradecyloxy group, a pentadecyloxy group, a hexadecyl group, a heptadecyl group, an octadecyl group, a 2-ethylhexyloxy group, a 3-ethylpentyloxy group, a phenoxy group, a 1-naphthyloxy group, a 2-naphthyloxy group, a 2-azulenyloxy group, a 2-furanyloxy group, a 2-thienyloxy group, a 2-indolyloxy group, a 3-indolyloxy group, a 2-benzofuryloxy group, a 2-benzothienyloxy group, an N-methylamino group, an n-ethylamino group, an n-propylamino group, an n-iso-propylamino group, an n-butylamino group, an n-iso-butylamino group, an n-sec-butylamino group, an n-tert-butylamino group, an n-pentylamino group, an n-hexylamino group, an N,N-dimethylamino group, an N,N-diethylamino group, an N,N-dipropylamino group, an N,N-di-iso-propylamino group, an N,N-dibutylamino group, an N,N-di-iso-butylamino group, an N,N-dipentylamino group, and an N,N-dihexylamino group;
a phenyl group, a pentalenyl group, an indenyl group, a naphthyl group, an anthracenyl group, an azulenyl group, a heptalenyl group, an acenaphthyl group, a phenalenyl group, a fluorenyl group, a spiro-bifluorenyl group, a benzofluorenyl group, a dibenzofluorenyl group, a phenanthrenyl group, a biphenyl group, a terphenyl group, a triphenylenyl group, a fluoranthenyl group, a pyrenyl group, a chrysenyl group, a picenyl group, a perylenyl group, a pentaphenyl group, a pentacenyl group, a tetraphenyl group, a hexaphenyl group, a hexacenyl group, a rubicenyl group, a trinaphthyl group, a heptaphenyl group, a pyranthrenyl group, a pyridinyl group, a pyrazinyl group, a pyridazinyl group, a pyrimidinyl group, a triazinyl group, a quinolinyl group, an isoquinolinyl group, a quinoxalinyl group, a quinazolinyl group, a naphthyridinyl group, an acridinyl group, a phenazinyl group, a benzoquinolinyl group, a benzoisoquinolinyl group, a phenanthridinyl group, a phenanthrolinyl group, a benzoquinonyl group, a cumarinyl group, an anthraquinonyl group, a fluorenonyl group, a furanyl group, a thienyl group, a silolyl group, a benzofuranyl group, a benzothienyl group, a benzosilolyl group, a dibenzofuranyl group, a dibenzothiophenyl group, a dibenzosilolyl group, a pyrrolyl group, an indolyl group, an isoindolyl group, a carbazolyl group, a benzocarbazolyl group, a dibenzocarbazolyl group, an imidazolyl group, a benzimidazolyl group, a pyrazolyl group, a triazolyl group, a tetrazolyl group, an indazolyl group, an oxazolyl group, an isoxazolyl group, a benzoxazolyl group, a benzisoxazolyl group, a thiazolyl group, an isothiazolyl group, a benzothiazolyl group, a benzisothiazolyl group, an imidazopyridinyl group, an imidazopyrimidinyl group, an imidazophenanthridinyl group, a benzimidazophenanthridinyl group, an azadibenzofuranyl group, an azacarbazolyl group, an azadibenzothienyl group, a diazadibenzofuranyl group, a diazacarbazolyl group, a diazadibenzothienyl group, a xanthonyl group, and a thioxanthonyl group; and
a phenyl group, a pentalenyl group, an indenyl group, a naphthyl group, an anthracenyl group, an azulenyl group, a heptalenyl group, an acenaphthyl group, a phenalenyl group, a fluorenyl group, a spiro-bifluorenyl group, a benzofluorenyl group, a dibenzofluorenyl group, a phenanthrenyl group, a biphenyl group, a terphenyl group, a triphenylenyl group, a fluoranthenyl group, a pyrenyl group, a chrysenyl group, a picenyl group, a perylenyl group, a pentaphenyl group, a pentacenyl group, a tetraphenyl group, a hexaphenyl group, a hexacenyl group, a rubicenyl group, a trinaphthyl group, a heptaphenyl group, a pyranthrenyl group, a pyridinyl group, a pyrazinyl group, a pyridazinyl group, a pyrimidinyl group, a triazinyl group, a quinolinyl group, an isoquinolinyl group, a quinoxalinyl group, a quinazolinyl group, a naphthyridinyl group, an acridinyl group, a phenazinyl group, a benzoquinolinyl group, a benzoisoquinolinyl group, a phenanthridinyl group, a phenanthrolinyl group, a benzoquinonyl group, a cumarinyl group, an anthraquinonyl group, a fluorenonyl group, a furanyl group, a thienyl group, a silolyl group, a benzofuranyl group, a benzothienyl group, a benzosilolyl group, a dibenzofuranyl group, a dibenzothiophenyl group, a dibenzosilolyl group, a pyrrolyl group, an indolyl group, an isoindolyl group, a carbazolyl group, a benzocarbazolyl group, a dibenzocarbazolyl group, an imidazolyl group, a benzimidazolyl group, a pyrazolyl group, a triazolyl group, a tetrazolyl group, an indazolyl group, an oxazolyl group, an isoxazolyl group, a benzoxazolyl group, a benzisoxazolyl group, a thiazolyl group, an isothiazolyl group, a benzothiazolyl group, a benzisothiazolyl group, an imidazopyridinyl group, an imidazopyrimidinyl group, an imidazophenanthridinyl group, a benzimidazophenanthridinyl group, an azadibenzofuranyl group, an azacarbazolyl group, an azadibenzothienyl group, a diazadibenzofuranyl group, a diazacarbazolyl group, a diazadibenzothienyl group, a xanthonyl group, and a thioxanthonyl group, each substituted with at least one selected from deuterium, —F, —Cl, —Br, —I, a hydroxyl group, a cyano group, a nitro group, an amidino group, a hydrazine group, a hydrazone group, a C₁-C₃₀ alkyl group, a C₁-C₃₀ alkoxy group, a phenyl group, a pentalenyl group, an indenyl group, a naphthyl group, an anthracenyl group, an azulenyl group, a heptalenyl group, an acenaphthyl group, a phenalenyl group, a fluorenyl group, a spiro-bifluorenyl group, a benzofluorenyl group, a dibenzofluorenyl group, a phenanthrenyl group, a biphenyl group, a terphenyl group, a triphenylenyl group, a fluoranthenyl group, a pyrenyl group, a chrysenyl group, a picenyl group, a perylenyl group, a pentaphenyl group, a pentacenyl group, a tetraphenyl group, a hexaphenyl group, a hexacenyl group, a rubicenyl group, a trinaphthyl group, a heptaphenyl group, a pyranthrenyl group, a pyridinyl group, a pyrazinyl group, a pyridazinyl group, a pyrimidinyl group, a triazinyl group, a quinolinyl group, an isoquinolinyl group, a quinoxalinyl group, a quinazolinyl group, a naphthyridinyl group, an acridinyl group, a phenazinyl group, a benzoquinolinyl group, a benzoisoquinolinyl group, a phenanthridinyl group, a phenanthrolinyl group, a benzoquinonyl group, a cumarinyl group, an anthraquinonyl group, a fluorenonyl group, a furanyl group, a thienyl group, a silolyl group, a benzofuranyl group, a benzothienyl group, a benzosilolyl group, a dibenzofuranyl group, a dibenzothiophenyl group, a dibenzosilolyl group, a pyrrolyl group, an indolyl group, an isoindolyl group, a carbazolyl group, a benzocarbazolyl group, a dibenzocarbazolyl group, an imidazolyl group, a benzimidazolyl group, a pyrazolyl group, a triazolyl group, a tetrazolyl group, an indazolyl group, an oxazolyl group, an isoxazolyl group, a benzoxazolyl group, a benzisoxazolyl group, a thiazolyl group, an isothiazolyl group, a benzothiazolyl group, a benzisothiazolyl group, an imidazopyridinyl group, an imidazopyrimidinyl group, an imidazophenanthridinyl group, a benzimidazophenanthridinyl group, an azadibenzofuranyl group, an azacarbazolyl group, an azadibenzothienyl group, a diazadibenzofuranyl group, a diazacarbazolyl group, a diazadibenzothienyl group, a xanthonyl group, and a thioxanthonyl group.

5. The condensed cyclic compound of claim 1, wherein
R₂₁ is selected from:
a phenyl group, a pentalenyl group, an indenyl group, a naphthyl group, an anthracenyl group, an azulenyl group, a heptalenyl group, an acenaphthyl group, a phenalenyl group, a fluorenyl group, a spiro-bifluorenyl group, a benzofluorenyl group, a dibenzofluorenyl group, a phenanthrenyl group, a biphenyl group, a terphenyl group, a triphenylenyl group, a fluoranthenyl group, a pyrenyl group, a chrysenyl group, a picenyl group, a perylenyl group, a pentaphenyl group, a pentacenyl group, a tetraphenyl group, a hexaphenyl group, a hexacenyl group, a rubicenyl group, a trinaphthyl group, a heptaphenyl group, a pyranthrenyl group, a pyridinyl group, a pyrazinyl group, a pyridazinyl group, a pyrimidinyl group, a triazinyl group, a quinolinyl group, an isoquinolinyl group, a quinoxalinyl group, a quinazolinyl group, a naphthyridinyl group, an acridinyl group, a phenazinyl group, a benzoquinolinyl group, a benzoisoquinolinyl group, a phenanthridinyl group, a phenanthrolinyl group, a benzoquinonyl group, a cumarinyl group, an anthraquinonyl group, a fluorenonyl group, a furanyl group, a thienyl group, a silolyl group, a benzofuranyl group, a benzothienyl group, a benzosilolyl group, a dibenzofuranyl group, a dibenzothiophenyl group, a dibenzosilolyl group, a pyrrolyl group, an indolyl group, an isoindolyl group, a carbazolyl group, a benzocarbazolyl group, a dibenzocarbazolyl group, an imidazolyl group, a benzimidazolyl group, a pyrazolyl group, a triazolyl group, a tetrazolyl group, an indazolyl group, an oxazolyl group, an isoxazolyl group, a benzoxazolyl group, a benzisoxazolyl group, a thiazolyl group, an isothiazolyl group, a benzothiazolyl group, a benzisothiazolyl group, an imidazopyridinyl group, an imidazopyrimidinyl group, an imidazophenanthridinyl group, a benzimidazophenanthridinyl group, an azadibenzofuranyl group, an azacarbazolyl group, an azadibenzothienyl group, a diazadibenzofuranyl group, a diazacarbazolyl group, a diazadibenzothienyl group, a xanthonyl group, and a thioxanthonyl group; and
a phenyl group, a pentalenyl group, an indenyl group, a naphthyl group, an anthracenyl group, an azulenyl group, a heptalenyl group, an acenaphthyl group, a phenalenyl group, a fluorenyl group, a spiro-bifluorenyl group, a benzofluorenyl group, a dibenzofluorenyl group, a phenanthrenyl group, a biphenyl group, a terphenyl group, a triphenylenyl group, a fluoranthenyl group, a pyrenyl group, a chrysenyl group, a picenyl group, a perylenyl group, a pentaphenyl group, a pentacenyl group, a tetraphenyl group, a hexaphenyl group, a hexacenyl group, a rubicenyl group, a trinaphthyl group, a heptaphenyl group, a pyranthrenyl group, a pyridinyl group, a pyrazinyl group, a pyridazinyl group, a pyrimidinyl group, a triazinyl group, a quinolinyl group, an isoquinolinyl group, a quinoxalinyl group, a quinazolinyl group, a naphthyridinyl group, an acridinyl group, a phenazinyl group, a benzoquinolinyl group, a benzoisoquinolinyl group, a phenanthridinyl group, a phenanthrolinyl group, a benzoquinonyl group, a cumarinyl group, an anthraquinonyl group, a fluorenonyl group, a furanyl group, a thienyl group, a silolyl group, a benzofuranyl group, a benzothienyl group, a benzosilolyl group, a dibenzofuranyl group, a dibenzothiophenyl group, a dibenzosilolyl group, a pyrrolyl group, an indolyl group, an isoindolyl group, a carbazolyl group, a benzocarbazolyl group, a dibenzocarbazolyl group, an imidazolyl group, a benzimidazolyl group, a pyrazolyl group, a triazolyl group, a tetrazolyl group, an indazolyl group, an oxazolyl group, an isoxazolyl group, a benzoxazolyl group, a benzisoxazolyl group, a thiazolyl group, an isothiazolyl group, a benzothiazolyl group, a benzisothiazolyl group, an imidazopyridinyl group, an imidazopyrimidinyl group, an imidazophenanthridinyl group, a benzimidazophenanthridinyl group, an azadibenzofuranyl group, an azacarbazolyl group, an azadibenzothienyl group, a diazadibenzofuranyl group, a diazacarbazolyl group, a diazadibenzothienyl group, a xanthonyl group, and a thioxanthonyl group, each substituted with at least one selected from deuterium, —F, —Cl, —Br, —I, a hydroxyl group, a cyano group, a nitro group, an amidino group, a hydrazine group, a hydrazone group, a C₁-C₃₀ alkyl group, a C₁-C₃₀ alkoxy group, a phenyl group, a pentalenyl group, an indenyl group, a naphthyl group, an anthracenyl group, an azulenyl group, a heptalenyl group, an acenaphthyl group, a phenalenyl group, a fluorenyl group, a spiro-bifluorenyl group, a benzofluorenyl group, a dibenzofluorenyl group, a phenanthrenyl group, a biphenyl group, a terphenyl group, a triphenylenyl group, a fluoranthenyl group, a pyrenyl group, a chrysenyl group, a picenyl group, a perylenyl group, a pentaphenyl group, a pentacenyl group, a tetraphenyl group, a hexaphenyl group, a hexacenyl group, a rubicenyl group, a trinaphthyl group, a heptaphenyl group, a pyranthrenyl group, a pyridinyl group, a pyrazinyl group, a pyridazinyl group, a pyrimidinyl group, a triazinyl group, a quinolinyl group, an isoquinolinyl group, a quinoxalinyl group, a quinazolinyl group, a naphthyridinyl group, an acridinyl group, a phenazinyl group, a benzoquinolinyl group, a benzoisoquinolinyl group, a phenanthridinyl group, a phenanthrolinyl group, a benzoquinonyl group, a cumarinyl group, an anthraquinonyl group, a fluorenonyl group, a furanyl group, a thienyl group, a silolyl group, a benzofuranyl group, a benzothienyl group, a benzosilolyl group, a dibenzofuranyl group, a dibenzothiophenyl group, a dibenzosilolyl group, a pyrrolyl group, an indolyl group, an isoindolyl group, a carbazolyl group, a benzocarbazolyl group, a dibenzocarbazolyl group, an imidazolyl group, a benzimidazolyl group, a pyrazolyl group, a triazolyl group, a tetrazolyl group, an indazolyl group, an oxazolyl group, an isoxazolyl group, a benzoxazolyl group, a benzisoxazolyl group, a thiazolyl group, an isothiazolyl group, a benzothiazolyl group, a benzisothiazolyl group, an imidazopyridinyl group, an imidazopyrimidinyl group, an imidazophenanthridinyl group, a benzimidazophenanthridinyl group, an azadibenzofuranyl group, an azacarbazolyl group, an azadibenzothienyl group, a diazadibenzofuranyl group, a diazacarbazolyl group, a diazadibenzothienyl group, a xanthonyl group, and a thioxanthonyl group.

6. The condensed cyclic compound of claim 1, wherein
R₂₁ is selected from:
a phenyl group, a biphenyl group, and a terphenyl group; and
a phenyl group, a biphenyl group, and a terphenyl group, each substituted with at least one selected from deuterium, —F, —Cl, —Br, —I, a cyano group, a C₁-C₂₀ alkyl group, and a C₁-C₂₀ alkoxy group.

7. The condensed cyclic compound of claim 1, wherein
i) a1 is 0 or 1, and Ar₁ and Ar₄ are each independently a group represented by *-(L₂₁)ₐ₂₁-R₂₁;
ii) a1 is 1, and Ar₃ and Ar₄ are each independently a group represented by *-(L₂₁)ₐ₂₁-R₂₁; or
iii) a1 is 1, and Ar₁, Ar₃, and Ar₄ are each independently a group represented by *-(L₂₁)ₐ₂₁-R₂₁.

8. The condensed cyclic compound of claim 1, wherein
each of at least one selected from R₁ to R₃ and R₁₄ to R₁₆ is independently selected from a substituted or unsubstituted C₆-C₆₀ aryl group, a substituted or unsubstituted C₁-C₆₀ heteroaryl group, a substituted or unsubstituted monovalent non-aromatic condensed polycyclic group, and a substituted or unsubstituted monovalent non-aromatic condensed heteropolycyclic group.

9. The condensed cyclic compound of claim 1, wherein
i) a1 is 0 or 1, and Ar₁ and Ar₄ are each independently a group represented by *-(L₂₁)ₐ₂₁-R₂₁, and
each of one selected from R₁ and R₂ and/or one selected from R₁₄ and R₁₅ is independently selected from a substituted or unsubstituted C₆-C₆₀ aryl group, a substituted or unsubstituted C₁-C₆₀ heteroaryl group, a substituted or unsubstituted monovalent non-aromatic condensed polycyclic group, and a substituted or unsubstituted monovalent non-aromatic condensed heteropolycyclic group;
ii) a1 is 1, and Ar₃ and Ar₄ are each independently a group represented by *-(L₂₁)ₐ₂₁-R₂₁, and
each of one selected from R₁ and R₂ and/or one selected from R₁₄ and R₁₅ is independently selected from a substituted or unsubstituted C₆-C₆₀ aryl group, a substituted or unsubstituted C₁-C₆₀ heteroaryl group, a substituted or unsubstituted monovalent non-aromatic condensed polycyclic group, and a substituted or unsubstituted monovalent non-aromatic condensed heteropolycyclic group; or
iii) a1 is 1, and Arₜ Ara, and Ar₄ are each independently a group represented by *-(L₂₁)ₐ₂₁-R₂₁, and
each of one selected from R₁ and R₂ and/or one selected from R₁₄ and R₁₅ is independently selected from a substituted or unsubstituted C₆-C₆₀ aryl group, a substituted or unsubstituted C₁-C₆₀ heteroaryl group, a substituted or unsubstituted monovalent non-aromatic condensed polycyclic group, and a substituted or unsubstituted monovalent non-aromatic condensed heteropolycyclic group.

10. The condensed cyclic compound of claim 1, wherein
each of at least one selected from R₆ and R₁₁ is independently selected from a substituted or unsubstituted C₆-C₆₀ aryl group, a substituted or unsubstituted C₁-C₆₀ heteroaryl group, a substituted or unsubstituted monovalent non-aromatic condensed polycyclic group, and a substituted or unsubstituted monovalent non-aromatic condensed heteropolycyclic group.

11. The condensed cyclic compound of claim 1, wherein
each of at least one selected from R₆ and R₁₁ is independently selected from a substituted or unsubstituted C₆-C₆₀ aryl group, a substituted or unsubstituted C₁-C₆₀ heteroaryl group, a substituted or unsubstituted monovalent non-aromatic condensed polycyclic group, and a substituted or unsubstituted monovalent non-aromatic condensed heteropolycyclic group, and
R₄, R₅, R₇ to R₉, R₁₀, R₁₂, R₁₃, R₁₇, and R₁₈ are each independently hydrogen.

12. The condensed cyclic compound of claim 1, wherein
the condensed cyclic compound is represented by Formula 1A: 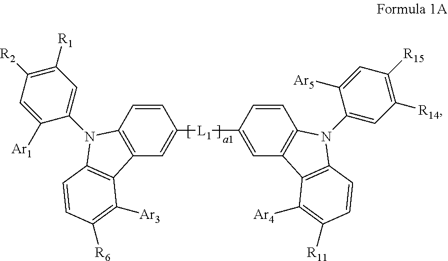
wherein, in Formula 1A,
i) each of at least one selected from Ar₁ and Ar₅ is independently a group represented by *-(L₂₁)ₐ₂₁-R₂₁, and one selected from Ar₃ and Ar₄ is a group represented by *-(L₂₁)ₐ₂₁-R₂₁; or
ii) Ar₃ and Ar₄ are each independently a group represented by *-(L₂₁)ₐ₂₁-R₂₁,
when a1 is 0, one selected from Ar₃ and Ar₄ is selected from hydrogen and deuterium, and
Li, a1, Ar₁, Ar₃ to Ar₅, R₁, R₂, R₆, R₁₁, R₁₄, and R₁₅ are each independently the same as described in Formula 1.

13. The condensed cyclic compound of claim 1, wherein
the condensed cyclic compound is represented by Formula 1-1 or 1-2: 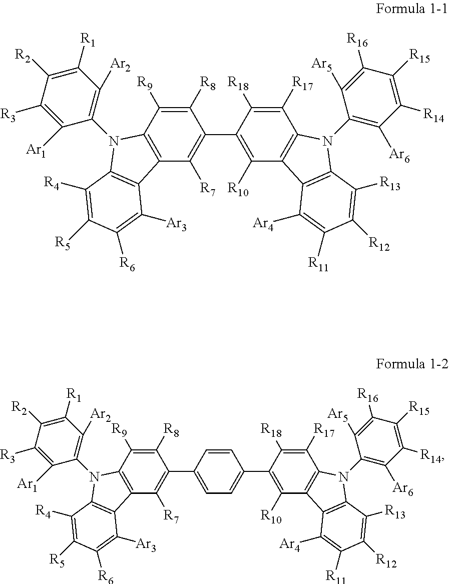
wherein, in Formula 1-1, one selected from Ar₃ and Ar₄ is selected from hydrogen and deuterium, and
in Formulae 1-1 and 1-2, Ar₁ to Ar₆ and R₁ to R₁₈ are each independently the same as described in Formula 1.

14. The condensed cyclic compound of claim 13, wherein
each of at least one selected from R₆ and R₁₁ is independently selected from a substituted or unsubstituted C₆-C₆₀ aryl group, a substituted or unsubstituted C₁-C₆₀ heteroaryl group, a substituted or unsubstituted monovalent non-aromatic condensed polycyclic group, and a substituted or unsubstituted monovalent non-aromatic condensed heteropolycyclic group.

15. The condensed cyclic compound of claim 1, wherein
the condensed cyclic compound is selected from Compounds 1 to 154:

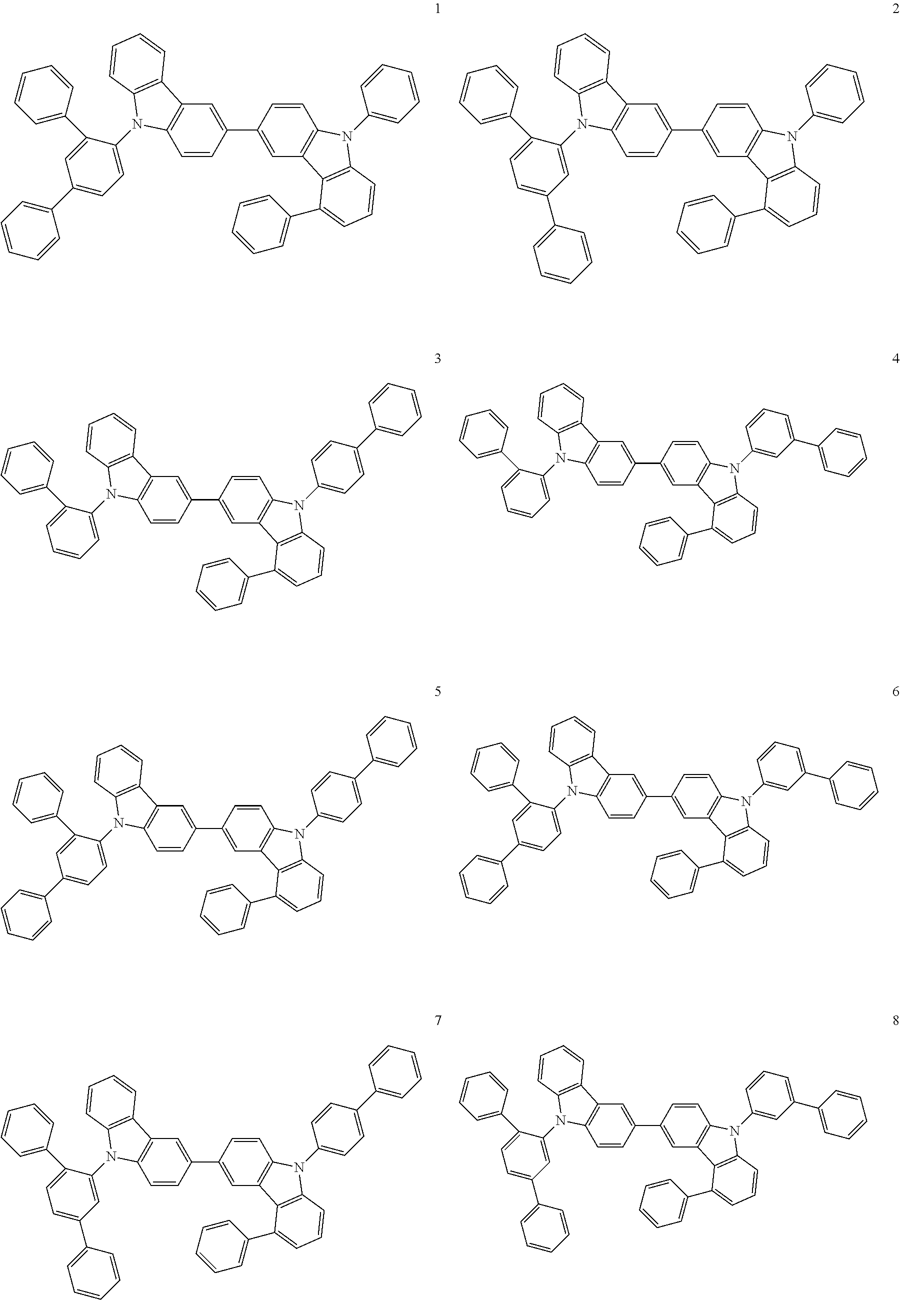

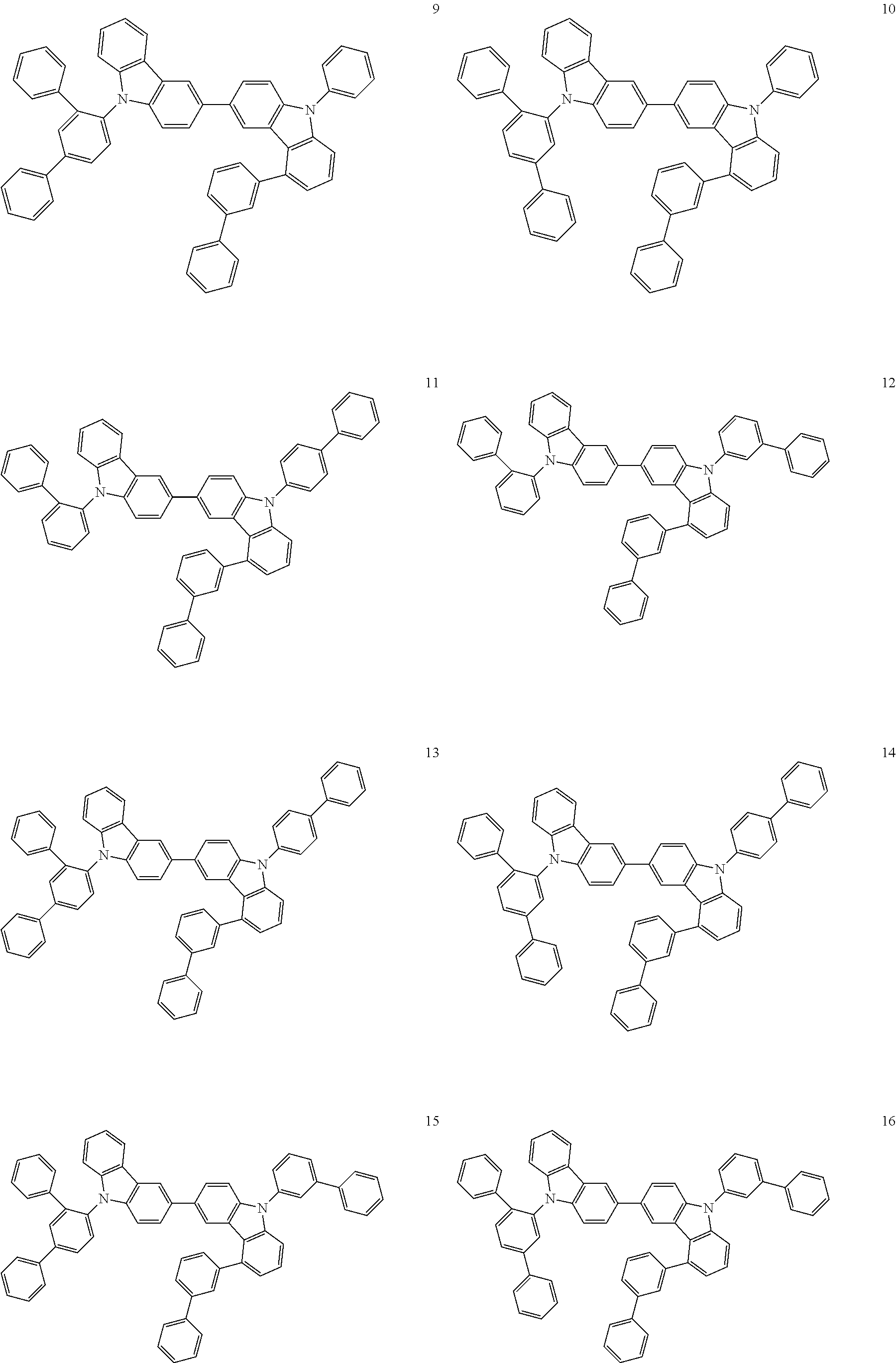

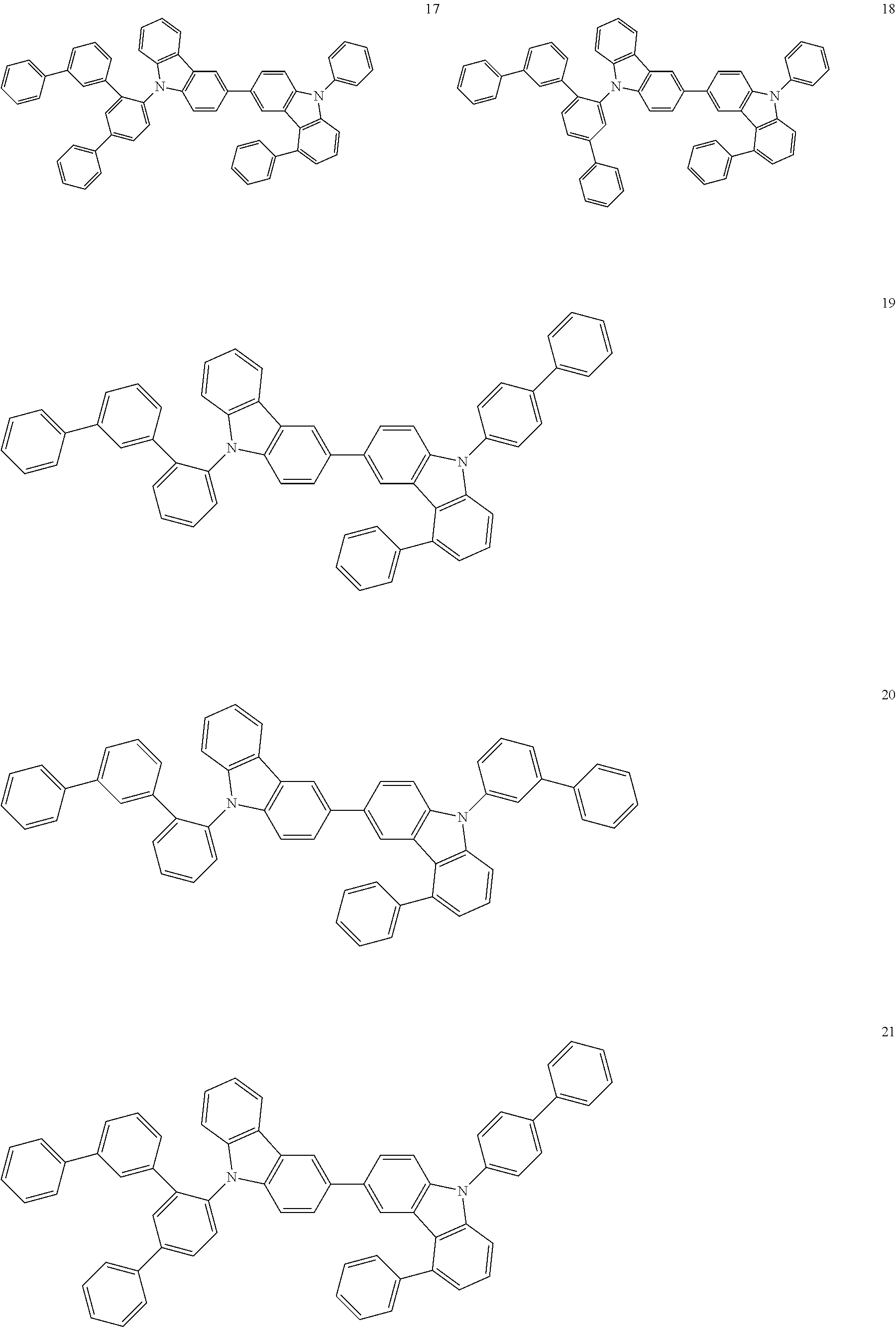

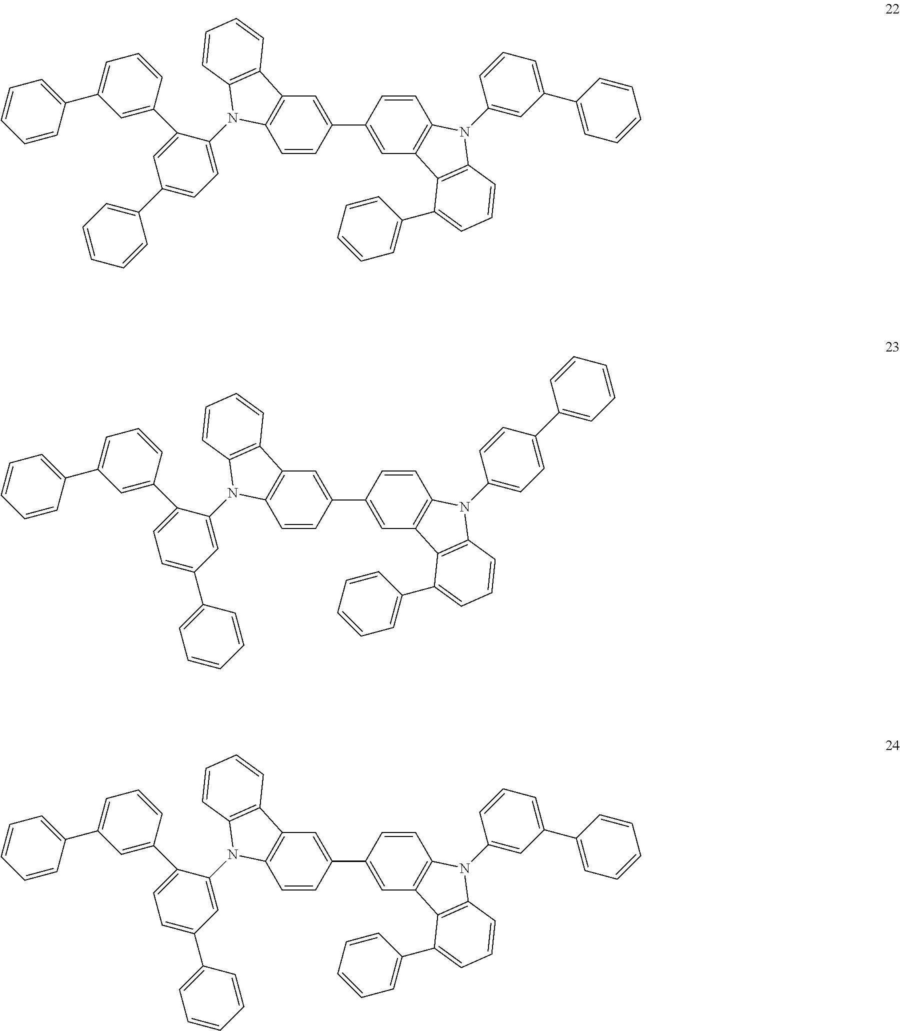 [SMILES: c1ccc(-c2cccc(-c3cc(-c4ccccc4)ccc3-n3c4ccccc4c4cc(-c5ccc6c(c5)c5c(-c7ccccc7)cccc5n6-c5cccc(-c6ccccc6)c5)ccc43)c2)cc1 ; c1ccc(-c2ccc(-n3c4ccc(-c5ccc6c(c5)c5ccccc5n6-c5cc(-c6ccccc6)ccc5-c5cccc(-c6ccccc6)c5)cc4c4c(-c5ccccc5)cccc43)cc2)cc1 ; c1ccc(-c2cccc(-c3ccc(-c4ccccc4)cc3-n3c4ccccc4c4cc(-c5ccc6c(c5)c5c(-c7ccccc7)cccc5n6-c5cccc(-c6ccccc6)c5)ccc43)c2)cc1]

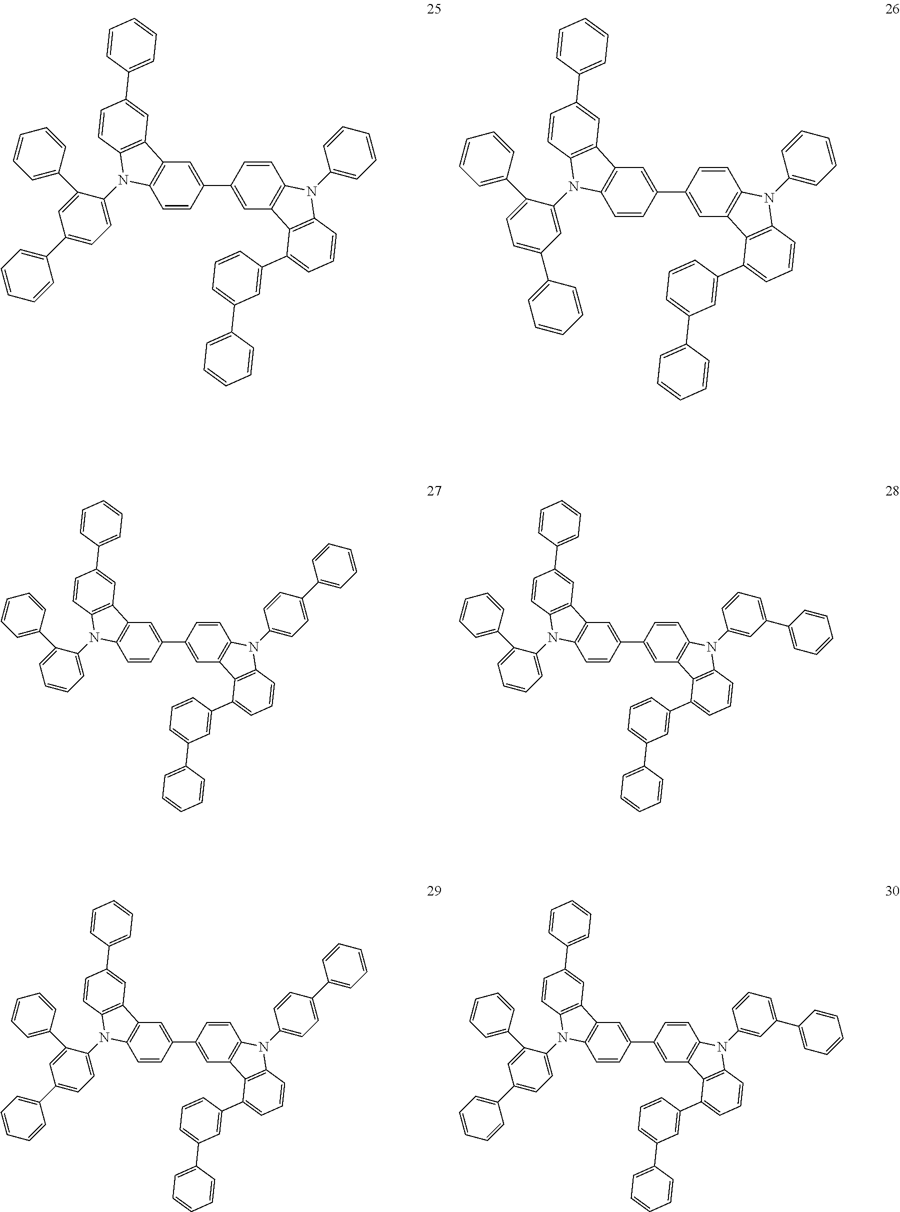

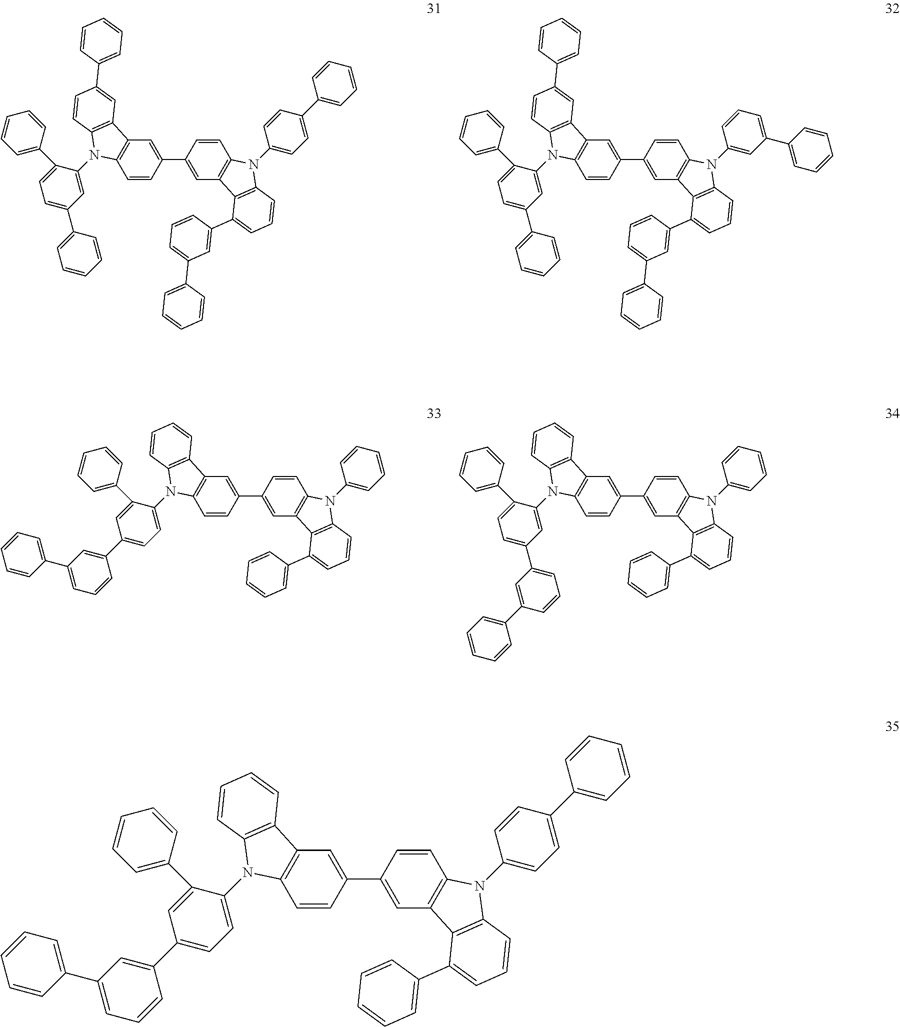

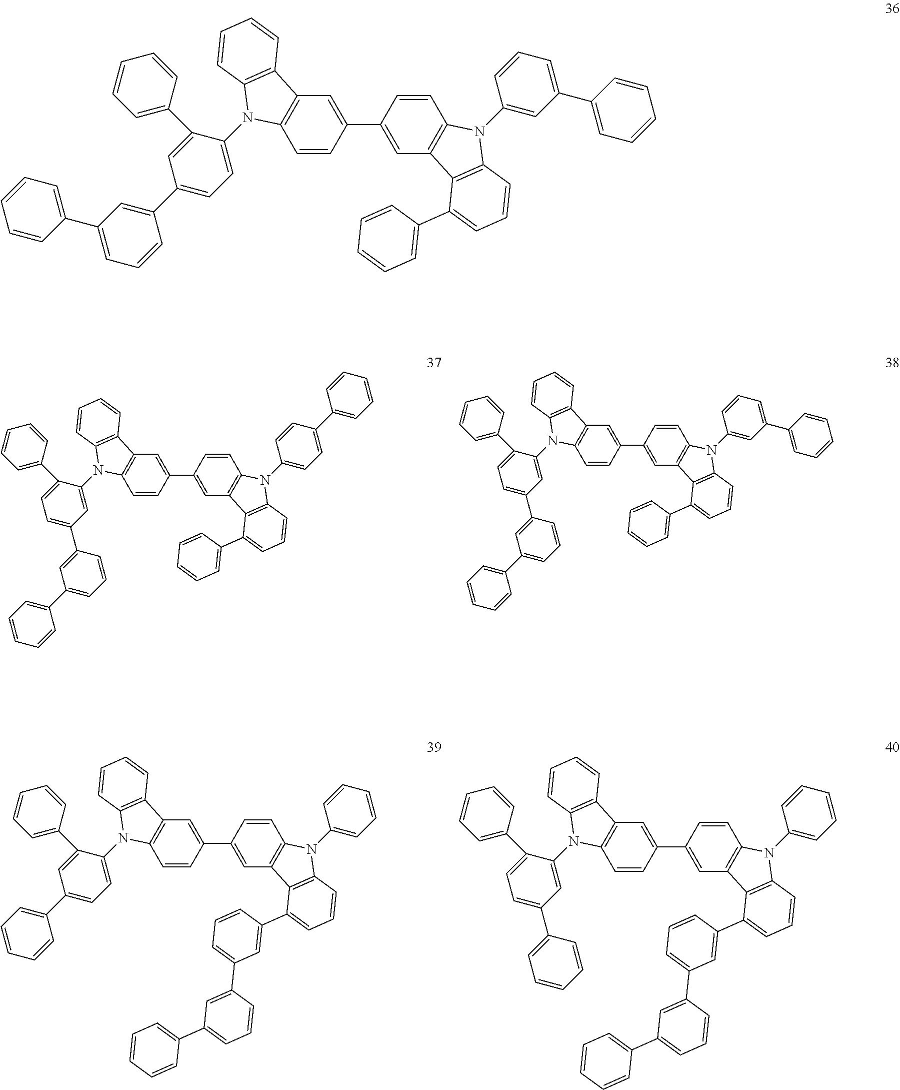

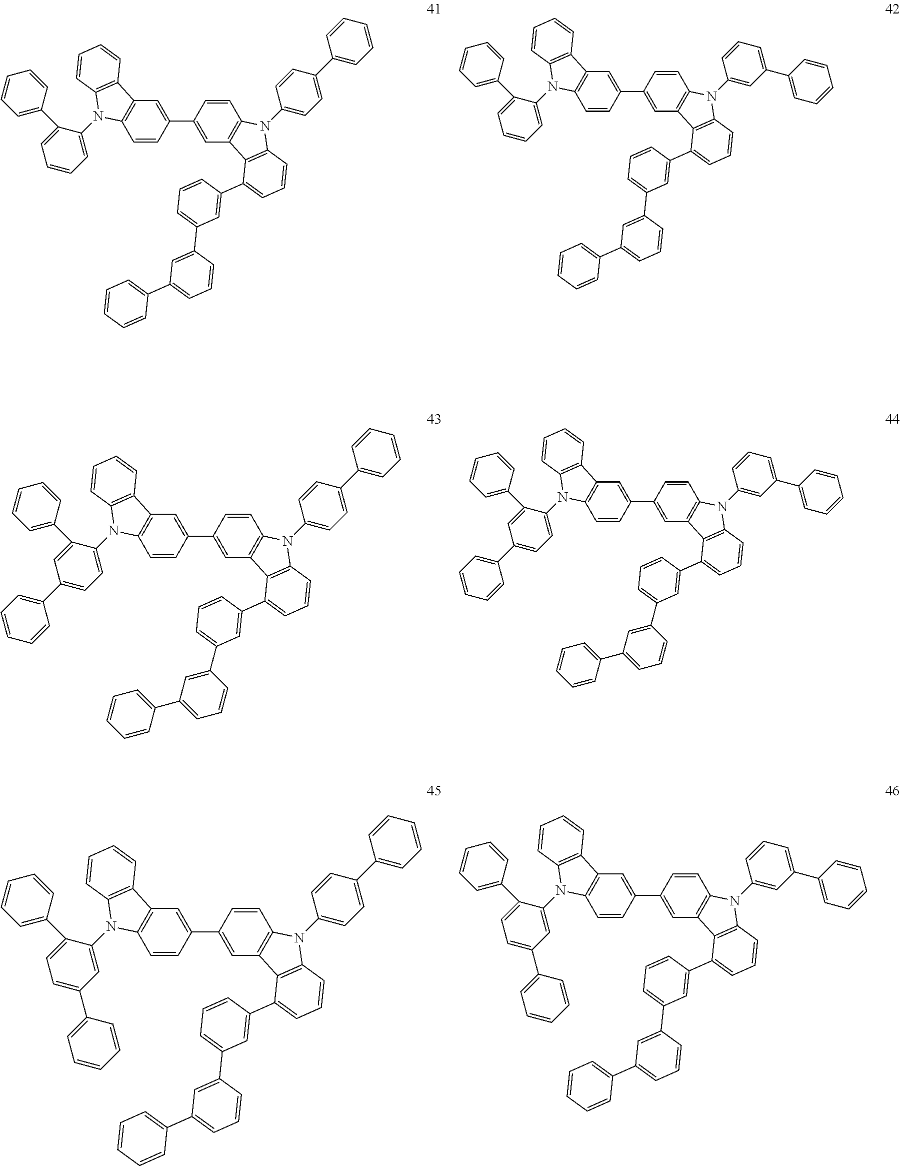

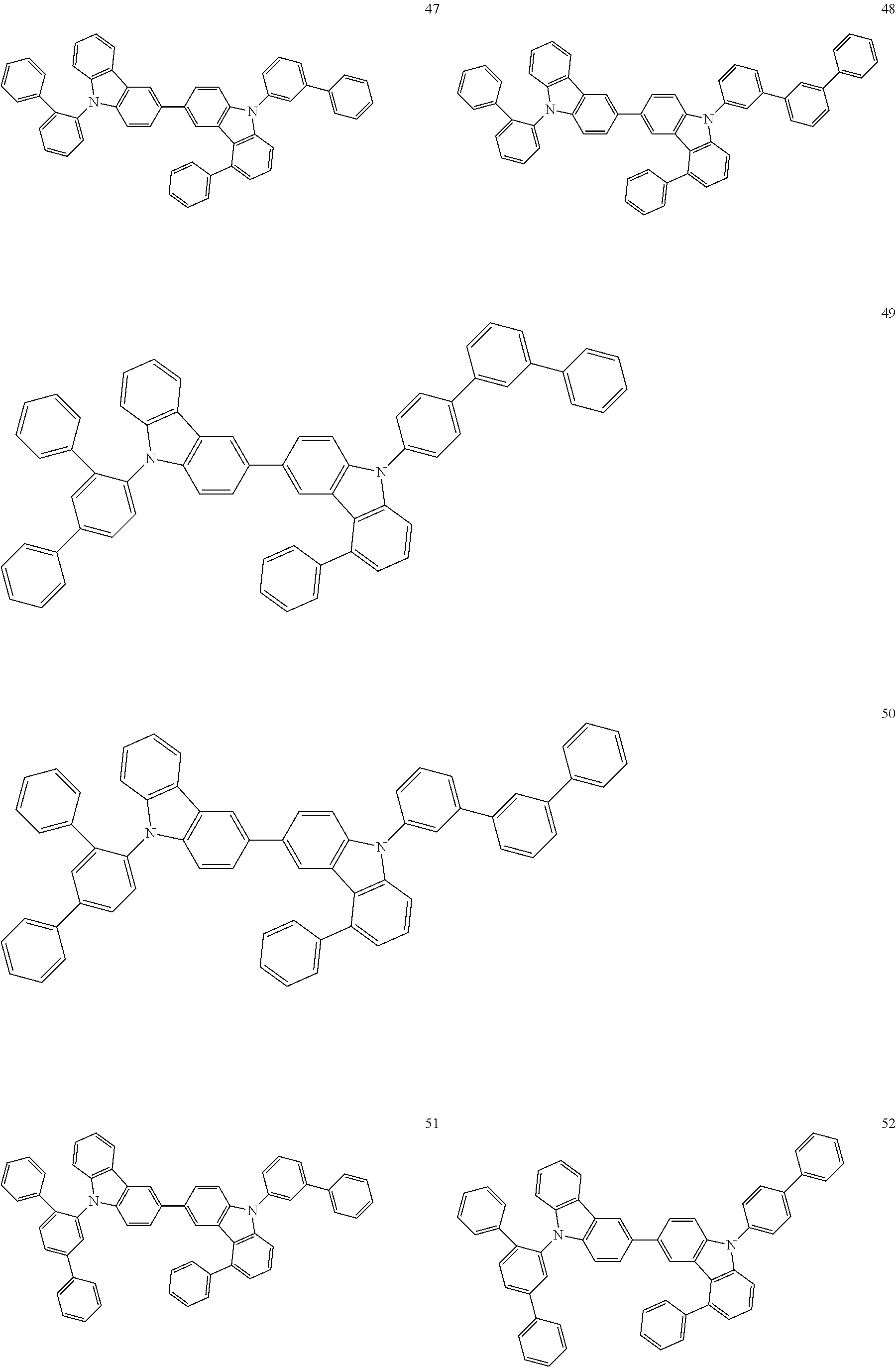

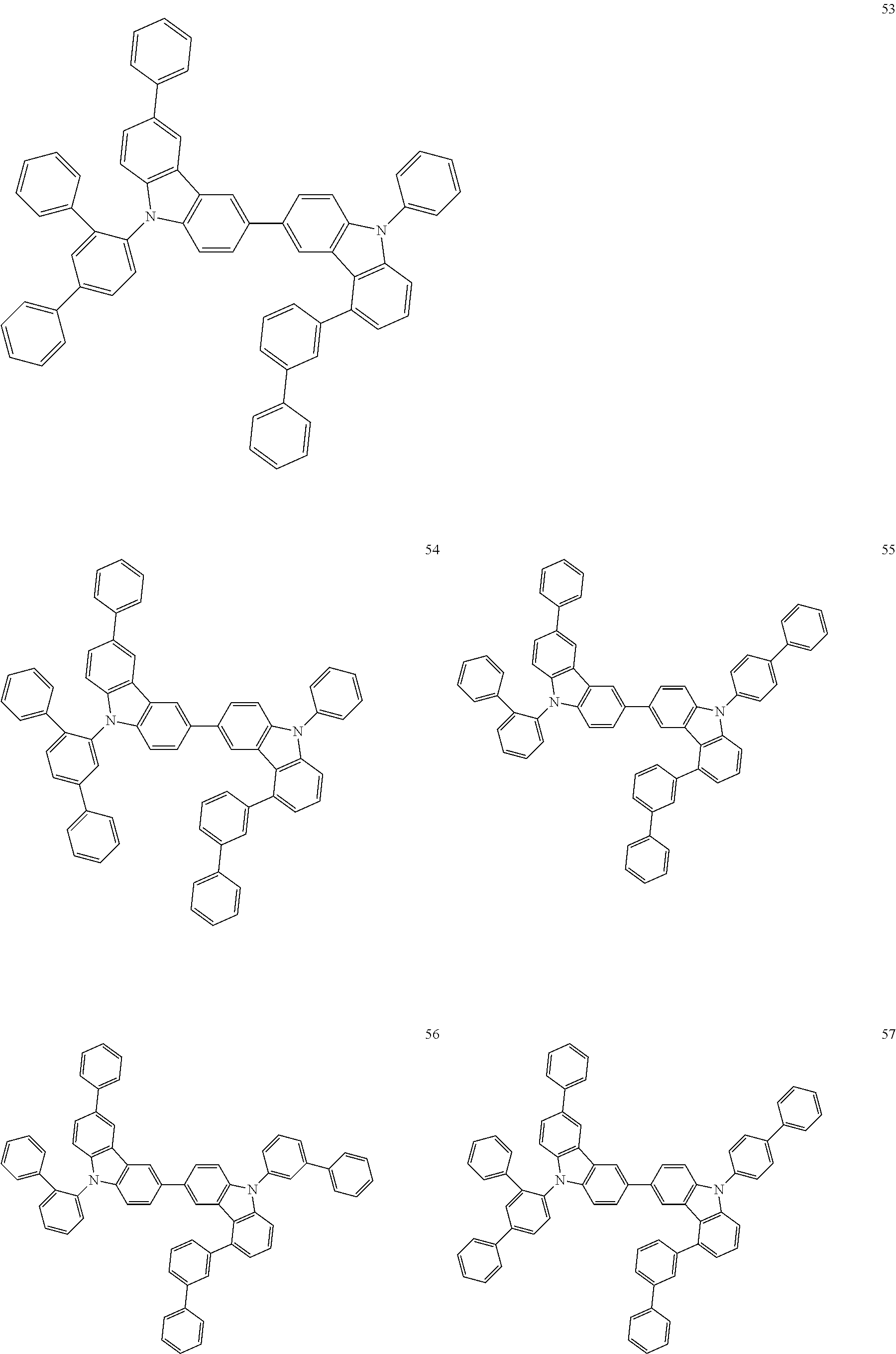

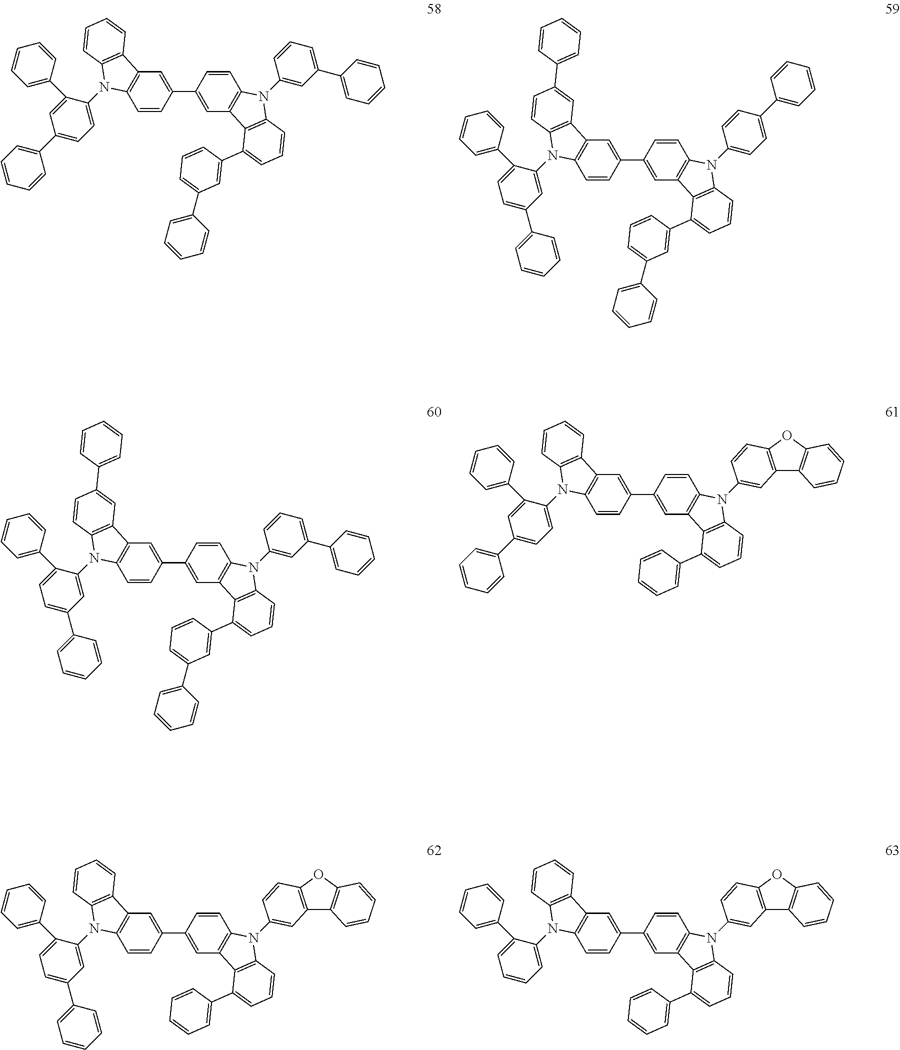

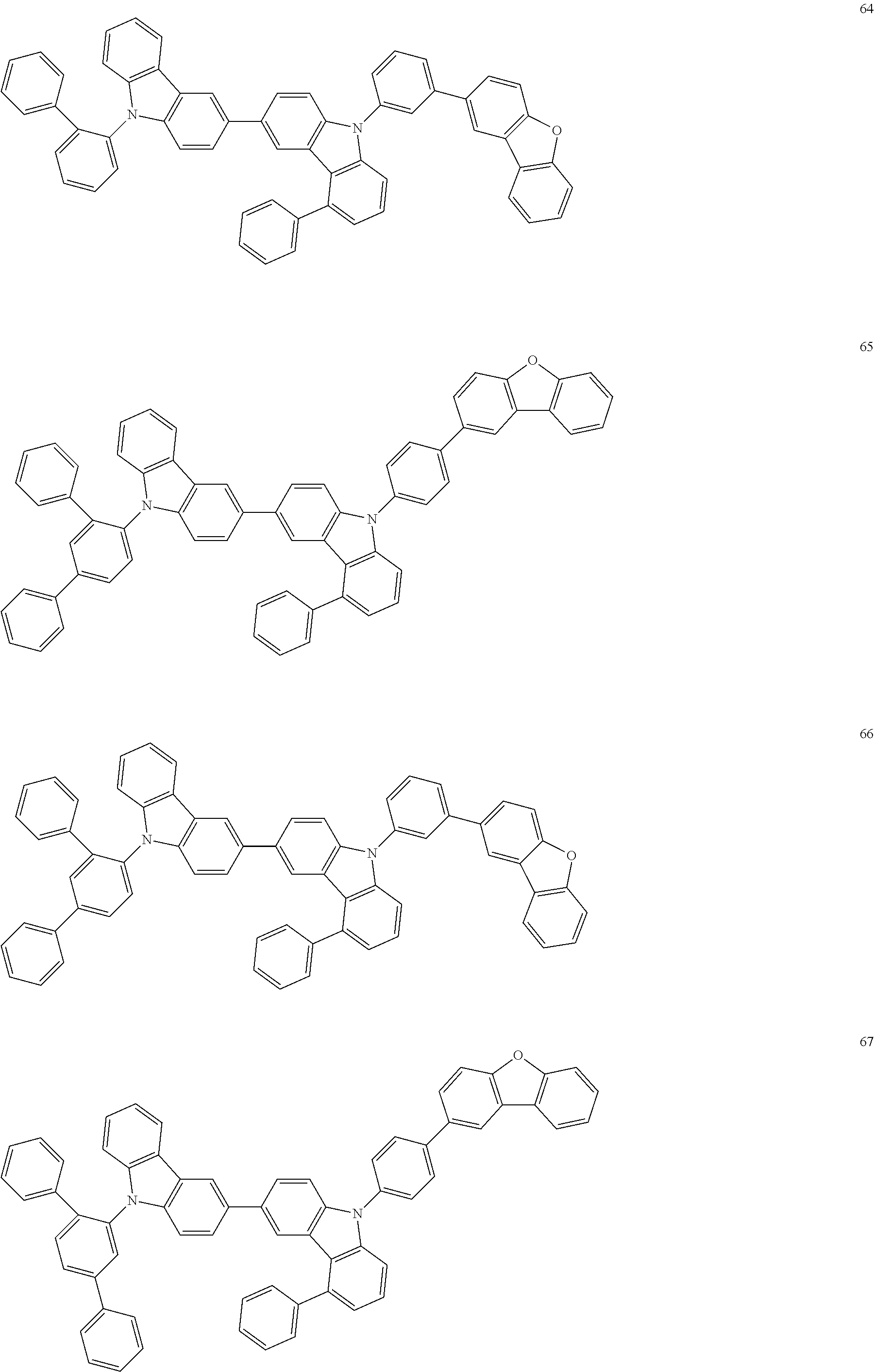 [SMILES: c1ccc(-c2ccccc2-n2c3ccccc3c3cc(-c4ccc5c(c4)c4c(-c6ccccc6)cccc4n5-c4cccc(-c5ccc6oc7ccccc7c6c5)c4)ccc32)cc1 ; c1ccc(-c2ccc(-n3c4ccccc4c4cc(-c5ccc6c(c5)c5c(-c7ccccc7)cccc5n6-c5ccc(-c6ccc7oc8ccccc8c7c6)cc5)ccc43)c(-c3ccccc3)c2)cc1 ; c1ccc(-c2ccc(-n3c4ccccc4c4cc(-c5ccc6c(c5)c5c(-c7ccccc7)cccc5n6-c5cccc(-c6ccc7oc8ccccc8c7c6)c5)ccc43)c(-c3ccccc3)c2)cc1 ; c1ccc(-c2ccc(-c3ccccc3)c(-n3c4ccccc4c4cc(-c5ccc6c(c5)c5c(-c7ccccc7)cccc5n6-c5ccc(-c6ccc7oc8ccccc8c7c6)cc5)ccc43)c2)cc1]

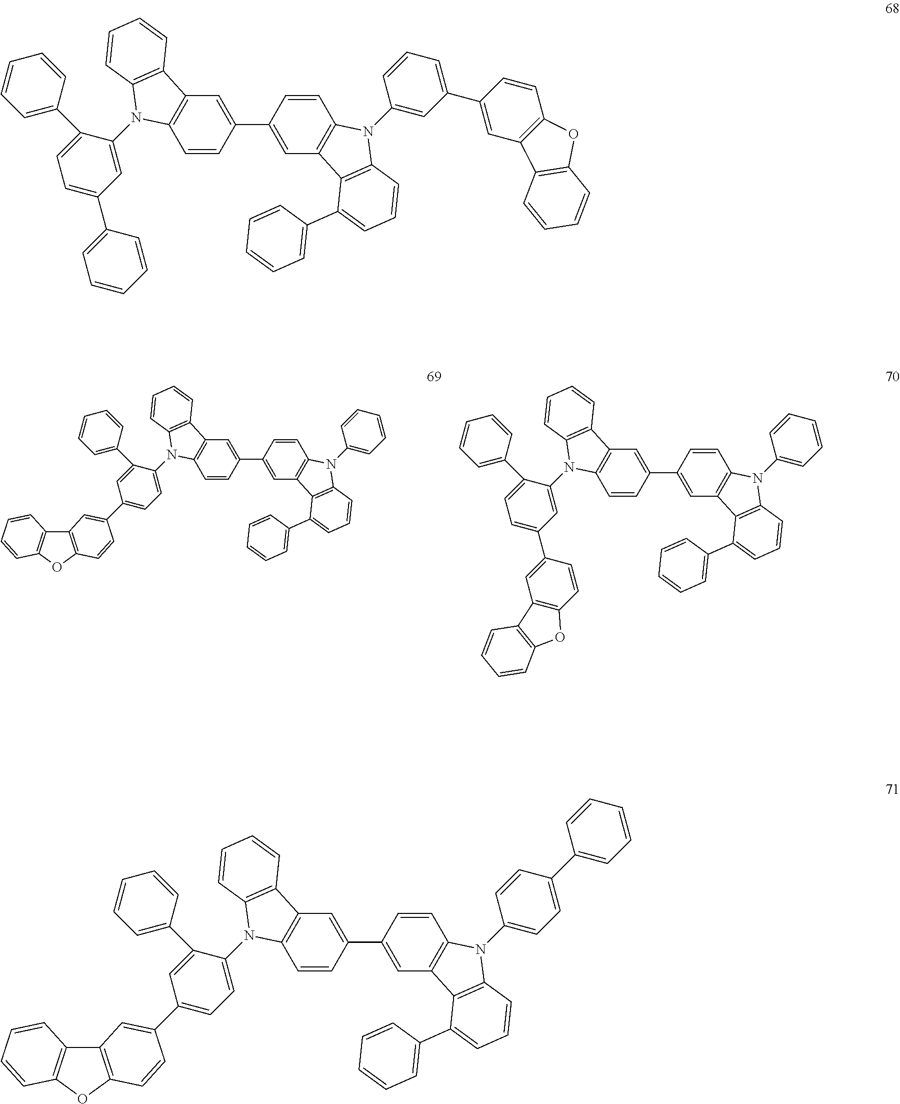 [SMILES: c1ccc(-c2ccc(-c3ccccc3)c(-n3c4ccccc4c4cc(-c5ccc6c(c5)c5c(-c7ccccc7)cccc5n6-c5cccc(-c6ccc7oc8ccccc8c7c6)c5)ccc43)c2)cc1 ; c1ccc(-c2cc(-c3ccc4oc5ccccc5c4c3)ccc2-n2c3ccccc3c3cc(-c4ccc5c(c4)c4c(-c6ccccc6)cccc4n5-c4ccccc4)ccc32)cc1 ; c1ccc(-c2ccc(-c3ccc4oc5ccccc5c4c3)cc2-n2c3ccccc3c3cc(-c4ccc5c(c4)c4c(-c6ccccc6)cccc4n5-c4ccccc4)ccc32)cc1 ; c1ccc(-c2ccc(-n3c4ccc(-c5ccc6c(c5)c5ccccc5n6-c5ccc(-c6ccc7oc8ccccc8c7c6)cc5-c5ccccc5)cc4c4c(-c5ccccc5)cccc43)cc2)cc1]

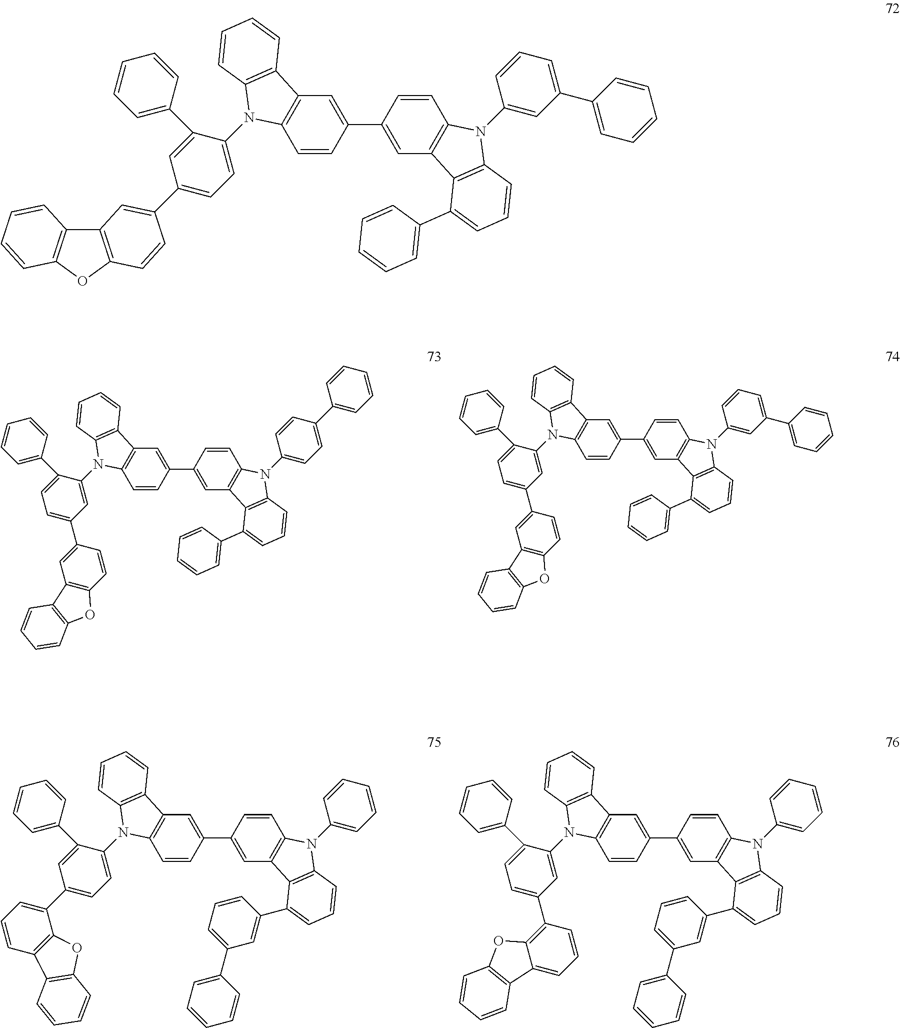

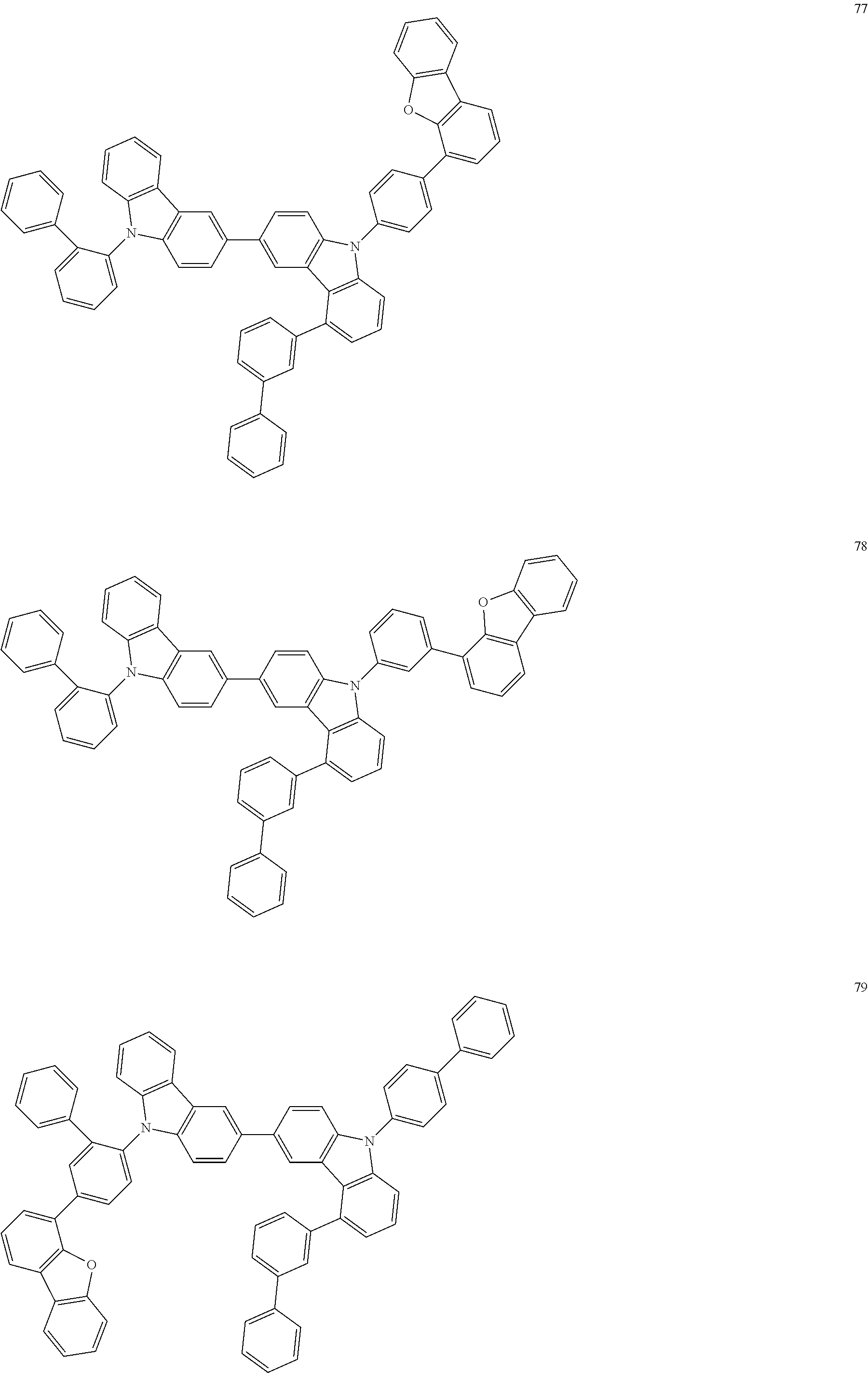 [SMILES: c1ccc(-c2cccc(-c3cccc4c3c3cc(-c5ccc6c(c5)c5ccccc5n6-c5ccccc5-c5ccccc5)ccc3n4-c3ccc(-c4cccc5c4oc4ccccc45)cc3)c2)cc1 ; c1ccc(-c2cccc(-c3cccc4c3c3cc(-c5ccc6c(c5)c5ccccc5n6-c5ccccc5-c5ccccc5)ccc3n4-c3cccc(-c4cccc5c4oc4ccccc45)c3)c2)cc1 ; c1ccc(-c2ccc(-n3c4ccc(-c5ccc6c(c5)c5ccccc5n6-c5ccc(-c6cccc7c6oc6ccccc67)cc5-c5ccccc5)cc4c4c(-c5cccc(-c6ccccc6)c5)cccc43)cc2)cc1]

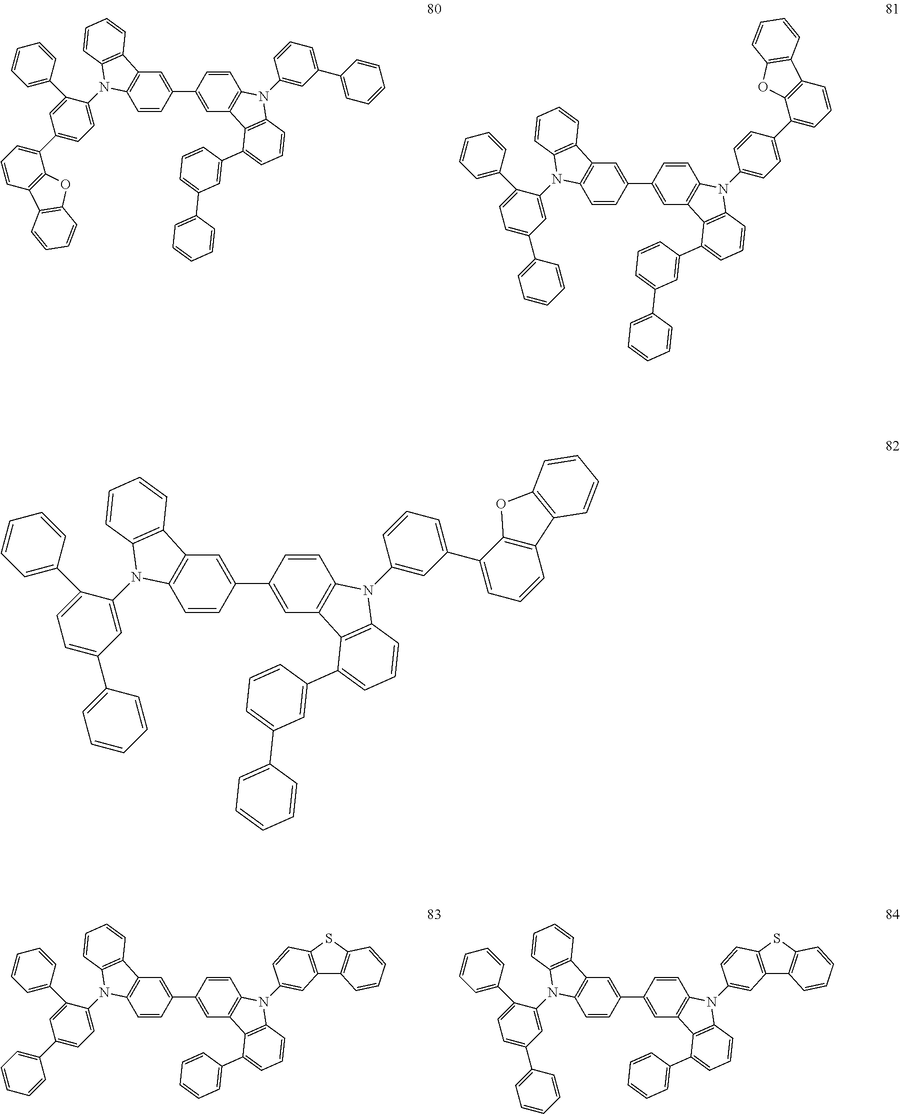

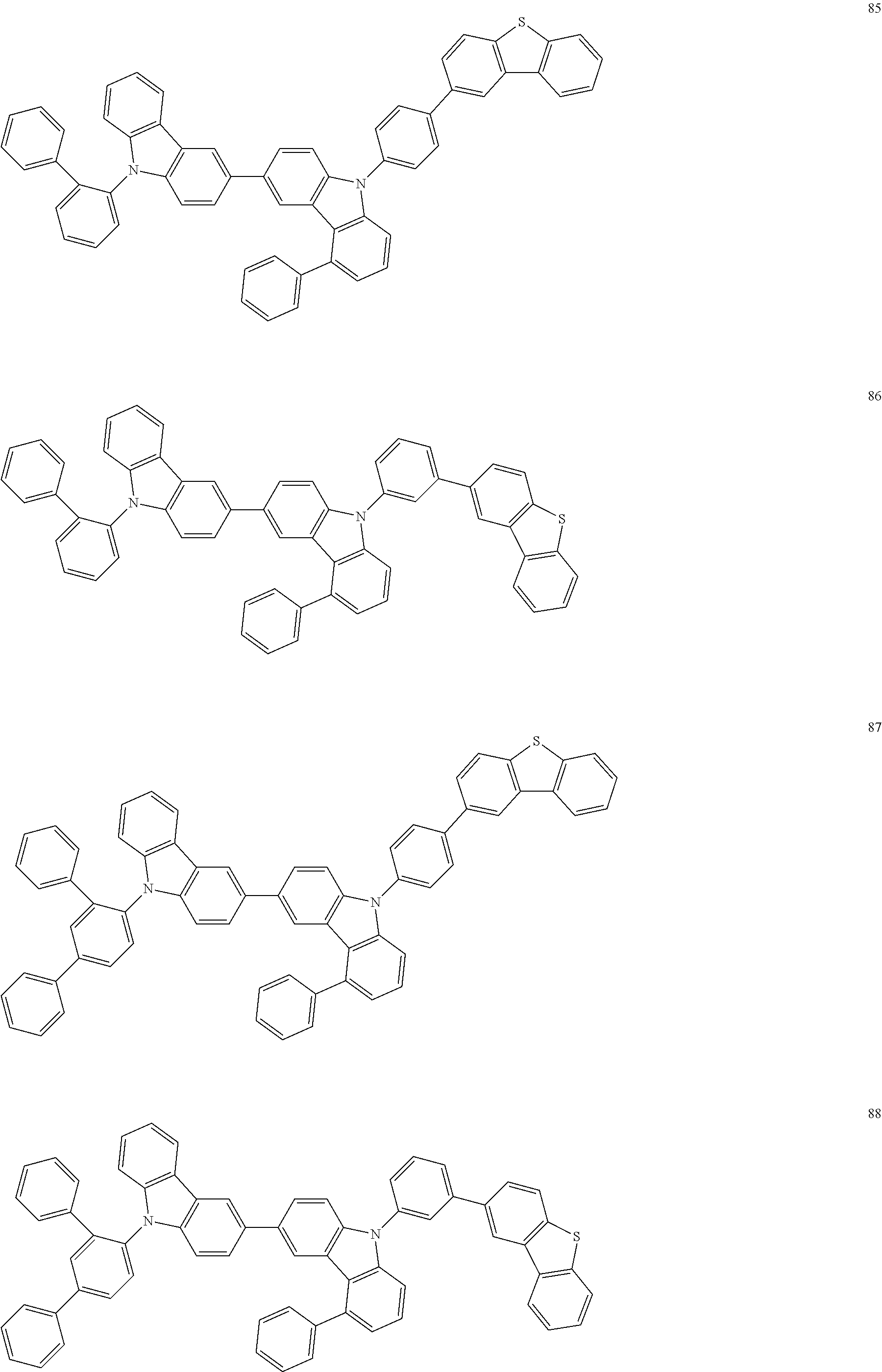 [SMILES: c1ccc(-c2ccccc2-n2c3ccccc3c3cc(-c4ccc5c(c4)c4c(-c6ccccc6)cccc4n5-c4ccc(-c5ccc6sc7ccccc7c6c5)cc4)ccc32)cc1 ; c1ccc(-c2ccccc2-n2c3ccccc3c3cc(-c4ccc5c(c4)c4c(-c6ccccc6)cccc4n5-c4cccc(-c5ccc6sc7ccccc7c6c5)c4)ccc32)cc1 ; c1ccc(-c2ccc(-n3c4ccccc4c4cc(-c5ccc6c(c5)c5c(-c7ccccc7)cccc5n6-c5ccc(-c6ccc7sc8ccccc8c7c6)cc5)ccc43)c(-c3ccccc3)c2)cc1 ; c1ccc(-c2ccc(-n3c4ccccc4c4cc(-c5ccc6c(c5)c5c(-c7ccccc7)cccc5n6-c5cccc(-c6ccc7sc8ccccc8c7c6)c5)ccc43)c(-c3ccccc3)c2)cc1]

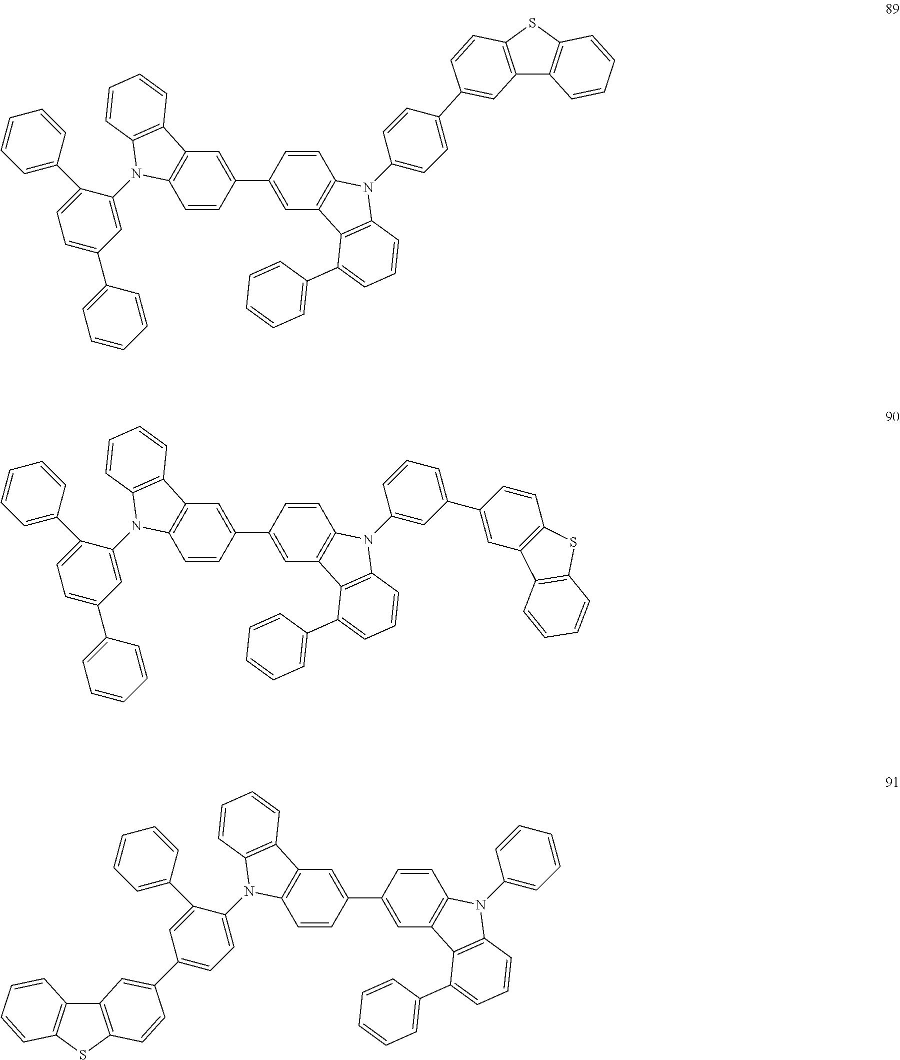 [SMILES: c1ccc(-c2ccc(-c3ccccc3)c(-n3c4ccccc4c4cc(-c5ccc6c(c5)c5c(-c7ccccc7)cccc5n6-c5ccc(-c6ccc7sc8ccccc8c7c6)cc5)ccc43)c2)cc1 ; c1ccc(-c2ccc(-c3ccccc3)c(-n3c4ccccc4c4cc(-c5ccc6c(c5)c5c(-c7ccccc7)cccc5n6-c5cccc(-c6ccc7sc8ccccc8c7c6)c5)ccc43)c2)cc1 ; c1ccc(-c2cc(-c3ccc4sc5ccccc5c4c3)ccc2-n2c3ccccc3c3cc(-c4ccc5c(c4)c4c(-c6ccccc6)cccc4n5-c4ccccc4)ccc32)cc1]

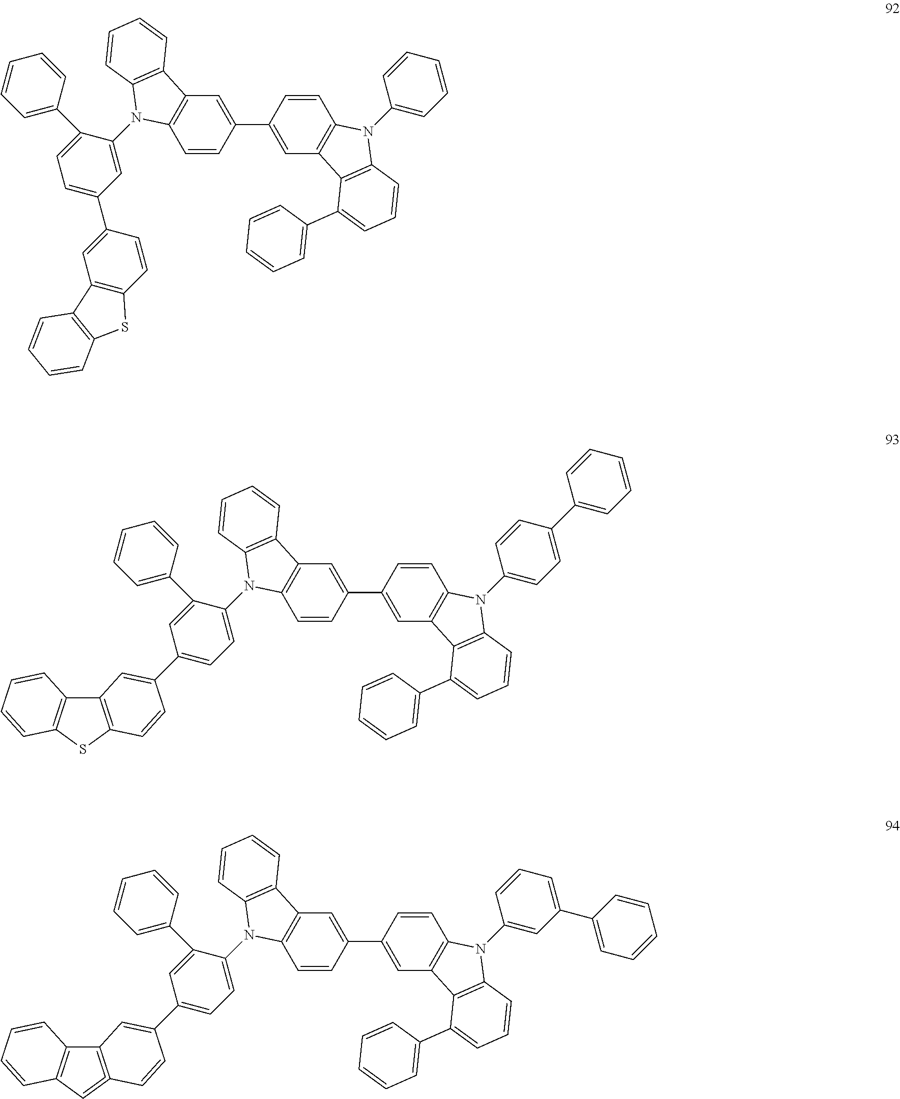

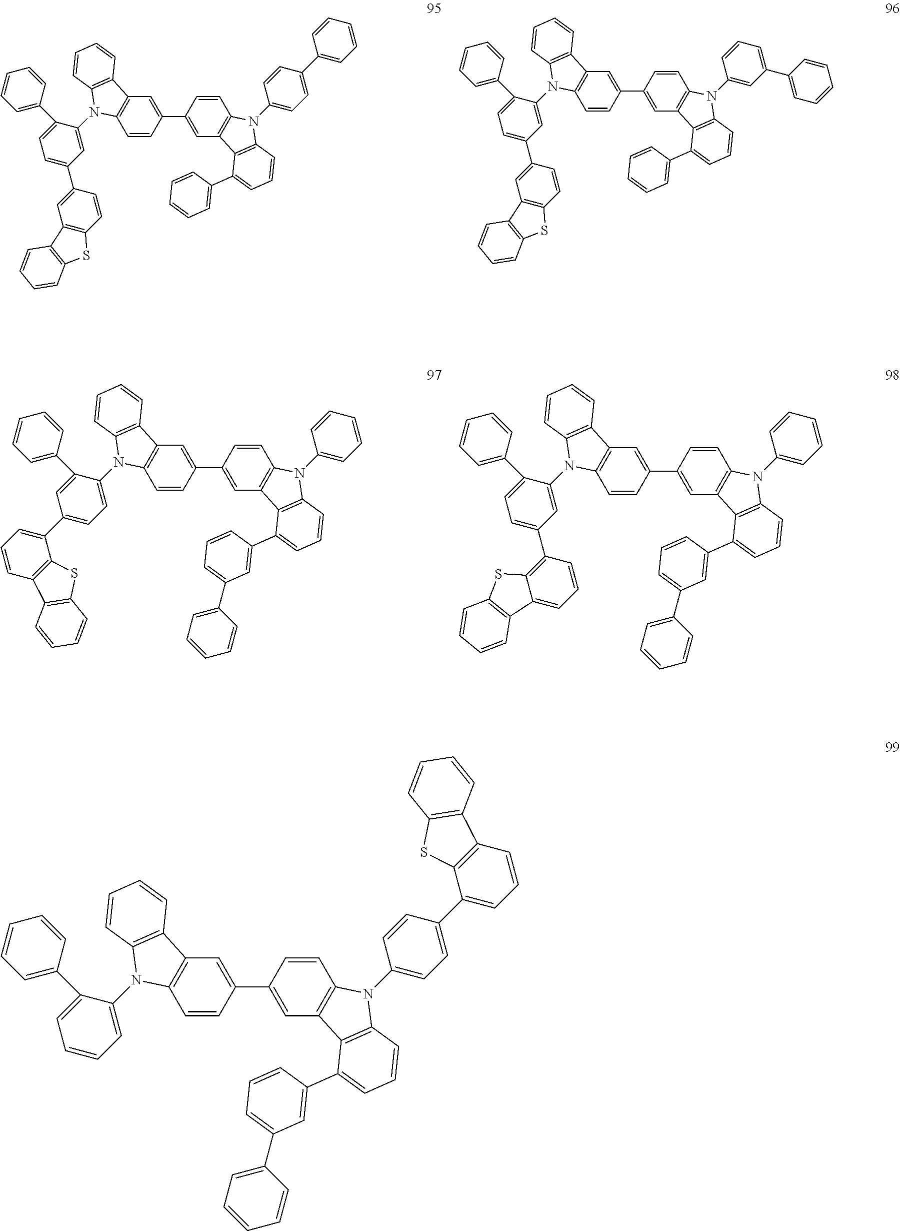

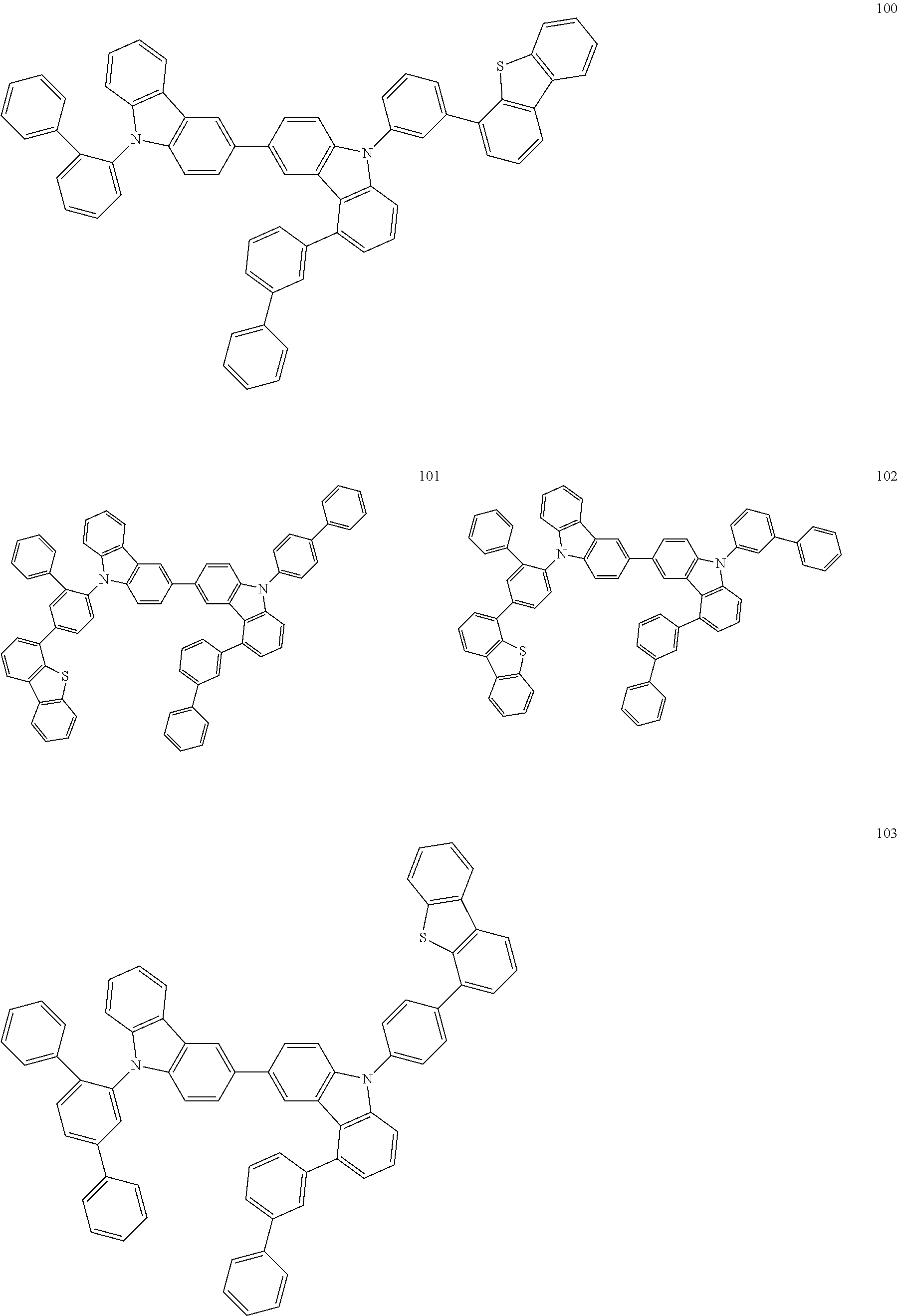 [SMILES: c1ccc(-c2cccc(-c3cccc4c3c3cc(-c5ccc6c(c5)c5ccccc5n6-c5ccccc5-c5ccccc5)ccc3n4-c3cccc(-c4cccc5c4sc4ccccc45)c3)c2)cc1 ; c1ccc(-c2ccc(-n3c4ccc(-c5ccc6c(c5)c5ccccc5n6-c5ccc(-c6cccc7c6sc6ccccc67)cc5-c5ccccc5)cc4c4c(-c5cccc(-c6ccccc6)c5)cccc43)cc2)cc1 ; c1ccc(-c2cccc(-c3cccc4c3c3cc(-c5ccc6c(c5)c5ccccc5n6-c5ccc(-c6cccc7c6sc6ccccc67)cc5-c5ccccc5)ccc3n4-c3cccc(-c4ccccc4)c3)c2)cc1 ; c1ccc(-c2cccc(-c3cccc4c3c3cc(-c5ccc6c(c5)c5ccccc5n6-c5cc(-c6ccccc6)ccc5-c5ccccc5)ccc3n4-c3ccc(-c4cccc5c4sc4ccccc45)cc3)c2)cc1]

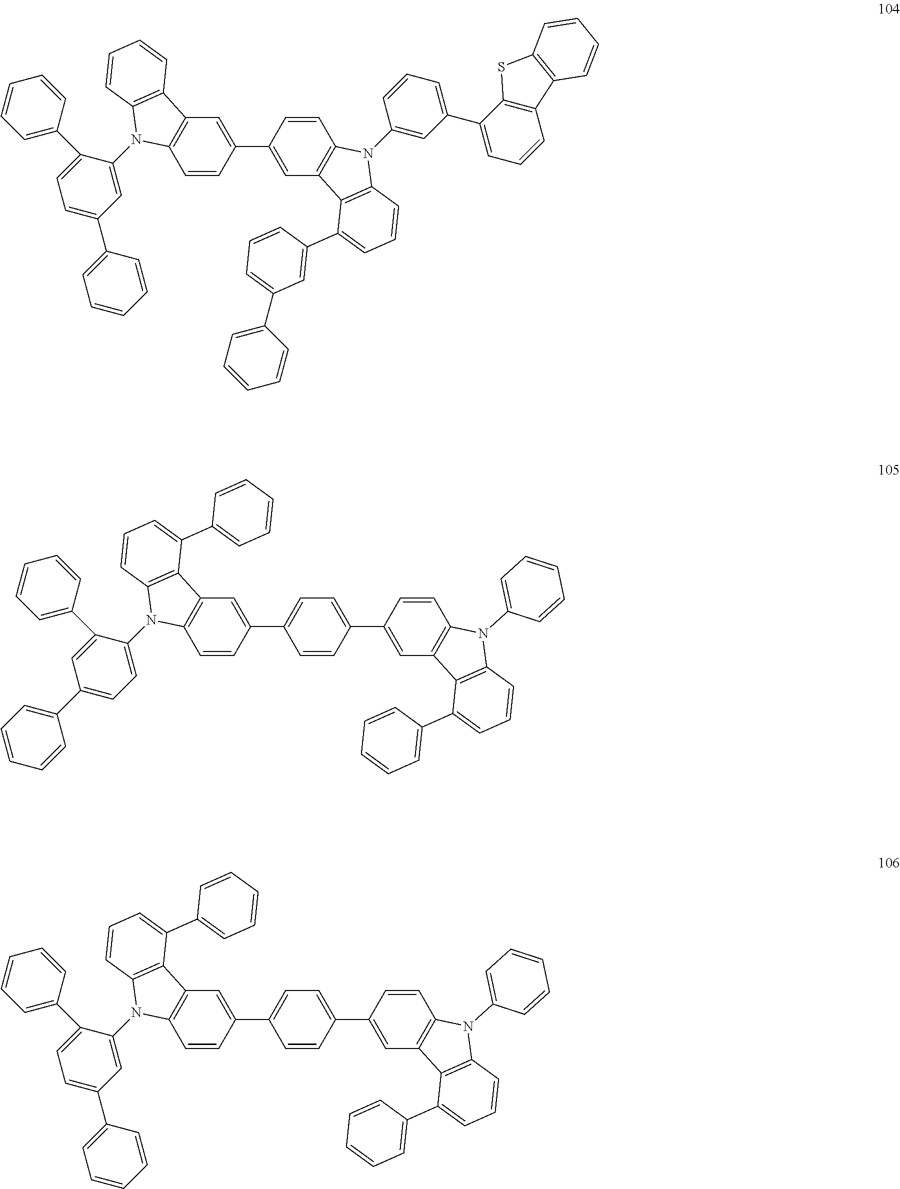 [SMILES: c1ccc(-c2cccc(-c3cccc4c3c3cc(-c5ccc6c(c5)c5ccccc5n6-c5cc(-c6ccccc6)ccc5-c5ccccc5)ccc3n4-c3cccc(-c4cccc5c4sc4ccccc45)c3)c2)cc1 ; c1ccc(-c2ccc(-n3c4ccc(-c5ccc(-c6ccc7c(c6)c6c(-c8ccccc8)cccc6n7-c6ccccc6)cc5)cc4c4c(-c5ccccc5)cccc43)c(-c3ccccc3)c2)cc1 ; c1ccc(-c2ccc(-c3ccccc3)c(-n3c4ccc(-c5ccc(-c6ccc7c(c6)c6c(-c8ccccc8)cccc6n7-c6ccccc6)cc5)cc4c4c(-c5ccccc5)cccc43)c2)cc1]

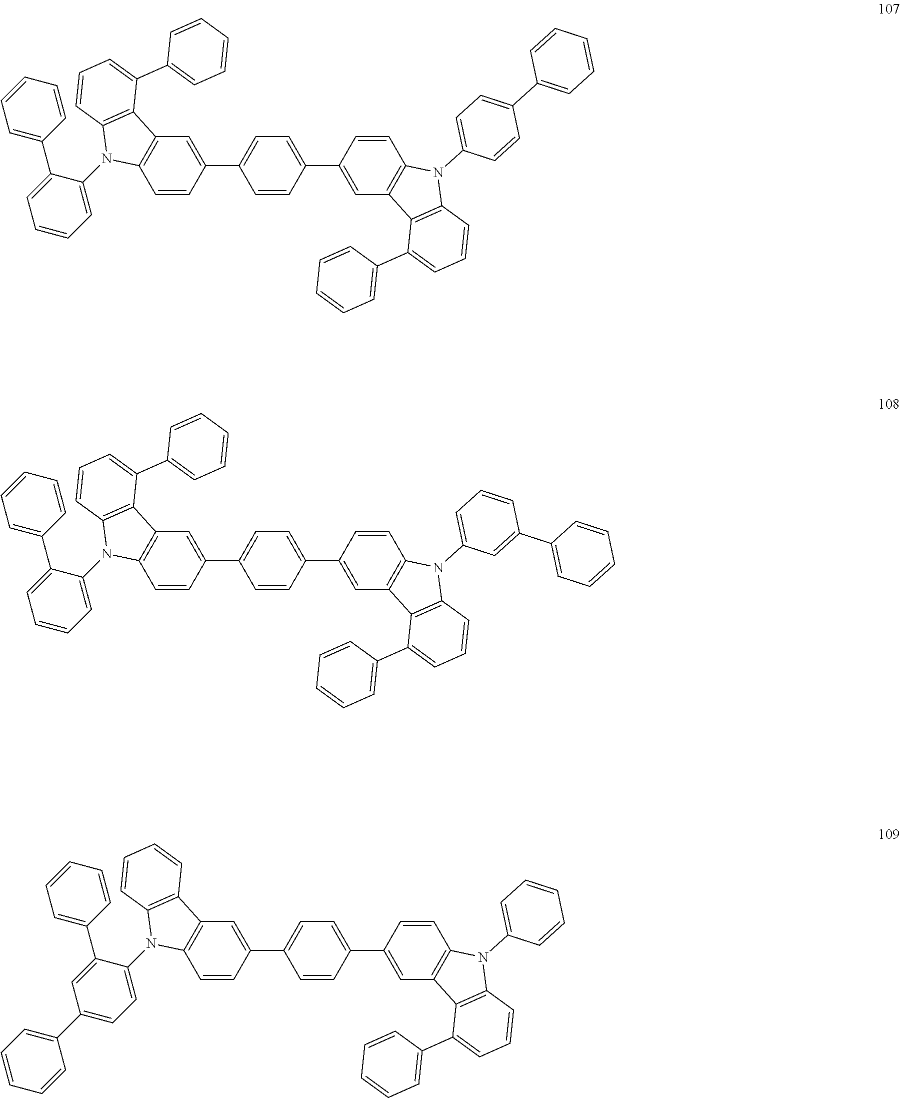 [SMILES: c1ccc(-c2ccc(-n3c4ccc(-c5ccc(-c6ccc7c(c6)c6c(-c8ccccc8)cccc6n7-c6ccccc6-c6ccccc6)cc5)cc4c4c(-c5ccccc5)cccc43)cc2)cc1 ; c1ccc(-c2cccc(-n3c4ccc(-c5ccc(-c6ccc7c(c6)c6c(-c8ccccc8)cccc6n7-c6ccccc6-c6ccccc6)cc5)cc4c4c(-c5ccccc5)cccc43)c2)cc1 ; c1ccc(-c2ccc(-n3c4ccccc4c4cc(-c5ccc(-c6ccc7c(c6)c6c(-c8ccccc8)cccc6n7-c6ccccc6)cc5)ccc43)c(-c3ccccc3)c2)cc1]

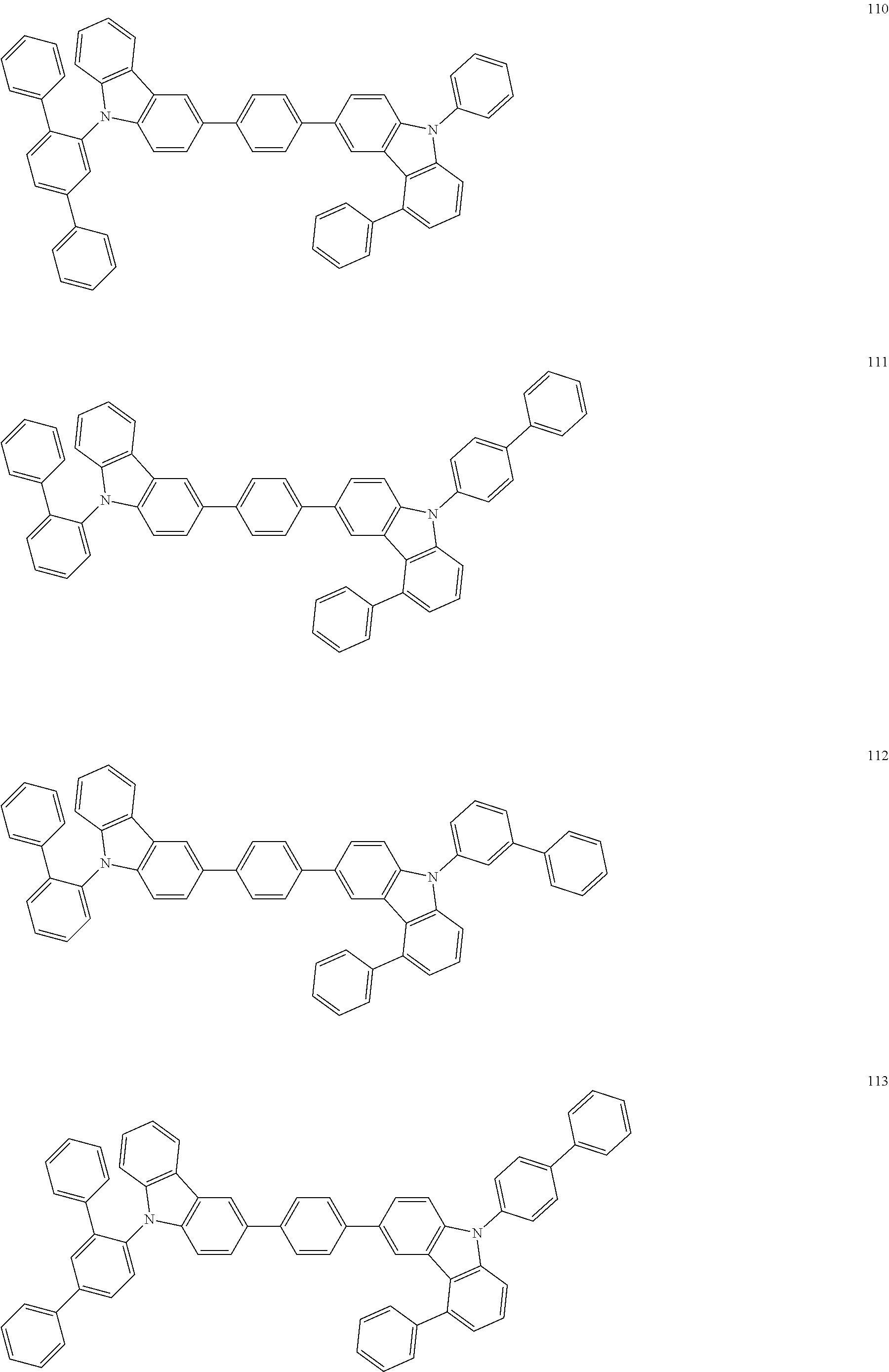 [SMILES: c1ccc(-c2ccc(-c3ccccc3)c(-n3c4ccccc4c4cc(-c5ccc(-c6ccc7c(c6)c6c(-c8ccccc8)cccc6n7-c6ccccc6)cc5)ccc43)c2)cc1 ; c1ccc(-c2ccc(-n3c4ccc(-c5ccc(-c6ccc7c(c6)c6ccccc6n7-c6ccccc6-c6ccccc6)cc5)cc4c4c(-c5ccccc5)cccc43)cc2)cc1 ; c1ccc(-c2cccc(-n3c4ccc(-c5ccc(-c6ccc7c(c6)c6ccccc6n7-c6ccccc6-c6ccccc6)cc5)cc4c4c(-c5ccccc5)cccc43)c2)cc1 ; c1ccc(-c2ccc(-n3c4ccc(-c5ccc(-c6ccc7c(c6)c6ccccc6n7-c6ccc(-c7ccccc7)cc6-c6ccccc6)cc5)cc4c4c(-c5ccccc5)cccc43)cc2)cc1]

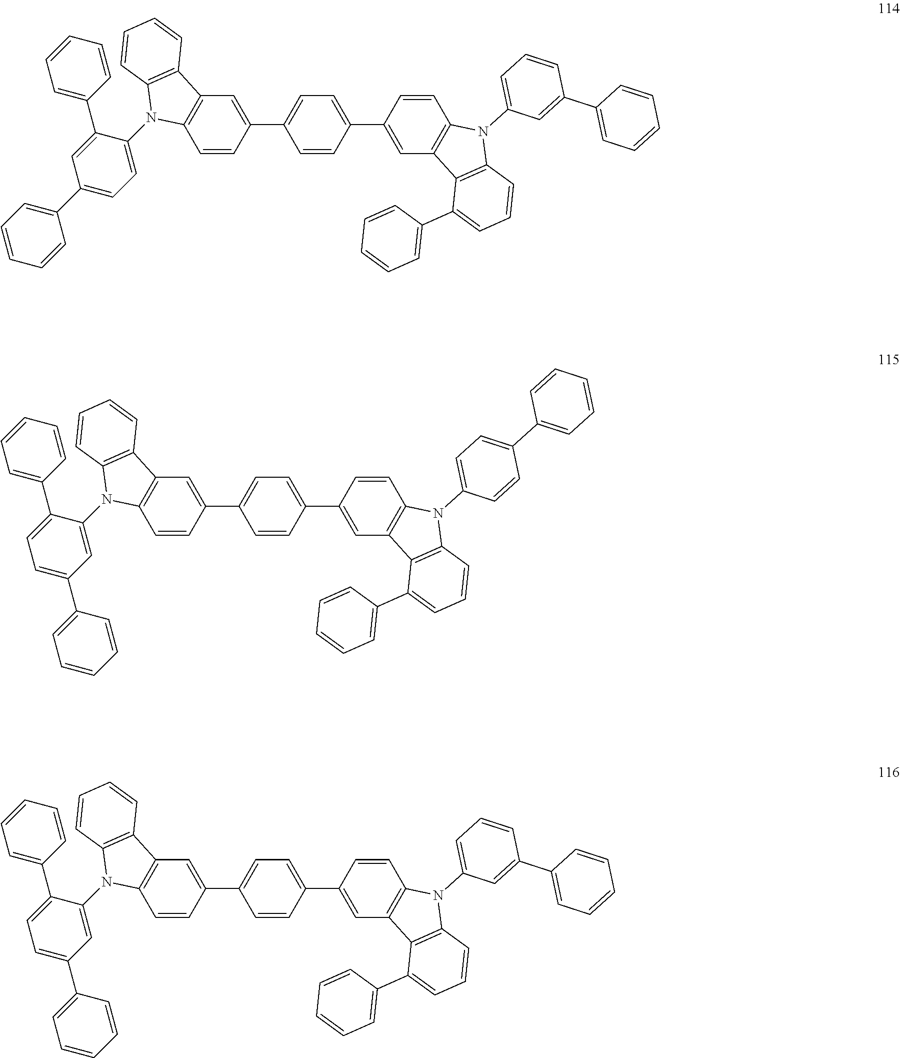 [SMILES: c1ccc(-c2cccc(-n3c4ccc(-c5ccc(-c6ccc7c(c6)c6ccccc6n7-c6ccc(-c7ccccc7)cc6-c6ccccc6)cc5)cc4c4c(-c5ccccc5)cccc43)c2)cc1 ; c1ccc(-c2ccc(-n3c4ccc(-c5ccc(-c6ccc7c(c6)c6ccccc6n7-c6cc(-c7ccccc7)ccc6-c6ccccc6)cc5)cc4c4c(-c5ccccc5)cccc43)cc2)cc1 ; c1ccc(-c2cccc(-n3c4ccc(-c5ccc(-c6ccc7c(c6)c6ccccc6n7-c6cc(-c7ccccc7)ccc6-c6ccccc6)cc5)cc4c4c(-c5ccccc5)cccc43)c2)cc1]

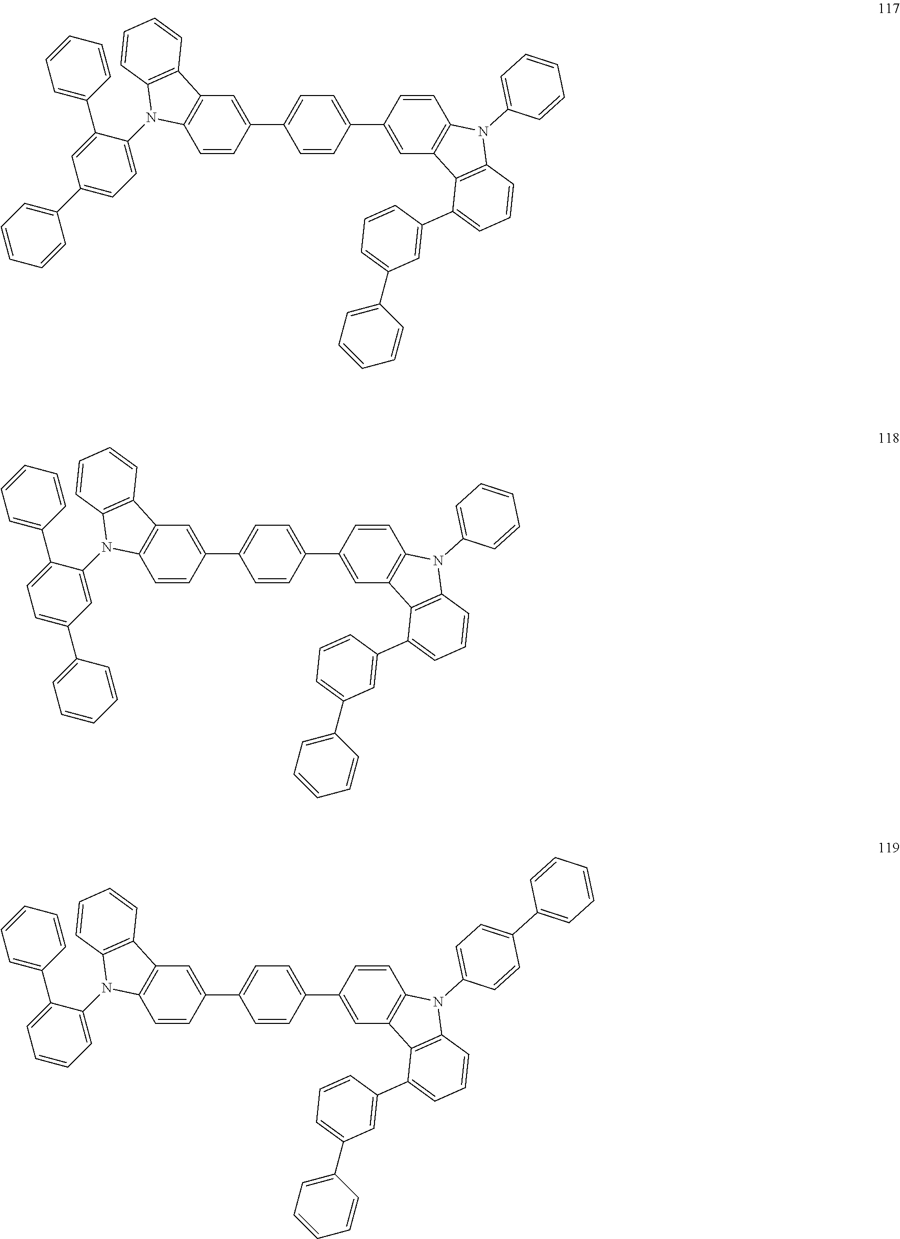 [SMILES: c1ccc(-c2cccc(-c3cccc4c3c3cc(-c5ccc(-c6ccc7c(c6)c6ccccc6n7-c6ccc(-c7ccccc7)cc6-c6ccccc6)cc5)ccc3n4-c3ccccc3)c2)cc1 ; c1ccc(-c2cccc(-c3cccc4c3c3cc(-c5ccc(-c6ccc7c(c6)c6ccccc6n7-c6cc(-c7ccccc7)ccc6-c6ccccc6)cc5)ccc3n4-c3ccccc3)c2)cc1 ; c1ccc(-c2ccc(-n3c4ccc(-c5ccc(-c6ccc7c(c6)c6ccccc6n7-c6ccccc6-c6ccccc6)cc5)cc4c4c(-c5cccc(-c6ccccc6)c5)cccc43)cc2)cc1]

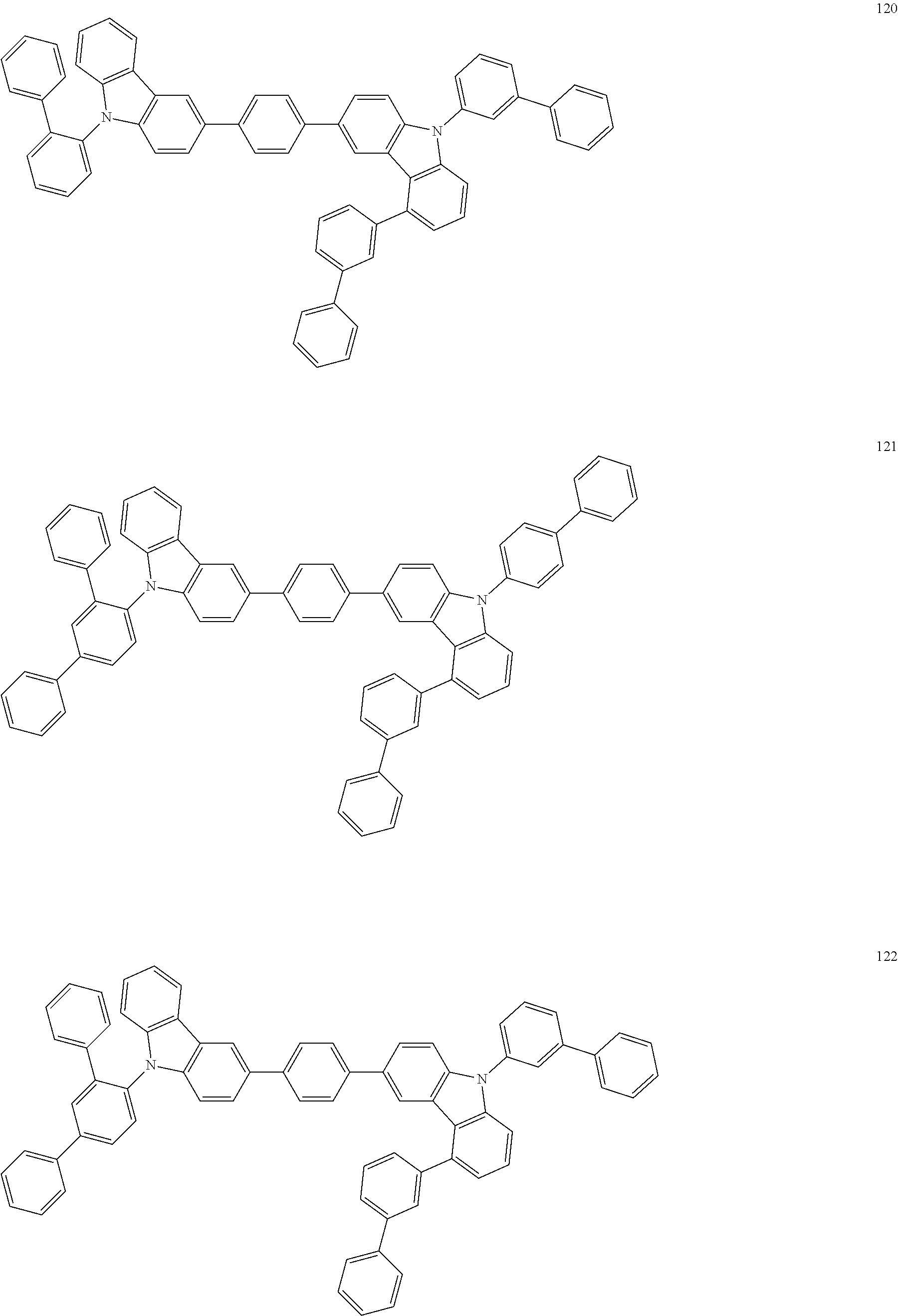 [SMILES: c1ccc(-c2cccc(-c3cccc4c3c3cc(-c5ccc(-c6ccc7c(c6)c6ccccc6n7-c6ccccc6-c6ccccc6)cc5)ccc3n4-c3cccc(-c4ccccc4)c3)c2)cc1 ; c1ccc(-c2ccc(-n3c4ccc(-c5ccc(-c6ccc7c(c6)c6ccccc6n7-c6ccc(-c7ccccc7)cc6-c6ccccc6)cc5)cc4c4c(-c5cccc(-c6ccccc6)c5)cccc43)cc2)cc1 ; c1ccc(-c2cccc(-c3cccc4c3c3cc(-c5ccc(-c6ccc7c(c6)c6ccccc6n7-c6ccc(-c7ccccc7)cc6-c6ccccc6)cc5)ccc3n4-c3cccc(-c4ccccc4)c3)c2)cc1]

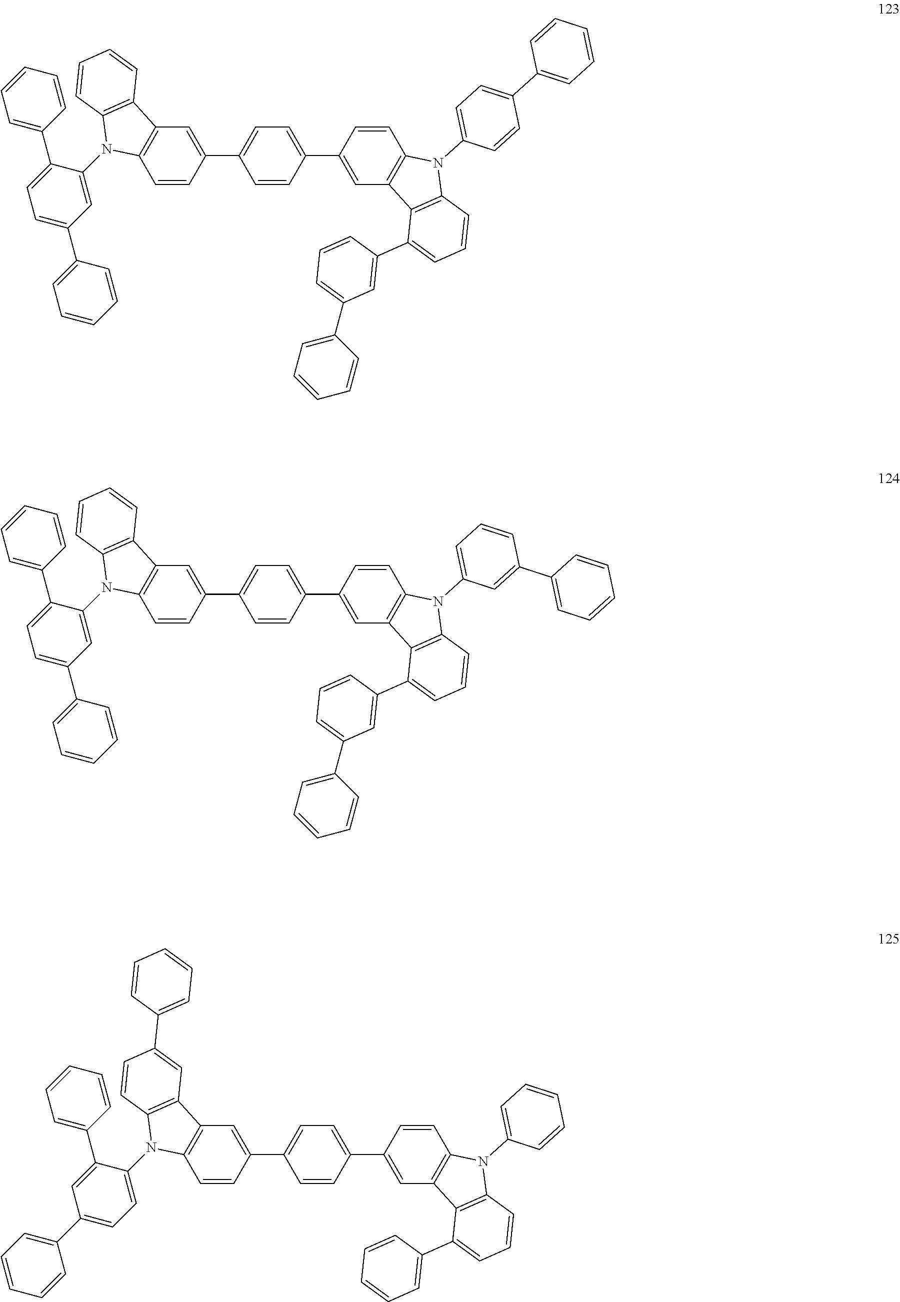 [SMILES: c1ccc(-c2ccc(-n3c4ccc(-c5ccc(-c6ccc7c(c6)c6ccccc6n7-c6cc(-c7ccccc7)ccc6-c6ccccc6)cc5)cc4c4c(-c5cccc(-c6ccccc6)c5)cccc43)cc2)cc1 ; c1ccc(-c2cccc(-c3cccc4c3c3cc(-c5ccc(-c6ccc7c(c6)c6ccccc6n7-c6cc(-c7ccccc7)ccc6-c6ccccc6)cc5)ccc3n4-c3cccc(-c4ccccc4)c3)c2)cc1 ; c1ccc(-c2ccc(-n3c4ccc(-c5ccccc5)cc4c4cc(-c5ccc(-c6ccc7c(c6)c6c(-c8ccccc8)cccc6n7-c6ccccc6)cc5)ccc43)c(-c3ccccc3)c2)cc1]

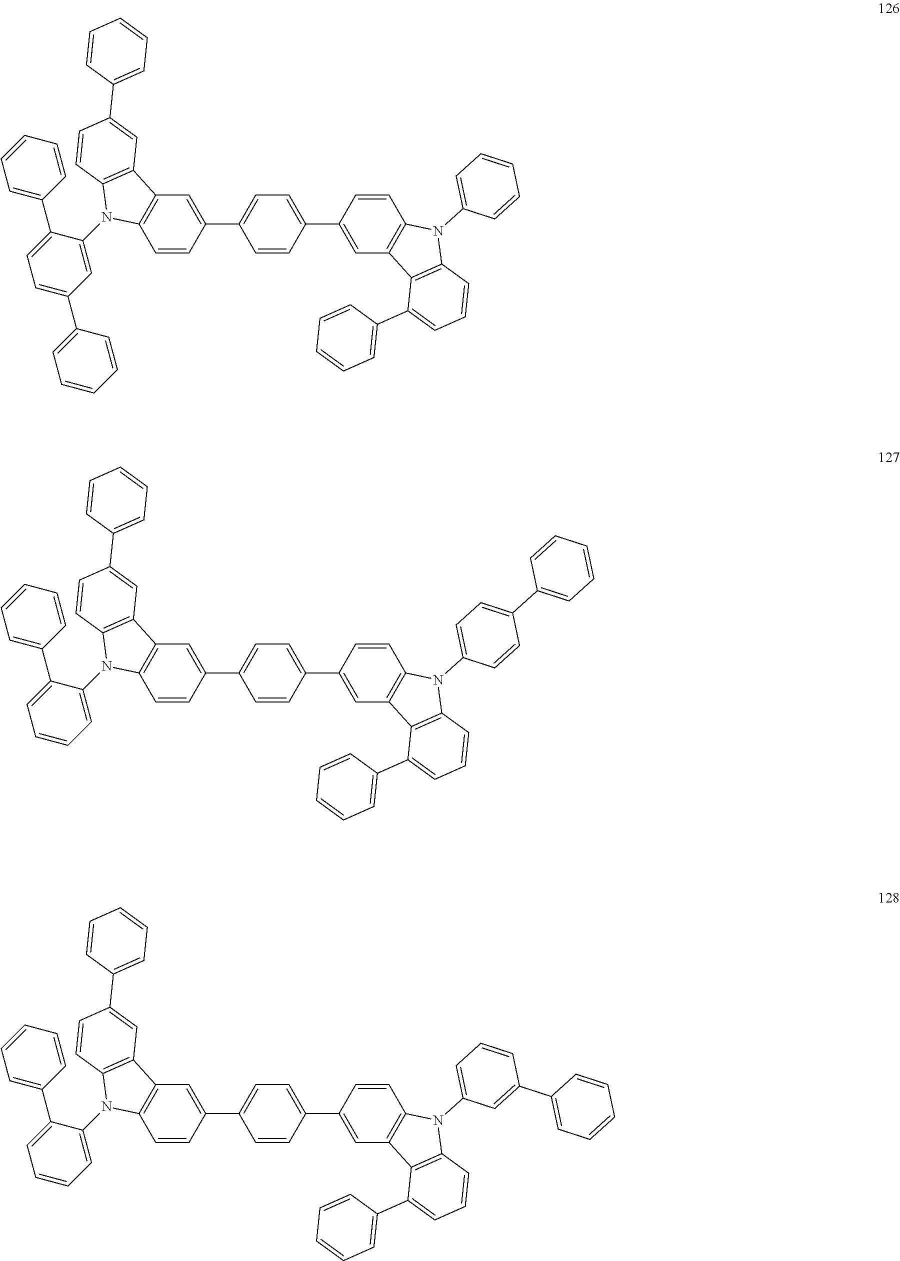 [SMILES: c1ccc(-c2ccc(-c3ccccc3)c(-n3c4ccc(-c5ccccc5)cc4c4cc(-c5ccc(-c6ccc7c(c6)c6c(-c8ccccc8)cccc6n7-c6ccccc6)cc5)ccc43)c2)cc1 ; c1ccc(-c2ccc(-n3c4ccc(-c5ccc(-c6ccc7c(c6)c6cc(-c8ccccc8)ccc6n7-c6ccccc6-c6ccccc6)cc5)cc4c4c(-c5ccccc5)cccc43)cc2)cc1 ; c1ccc(-c2cccc(-n3c4ccc(-c5ccc(-c6ccc7c(c6)c6cc(-c8ccccc8)ccc6n7-c6ccccc6-c6ccccc6)cc5)cc4c4c(-c5ccccc5)cccc43)c2)cc1]

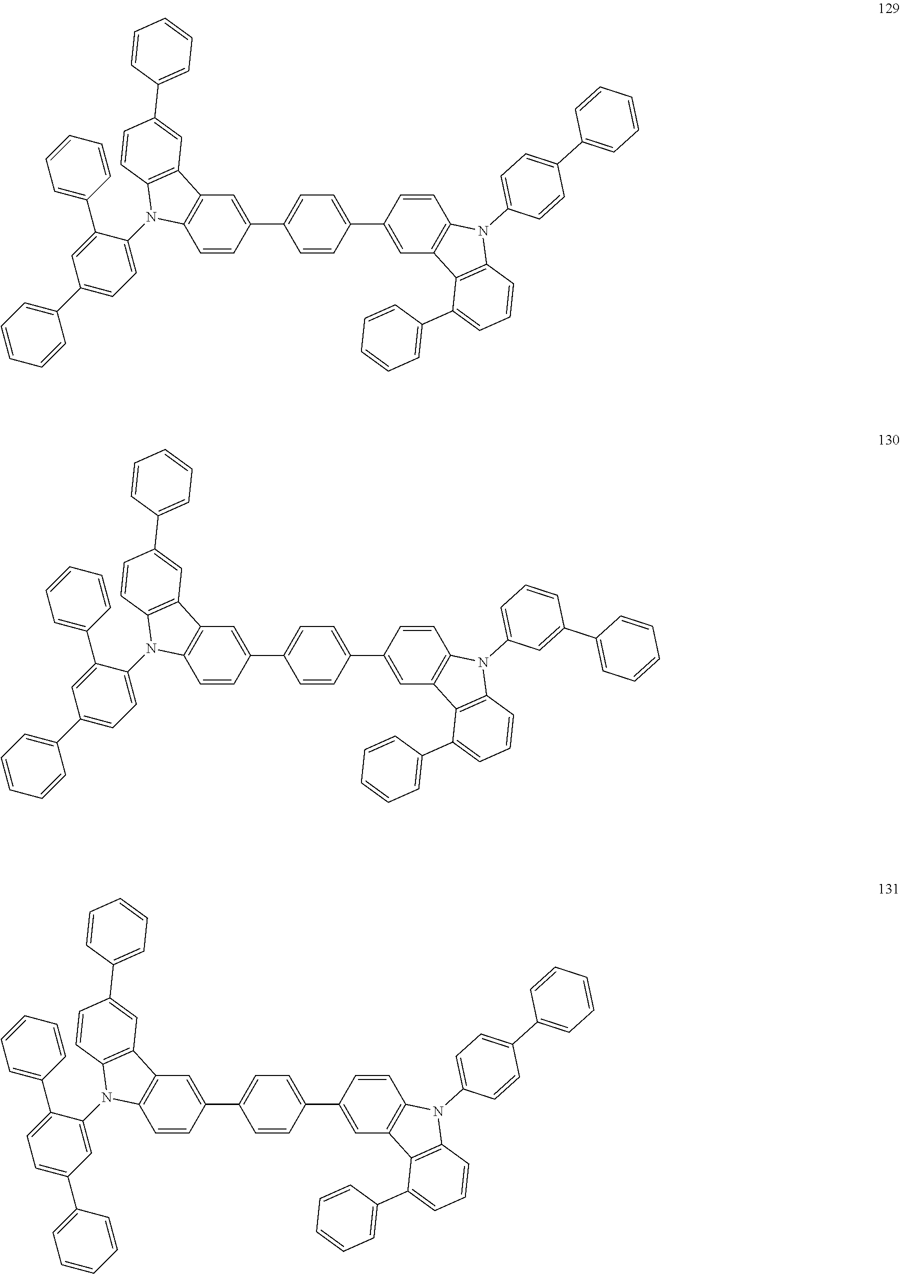 [SMILES: c1ccc(-c2ccc(-n3c4ccc(-c5ccc(-c6ccc7c(c6)c6cc(-c8ccccc8)ccc6n7-c6ccc(-c7ccccc7)cc6-c6ccccc6)cc5)cc4c4c(-c5ccccc5)cccc43)cc2)cc1 ; c1ccc(-c2cccc(-n3c4ccc(-c5ccc(-c6ccc7c(c6)c6cc(-c8ccccc8)ccc6n7-c6ccc(-c7ccccc7)cc6-c6ccccc6)cc5)cc4c4c(-c5ccccc5)cccc43)c2)cc1 ; c1ccc(-c2ccc(-n3c4ccc(-c5ccc(-c6ccc7c(c6)c6cc(-c8ccccc8)ccc6n7-c6cc(-c7ccccc7)ccc6-c6ccccc6)cc5)cc4c4c(-c5ccccc5)cccc43)cc2)cc1]

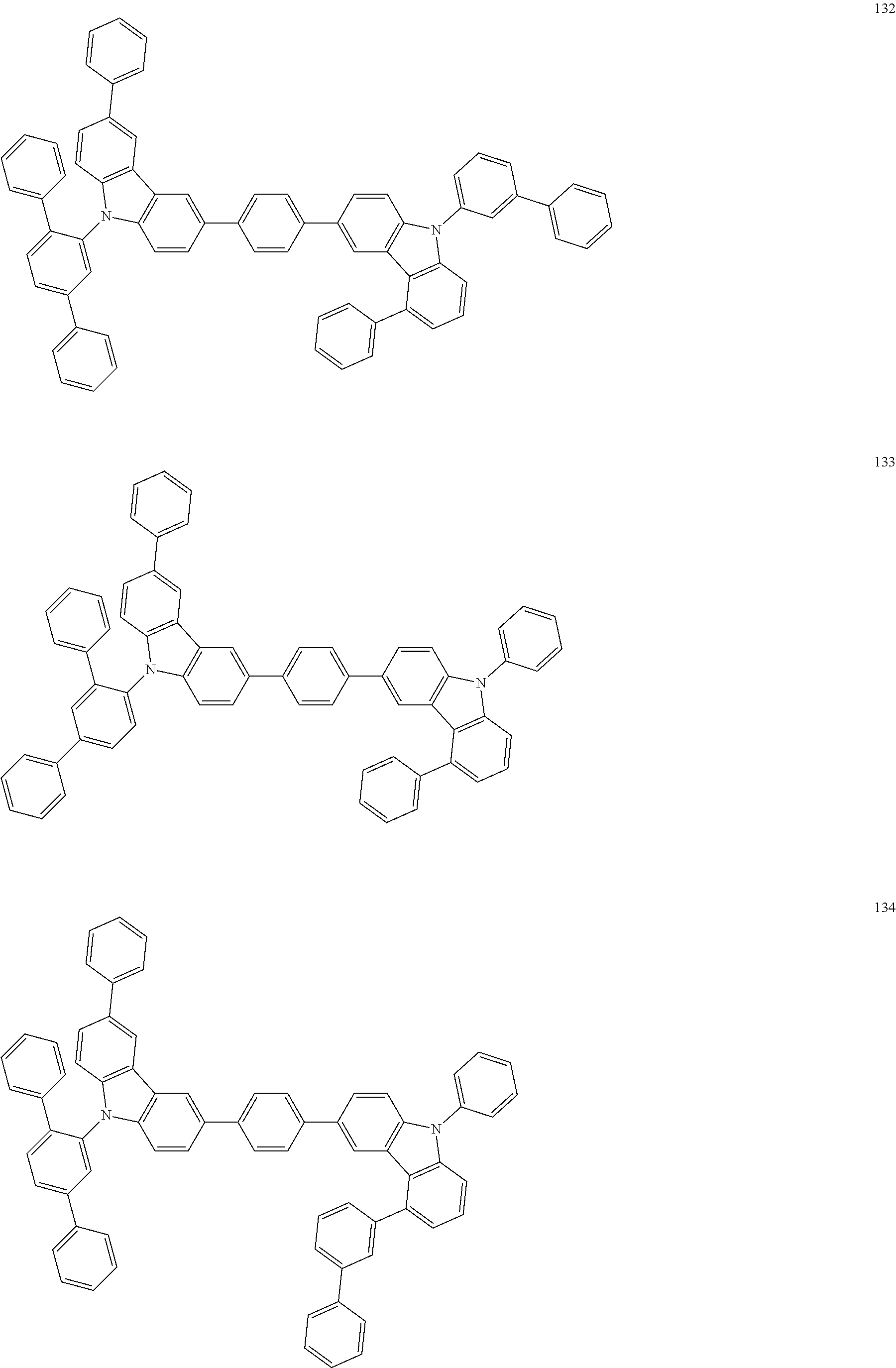 [SMILES: c1ccc(-c2cccc(-n3c4ccc(-c5ccc(-c6ccc7c(c6)c6cc(-c8ccccc8)ccc6n7-c6cc(-c7ccccc7)ccc6-c6ccccc6)cc5)cc4c4c(-c5ccccc5)cccc43)c2)cc1 ; c1ccc(-c2ccc(-n3c4ccc(-c5ccccc5)cc4c4cc(-c5ccc(-c6ccc7c(c6)c6c(-c8ccccc8)cccc6n7-c6ccccc6)cc5)ccc43)c(-c3ccccc3)c2)cc1 ; c1ccc(-c2cccc(-c3cccc4c3c3cc(-c5ccc(-c6ccc7c(c6)c6cc(-c8ccccc8)ccc6n7-c6cc(-c7ccccc7)ccc6-c6ccccc6)cc5)ccc3n4-c3ccccc3)c2)cc1]

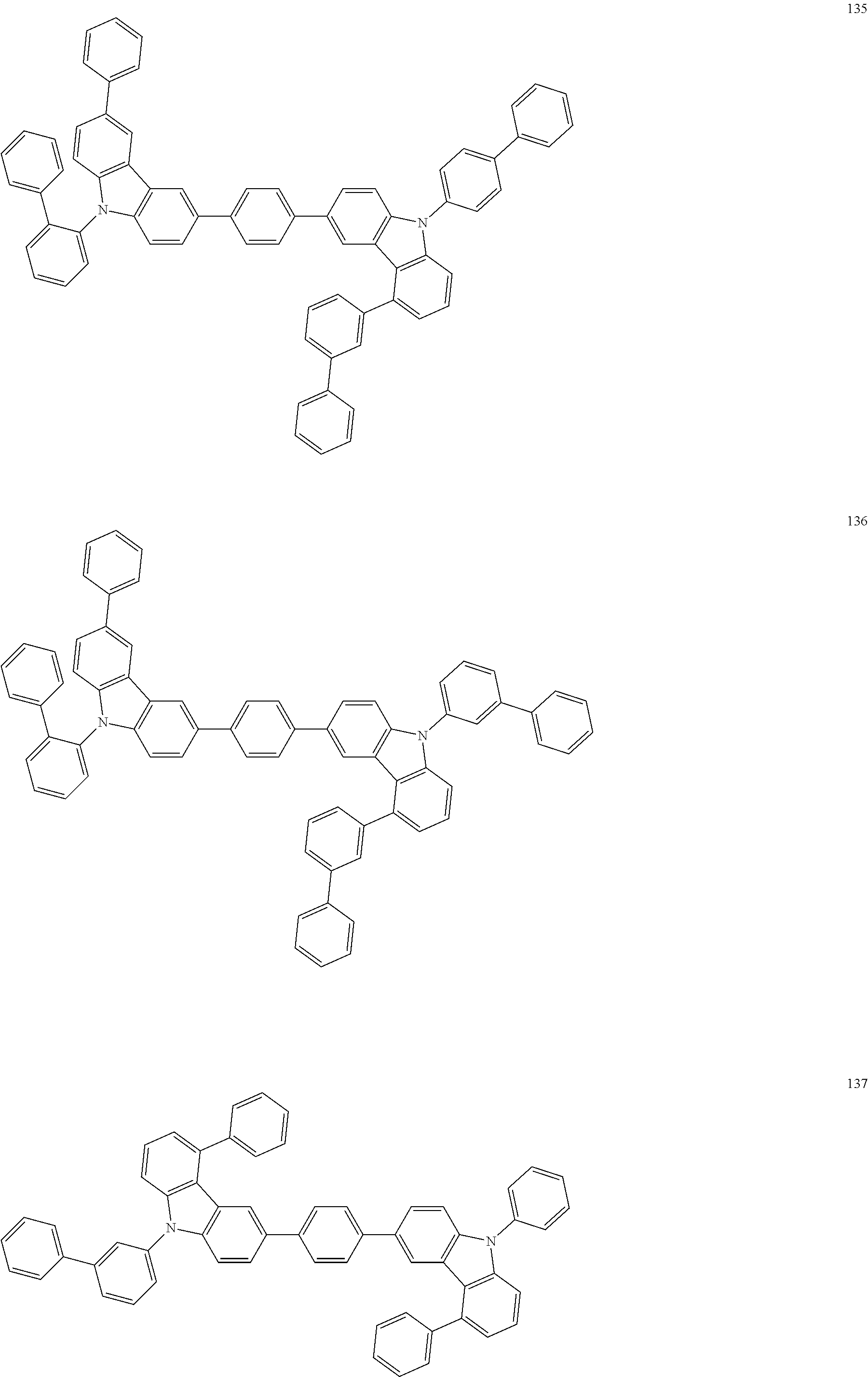 [SMILES: c1ccc(-c2ccc(-n3c4ccc(-c5ccc(-c6ccc7c(c6)c6cc(-c8ccccc8)ccc6n7-c6ccccc6-c6ccccc6)cc5)cc4c4c(-c5cccc(-c6ccccc6)c5)cccc43)cc2)cc1 ; c1ccc(-c2cccc(-c3cccc4c3c3cc(-c5ccc(-c6ccc7c(c6)c6cc(-c8ccccc8)ccc6n7-c6ccccc6-c6ccccc6)cc5)ccc3n4-c3cccc(-c4ccccc4)c3)c2)cc1 ; c1ccc(-c2cccc(-n3c4ccc(-c5ccc(-c6ccc7c(c6)c6c(-c8ccccc8)cccc6n7-c6ccccc6)cc5)cc4c4c(-c5ccccc5)cccc43)c2)cc1]

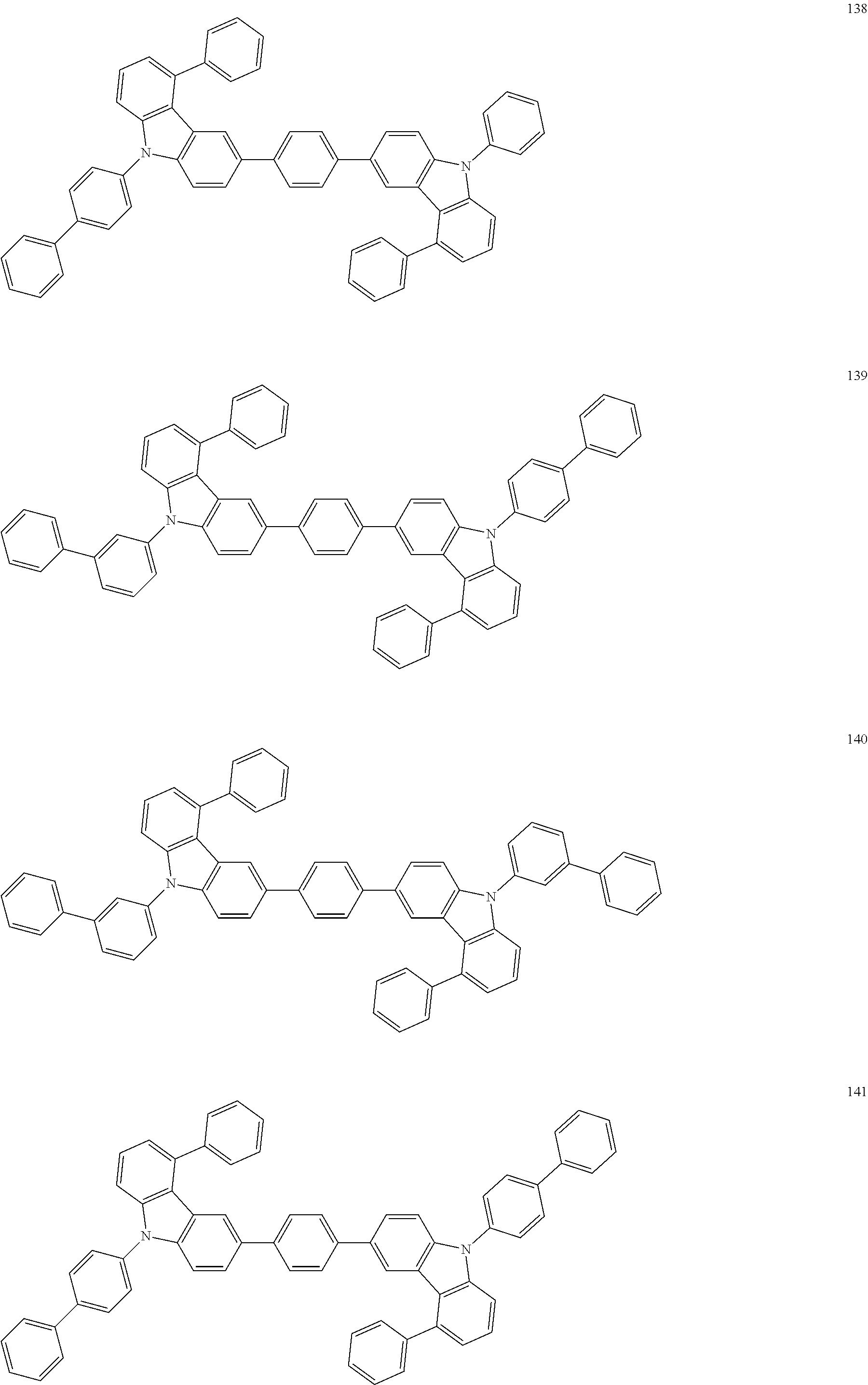 [SMILES: c1ccc(-c2ccc(-n3c4ccc(-c5ccc(-c6ccc7c(c6)c6c(-c8ccccc8)cccc6n7-c6ccccc6)cc5)cc4c4c(-c5ccccc5)cccc43)cc2)cc1 ; c1ccc(-c2ccc(-n3c4ccc(-c5ccc(-c6ccc7c(c6)c6c(-c8ccccc8)cccc6n7-c6cccc(-c7ccccc7)c6)cc5)cc4c4c(-c5ccccc5)cccc43)cc2)cc1 ; c1ccc(-c2cccc(-n3c4ccc(-c5ccc(-c6ccc7c(c6)c6c(-c8ccccc8)cccc6n7-c6cccc(-c7ccccc7)c6)cc5)cc4c4c(-c5ccccc5)cccc43)c2)cc1 ; c1ccc(-c2ccc(-n3c4ccc(-c5ccc(-c6ccc7c(c6)c6c(-c8ccccc8)cccc6n7-c6ccc(-c7ccccc7)cc6)cc5)cc4c4c(-c5ccccc5)cccc43)cc2)cc1]

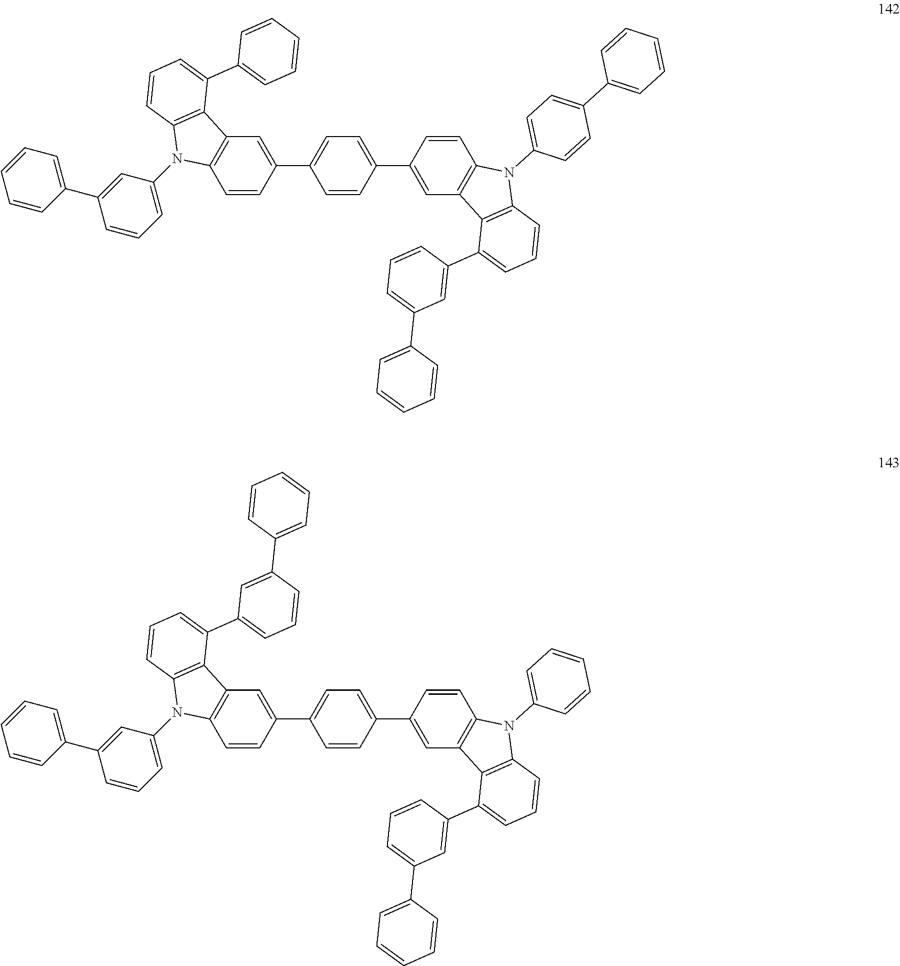 [SMILES: c1ccc(-c2ccc(-n3c4ccc(-c5ccc(-c6ccc7c(c6)c6c(-c8ccccc8)cccc6n7-c6cccc(-c7ccccc7)c6)cc5)cc4c4c(-c5cccc(-c6ccccc6)c5)cccc43)cc2)cc1 ; c1ccc(-c2cccc(-c3cccc4c3c3cc(-c5ccc(-c6ccc7c(c6)c6c(-c8cccc(-c9ccccc9)c8)cccc6n7-c6cccc(-c7ccccc7)c6)cc5)ccc3n4-c3ccccc3)c2)cc1]

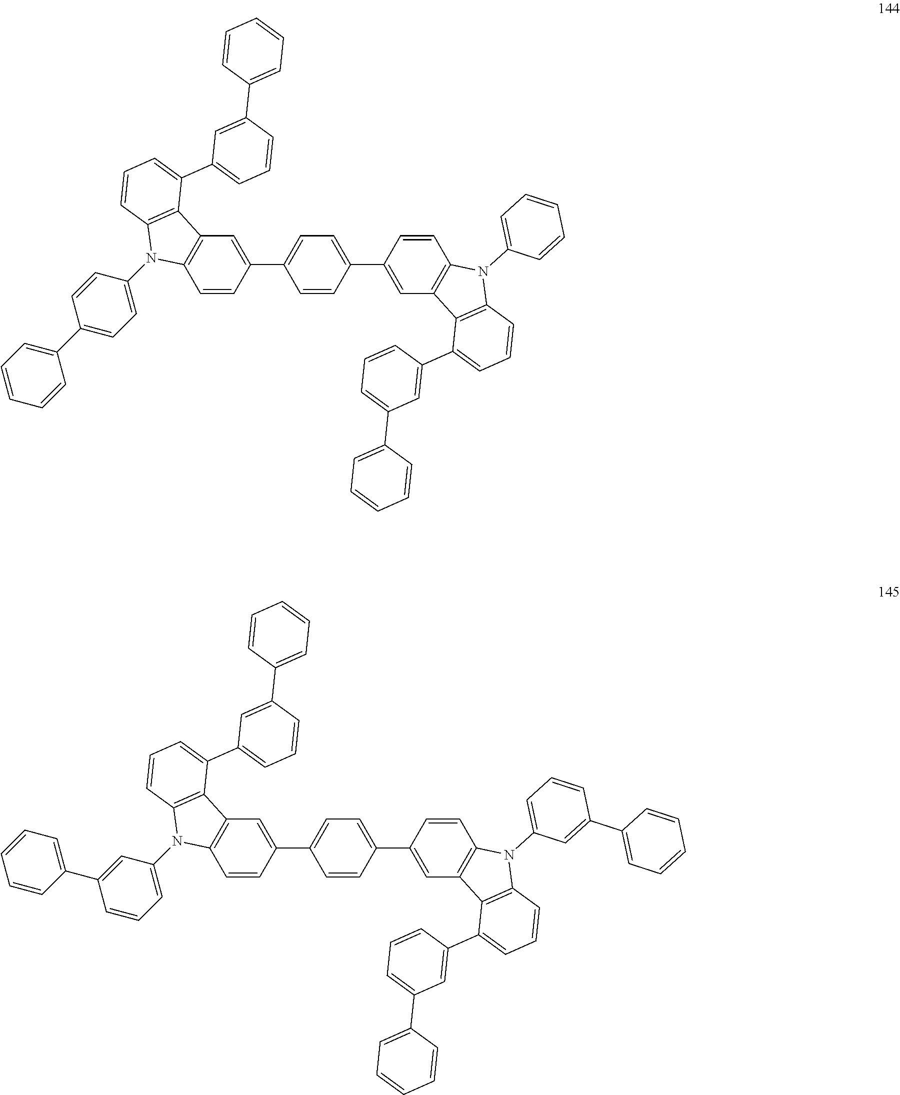 [SMILES: c1ccc(-c2ccc(-n3c4ccc(-c5ccc(-c6ccc7c(c6)c6c(-c8cccc(-c9ccccc9)c8)cccc6n7-c6ccccc6)cc5)cc4c4c(-c5cccc(-c6ccccc6)c5)cccc43)cc2)cc1 ; c1ccc(-c2cccc(-c3cccc4c3c3cc(-c5ccc(-c6ccc7c(c6)c6c(-c8cccc(-c9ccccc9)c8)cccc6n7-c6cccc(-c7ccccc7)c6)cc5)ccc3n4-c3cccc(-c4ccccc4)c3)c2)cc1]

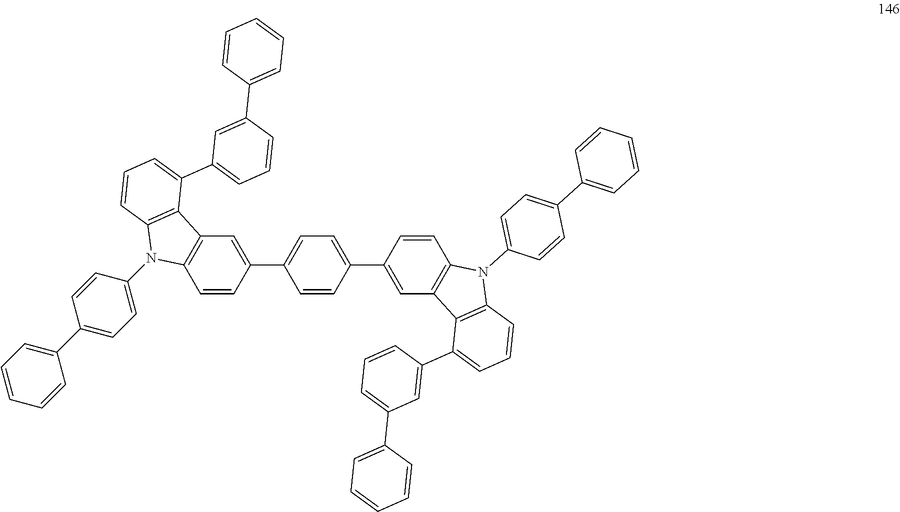 [SMILES: c1ccc(-c2ccc(-n3c4ccc(-c5ccc(-c6ccc7c(c6)c6c(-c8cccc(-c9ccccc9)c8)cccc6n7-c6ccc(-c7ccccc7)cc6)cc5)cc4c4c(-c5cccc(-c6ccccc6)c5)cccc43)cc2)cc1]

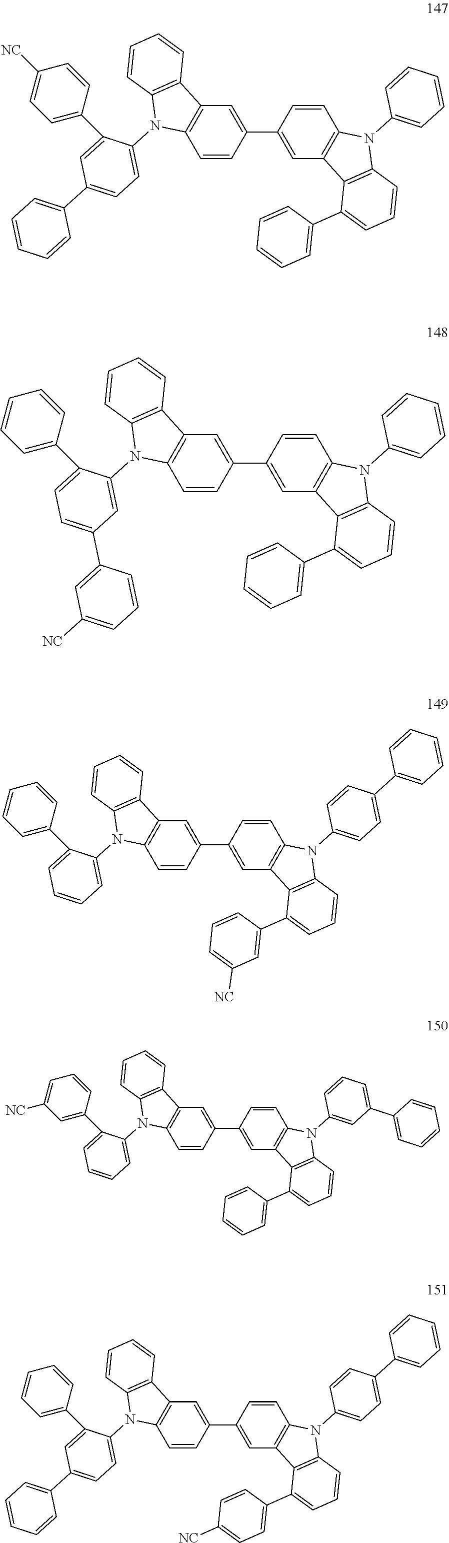

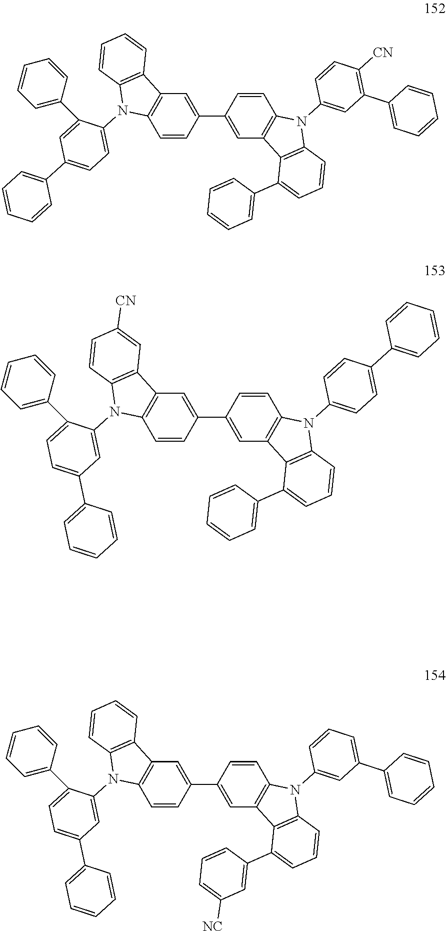 [SMILES: N#Cc1ccc(-n2c3ccc(-c4ccc5c(c4)c4ccccc4n5-c4ccc(-c5ccccc5)cc4-c4ccccc4)cc3c3c(-c4ccccc4)cccc32)cc1-c1ccccc1 ; N#Cc1ccc2c(c1)c1cc(-c3ccc4c(c3)c3c(-c5ccccc5)cccc3n4-c3ccc(-c4ccccc4)cc3)ccc1n2-c1cc(-c2ccccc2)ccc1-c1ccccc1 ; N#Cc1cccc(-c2cccc3c2c2cc(-c4ccc5c(c4)c4ccccc4n5-c4cc(-c5ccccc5)ccc4-c4ccccc4)ccc2n3-c2cccc(-c3ccccc3)c2)c1]

16. A composition comprising the condensed cyclic compound of claim 1 and a solvent.

17. An organic light-emitting device comprising:
a first electrode;
a second electrode; and
an organic layer disposed between the first electrode and the second electrode,
wherein the organic layer comprises an emission layer and at least one condensed cyclic compound of claim 1.

18. The organic light-emitting device of claim 17, wherein
the emission layer comprises the condensed cyclic compound.

19. The organic light-emitting device of claim 17, wherein
the emission layer comprises a light-emitting material capable of emitting light from triplet excitons.

20. The organic light-emitting device of claim 18, wherein
the condensed cyclic compound comprised in the emission layer is a hole transport host, and
the emission layer further comprises an electron transport host and an emission material.
